(19) United States
(12) Patent Application Publication
(10) Pub. No.: US 2024/0279329 A1
(43) Pub. Date: Aug. 22, 2024

(54) METHODS OF USE OF ANTI-IL-2 ANTIBODIES

(71) Applicant: Aulos Bioscience, Inc., Larkspur, CA (US)

(72) Inventors: James Robert VASSELLI, San Francisco, CA (US); Timothy WYANT, Bellingham, MA (US); Yanay OFRAN, Tel Aviv (IL); Aron Marc KNICKERBOCKER, Mill Valley, CA (US)

(73) Assignee: Aulos Bioscience, Inc., Larkspur, CA (US)

(21) Appl. No.: 18/404,093

(22) Filed: Jan. 4, 2024

Related U.S. Application Data
(63) Continuation-in-part of application No. PCT/US23/79221, filed on Nov. 9, 2023.
(60) Provisional application No. 63/383,086, filed on Nov. 10, 2022.
(60) Provisional application No. 63/503,481, filed on May 21, 2023.
(60) Provisional application No. 63/503,977, filed on May 24, 2023.
(60) Provisional application No. 63/589,659, filed on Oct. 12, 2023.

(51) Int. Cl. C07K 16/24; A61K 39/00; A61K 45/06; A61P 35/00

(52) U.S. Cl. CPC C07K 16/246; A61K 45/06; A61P 35/00; A61K 2039/505; A61K 2039/545; C07K 2317/565; C07K 2317/71

(57) ABSTRACT

Described herein are therapeutic methods of use of engineered anti-IL-2 antibodies including dosages and administration regimes. The engineered antibodies may confer modified receptor binding specificity to an IL-2-anti-IL2 antibody complex, inhibiting the binding of IL-2 to CD25. The engineered anti-IL-2 antibodies may facilitate expansion of subsets of effector immune cells and decrease undesirable effects caused by IL-2. Therapeutic methods of use of the engineered anti-IL-2 antibodies includes treating cancer, for example treating cancers presenting as solid tumors and metastases thereof.

### CROSS-REFERENCE TO RELATED APPLICATIONS

This Application is a Continuation-in-Part Application of PCT International Application No. PCT/US23/79221, International Filing Date Nov. 9, 2023, claiming the benefit of priority of U.S. Provisional Application No. 63/589,659 filed Oct. 12, 2023, U.S. Provisional Application No. 63/503,977 filed May 24, 2023, U.S. Provisional Application No. 63/503,481 filed May 21, 2023, and U.S. Provisional Application No. 63/383,086 filed Nov. 10, 2022, which are all hereby incorporated by reference in their entirety.

### SEQUENCE LISTING STATEMENT

The instant application contains a Sequence Listing which has been submitted electronically in XML format and is hereby incorporated by reference in its entirety. The XML formatted sequence listing, created on Nov. 10, 2022, is named P-621284-USP_10NOV22.XML and is 97.7 kilobytes in size.

### FIELD OF THE INVENTION

The disclosure relates in general to the field of antibodies. In one embodiment, the present disclosure describes the making and uses of engineered anti-IL-2 antibodies that would confer modified receptor binding specificity to IL-2. In certain embodiments, the present disclosure describes treating cancers, including cancers presenting as solid tumors.

### BACKGROUND

Interleukin 2 (IL-2) is a 15.4 kDa type I cytokine with a four helix bundle structure. Since its discovery more than 30 years ago, the importance of IL-2 in regulation of the immune system has been illustrated many times. IL-2 is mostly produced and secreted by CD4+ T cells that are activated by an antigen. To a lesser extent, IL-2 is also produced by CD8+ T cells, natural killer (NK) cells, dendritic cells and mast cells.

IL-2 signaling has two opposite effects. IL-2 can enhance immune response by activation of effector cells and induce their proliferation. Alternatively, IL-2 can tune down immune response by activation and proliferation of CD4+ regulatory T (Treg) cells. To facilitate these functions, IL-2 mediates its effect by binding to two forms of IL-2 receptor: i) trimeric receptors made up of IL-2Rα (CD25), IL-2Rβ (CD122), and a common IL-2Rγ (γc, CD132) chains, or ii) a dimeric receptor that consists of only the IL-2Rβ and IL-2Rγ subunits. Both the dimeric and trimeric receptors are able to transmit IL-2 binding signaling via the STAT5 pathway. However, IL-2 binds the αβγ receptor trimer a 100 fold tighter than the βγ receptor dimer. It has been demonstrated that the binding affinity of hIL-2 to the αβγ trimer is approximately 10 pM, whereas the hIL-2 affinity to the βγ dimer is 1 nM.

The difference in affinities to the dimeric and trimeric forms of the IL-2 receptor is one of the key mechanisms in charge of keeping immunological homeostasis in vivo. Activation of the trimeric receptor is associated with FoxP3-mediated transcription in Tregs, which express on its membrane more of the αβγ trimeric IL-2 receptor. In contrast, binding of IL-2 to the βγ dimer is associated with activation of NK cells and memory phenotype (MP) CD8+ cells, which express relatively high levels of the βγ dimer and very low levels the αβγ trimeric IL-2 receptor. Since in normal physiological condition the natural levels of IL-2 are relatively low, it seems the main function of IL-2 is to facilitate immune tolerance by acting as a Treg activating and proliferative factor. On the other hand, once the immune system is activated, IL-2 levels are rising and subsequently IL-2 is able to bind the βγ dimer and facilitate memory phenotype effector T cells (MP) CD8+ and NK cells activation and proliferation.

Since the early 1990's, high dose IL-2 therapy has been used to treat melanoma and metastatic renal cell carcinoma with 10%-15% response rate. While efficacious, this approach has not been adopted to other cancers, since IL-2-dependent adverse effects like the potentially lethal Vascular Leak Syndrome (VLS) excludes many patients from being considered for this therapy. The short half-life of administered IL-2 requires very frequent administration, resulting in repeated spikes in the level of circulating IL-2, thereby exacerbating adverse effects. Finally, since wild type IL-2 is not selective it could also enhance a non-desired activation of Treg cells.

It has been discovered that certain antibodies could bind to IL-2 and modulate binding to the βγ dimeric or the αβγ trimeric IL-2 receptors. IL-2 complexed with these antibodies would have relatively longer half-life, and these IL-2 complexes would activate specific subsets of effector or immune cells. For example, the antibody S4B6-mouse IL-2 complex preferentially activates mouse effector cells in vivo, whereas the antibody JES6.1-mouse IL-2 complex preferentially activates mouse T regulatory cells in vivo. The mechanism of the modulation by the JES-6.1 antibody has been elucidated. It has been shown that the JES6.1-mIL-2 complex binds CD25 but not CD122 in vitro.

Exogenous IL-2 therapies, even “non-alpha” therapies that do not bind to the CD25 alpha subunit of the IL-2 trimeric receptor, lead to production of endogenous IL-2. The newly secreted endogenous IL-2 binds preferentially to the trimeric IL-2 receptor on Tregs leading to expansion of immunosuppressive Treg cells via a negative feedback loop.

Increased IL-2 has been implicated to play a role in viral infection. SARS-CoV-2 is a positive stranded RNA virus of the coronaviridae family of respiratory viruses. The virus gains entry into the host by binding angiotensin converting enzyme 2 (ACE2) on lung and gastrointestinal tissues. The course of infection is characterized by a ˜ 7-14 day incubation period followed by symptoms of a dry cough, fever, and shortness of breath. Up to 20% of symptomatic individuals develop severe symptoms and on average 3% of the cases are fatal due to pulmonary failure. Earlier studies with members of the coronavirus family demonstrated that coronavirus infections result in an increase in regulatory T lymphocytes, which likely contributes to delayed viral clearance. More recent studies on COVID-19 patients showed patients in the ICU had higher levels of IL-2, IL-7, IL-10, GSCF, IP10, MCP1, MIP1A, and TNF-α than non-ICU patients, suggesting a role of immunopathology in severe disease. Investigations into direct evidence of alterations in leukocyte homeostasis using the immunological characteristics of peripheral blood leukocytes from SARS-CoV2 infected patients indicate that in COVID-19, similar to some chronic infections, damages to the function of CD4+ T cells promotes excessive activation and possibly subsequent exhaustion of CD8+ T cells. These perturbations of T cell subsets may eventually diminish host antiviral immunity. Therapeutics that either slow viral growth or enhance the immune response to eliminate viral load while reducing some of the associated immune pathologies would therefore be of great benefit.

Immune responses to viruses consist of both the innate and acquired arms of the immune system. The innate system uses toll-like receptors (TLR) and retinoic acid inducible gene I (RIG-I) proteins to sense viral RNA/DNA and induce an initial response. This response includes the production of antiviral cytokines (such as interferon α), chemokines to bring the immune system to the site of the infection and mobilization of the macrophage/dendritic cell arm. Natural Killer (NK) cells (innate lymphoid) directly kill virus infected cells in the absence of MHC class-I expression. This can occur even when the virus has interfered with the MHC class I presentation system.

In addition, during an infection response NK cells produce interferon-□ (IFN-□), thereby increasing the expression of MHC Class I on cells and enhancing the ability of the acquired immune system to respond. The acquired immune system consists of T cells (CD4 and CD8) and B cells. CD4+ T cells recognize viral antigens in the context of MHC-II on antigen presenting cells to both amplify the immune response (through cytokines) and induce B cell class switching and subsequent production of anti-viral antibodies. Activation of CD4 cells, in particular Th1 cells, also releases IFN-□, thus enhancing the presentation of viral antigens. CD8+ T cells exhibit direct lytic effects to virally infected cells which are presenting viral peptides in the context of MHC-I. The initial induction phase of the immune response typically takes 7-10 days to expand the T cell population and generates the cells required to clear the viruses.

IL-2 is a key mediator in the expansion and activation of T cells and NK cells. IL-2 is commonly thought to play a major role in the secondary signals required for T cell activation. The expression of the dimeric (βγ) and trimeric (αβγ) IL-2 receptor complexes show lineage selectivity in that the trimeric receptor containing CD25 (the a subunit) is found highly expressed on regulatory T cells and a subset of activated short lived cytotoxic effector T cells, whereas the dimeric receptor is found on naive T cells, memory T cells, and NK cells. Consequently, naive T cells, memory T cells, and NK cells can receive signaling via IL-2 binding to the dimeric receptor. Regulatory T cells rely on the high affinity trimeric receptor complex to enhance their functions, which include sequestering IL-2 away from binding to memory and naive T cells, and thereby reducing the function of these populations of cells. IL-2 mechanism of action is described in FIG. 1.

Effector T cell subsets also express the trimeric IL-2 receptor complex. While these cells are highly active, binding of IL-2 to a subset of effector T cells induces activation-induced cell death (AICD). Moreover, CD25 has been shown to be expressed on lung endothelium and on vascular endothelium. This expression was correlated with pulmonary edema and vascular leaking in mouse models using high dose IL-2. It was suggested that the expression of CD25 on lung cells was the reason for pulmonary toxicity of high dose IL-2 therapy. Additionally, while lung endothelial cells express CD25 under steady-state conditions, expression levels of CD25 on these cells increased in vivo upon injection of mice with IL-2. It has been shown that knocking out CD25 on non-immune cells or interfering with the CD25 binding epitope of IL-2 by the use of immune complexes of IL-2 and anti-IL-2 antibody (IL-2/mAb) was able to prevent IL-2-induced pulmonary edema and vascular leak syndrome. It was also demonstrated in mice genetically modified to lack T and B cells, and sub-lethally irradiated to remove the remaining immune cells (NK, monocytes, DC, and granulocytes), addition of high dose IL-2 resulted in significant pulmonary edema, indicating a non-immune component.

There has been much research on the dual role of IL-2 in the ability to clear viral infections of the lung. It has been demonstrated that IL-2 is required for the expansion of CD8+ T cell for viral clearance. It has also been shown that IL-2 can mediate lung edema. For example, it has been demonstrated in a mouse influenza model of influenza viral lung infection that memory CD4+ T cells produce high levels of IL-2 and the presence of this IL-2 worsens disease. Regulatory T cells are important for reducing pathological damage to lung tissue in viral infection. It has been hypothesized and demonstrated that one mechanism to control CD8+ effector cells by Treg is by high affinity consumption of IL-2 via CD25 trimeric receptor on Tregs. This in effect removes IL-2 from the expanding effector cells, and subsequently limits the availability of effector cells and potentially reduces viral clearance. It is likely that Tregs also limit the effects of IL-2 on lung endothelium by sequestering away IL-2 from CD25+ endothelial cells. The outcome may be dependent on the ratio of Teff/Treg. High levels of Teff (effector T cells) may lead to viral clearance but also to excessive levels of IL-2 secreted by the immune activated cells, thus leading to lung edema. In contrast, high Treg expansion may reduce pathology of lung edema but also reduce viral clearance and lead to a prolonged viral infection.

Recent data from COVID-19 patients suggest that higher viral loads lead to poorer outcomes; therefore, reduction in viral clearance would be associated with worse outcomes. In mouse models, the role of Tregs in reducing viral clearance was demonstrated using Influenza A virus (IAV) infection model where mice infected with IAV showed higher levels of Tregs in the lungs, spleens and lymph nodes together with higher levels of viral load in the lungs tissue. This was observed even 6 weeks after the initiation of the infection. It was suggested that Influenza A induces Treg expansion to avoid clearance by the immune response. To evaluate whether boosting immune response would increase clearance of IAV infection in the lungs, investigators used mice previously infected with IAV and subsequently infected the mice with lymphocytic choriomeningitis virus (LCMV) that triggers a vigorous cytotoxic T lymphocytes response. Extensive immune response in IAV-infected lungs also led to pulmonary edema and extensive lung tissue damage. Protection from severe pulmonary edema was achieved by treating IAV bearing mice with anti-CD25 blocking antibody prior to administration of LCMV. These data demonstrate that boosting immune response in a situation where Tregs have slowed viral clearance can induce viral clearance. In addition, it demonstrates that blocking IL-2 binding to CD25+ cells reduces the risk of immune mediated pulmonary edema during viral clearance.

IL-2 given as single agent therapy has been shown to enhance antiviral immune responses. Examining the effect of IL-2 therapy during the expansion, contraction and memory phase of T cells in LCMV-infected mice demonstrated that IL-2 treatment during the expansion phase was detrimental to the survival of rapidly dividing effector T cells that have transiently up-regulated the expression of CD25. These effector T cells were subsequently directed to AICD. In contrast, IL-2 therapy was highly beneficial during the contraction phase and resulted in virus-specific T cells survival and activation. It was observed that IL-2 treatment enhanced activation and proliferation of resting memory T-cells. However, IL-2 therapy has its disadvantages. The half-life of IL-2 is short, thus multiple administrations are required, for example a daily loading dose followed by weekly dosing, leading to the risk of additional related adverse events and increased immunogenicity. In addition, the administration of exogenous high dose IL-2 would be expected to bind CD25 positive endothelial cells. Indeed, pulmonary edema and vascular leak syndrome are the main severe adverse events for high dose IL-2 therapy in oncology. Developing technologies to overcome these limitations is critical to the use of IL-2 as a therapy.

One of ordinary skill in the art would recognize that the principles discussed above with regard to IL-2 and treating viral infections would equally apply to IL-2 and treating bacterial infections, or treating cancer.

Advances in the field of biomolecular engineering present researchers with unprecedented opportunities to apply molecular design strategies to modify naturally occurring proteins and generate new molecules for targeted disease therapy. In one area, the development of immunotherapeutics such as cytokine-based or antibody-based drugs has been empowered by evolving technologies and insights from protein engineering. Thus, there is a need to develop engineered anti-IL-2 antibodies that would be used to modulate the functions of IL-2 in certain disease states, for example but not limited to viral or bacterial infections, and cancer.

### SUMMARY

In one aspect, disclosed herein is a method of treating cancer in a subject comprising a step of administering to the subject a composition comprising an anti-IL-2 antibody, said IL-2 antibody comprising a heavy chain variable region (VH) and a light chain variable region (VL), wherein said VH comprises heavy chain complementarity determining regions (HCDRs) HCDR1, HCDR2 and HCDR3, said VL comprises light chain complementarity determining regions (LCDRs) LCDR1, LCDR2 and LCDR3, wherein said CDRs have the amino acid sequences of
HCDR1 comprises the amino acid sequence of SEQ ID NO:62, HCDR2 comprises the amino acid sequence of SEQ ID NO:63, HCDR3 comprises the amino acid sequence of SEQ ID NO:64, LCDR1 comprises the amino acid sequence of SEQ ID NO:65, LCDR2 comprises the amino acid sequence of DAS, LCDR3 comprises the amino acid sequence of SEQ ID NO:67,
   wherein the dose of said anti-IL-2 antibody is between about 0.5 mg/kg-12 mg/kg of said subject, and wherein said antibody promotes differential growth of subsets of immune cells and decreases undesirable effects caused by IL-2, thereby treating said cancer in said subject.

In another aspect, disclosed herein is a method of treating cancer in a subject comprising a step of administering to the subject an anti-IL-2 antibody and a IL-2, said IL-2 antibody comprising a heavy chain variable region (VH) and alight chain variable region (VL), wherein said VH comprises heavy chain complementarity determining regions (HCDRs) HCDR1, HCDR2 and HCDR3, said VL comprises light chain complementarity determining regions (LCDRs) LCDR1, LCDR2 and LCDR3, wherein said CDRs have the amino acid sequences of
HCDR1 comprises the amino acid sequence of SEQ ID NO:62, HCDR2 comprises the amino acid sequence of SEQ ID NO:63, HCDR3 comprises the amino acid sequence of SEQ ID NO:64, LCDR1 comprises the amino acid sequence of SEQ ID NO:65, LCDR2 comprises the amino acid sequence of DAS, LCDR3 comprises the amino acid sequence of SEQ ID NO:67,
   wherein said IL-2 is administered by subcutaneous injection, wherein the dose of said anti-IL-2 antibody is between about 0.5 mg/kg-12 mg/kg of said subject, and wherein said antibody promotes differential growth of subsets of immune cells and decreases undesirable effects caused by IL-2, thereby treating said cancer in said subject.

In another aspect, disclosed herein is a method of treating cancer in a subject comprising a step of administering to the subject an anti-IL-2 antibody, an IL-2, and a checkpoint inhibitor, said IL-2 antibody comprising a heavy chain variable region (VH) and a light chain variable region (VL), wherein said VH comprises heavy chain complementarity determining regions (HCDRs) HCDR1, HCDR2 and HCDR3, said VL comprises light chain complementarity determining regions (LCDRs) LCDR1, LCDR2 and LCDR3, wherein said CDRs have the amino acid sequences of
HCDR1 comprises the amino acid sequence of SEQ ID NO:62, HCDR2 comprises the amino acid sequence of SEQ ID NO:63, HCDR3 comprises the amino acid sequence of SEQ ID NO:64, LCDR1 comprises the amino acid sequence of SEQ ID NO:65, LCDR2 comprises the amino acid sequence of DAS, LCDR3 comprises the amino acid sequence of SEQ ID NO:67,
   wherein said IL-2 is administered by subcutaneous injection, wherein the dose of said anti-IL-2 antibody is between about 0.5 mg/kg-12 mg/kg of said subject, wherein said antibody promotes differential growth of subsets of immune cells and decreases undesirable effects caused by IL-2, and wherein said checkpoint inhibitor comprises PD-1, PDL-1PD-L1, CTLA-4, TIGIT, TIM-3, B7-H3, CD73, LAG3, CD27, CD70, 4-1BB, GITR, OX40, SIRP-alpha (CD47), CD39, ILDR2, VISTA, BTLA, or VTCN-1, thereby treating said cancer in said subject.

In another aspect, disclosed herein is a method of treating a solid cancer in a subject comprising a step of administering to the subject a composition comprising an anti-IL-2 antibody, said IL-2 antibody comprising a heavy chain variable region (VH) and a light chain variable region (VL), wherein said VH comprises heavy chain complementarity determining regions (HCDRs) HCDR1, HCDR2 and HCDR3, said VL comprises light chain complementarity determining regions (LCDRs) LCDR1, LCDR2 and LCDR3, wherein said CDRs have the amino acid sequences of
HCDR1 comprises the amino acid sequence of SEQ ID NO:62, HCDR2 comprises the amino acid sequence of SEQ ID NO:63, HCDR3 comprises the amino acid sequence of SEQ ID NO:64, LCDR1 comprises the amino acid sequence of SEQ ID NO:65, LCDR2 comprises the amino acid sequence of DAS, LCDR3 comprises the amino acid sequence of SEQ ID NO:67,
   wherein the dose of said anti-IL-2 antibody is between about 0.5 mg/kg-12 mg/kg of said subject, and wherein said antibody promotes differential growth of subsets of immune cells and decreases undesirable effects caused by IL-2, thereby treating said cancer in said subject.

In a related aspect of methods disclosed herein, multiple doses of the composition comprising anti-IL2 antibody are administered.

In another related aspect of methods disclosed herein, the method further comprises administering at least a single low dose of IL-2, wherein said low dose of IL-2 comprises between about 15×10³ IU/Kg-500×10³ IU/Kg of said subject. In a further related aspect, administration of IL-2 comprises subcutaneous administration. In still a further related aspect, said IL-2 is administered prior to, concurrent with, or following the administration of said anti-IL-2 antibody. In yet another further related aspect, said IL-2 is administered as multiple doses. In still another further related aspect, multiple doses of IL-2 are administered prior to, concurrent with, or following the administration of said anti-IL-2 antibody, or any combination thereof.

In another related aspect of methods disclosed herein, the method further comprises administering a checkpoint inhibitor. In a further related aspect, the checkpoint comprises PD-L1, PD-1, CTLA-4, TIGIT, TIM-3, B7-H3, CD73, LAG3, CD27, CD70, 4-1BB, GITR, OX40, SIRP-alpha (CD47), CD39, ILDR2, VISTA, BTLA, or VTCN-1.

In another related aspect of a method disclosed here, the solid cancer comprises an unresectable locally advanced or metastatic cancer. In a further related aspect, an unresectable locally advanced or metastatic cancer comprises a melanoma, a renal cell carcinoma (RCC), a non-small cell lung cancer (NSCLC), a head and neck squamous cell carcinoma (HNSCC), gastric or gastro-esophageal cancer, esophageal squamous cell carcinoma, cutaneous squamous cell carcinoma (cSCC), pancreatic adenocarcinoma, cholangiocarcinoma (bile duct cancer), hepato-cellular carcinoma (HCC), colorectal cancer (CRC), epithelial ovarian cancer, cervical cancer, endometrial cancer, thyroid cancer having follicular or papillary histology, urothelial cancer, bladder cancer, uterine cancer, gallbladder cancer, or Merkel cell carcinoma.

In another related aspect of a method disclosed herein, the solid cancer comprises a melanoma, a metastatic melanoma, a primary melanoma and metastatic melanoma, a renal cell carcinoma (RCC), anon-small cell lung cancer (NSCLC), a bladder cancer, ahead and neck cancer, a head and neck squamous cell carcinoma (HNSCC), a nasopharyngeal carcinoma, a urothelial cancer, an adrenal cortical carcinoma, a clear cell renal cell carcinoma (ccRCC), a triple-negative breast cancer, a gastric or gastro-esophageal cancer, an esophageal squamous cell carcinoma, a cutaneous squamous cell carcinoma (cSCC), a pancreatic cancer, a pancreatic adenocarcinoma, a cholangiocarcinoma (bile duct cancer), a hepato-cellular carcinoma (HCC), a colorectal cancer (CRC), an epithelial ovarian cancer, a cervical cancer, an endometrial cancer, a thyroid cancer (follicular or papillary histology), a lung cancer, a uterine cancer, a gallbladder cancer, or a Merkel cell carcinoma, or any tumors that are microsatellite instabilities (MSI)-high tumors. In yet another related aspect of a method disclosed herein, the solid cancer comprises a melanoma, a metastatic melanoma, a primary melanoma and metastatic melanoma, a renal cell carcinoma (RCC), a non-small cell lung cancer (NSCLC), a head and neck cancer, a head and neck squamous cell carcinoma (HNSCC), a pancreatic cancer, a lung cancer, a thyroid cancer, a bladder cancer, a nasopharyngeal carcinoma, a colorectal cancer (CRC), cholangiocarcinoma (bile duct cancer), a uterine cancer, a cervical cancer, a gallbladder cancer, a cutaneous squamous carcinoma. In another related aspect, the solid cancer comprises an immune sensitive cancer.

In yet another related aspect of a method disclosed herein the method comprises a first line treatment, a second line treatment, or a third line treatment, or a combination thereof.

In another related aspect of a method disclosed herein, treating said subject reduces the size of the tumor, inhibits or reduces growth of the tumor, or inhibits or reduces metastases of said tumor, or any combination thereof.

In still another related aspect of a method disclosed herein, the amino acid sequences of the VH comprises the amino acid sequence set forth in SEQ ID NO:26 and the VL comprises the amino acid sequence set forth in SEQ ID NO:27.

In a further related aspect, the antibody comprises an IgG, IgA, IgM, IgE, IgD, a Fv, a scFv, a Fab, a F(ab′)₂, a minibody, a diabody, or a triabody. In yet another further related aspect, the antibody comprises a heavy chain comprising a mutation that reduces binding to an Fcγ receptor. In still a further related aspect, the mutation comprises L234A, L235A mutations. In still another further related aspect, the amino acid sequence of the full length heavy chain is set forth in SEQ ID NO: 72 and the amnio acid sequence of the full length light chain is set forth in SEQ ID NO: 73.

In another aspect, disclosed herein is a method of treating cancer in a subject comprising a step of administering to the subject an anti-IL-2 antibody and a IL-2, said IL-2 antibody comprising a heavy chain variable region (VH) and alight chain variable region (VL), wherein said VH comprises heavy chain complementarity determining regions (HCDRs) HCDR1, HCDR2 and HCDR3, said VL comprises light chain complementarity determining regions (LCDRs) LCDR1, LCDR2 and LCDR3, wherein said CDRs have the amino acid sequences of
HCDR1 comprises the amino acid sequence of SEQ ID NO:62, HCDR2 comprises the amino acid sequence of SEQ ID NO:63, HCDR3 comprises the amino acid sequence of SEQ ID NO:64, LCDR1 comprises the amino acid sequence of SEQ ID NO:65, LCDR2 comprises the amino acid sequence of DAS, LCDR3 comprises the amino acid sequence of SEQ ID NO:67,
   wherein said IL-2 is administered by subcutaneous injection, wherein the dose of said anti-IL-2 antibody is between about 0.5 mg/kg-12 mg/kg of said subject, and wherein said antibody promotes differential growth of subsets of immune cells and decreases undesirable effects caused by IL-2, thereby treating said cancer in said subject.

In yet another aspect, disclosed herein is a method of treating solid cancer in a subject comprising a step of administering to the subject an anti-IL-2 antibody, an IL-2, and a checkpoint inhibitor, said IL-2 antibody comprising a heavy chain variable region (VH) and a light chain variable region (VL), wherein said VH comprises heavy chain complementarity determining regions (HCDRs) HCDR1, HCDR2 and HCDR3, said VL comprises light chain complementarity determining regions (LCDRs) LCDR1, LCDR2 and LCDR3, wherein said CDRs have the amino acid sequences of
HCDR1 comprises the amino acid sequence of SEQ ID NO:62, HCDR2 comprises the amino acid sequence of SEQ ID NO:63, HCDR3 comprises the amino acid sequence of SEQ ID NO:64, LCDR1 comprises the amino acid sequence of SEQ ID NO:65, LCDR2 comprises the amino acid sequence of DAS, LCDR3 comprises the amino acid sequence of SEQ ID NO:67,
   wherein said IL-2 is administered by subcutaneous injection, wherein the dose of said anti-IL-2 antibody is between about 0.5 mg/kg-12 mg/kg of said subject, wherein said antibody promotes differential growth of subsets of immune cells and decreases undesirable effects caused by IL-2, and wherein said checkpoint inhibitor comprises PD-L1, PD-1, CTLA-4, TIGIT, TIM-3, B7-H3, CD73, LAG3, CD27, CD70, 4-1BB, GITR, OX40, SIRP-alpha (CD47), CD39, ILDR2, VISTA, BTLA, or VTCN-1, thereby treating said cancer in said subject. In a related aspect, the checkpoint inhibitor comprises a PD-L1 checkpoint inhibitor.

In another aspect, disclosed herein is a method of treating non-small cell lung cancer (NSCLC) in a subject comprising a step of administering to the subject a composition comprising an anti-IL-2 antibody, said IL-2 antibody comprising a heavy chain variable region (VH) and a light chain variable region (VL), wherein said VH comprises heavy chain complementarity determining regions (HCDRs) HCDR1, HCDR2 and HCDR3, said VL comprises light chain complementarity determining regions (LCDRs) LCDR1, LCDR2 and LCDR3, wherein said CDRs have the amino acid sequences of
HCDR1 comprises the amino acid sequence of SEQ ID NO:62, HCDR2 comprises the amino acid sequence of SEQ ID NO:63, HCDR3 comprises the amino acid sequence of SEQ ID NO:64, LCDR1 comprises the amino acid sequence of SEQ ID NO:65, LCDR2 comprises the amino acid sequence of DAS, LCDR3 comprises the amino acid sequence of SEQ ID NO:67,
   wherein the dose of said anti-IL-2 antibody is between about 0.5 mg/kg-12 mg/kg of said subject, and wherein said antibody promotes differential growth of subsets of immune cells and decreases undesirable effects caused by IL-2, thereby treating said NSCLC in said subject.

In another aspect, disclosed herein is a method of treating non-small cell lung cancer (NSCLC) in a subject comprising a step of administering to the subject an anti-IL-2 antibody and IL-2, said IL-2 antibody comprising a heavy chain variable region (VH) and alight chain variable region (VL), wherein said VH comprises heavy chain complementarity determining regions (HCDRs) HCDR1, HCDR2 and HCDR3, said VL comprises light chain complementarity determining regions (LCDRs) LCDR1, LCDR2 and LCDR3, wherein said CDRs have the amino acid sequences of
HCDR1 comprises the amino acid sequence of SEQ ID NO:62, HCDR2 comprises the amino acid sequence of SEQ ID NO:63, HCDR3 comprises the amino acid sequence of SEQ ID NO:64, LCDR1 comprises the amino acid sequence of SEQ ID NO:65, LCDR2 comprises the amino acid sequence of DAS, LCDR3 comprises the amino acid sequence of SEQ ID NO:67,
   wherein the dose of said anti-IL-2 antibody is between about 0.5 mg/kg-12 mg/kg of said subject, and wherein said antibody promotes differential growth of subsets of immune cells and decreases undesirable effects caused by IL-2, and wherein said IL-2 is administered subcutaneously, thereby treating said NSCLC in said subject.

In another aspect, disclosed herein is a method of treating non-small cell lung cancer (NSCLC) in a subject comprising a step of administering to the subject an anti-IL-2 antibody, an IL-2, and a checkpoint inhibitor, said IL-2 antibody comprising a heavy chain variable region (VH) and a light chain variable region (VL), wherein said VH comprises heavy chain complementarity determining regions (HCDRs) HCDR1, HCDR2 and HCDR3, said VL comprises light chain complementarity determining regions (LCDRs) LCDR1, LCDR2 and LCDR3, wherein said CDRs have the amino acid sequences of
HCDR1 comprises the amino acid sequence of SEQ ID NO:62, HCDR2 comprises the amino acid sequence of SEQ ID NO:63, HCDR3 comprises the amino acid sequence of SEQ ID NO:64, LCDR1 comprises the amino acid sequence of SEQ ID NO:65, LCDR2 comprises the amino acid sequence of DAS, LCDR3 comprises the amino acid sequence of SEQ ID NO:67,
   wherein the dose of said anti-IL-2 antibody is between about 0.5 mg/kg-12 mg/kg of said subject, and wherein said antibody promotes differential growth of subsets of immune cells and decreases undesirable effects caused by IL-2, wherein said IL-2 is administered subcutaneously, and wherein said checkpoint inhibitor comprises PD-1, PDL-1PD-L1, CTLA-4, TIGIT, TIM-3, B7-H3, CD73, LAG3, CD27, CD70, 4-1BB, GITR, OX40, SIRP-alpha (CD47), CD39, ILDR2, VISTA, BTLA, or VTCN-1, thereby treating said NSCLC in said subject.

In another aspect, disclosed herein is a method of treating renal cell carcinoma (RCC) in a subject comprising a step of administering to the subject an anti-IL-2 antibody, said IL-2 antibody comprising a heavy chain variable region (VH) and a light chain variable region (VL), wherein said VH comprises heavy chain complementarity determining regions (HCDRs) HCDR1, HCDR2 and HCDR3, said VL comprises light chain complementarity determining regions (LCDRs) LCDR1, LCDR2 and LCDR3, wherein said CDRs have the amino acid sequences of
HCDR1 comprises the amino acid sequence of SEQ ID NO:62, HCDR2 comprises the amino acid sequence of SEQ ID NO:63, HCDR3 comprises the amino acid sequence of SEQ ID NO:64, LCDR1 comprises the amino acid sequence of SEQ ID NO:65, LCDR2 comprises the amino acid sequence of DAS, LCDR3 comprises the amino acid sequence of SEQ ID NO:67,
   wherein the dose of said anti-IL-2 antibody is between about 0.5 mg/kg-12 mg/kg of said subject, and wherein said antibody promotes differential growth of subsets of immune cells and decreases undesirable effects caused by IL-2, thereby treating said RCC in said subject.

In another aspect, disclosed herein is a method of treating melanoma in a subject comprising a step of administering to the subject a composition comprising an anti-IL-2 antibody, said IL-2 antibody comprising a heavy chain variable region (VH) and a light chain variable region (VL), wherein said VH comprises heavy chain complementarity determining regions (HCDRs) HCDR1, HCDR2 and HCDR3, said VL comprises light chain complementarity determining regions (LCDRs) LCDR1, LCDR2 and LCDR3, wherein said CDRs have the amino acid sequences of
HCDR1 comprises the amino acid sequence of SEQ ID NO:62, HCDR2 comprises the amino acid sequence of SEQ ID NO:63, HCDR3 comprises the amino acid sequence of SEQ ID NO:64, LCDR1 comprises the amino acid sequence of SEQ ID NO:65, LCDR2 comprises the amino acid sequence of DAS, LCDR3 comprises the amino acid sequence of SEQ ID NO:67,
   wherein said melanoma comprises a primary tumor or metastatic melanoma or a combination thereof, wherein the dose of said anti-IL-2 antibody is between about 0.5 mg/kg-12 mg/kg of said subject, and wherein said antibody promotes differential growth of subsets of immune cells and decreases undesirable effects caused by IL-2, thereby treating said melanoma in said subject.

In another aspect, disclosed herein is a method of treating cancer in a subject comprising a step of administering to the subject an anti-IL-2 antibody, IL-2, and a checkpoint inhibitor, wherein said IL-2 antibody comprising a heavy chain variable region (VH) and a light chain variable region (VL), wherein said VH comprises heavy chain complementarity determining regions (HCDRs) HCDR1, HCDR2 and HCDR3, said VL comprises light chain complementarity determining regions (LCDRs) LCDR1, LCDR2 and LCDR3, wherein said CDRs have the amino acid sequences of
HCDR1 comprises the amino acid sequence of SEQ ID NO:62, HCDR2 comprises the amino acid sequence of SEQ ID NO:63, HCDR3 comprises the amino acid sequence of SEQ ID NO:64, LCDR1 comprises the amino acid sequence of SEQ ID NO:65, LCDR2 comprises the amino acid sequence of DAS, LCDR3 comprises the amino acid sequence of SEQ ID NO:67,
   wherein the dose of said anti-IL-2 antibody is between about 0.5 mg/kg-12 mg/kg of said subject, wherein said dose of IL-2 is a low dose, wherein said checkpoint inhibitor is a PD-L1 checkpoint inhibitor, and wherein said antibody promotes differential growth of subsets of immune cells and decreases undesirable effects caused by IL-2, thereby treating said cancer in said subject.

In another related aspect, the method further comprises administering an IL-2. In a further related aspect, the administration of IL-2 comprises subcutaneous administration. In another further related aspect, the IL-2 is administered as a single dose. In yet another further related aspect, the IL-2 is administered prior to, concurrent with, or following the administration of said anti-IL-2 antibody. In still another further related aspect, the IL-2 is administered as multiple doses. In another further related aspect, the multiple doses of IL-2 are administered prior to, concurrent with, or following the administration of said anti-IL-2 antibody, or any combination thereof. In yet another further related aspect, the dose of IL-2 is between about 15×10³ IU/Kg-270×10³ IU/Kg of said subject.

In another related aspect, the method further comprises administering a checkpoint inhibitor. In another related aspect, the method further comprises administering IL-2 and a checkpoint inhibitor.

The method of claim 10, wherein said checkpoint comprises PD-L1, PD-1, CTLA-4, TIGIT, TIM-3, B7-H3, CD73, LAG3, CD27, CD70, 4-1BB, GITR, OX40, SIRP-alpha (CD47), CD39, ILDR2, VISTA, BTLA, or VTCN-1. In another further related aspect, said checkpoint is PD-L1. In still another further related aspect, the PD-L1 checkpoint inhibitor is avelumab.

In another related aspect, the cancer comprises a solid cancer. In a further related aspect the solid cancer comprises a non-small cell lung cancer (NSCLC), a head and neck cancer, a head and neck squamous cell carcinoma (HNSCC), a pancreatic cancer, a lung cancer, a thyroid cancer, a bladder cancer, a nasopharyngeal carcinoma, a melanoma, a metastatic melanoma, a primary melanoma and metastatic melanoma, a colorectal cancer (CRC), a bladder cancer, cholangiocarcinoma (bile duct cancer), a uterine cancer, a cervical cancer, a gallbladder cancer, or a renal cell carcinoma (RCC). In another further related aspect, the solid cancer comprises a non-small cell lung cancer (NSCLC), a melanoma, a metastatic melanoma, a primary melanoma and metastatic melanoma, or a renal cell carcinoma (RCC). In still another further related aspect, the solid cancer comprises a non-small cell lung cancer (NSCLC). In another further related aspect, the non-small cell lung cancer (NSCLC) comprises a unresectable advanced cancer or a metastatic cancer.

In a related aspect, the method comprises a first line treatment, a second line treatment or a third line treatment, or a combination thereof.

In another related aspect, the anti-IL2 antibody is administered for between about 3 months and 1 year.

In another related aspect, the method of treating said subject reduces the size of the tumor, inhibits or reduces growth of the tumor, or inhibits or reduces metastases of said tumor, or any combination thereof.

In another related aspect, the VH and VL have the amino acid sequences as set forth, wherein
the VH comprises the amino acid sequence of SEQ ID NO:26, and the VL comprises the amino acid sequence of SEQ ID NO:27.

In another related aspect, the antibody comprises an IgG, IgA, IgM, IgE, IgD, a Fv, a scFv, a Fab, a F(ab′)₂, a minibody, a diabody, or a triabody. In a further related aspect, the antibody comprises a heavy chain comprising a mutation that reduces binding to an Fcγ receptor. In yet a further related aspect, the mutation comprises L234A, L235A mutations.

In another related aspect, the amino acid sequence of the full length heavy chain is set forth in SEQ ID NO: 72 and the amnio acid sequence of the full length light chain is set forth in SEQ ID NO: 73.

In another related aspect, the undesirable effect caused by IL-2 comprises one or more of activation of regulatory T cells, apoptosis of CD25+T effector cells, IL-2 induced pulmonary edema, IL-2 induced pneumonia, or IL-2-induced vascular leakage. In a further related aspect, the anti-IL2 antibody binds to IL-2 and wherein said binding reduces or eliminates IL-2 binding to CD25. In yet another further related aspect, the IL-2 binding to CD132/CD122 is not reduced or inhibited.

### BRIEF DESCRIPTION OF THE DRAWINGS

The patent or patent application file contains at least one drawing executed in color. Copies of this patent or patent application publication with color drawing(s) will be provided by the Office upon request and payment of the necessary fee.

The present disclosure of engineered anti-IL-2 antibodies, both as to their generation and method of use, together with objects, features, and advantages thereof, may best be understood by reference to the following detailed description when read with the accompanying drawings in which:

FIG. 1. presents a schematic representation of IL-2 mechanism of action and its dual role in controlling immune response.

FIG. 2. presents a schematic representation of anti-IL-2 antibodies-directed immunotherapy.

FIGS. 3A and 3B present a schematic representation of the progression of COVID-19 infection and potential anti-IL-2 therapy as an adjuvant intervention. FIG. 3A is adapted from Shi Y et al., (2020) COVID-19 infection: the perspectives on immune responses. Cell Death & Differentiation volume 27, pages 1451-1454 (doi:10.1038/s41418-020-0530-3), FIG. 1.

FIGS. 4A-4D present representative SPR sensorgram of JES6.1 antibody binding to human IL-2 (FIG. 4A), mouse IL-2 (FIG. 4B), and of JES6.1RMC antibody binding to human IL-2 (FIG. 4C) and mouse IL-2 (FIG. 4D).

FIGS. 5A-5C present results of IL-2 binding of YSD clones expressing JES6.1 antibody in scFv format. X axis fluorescence levels correspond to scFv expression level of Jes6.1, Y axis fluorescence levels correspond to binding of human or mouse IL-2. FIG. 5A shows negative control without IL-2. FIG. 5B shows JES6.1 YSD clones with 1000 nM human IL-2. FIG. 5C shows YSD expressing mouse IL-2 incubated with 100 nM labeled JES6.1.

FIG. 6A presents binding of isolated yeast-surface display clones to IL-2 (0.1 nM). Mean Fluorescence Intensity (Em 655 nM) was normalized to the yeast surface expression levels. Negative YSD clones were labeled with 500 nM hIL-2.

FIG. 6B presents non-specific binding of YSD clones to mixture of OX40/PD-1/TNFR2. The clones were labeled with the 500 nM mixture. TNFR2 binding yeast clone served as positive control.

FIG. 7 shows purification of BDG17.023 IgG. The antibody was run on a GE superdex 200 10/300 increase (CV=25 ml) in PBS buffer at 0.5 ml/min. The leading peak (0.38CV) corresponds to a typical aggregate, and a second peak (0.51CV) with retention of approximately 12.9 ml is typical of an ordinary human IgG.

FIGS. 8A-8B present binding kinetic of BDG17.023 IgG to hIL-2 (FIG. 8A) and mIL-2 (FIG. 8B).

FIG. 9 presents receptor discrimination by BDG17.023-IL-2 complex with traces of SPR response. BDG17.023 was immobilized to the CM5 chip, and hIL-2 (60 RU), CD122 (20RU) and CD25 (0RU) were streamed as indicated with an arrow.

FIGS. 10A-10D present spleen immune cell populations of mice treated with JES6.1-mIL-2 complex and BDG17.023-hIL-2 complex. FIG. 10A shows percentages of immune cell populations from mice treated with JES6.1-mIL-2 complex. FIG. 10B shows memory phenotype effector T cells (MP) CD8+/Tregs ratios of mice treated with JES6.1-mIL-2 complex. FIG. 10C shows percentages of immune cell populations from mice treated with BDG17.023-hIL-2 complex. FIG. 10D shows MP CD8+/Tregs ratios of mice treated with BDG17.023-hIL-2 complex.

FIG. 11 presents the alignment of amino acid sequences of the heavy chain variable region of JES6.1, clone 1 (17.021), clone 2 (17.022), clone 4 (17.023), clone 5 (17.030), and clone 6 (17.035).

FIG. 12 presents the alignment of amino acid sequences of the light chain variable region of JES6.1, clone 1 (17.021), clone 2 (17.022), clone 4 (17.023), clone 5 (17.030), and clone 6 (17.035).

FIGS. 13A and 13B present the alignment of amino acid sequences of the heavy chain variable region (FIG. 13A) and the light chain variable region (FIG. 13B) of the humanized clone 17.014, clone 17.038, clone 17.043, clone 17.053, and clone 17.054. Black triangles denote IMGT CDR positions. Bold/Italic font denotes ABR/CDR positions, respectively.

FIGS. 14A-14G. Binding kinetics of indicated antibodies to human IL-2. Surface plasmon resonance (SPR) sensogram traces of binding kinetics of anti-IL-2 antibody clones BDG17.038 (FIG. 14A), BDG 17.043 (FIG. 14B), BDG17.053 (FIG. 14C), 17.054 (FIG. 14D), BDG17.066 (FIG. 14E), BDG17.067 (FIG. 14F), and BDG17.069 (FIG. 14G), to human IL-2. BDG 17.038, BDG 17.043, BDG 17.066, BDG 17.067 and BDG 17.069 binding kinetics were determined by the multi-cycle method. BDG 17.053 and BDG 17.054 binding kinetics were determined by the single-cycle method.

FIGS. 15A-15B. Binding kinetics of indicated antibodies to cynomolgus monkey IL-2. Surface plasmon resonance (SPR) sensogram traces of binding kinetics of anti-IL-2 antibody clones BDG17.067 (FIG. 15A), and BDG17.069 (FIG. 15B), to cynomolgus monkey IL-2.

FIGS. 16A-16G. Differential Scanning Fluorimetry (DSF) analysis of the indicated IgGs' melting point. Light dashed line indicates Tonset and bold dashed lines indicate Tm1, and where applicable Tm2. Anti-IL-2 clones analyzed are BDG17.038 (FIG. 16A), BDG17.043 (FIG. 16B), BDG 17.053 (FIG. 16C), BDG 17.054 (FIG. 16D), BDG17.066 (FIG. 16E), BDG17.067 (FIG. 16F), and BDG17.069 (FIG. 16G).

FIGS. 17A-17G. Present receptor discrimination of indicated antibody/IL-2 complex by tracing of SPR response. Antibody was immobilized to the CM5 chip, and hIL-2, CD122, and CD25 were streamed as indicated with an arrow. FIG. 17A presents a schematic order of injection of compounds to SPR chip, which represents sequential anti-IL-2 antibodies complexed with human IL-2 (hIL2), binding to CD122 but not to CD25. FIGS. 17B-17G present the SPR response: BDG17.038 (FIG. 17B), BDG017.043 (FIG. 17C), 17.054 (FIG. 17D), BDG17.066 (FIG. 17E), BDG17.067 (FIG. 17F), and BDG17.069 (AU-007; FIG. 17G).

FIGS. 18A and 18B show anti-human IL-2 antibodies (clones 17.043 and 17.054) demonstrate potent immune stimulating effect in vivo. The anti-IL-2 antibody/hIL-2 complexes increase effector cell populations with no observed effect on regulatory T cells. FIG. 18A shows C57BL/6 mice were administered daily with anti-IL-2 antibody (10 ug) pre-complexed with 0.5 ug of hIL-2 for four days. FIG. 18B shows C57BL/6 mice were administered daily with anti-IL-2 antibody (25 ug) pre-complexed with 1.25 ug of hIL-2 for four days. On day 5, splenocytes were isolated and immune cell populations were analyzed by flow cytometry. Presented are mean values for each experimental group (n=6 per group). Lymphocytes were gated according to side-scatter and forward-scatter parameters and subsequent immune cells subpopulations were gated as follows: Tregs (CD45+, CD3+, CD4+, CD25+, FoxP3+), CD8 T cells (CD45+, CD3+, CD8+, CD122+, CD25−), NKT-cells (CD45+, CD3+, CD49b+, NK1.1+), NK cells (CD45+, CD3−, CD49b+, NK1.1+).

FIGS. 19A and 19B show anti-human IL-2 antibodies (clones 17.043 and 17.054) demonstrate potent dose dependent immune stimulating effect in vivo. FIG. 19A shows C57BL/6 healthy mice were administered daily with anti-IL-2 antibody/hIL-2 complex (25 ug/1.25 ug respectively) for four days. On day 5, splenocytes were isolated and immune cells populations were analyzed by flow cytometry. FIG. 19B shows anti-human IL-2 antibodies demonstrate potent in vivo immune stimulating effect in a dose dependent manner. C57BL/6 healthy mice were administered daily with increasing doses of anti-IL-2 antibody/hIL-2 complex as indicated. On day 5, splenocytes were isolated and immune cells populations were analyzed using flow cytometry. Lymphocytes were gated according to side-scatter and forward-scatter parameters and subsequent immune cells subpopulations were gated as follows: Tregs (CD45+, CD3+, CD4+, CD25+, FoxP3+), CD8 T cells (CD45+, CD3+, CD8+, CD122+, CD25−), NKT-cells (CD45+, CD3+, CD49b+, NK1.1+), NK cells (CD45+, CD3−, CD49b+, NK1.1+).

FIGS. 20A and 20B show anti-human IL-2 antibodies (clones 17.043 and 17.054) demonstrate safe dose regimen in vivo. FIG. 20A shows C57BL/6 healthy mice were administered daily with anti-IL-2 antibody/hIL-2 complex (10 ug/0.5 ug respectively) for four days. FIG. 20B shows C57BL/6 healthy mice were administered daily with anti-IL-2 antibody/hIL-2 complex (25 ug/1.25 ug respectively) for four days. At the end of the experiments, mice were weighed, and percent of body weight changes were calculated in respective to the weight of each mouse at the beginning of the study. Presented are mean percent of body weight (BW) change for each experimental group (n=6 per group).

FIGS. 21A and 21B show mean tumor volume results. Anti IL-2 antibodies (clones 17.038, 17.043, 17.053, and 17.054) inhibit tumor growth in an I/O resistant tumor model with a tolerable safety profile. C57BL/6 healthy mice were inoculated with B16F10 melanoma tumor cells on day 0. On day 5, the mice were randomized to experimental groups (n=10 per group) and administered daily with anti-IL-2 antibody/hIL-2 complex (20 ug/1 ug respectively) or with PBS for four days. 17.054 is the parent antibody of 17.069 (AU-007), but lacks the LALA effector silent mutation in the Fc domain that has been engineered into AU-007. FIG. 21A shows changes in tumor volume for each experimental group. FIG. 21B shows changes in body weight for 17.043 and 17.054 experimental groups. Percent body weight changes were calculated in respective to the weight of each mouse at the beginning of the study.

FIGS. 22A-22G show the results of analyzing the different formulations of anti-IL-2 antibody clone BDG 17.069. FIG. 22A presents the BDG 17.069 parameters at T=0. FIG. 22B presents BDG 17.069 appearance, pH, protein concentration, and sub-visual particle formation at T=0 and after incubation at 40° C. for 1 and 2 weeks. FIG. 22C presents BDG 17.069 SEC, caliper-SDS and capillary isoelectric focusing analysis at T=0 and after incubation at 40° C. for 1- and 2-weeks. FIG. 22D presents BDG 17.069 appearance, pH, protein concentration, and sub-visual particle formation at T=0 and post agitation at 300 rpm for 3 days. FIG. 22E presents BDG 17.069 SEC, caliper-SDS, and capillary isoelectric focusing analysis at T=0 and post agitation at 300 rpm for 3 days. FIG. 22F presents BDG 17.069 appearance, pH, protein concentration, and sub particle formation at T=0 and after five cycles of Freeze/Thaw. FIG. 22G presents BDG 17.069 SEC, caliper-SDS, and capillary isoelectric focusing analysis at T=0 and after five cycles of Freeze/Thaw.

FIG. 23 graphically presents the administration and dosage schemes for BDG17.069 Q2w Monotherapy (left-A), combination BDG17.069+IL-2 Loading Dose (center-B), and combination BDG17.069 Q2w+IL-2 Q2w (right-C).

FIG. 24 presents Duration of Treatment and Efficacy Details. The patient in Cohort 1 received 0.5 mg/kg BDG 17.069, while the patients in Cohort 2 received 1.5 mg/kg BDG 17.069.

FIG. 25 presents pharmacokinetics data showing the early pharmacokinetic profile of the first 3 patients administered monotherapy BGD 17.096

FIGS. 26A-26D graphically present Pharmacodynamic (PD) data showing the effects of BDG 17.069 and the mechanism of action. FIG. 26A presents % change in CD4+ Treg population.

FIG. 26B presents CD8/Treg Ratio and includes a key for FIGS. 26A-26B. FIG. 26C presents Total Circulating IFN-γ. FIG. 26D presents the eosinophil count and a key for FIGS. 26C-26D. (Circle—patient 1; Triangle—patient 2; Square—patient 3).

FIGS. 27A-27H present technical characteristics of 17.069 (AU-007) antibody, schematic diagrams of injection schedules including SPR sensogram traces. FIGS. 27A and 27B-27D: AU-007 binds human IL-2 with high affinity and inhibits the binding to hCD25 while preserving the binding to hCD122. Affinity and epitope binding site was assessed using Surface Plasmon Resonance (SPR). FIG. 27A presents SPR sensorgram traces and calculated binding kinetics of a CM5 chip-bound AU-007 with hIL-2 serving as analyte. FIG. 27B presents illustrative models of IL-2 binding as part of a human IL-2-Trimeric Receptor Complex compared with AU-007 blocking the access of IL-2 to CD25. FIGS. 27C and 27D present AU-007 Epitope binding analysis: AU-007 was captured on a CM5 chip and soluble hIL-2 was injected, forming a complex. Subsequently, soluble hCD25 was injected followed by the injection of soluble hCD122. Diagrams represent SPR sensorgram traces of complex formation of Ab/IL-2/IL-2R. Arrows indicate where hIL-2, hCD25, and hCD122 were injected. FIGS. 27E-27H: naIL-2 (hIL-2/hCD25 conjugate) inhibits the binding to hCD25 while preserving the binding to hCD122. FIGS. 27E-27F present a schematic showing biotinylated hCD25 was captured on a CM5 chip and soluble hIL-2, soluble hCD25, and naIL-1 were injected subsequently. FIGS. 27G-27H present a schematic showing Fc tagged hCD122 was captured on a CM5 chip and soluble hIL-2, soluble hCD25, and soluble naIL-2 were injected subsequently. Diagrams represent SPR sensorgram traces of complex formation of IL-2R/Cytokine. Arrows indicate where hIL-2, hCD25, and hCD122 were injected.

FIGS. 28A-28H demonstrate that AU-007 can capture and redirect endogenous IL-2 to break the auto-inhibitory loop in hPBMCs while HD IL-2 or naIL-2 cannot. AU-007 promotes the expansion of NKs and CD8 T-cells while completely inhibiting the expansion of regulatory T-cells. FIGS. 28A-28E (Key in FIG. 28A): naïve hPBMCs were treated once on day 0 with 1 nM of naIL-2 (purple) or with HD IL-2 (1 nM) combined with 1 uM of isotype control Ab (black) or with either 1 uM AU-007 (red) or 10 uM AU-007 (turquoise). The culture was monitored for 7 days, and immune cell subpopulations were analyzed daily by flow cytometry. Values were normalized to untreated samples (UNT) at each day. While naIL-2 expands NKs similarly to AU-007 it fails to inhibit Tregs expansion. AU-007 completely inhibits Tregs expansion in culture (FIG. 28A) and significantly increases the Teffs:Tregs ratio (FIG. 28B), without hindering NKs (FIG. 28C). AU-007 downregulates the suppressive markers of CD4+ Treg from FIG. 28A, as defined by a significant reduction in MFI of CD25 and FoxP3 (FIGS. 28D-28E). FIGS. 28F-28H (key in FIG. 28H): AU-007 rescues activated lymphocyte viability decreased by treatment with HD IL-2. hPBMCs culture was stimulated once with anti-CD3/anti-CD28 Abs with or without 10 uM of AU-007. Three (3) days post-stimulation all samples were given HD IL-2 (1 nM) and were monitored daily for cell viability using flow cytometry.

FIGS. 29A-29K show AU-007 binds to endogenous IL-2 and breaks the negative feedback loop in human PBMCs. FIGS. 29A-29E (Key in FIG. 29E): naive hPBMCs were treated once at day 0 with either 1 uM AU-007 (red) or with an isotype control antibody (blue). No exogenous IL-2 was added. The culture was monitored for 7 days, and immune cell subpopulations were analyzed daily by flow cytometry. Values were normalized to untreated samples (UNT) at each day. AU-007 completely inhibits Tregs expansion (FIG. 29A) and significantly increases Teffs:Tregs ratio (FIG. 29B), without hindering NKs (FIG. 29C). AU-007 downregulates the suppressive markers of CD4+ Treg from FIG. 29A, as defined by a significant reduction in mean fluorescence intensity (MFI) of CD25 and FoxP3 (FIG. 29D-2E). FIGS. 29F-29K: Total hPBMCs were stimulated for 24 h with anti-CD3/anti-CD28 (stimulation only) or stimulated with anti-CD3/anti-CD28 in the presence of 200 nM of AU-007 mAb or with 200 nM of isotype control mAb (blue). No exogenous IL-2 was added. Immune cells subpopulations were analyzed by flow cytometry. AU-007 inhibits Tregs without hindering effector cells and NKs (FIGS. 29F-29I). AU-007 downregulates the suppressive markers of CD4+ Treg from panel G, as defined by a significant reduction in MFI of CD25 and FoxP3 (FIGS. 29J-29K).

FIGS. 30A-30C show that AU-007 and the naIL-2 do not hinder CD122/CD132-STAT5 signaling activity. A HEK239-dimer-STAT5-SEAP reporter cell line that stably expresses the human IL-2 dimer receptor (CD122/CD132) with no expression of CD25 and drives the expression of secreted embryonic alkaline phosphatase (SEAP) under a STAT5 promoter, was used to detect IL-2/IL-2-dimeric receptor signaling. FIG. 30A shows CD25, CD122, and CD132 expression levels were detected using flow cytometry verifying the exclusive expression of the dimeric receptor. FIG. 30B presents dose response curves of IL-2 alone (circles) or in the presence of 200 nM AU-007 (squares) or of 200 nM of an anti-IL-2 antibody that inhibits interactions with the dimeric receptor (triangles). FIG. 30C presents a dose response curve of naIL-2 (circles). HEK239-dimmer-STAT5-SEAP reporter cells were treated with increasing concentrations of hIL-2 alone or with indicated anti-hIL-2 antibodies (FIG. 30B) or with increasing concentrations of naIL-2 (FIG. 30C), 24 h post-treatment accumulated levels of SEAP were measured from cells media and functional EC-50 was calculated using GraphPad (FIGS. 30B-30C).

FIGS. 31A-31G show that AU-007 captures endogenous IL-2 and demonstrates in-vivo potent immune-stimulating effects even 8 days after a single treatment. NOG-EXL mice were engrafted with hPBMCs from 3 human donors to examine donor-to-donor variability. 10 days post hPBMCs engraftment mice were randomized to the study groups (9 mice per cohort, each composed of 3 mice from each donor, colored dots represent the average of each donor for every cohort). Study groups mice were treated once with 20 mg/Kg AU-007 or with 20 mg/Kg of isotype control Ab (hIgG1-LALA), no exogenous IL-2 was added. FIG. 31A. Experiment outline. FIG. 31B. AU-007/hIL-2 complex detection from mouse serum using ELISA n=9±SD. FIGS. 31C-31G. Immune cell analysis of splenocytes using flow cytometry n=9±SE. Human immune cells were defined from total lymphocytes as hCD45+ hCD3+, and subpopulations were defined using anti-human antibodies as indicated. Statistical analysis was done using the 2way ANOVA test, * p<0.05, **** p<0.0001. (Key below FIGS. 31F-31G)

FIGS. 32A-32C present that an IL-2 negative feedback loop caused by endogenous IL-2 limits the activity of modified IL-2-based therapies. FIG. 32A. Schematic representation of IL-2 role as an immunomodulator in homeostasis and inflammation. The Headers in FIG. 32A indicate status/functionality for FIGS. 32B and 32C as well. FIG. 32B. Exogenous administration of modified IL-2 with bias selectivity to dimer-expressing cells promotes the expansion of CD25 negative (CD25) effector cells yet is undermined by the endogenous IL-2 that pushes the system back to homeostasis.

FIG. 32C. AU-007 captures and redirects endogenous IL-2, allowing it to expand CD25 negative (CD25−) effector cells while breaking the auto-inhibitory loop and expanding the inflammation & immune stimulation stage.

FIGS. 33A-33B present HEK-293 cells expressing the IL-2 dimer receptor are incubated with IL-2 (red) or IL2+AU-007 (17.069) (blue) or a control antibody with known dimer inhibition properties (green). The read out is production of secreted embryonic alkaline phosphatase (SEAP) after stimulation. SEAP was detected using a phosphatase substrate and absorbance was measured at 620 nm. AU-007 did not inhibit the ability of IL-2 to stimulate the dimeric receptor for IL-2 on the HEK293 cells (EC50 of IL-2 alone=0.71 pM and 0.78 pM in the presence of AU-007).

FIGS. 34A-34D present pSTAT5 IC50 assay in human peripheral blood mononuclear cells (PBMC) results. Human PBMC were incubated with IL-2 in the presence or absence of AU-007. The data demonstrate that AU-007 inhibits the ability of IL-2 to induce phosphorylation of STAT5 in IL-2 trimeric receptor expressing regulatory T cells (CD3+CD4+CD25+CD127-FoxP3+) (FIG. 34A) but not in dimeric receptor expressing CD3-CD56+NK cells (FIG. 34B), memory phenotype (MP) CD8+ T cells (FIG. 34C) or CD3+CD8+CD56+ NKT cells (FIG. 34D).

FIGS. 35A-35E present the percent cells (%) from parent of CD8 MP Teff (FIG. 35A), NK (FIG. 35B), NKT (FIG. 35C), CD4+ Treg (FIG. 35D), and CD8+ Treg (FIG. 35E) cells following administration of AU-007/hIL-2. Briefly, AU-007, IL-2+AU007, or IL2+ control antibody were injected into C57Bl/6 mice daily for 4 days and spleens were harvested on day 5. Splenocytes were phenotyped for the percentages of lymphocyte populations. AU-007 significantly expanded the CD8+ memory cells, NK cells and NKT cells but did not expand regulatory T cells.

FIGS. 36A-36C present the percent cells (%) from parent of CD8 MP Teff (FIG. 35A), NK (FIG. 36B), and NKT (FIG. 36C) cells following administration of BDG17.054/hIL-2.

FIGS. 37A and 37B present data showing administration of BDG17.069+ human IL-2 to MC38 colon cancer bearing mice induces regressions and in the presence of either anti-PD-1 (FIG. 37A) or anti-PD-L1 (FIG. 37B) induces tumor eradications.

FIGS. 38A and 38B present data showing administration of BDG17.069+ human IL-2 inhibits tumor growth in LL/2 (Lung) cancer model. BDG17.069 (AU-007) decreased LL/2 tumor growth by 74% relative to saline control. Please note, human IL-2 is administered with AU-007 since AU-007 does not bind mouse IL-2.

FIG. 39 presents a schematic of the update Phase I Dose Escalation Regime. (Solid border indicates work performed and or in progress. Dashed line indicates next step progression (as of November 2023).

FIG. 40 presents AU-007 Monotherapy: (Arm 1A) Treatment Duration and Best Response, as of Oct. 13, 2023.

FIGS. 41A and 41B present AU-007+IL-2 (Arm 1B (AU-007+1 Loading Dose Aldesleukin—FIG. 41A); Arm 1C (AU-007+Aldesleukin (Q2W)−FIG. 41B)) Treatment Duration and Best Response, as of October, 2023.

FIGS. 42A, 42B, and 42C present Safety Data. FIG. 42A presents the Safety Data for Arm 1A (first 4 cohorts) AU-007 Monotherapy: Mild Toxicity Profile. FIG. 42B presents the population statistics for all three Arms (1A, 1B, and 1C) as of Oct. 13, 2023. * All Grade ¾ drug-related AEs were transient (3-7 days) lymphopenia. ** A single drug-related SAE of transient (˜12 hours) Grade 2 CRS occurred in a patient with cutaneous squamous carcinoma receiving AU-007+Q2W 135K IU/kg aldesleukin. The patient became symptomatic with fever and mild hypotension starting 6 hours after receiving the initial aldesleukin dose. The patient had a pre-treatment pneumonia with RUL consolidation treated with oral antibiotics. The patient continued therapy with mild symptoms on receiving the second doses of AU-007+aldesleukin. FIG. 42C presents a chart detailing Drug-Related Adverse Events for all three Arms (1A, 1B, and 1C) as of Oct. 13, 2023.

FIGS. 43A and 43B present Arm 1B AU-007+Proleukin® (aldesleukin): Mild Toxicity Profile of Drug Related Adverse Events (AEs). In FIG. 43B, dMMR indicates mismatch repair deficient.

FIGS. 44A, 44B, and 44C present AU-007 Objective Response: Waterfall Plot showing the ongoing results for patients within the study suffering from different cancers, as of Oct. 13, 2023. Status of tumors and subject participation within the trial, AU-007 Monotherapy (Arm 1A): Best % Change vs. Baseline, is presented in FIG. 44A. FIG. 44B shows AU-007+Aldesleukin: Best % Change vs. Baseline for all response evaluable patients who received AU-007+aldesleukin. ** Patient had a new brain lesion stabilized with radiation. FIG. 44C presents the data for AU-007+Aldesleukin: Best % Change vs. Baseline Immune Sensitive Tumors (G.I. Cancers Excluded). This includes all response evaluable patients with non-G.I. cancer who received AU-007+aldesleukin.

FIGS. 45A and 45B present AU-007 Spider Plot (FIG. 45A) and AU-007+Aldesleukin (IL-2) (FIG. 45B): Percent (%) Tumor Changes Over Time. FIG. 45B includes all response evaluable patients who received AU-007+aldesleukin as of Oct. 13, 2023.

FIGS. 46A-46C presents tumor assessments by computed tomography scans (baseline and 8-week scans) from melanoma patient, wherein the cancer did not respond to checkpoint inhibitors anti-PD-1 and/or CTLA4.

FIG. 47 presents graphs of AU-007 concentration over time. The left-side figure is an expansion of the first 60 hours demonstrating the Tmax and C-max. The right-side figure represents the data set as of June 2023. Note that not all cohorts have complete data as this data is currently being acquired. Overall, the data show AU007 demonstrates typical IgG1 therapeutic characteristics.

FIGS. 48A and 48B show that AU-007 pharmacodynamic data demonstrating AU-007 continues to decrease peripheral blood circulating Tregs (as measured by flow cytometry) over time (days) following administration of AU-007. Percent change in the absolute number of circulating regulatory T cells. Regulatory T cells were defined as CD3+CD4+CD25+CD1271o of the CD45+cells. Consistent with the mechanism of action of inhibiting IL-2 from interacting with the trimeric receptor, regulatory T cells decreased in the peripheral circulation. This was observed in both the monotherapy arm and the arms which also included Proleukin and was consistent among patients. FIG. 48A presents the data per individual patient (receiving AU-007+/− at least one dose of Proleukin—See Key in FIG. 49) and FIG. 48B presents the data per dosage group (AU-007 only). The values represent the change from baseline of the absolute (abs) cell counts.

FIG. 49 presents average percent change/days in peripheral blood Tregs for cohorts receiving at least one dose of Proleukin® (aldesleukin) along with 4.5 mg/kg of AU-007. The values represent the change from baseline of the absolute (abs) cell counts. Data points are continued to be collected.

FIGS. 50A and 50B show changes in absolute peripheral blood CD8 cells over time (days) following administration of AU-007. FIG. 50A presents the data per individual patient and FIG. 50B presents the data per dosage group. The values represent the change from baseline of the absolute (abs) cell counts. Data collection is ongoing.

FIG. 51 shows average percent change/day in peripheral blood CD8 for cohorts receiving Proleukin® (aldesleukin) along with administration of AU-007. The values represent the change from baseline of the absolute (abs) cell counts. Data collection is ongoing.

FIGS. 52A and 52B show changes in absolute peripheral blood NK cells over time (days) following administration of AU-007. FIG. 52A presents the data per individual patient and FIG. 52B presents the data per dosage group. The values represent the change from baseline of the absolute (abs) cell counts. Data collection is ongoing.

FIG. 53 shows change in peripheral blood NK cells for cohorts receiving Proleukin® (aldesleukin) over time (days) following administration of AU-007. The values represent the change from baseline of the absolute (abs) cell counts. Data as of September 2023

FIGS. 54A and 54B show absolute numbers of eosinophils overtime (days) over an extended time period following administration of AU-007. FIG. 54A Individual Peripheral Blood Eosinophil Counts in AU-007-Only Cohorts. FIG. 54B Individual Peripheral Blood Eosinophil Counts in AU-007+Proleukin Cohorts. FIGS. 54A and 54B show changes over time in the circulating number of eosinophils. FIG. 54A are the cohorts receiving only AU-007 monotherapy and FIG. 54B are cohorts receiving AU-007 with at least 1 dose of Proleukin. All but one patient in the AU-007 monotherapy and AU-007 with Proleukin arms demonstrated a decrease or no change in the circulating levels of eosinophils. A patient in the 9 mg/kg cohort had severe seasonal allergies requiring treatment during time on AU-007 treatment and is consistent with a history of being treated for seasonal allergies. The rise in eosinophils was attributed to the allergy reaction. All patients given AU-007 with Proleukin showed stable or a decrease in circulating eosinophils. This is consistent with the mechanism of action of AU-007 preventing IL-2 from interacting with the IL-2 trimeric receptor on eosinophils. The values represent the change from baseline of the absolute (abs) cell counts. Data collection is ongoing.

FIGS. 55A-55C show the CD8: Treg ratios over time (days) over an extended time period in the periphery, following administration of AU-007. FIG. 55A shows all available data per individual patient. (For key, see FIG. 55C) FIG. 55B presents the data per dosage group. FIG. 55C shows data per individual patient also receiving Proleukin® (aldesleukin). Consistent with the observations seen in the changes in Tregs and CD8+ T cells, there is an observed trend to an increase in the CD8+/Treg ratio with monotherapy. In the presence of Proleukin, an increase in the CD8+/Treg ratio was observed, particularly at higher doses of Proleukin. Consistent with the mechanism of action, higher doses (of low dose IL-2) and longer exposure trend to higher CD8+/Treg ratios with no observed drug-related toxicity. It is anticipated that increasing doses of Proleukin will further enhance the peripheral response. The values represent the change from baseline of the absolute (abs) cell counts. Data collection is ongoing.

FIGS. 56A and 56B present the fold change in the expression of IFN-γ in patients dosed with AU-007+/−Proleukin. A heat map of the change from baseline in the circulating levels of interferon gamma (IFN-γ). Light green represents a 20%-2-fold change, mid-green a 2-5-fold change and dark green >5-fold change. These preliminary results demonstrate that the longer a patient is on monotherapy (FIG. 56A), the more likely the patients is to have increases in circulating IFN-γ. This is consistent with the observations in circulating cell populations, particularly Treg and NK cells. The addition of low dose IL-2 in the presence of AU-007 (FIG. 56B) consistently increases IFN-γ in the peripheral circulation.

FIG. 57 presents the hPBMCs expansion assay protocol.

FIGS. 58A and 58B present the fold change of CD4+Tregs from CD4+ cells over time, in vitro, in presence of a control antibody (black), AU-007 (red), or CD25-IL-2 conjugate (green). FIG. 58A presents the results of low dose administration (10 pM) and FIG. 58B presents the results of high dose administration (1 nM). The data demonstrate that while both IL-2 alone or the non-alpha CD25 conjugated IL2 expand the Treg population, in the presence of AU-007 there is no Treg expansion. Since the non-alpha IL-2 does not bind the trimeric receptor on Tregs, the Treg population expansion is a direct result of IL-2 produced by the T cells (endogenous IL-2). AU-007 not only inhibits the exogenous IL-2 but endogenous IL-2 as well.

FIGS. 59A and 59B present the fold change of CD4+Tregs from CD4+ cells over time, in vitro, in presence of a control antibody (blue) or AU-007 (green), in the absence of IL-2. Control wells from Low (FIG. 59A) and High dose (FIG. 59B) IL-2 experiments where no IL-2 was added. The graphs were normalized to wells with no treatment (i.e., media alone). The results demonstrate that AU-007 inhibits the ability of Tregs to use the low levels of endogenous IL-2.

FIGS. 60A and 60B present the fold change of CD4+ Regulatory T cells—(CD3+CD4+CD127-CD25+FoxP3+) (FIG. 60A) and NK (FIG. 60B) cells over time, in vitro, in presence of a control antibody (black), AU-007 (red), or CD25-IL-2 conjugate (green). (IL-2 and CD25-IL-2 conjugate were at 1 nM, Isotype control antibody and AU-007 were at 1 uM; Key in FIG. 60B)

FIGS. 61A-61C present the fold change over time using mean fluorescence intensity (MFI) to show change of Tregs' suppressive markers (CD25 & FoxP3; FIGS. 61A and 61B, respectively) and NK cells activation marker (CD56; FIG. 61C). Observation was 3-6 days post-treatment.

FIGS. 62A and 62B present the ratio of ΔCD8+ Teffs:ΔTregs in the presence of low dose IL-2 (10 pM; FIG. 62A) and high dose IL-2 (1 nM; FIG. 62B)

FIG. 63 presents the experimental design scheme for assaying restimulation-induced cell death (RICD).

FIG. 64 presents a graph showing percent (%) live lymphocytes over time (days), following administration of 1 nM IL-2 (black) or 1 nM IL-2 and 10 μM AU-007 (green).

FIG. 65 presents the experimental design scheme for assaying tetanus toxoid (TT).

FIGS. 66A-66C graphically present the results of the TT assay at low dose TT (0.9 g/ml TT;

FIG. 66A) and high dose TT (9.0 pg/ml TT; FIG. 66B). FIG. 66C shows the fold change of Tregs (CD25+) overtime.

FIGS. 67A and 67B graphically present restimulation induced cell death (RICD) of CD8+cells in cultures stimulated with the protein antigen Tetanus Toxoid (high dose—9.0 pg/ml; FIG. 67A and low dose—0.9 pg/ml; FIG. 67B).

FIG. 68 presents toxicokinetics (TK) from an acute toxicity study in cynomolgus (cyno) monkeys.

FIGS. 69A-69C show AU-007 decreases CD4+CD25+ cells relative to placebo in cynomolgus monkey studies. FIG. 69A-PBS (n=12) vs 5 mg/kg (n=8); FIG. 69B-PBS (n=12) vs 25 mg/kg (n=8); and FIG. 69C-PBS (n=12) vs 100 mg/kg (n=12). PBS values plotted in black in each graph. Each point represents a pre-dose value on each dosing day. Mean is +/− SEM. Each plot represents the percentage of CD25+ cells of the CD4+ population per cohort.

FIGS. 70A-70C show AU-007 increases peripheral NK cells relative to baseline in cynomolgus monkey studies. FIG. 70A—PBS (n=12) vs 5 mg/kg (n=8); FIG. 70B—PBS (n=12) vs 25 mg/kg (n=8); and FIG. 70C—PBS (n=12) vs 100 mg/kg (n=12). PBS values plotted in black in each graph. Each point represents a pre-dose value on each dosing day. Mean is +/− SEM.

FIG. 71 shows that in cyno monkeys given AU-007, IL-2 increases in a dose dependent manner compared with saline (PBS) control.

FIG. 72 presents simulated human pharmacokinetic (PK) profiles following Q2W dosing regimens.

FIG. 73 presents simulated steady-state human PK profiles based on the Q2W dosing regimens.

FIG. 74 shows a table presenting the details of human PK modeling of Q2W AU-007 dosing and il-2 coverage. Reference superscripts—a: Amount of IL-2 (in IU/cc) that could bind to AU-007 on a straight one to one molecule stoichiometric basis; b: Amount of HD IL-2 (600,000 IU/kg) based on 80 kg patient (5 L blood volume) that could bind to AU-007 on a straight one to one molecule stoichiometric basis; c: Between 6th and 7th dose: 12-14 weeks; and d: Amount of HD IL-2 (600,000 IU/kg) based on 80 kg patient (5 L blood volume) that could bind to AU-007 considering 2 IL-2 molecules to 1 AU-007 molecule.

FIG. 75 shows that administration of AU-007 in combination with subcutaneous administration of IL-2 (Proleukin® (aldesleukin)) is projected to send much more daily IL-2 to dimeric receptors on Teff and NK cells, than competing products can achieve.

FIG. 76 presents the Phase ½ clinical study with a focus now on the Phase 1 Dose Escalation portion (IC).

FIG. 77 presents an updated AU-007+Aldesleukin Waterfall Plot: Best % Change vs. Baseline.

FIG. 78 presents an updated AU-007+Aldesleukin Spider Plot: % Change vs. Baseline Over Time, pointing out the greater than 30% cancer reduction in a patient with nasopharyngeal cancer. The data shown includes all response evaluable patients who received AU-007+aldesleukin (IL-2).

FIG. 79 presents an updated AU-007+Aldesleukin Waterfall Plot: Best % Change in Immune Sensitive Tumors. The data shown includes all response evaluable patients with non-G.I. cancer who received AU-007+aldesleukin (IL-2).

FIG. 80 presents computed tomography scans showing 40% shrinkage in the target lesions of a melanoma patient whose tumors progressed through prior anti-PD-1+CTLA4 therapy.

FIG. 81 presents computed tomography scans showing 20% shrinkage in first 8 weeks in the target lesions of a RCC patient whose tumors progressed through prior anti-PD-1 therapy.

### DETAILED DESCRIPTION

The present disclosure provides engineered anti-human IL-2 antibodies that bind human IL-2 with high affinity (e.g., 12.7 pM to 48 pM) to a pre-defined binding epitope. The antibodies bind to IL-2 in a manner that completely prevents CD25 binding, yet spares the binding of IL-2 to CD122, thereby modulating immune responses towards immune stimulation by directly activating and expanding effector cells without interacting with CD25-expressing cells (e.g., regulatory T-cells, short lived cytotoxic T-cells, pulmonary endothelial cells and vascular endothelial cells). Thus, the antibody/IL-2 complex would drive a robust immune response to clear viral load or tumor by expanding and activating effector cells such as NK cells, central memory T cells and virus or tumor-specific T-cells while inhibiting IL-2 activation induced cell death of the short lived CD25+ cytotoxic T-cells that are important for viral/tumor clearance. The antibody/IL-2 complex would also decrease immunosuppression caused by the regulatory arm of the immune system. Moreover, the antibody/IL-2 complex would prevent undesired interactions of IL-2 with vascular and pulmonary CD25-expressing cells, thereby preventing severe syndromes of IL-2 induced vascular leakage and IL-2 induced pulmonary edema frequently seen in models of viral lung infections. In some embodiments, the activity of the engineered anti-IL-2 antibodies described herein is dependent on the pre-defined epitope to which they are designed to bind.

In some embodiments, the IL-2 antibodies disclosed herein, block IL-2 binding to CD25. In some embodiments, the IL-2 antibodies disclosed herein binds IL-2 and prevents newly secreted endogenous IL-2 from binding to Tregs, effectively blocking the negative feedback loop of IL-2 to Tregs. In some embodiments, the IL-2 antibodies disclosed herein prevent Treg expansion. In some embodiments, the IL-2 antibodies disclosed herein block IL-2 binding to vascular endothelium. In some embodiments, the IL-2 antibodies disclosed herein block IL-2 binding to pulmonary endothelium. In some embodiments, the IL-2 antibodies disclosed herein block IL-2 binding to vascular and pulmonary endothelium.

A skilled artisan would appreciate that in certain embodiments, the term “anti-IL-2 antibody” as used herein is interchangeable with the term “anti-human-IL-2 antibody”, having all the same qualities and meanings. Similarly, as used throughout, in certain embodiments, the term “IL-2” is interchangeable with the term “human IL-2”, having all the same qualities and meanings.

In some embodiments, an anti-human IL-2 antibody described herein inhibits binding of IL-2 with an IL-2 receptor alpha (IL-2 Ra, i.e., CD25) subunit and therefore inhibits binding to the trimer IL-2 Rαβγ receptor. In certain embodiments, anti-IL-2 antibodies that inhibit binding of IL-2 with a trimer IL-2 receptor (IL-2 Rαβγ) do not inhibit binding of IL-2 with the dimer IL-2 receptor (IL-2 Rβγ).

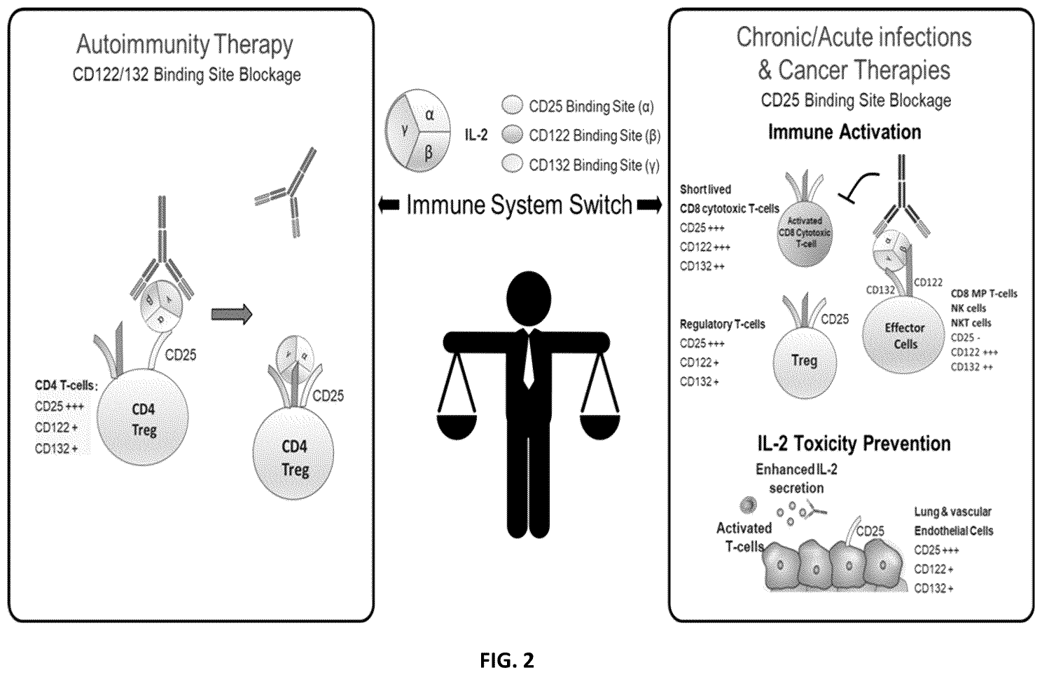

FIG. 2 presents a schematic of anti-IL-2 antibodies directed immunotherapy. Targeting IL-2 to different cell populations can be used to either modulate the immune response toward immunosuppression or towards immune activation. The anti-human IL-2 antibodies disclosed herein are designed to bind with high affinity to an IL-2 epitope that blocks IL-2 binding to CD25. As a result, IL-2 is prevented from binding to short-lived CD8+ cytotoxic T cells or regulatory T cells that express high level of CD25 but is redirected to preferentially bind to effector T cells to stimulate enhanced immune response to improve viral or bacterial clearance. Moreover, since IL-2 binding to CD25-expressing endothelial cells is also blocked, IL-2 induced pulmonary edema and vascular leaking would also be prevented.

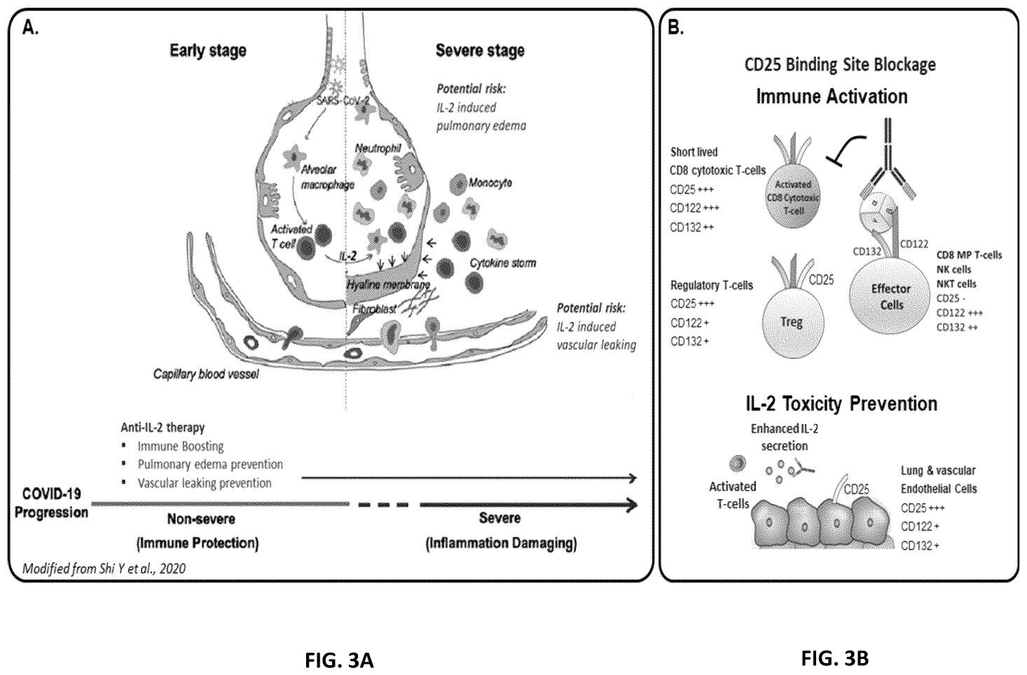

In one embodiment, the present disclosure provides a method of treating a disease (e.g., viral infection, bacterial infection, or cancer), or a condition (e.g., an undesirable condition caused by IL-2, for example but not limited to lung edema) with an anti-IL-2 antibody designed to enhance T cell immune response and to prevent severe edema symptoms of acute pneumonia induced by IL-2. The anti-IL-2 antibody would bind specifically to human IL-2 with high affinity at a pre-defined epitope that blocks IL-2 binding to the alpha chain of the IL-2 receptors (CD25) while sparing binding to the main signaling beta chain and gamma chain complex of the receptor (CD122/CD132). Consequently, in the presence of such antibody, IL-2 would be directed to immune cells responsible for viral/tumor clearance and away from cells that slow the immune response or cause the edema. The formation of this IL-2/antibody immunocomplex will direct IL-2 to bind and activate exclusively naive and memory T lymphocytes, NK cells, and Natural Killer T lymphocytes while preventing activation of regulatory T cells and apoptosis of short-lived CD25+ cytotoxic T effector cells. Altogether, the end result is an effective immune response, for example, viral or tumor clearance. In addition, this treatment will prevent the toxicity caused by IL-2 binding to endothelial CD25 expressing cells. Thus, in one embodiment, targeting IL-2 with the anti-IL-2 antibodies disclosed herein would be an effective treatment for respiratory diseases caused by viral or bacterial infections. In another embodiment, treatment with the anti-IL-2 antibodies disclosed herein would be effective in preventing the toxicity caused by IL-2 binding to endothelial CD25 expressing cells, e.g., pulmonary edema, or IL-2-induced vascular leakage. More importantly, the enhancement of IL-2 immune stimulation towards general immune activation and expansion of immune effector cells independent of a specific pathogen (e.g., a viral antigen) would be an effective strategy against future viral or bacterial pandemics caused by an unknown pathogen (FIGS. 3A and 3B).

In one embodiment, the method disclosed herein would be useful against infection caused by SARS Co-V2. The SARS Co-V2 binds angiotensin converting enzyme 2 of lung cells that allow for viral entry and replication. The immune response to viral infections of the lung consists of both the innate and acquired arms of the immune system. As in the cases with many respiratory viruses, clearance of SARS-CoV2 from the lung is expected to be dependent on T cell immune response. The cytokine IL-2 is critical for the expansion of T cells and plays an important role in immune responses to viruses. However, in addition to its pro-stimulatory role IL-2 also induces some adverse side effects like lung edema and vascular leak syndrome through its binding to endothelium expressing the CD25 receptor.

FIGS. 3A and 3B present a schematic of the progression of COVID-19 infection and potential anti-IL-2 therapy as an adjuvant intervention. FIG. 3A shows the invading SARS Co-V2 causes non-severe symptoms and elicits protective immune responses after an incubation period. Successful elimination of the infection relies on the health status of the infected individual. Individuals with poor immune responses to the virus would have difficulty in clearing the virus while individuals with an over robust immune response may lead to pulmonary edema and other cytokine mediated adverse effects. Therefore, strategies that boost immune response and prevent pulmonary edema are desired. While high concentrations of IL-2 would be beneficial for viral clearance, particularly at the early stage, high levels of IL-2 could lead to IL-2-induced pulmonary edema and vascular leaking through interactions between IL-2 and CD25-expressing endothelial cells. FIG. 3B shows that an anti-human IL-2 antibody designed to bind and block the CD25/IL-2 interaction is predicted to enhance expansion of immune effector cells to improve viral clearance and reduce the negative effects of IL-2 binding to CD25 expressed on endothelial cells, thereby preventing IL-2 induced pulmonary edema and vascular leaking.

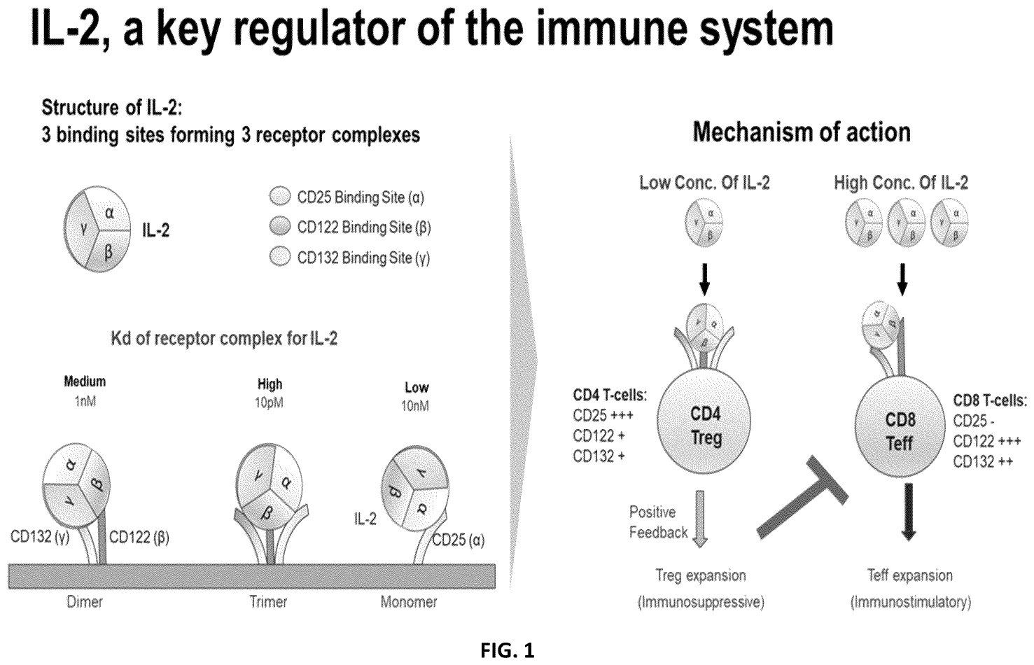

FIG. 1 presents a schematic for the mechanism of action of IL-2 and its dual role in controlling immune response. The left panel shows IL-2 consists of three binding epitope sites (α, β, γ) that interact with different forms of IL2-R (CD25, CD122 and CD132) with different affinities. The right panel shows different IL-2R complexes are expressed on different T cell populations, and their different affinities to IL2 allow immunosuppression under conditions of low local concentrations of IL-2 and immune stimulation when IL-2 local concentration rises.

In the following detailed description, numerous specific details are set forth in order to provide a thorough understanding of the antibodies disclosed herein. However, it will be understood by those skilled in the art that preparation and uses of antibodies disclosed herein may in certain cases be practiced without these specific details. In other instances, well-known methods, procedures, and components have not been described in detail so as not to obscure the disclosure presented herein.

Throughout this application, various references or publications are cited. Disclosures of these references or publications in their entireties are hereby incorporated by reference into this application in order to more fully describe the state of the art to which this invention pertains.

As used herein, the term “antibody” may be used interchangeably with the term “immunoglobulin”, having all the same qualities and meanings. An antibody binding domain or an antigen binding site can be a fragment of an antibody or a genetically engineered product of one or more fragments of the antibody, which fragment is involved in specifically binding with a target antigen. By “specifically binding” is meant that the binding is selective for the antigen of interest and can be discriminated from unwanted or nonspecific interactions. For example, an antibody is said to specifically bind an IL-2 epitope when the equilibrium dissociation constant is ≤10⁻⁵, 10⁻⁶, or 10⁻⁷ M. In some embodiments, the equilibrium dissociation constant may be ≤10⁻⁸ M or 10⁻⁹ M. In some further embodiments, the equilibrium dissociation constant may be ≤10⁻¹⁰ M, 10⁻¹¹ M, or 10⁻¹²M. In some embodiments, the equilibrium dissociation constant may be in the range of ≤10⁻⁵ M to 10⁻¹²M.

As used herein, the term “antibody” encompasses an antibody fragment or fragments that retain binding specificity including, but not limited to, IgG, heavy chain variable region (VH), light chain variable region (VL), Fab fragments, F(ab′)₂ fragments, scFv fragments, Fv fragments, a nanobody, minibodies, diabodies, triabodies, tetrabodies, and single domain antibodies (see, e.g., Hudson and Souriau, Nature Med. 9: 129-134 (2003)). Also encompassed are humanized, primatized, and chimeric antibodies as these terms are generally understood in the art.

As used herein, the term “heavy chain variable region” may be used interchangeably with the term “VH domain” or the term “VH”, having all the same meanings and qualities. As used herein, the term “light chain variable region” may be used interchangeably with the term “VL domain” or the term “VL”, having all the same meanings and qualities. A skilled artisan would recognize that a “heavy chain variable region” or “VH” with regard to an antibody encompasses the fragment of the heavy chain that contains three complementarity determining regions (CDRs) interposed between flanking stretches known as framework regions. The framework regions are more highly conserved than the CDRs, and form a scaffold to support the CDRs. Similarly, a skilled artisan would also recognize that a “light chain variable region” or “VL” with regard to an antibody encompasses the fragment of the light chain that contains three CDRs interposed between framework regions.

As used herein, the term “complementarity determining region” or “CDR” refers to the hypervariable region(s) of a heavy or light chain variable region. Proceeding from the N-terminus, each of a heavy or light chain polypeptide has three CDRs denoted as “CDR1,” “CDR2,” and “CDR3”. Crystallographic analysis of a number of antigen-antibody complexes has demonstrated that the amino acid residues of CDRs form extensive contact with a bound antigen, wherein the most extensive antigen contact is with the heavy chain CDR3. Thus, the CDR regions are primarily responsible for the specificity of an antigen-binding site. In one embodiment, an antigen-binding site includes six CDRs, comprising the CDRs from each of a heavy and a light chain variable region.

As used herein, the term “framework region” or “FR” refers to the four flanking amino acid sequences which frame the CDRs of a heavy or light chain variable region. Some FR residues may contact bound antigen; however, FR residues are primarily responsible for folding the variable region into the antigen-binding site. In some embodiments, the FR residues responsible for folding the variable regions comprise residues directly adjacent to the CDRs. Within FRs, certain amino residues and certain structural features are very highly conserved. In this regard, all variable region sequences contain an internal disulfide loop of around 90 amino acid residues. When a variable region folds into an antigen binding site, the CDRs are displayed as projecting loop motifs that form an antigen-binding surface. It is generally recognized that there are conserved structural regions of FR that influence the folded shape of the CDR loops into certain “canonical” structures regardless of the precise CDR amino acid sequence. Furthermore, certain FR residues are known to participate in non-covalent interdomain contacts which stabilize the interaction of the antibody heavy and light chains.

Wu and Kabat (Tai Te Wu, Elvin A. Kabat. An analysis of the sequences of the variable regions of bence jones proteins and myeloma light chains and their implications for antibody complementarity. Journal of Experimental Medicine, 132, 2, 8 (1970); Kabat E A, Wu T T, Bilofsky H, Reid-Miller M, Perry H. Sequence of proteins of immunological interest. Bethesda: National Institute of Health; 1983. 323 (1983)) pioneered the alignment of antibody peptide sequences, and their contributions in this regard were several-fold: Firstly, through study of sequence similarities between variable domains, they identified correspondent residues that to a greater or lesser extent were homologous across all antibodies in all vertebrate species, inasmuch as they adopted similar three-dimensional structure, played similar functional roles, interacted similarly with neighboring residues, and existed in similar chemical environments. Secondly, they devised a peptide sequence numbering system in which homologous immunoglobulin residues were assigned the same position number. One skilled in the art can unambiguously assign to any variable domain sequence what is now commonly called Kabat numbering without reliance on any experimental data beyond the sequence itself. Thirdly, Kabat and Wu calculated variability for each Kabat-numbered sequence position, by which is meant the finding of few or many possible amino acids when variable domain sequences are aligned. They identified three contiguous regions of high variability embedded within four less variable contiguous regions. Kabat and Wu formally demarcated residues constituting these variable tracts, and designated these “complementarity determining regions” (CDRs), referring to chemical complementarity between antibody and antigen. A role in three-dimensional folding of the variable domain, but not in antigen recognition, was ascribed to the remaining less-variable regions, which are now termed “framework regions”. Fourth, Kabat and Wu established a public database of antibody peptide and nucleic acid sequences, which continues to be maintained and is well known to those skilled in the art.

Chothia and coworkers (Cyrus Chothia, Arthur M. Lesk. Canonical structures for the hypervariable regions of immunoglobulins. Journal of Molecular Biology, 196, 4, 8 (1987)) found that certain sub portions within Kabat CDRs adopt nearly identical peptide backbone conformations, despite having great diversity at the level of amino acid sequence. These sub portions were designated as L1, L2 and L3 or H1, H2 and H3, where the “L” and the “H” designates the light chain and the heavy chains regions, respectively. These regions may be referred to as Chothia CDRs, which have boundaries that overlap with Kabat CDRs.

More recent studies have shown that virtually all antibody binding residues fall within regions of structural consensus. (Kunik, V. et al., PloS Computational Biology 8 (2):e1002388 (February 2012)). In some embodiments, these regions are referred to as antibody binding regions. It was shown that these regions can be identified from the antibody sequence as well. “Paratome”, an implementation of a structural approach for the identification of structural consensus in antibodies, was used for this purpose. (Ofran, Y. et al., J. Immunol. 757:6230-6235 (2008)). While residues identified by Paratome cover virtually all the antibody binding sites, the CDRs (as identified by the commonly used CDR identification tools) miss significant portions of them. Antibody binding residues which were identified by Paratome but were not identified by any of the common CDR identification methods are referred to as Paratome-unique residues. Similarly, antibody binding residues that are identified by any of the common CDR identification methods but are not identified by Paratome are referred to as CDR-unique residues. Paratome-unique residues make crucial energetic contributions to antibody-antigen interactions, while CDRs-unique residues make a rather minor contribution. These results allow for better identification of antigen binding sites.

IMGT® is the international ImMunoGeneTics information system®, (See, Nucleic Acids Res. 2015 January; 43 (Database issue):D413-22. doi: 10.1093/nar/gku1056. Epub 2014 Nov. 5 Free article. PMID: 25378316 LIGM: 441 and Dev Comp Immunol. 2003 January; 27(1):55-77). IMGT is a unique numbering system for immunoglobulin and T cell receptor variable domains and Ig superfamily V-like domains (Lefranc et al., Dev Comp Immunol. 27: 55-77 (2003)). IMGT® presents a uniform numbering system for these IG and TcR variable domain sequences, based on aligning 5 or more IG and TcR variable region sequences, taking into account and combining the Kabat definition of FRs and CDRs, structural data, and Chothia's characterization of the hypervariable loops. IMGT is considered well known in the art as a universal numbering scheme for antibodies.

In some embodiments, identification of potential variant amino acid positions in the VH and VL domains uses the IMGT system of analysis. In some embodiments, identification of potential variant amino acid positions in the VH and VL domains uses the Paratome system of analysis. In some embodiments, identification of potential variant amino acid positions in the VH and VL domains uses the Kabat system of analysis. In some embodiments, identification of potential variant amino acid positions in the VH and VL domains uses the Clothia system of analysis.

In describing variant amino acid positions present in the VH and VL domains, in some embodiments the IMGT numbering is used. In describing variant amino acid positions present in the VH and VL domains, in some embodiments the Paratome numbering is used. In describing variant amino acid positions present in the VH and VL domains, in some embodiments the Kabat numbering is used. In describing variant amino acid positions present in the VH and VL domains, in some embodiments the Clothia numbering is used.

Antigen binding sequences are conventionally located within the heavy chain and light chain variable regions of an antibody. These heavy and light chain variable regions may, in certain instances, be manipulated to create new binding sites, for example to create antibodies or fragments thereof, that bind to a different antigen or to a different epitope of the same antigen. In some embodiments, as described herein, manipulating the sequences of a heavy chain variable region or the sequences of a light chain variable region, or both, would create a new binding site for a second antigen.

An antibody may exist in various forms or having various domains including, without limitation, a complementarity determining region (CDR), a variable region (Fv), a VH domain, a VL domain, a single chain variable region (scFv), and a Fab fragment.

A person of ordinary skill in the art would appreciate that a scFv is a fusion polypeptide comprising the variable heavy chain (VH) and variable light chain (VL) regions of an immunoglobulin, connected by a short linker peptide, the linker may have, for example, 10 to about amino acids.

A skilled artisan would also appreciate that the term “Fab” with regard to an antibody generally encompasses that portion of the antibody consisting of a single light chain (both variable and constant regions) bound to the variable region and first constant region of a single heavy chain by a disulfide bond, whereas F(ab′)₂ comprises a fragment of a heavy chain comprising a VH domain and a light chain comprising a VL domain.

In some embodiments, an antibody encompasses whole antibody molecules, including monoclonal and polyclonal antibodies. In some embodiments, an antibody encompasses an antibody fragment or fragments that retain binding specificity including, but not limited to, variable heavy chain (VH) fragments, variable light chain (VL) fragments, Fab fragments, F(ab′)₂ fragments, scFv fragments, Fv fragments, minibodies, diabodies, triabodies, and tetrabodies.

### Engineered Anti-IL-2 Antibodies

In one embodiment, the present disclosure provides engineered anti-IL-2 antibodies resulted from introducing amino acid variations to a parent anti-IL-2 antibody. In one embodiment, the one or more of the amino acid variations are introduced in a CDR region. In another embodiment, the one or more amino acid variations are introduced within a framework (FR) region. In yet another embodiment, the amino acid variations are introduced to both the CDR region and the framework (FR) region. One of ordinary skill in the art would readily employ various standard techniques known in the art to introduce amino acid variations into an anti-IL-2 antibody and then test the resulting modified antibodies for any changes of binding to IL-2. While standard techniques may be used, the resultant binding pattern of the newly created antibodies is not predictable and must be analyzed to determine functionality.

In certain embodiments, the present disclosure provides polypeptides comprising the VH and VL domains which could be dimerized under suitable conditions. For example, the VH and VL domains may be combined in a suitable buffer and dimerized through appropriate interactions such as hydrophobic interactions. In another embodiment, the VH and VL domains may be combined in a suitable buffer containing an enzyme and/or a cofactor which can promote dimerization of the VH and VL domains. In another embodiment, the VH and VL domains may be combined in a suitable vehicle that allows them to react with each other in the presence of a suitable reagent and/or catalyst.

In certain embodiments, the VH and VL domains may be contained within longer polypeptide sequences, which may include for example but not limited to, constant regions, hinge regions, linker regions, Fc regions, or disulfide binding regions, or any combination thereof. A constant domain is an immunoglobulin fold unit of the constant part of an immunoglobulin molecule, also referred to as a domain of the constant region (e.g., CH1, CH2, CH3, CH4, Ck, Cl). In some embodiments, the longer polypeptides may comprise multiple copies of one or both of the VH and VL domains generated according to the method disclosed herein; for example, when the polypeptides generated herein are used to forms a diabody or a triabody.

In some embodiments, the Fc region comprises at least one mutation that reduces Fc-gamma binding, i.e., binding to a Fcγ receptor (FcγRs). In some embodiments, reduced binding is abolished binding, which binding to the Fcγ receptor is not detectable. In some embodiments, reduced binding reduces the binding affinity to a Fcγ receptor. In some embodiments, reduced binding reduces the on rate for binding to a Fcγ receptor. In some embodiments, reduced binding reduces the off rate of binding to a Fcγ receptor. In some embodiments, a mutation that reduces the Fc-gamma binding comprises L234A, L235A mutations, also known as LALA mutations. In some embodiments, a mutation that reduces the Fc-gamma binding comprises a P329G mutation in addition to the L234A, L235A mutations. In some embodiments, an antibody described herein comprises a heavy chain comprising a mutation that reduces binding to Fcγ receptor.

In one embodiment, the present disclosure provides an engineered (or modified) anti-IL-2 antibody, wherein the antibody comprises a heavy chain variable region having the sequence of one of SEQ ID NOs:10, 12, 14, 16, 18, 20, 22, 24, 26, or 36. In one embodiment, the engineered antibody can be an IgG, IgA, IgM, IgE, IgD, a Fv, a scFv, a Fab, or a F(ab′)₂. The IgG can be of the subclass of IgG1, IgG2, IgG3, or IgG4. In another embodiment, the engineered antibody can be part of a minibody, a diabody, or a triabody antibody.

In one embodiment, the present disclosure provides an engineered (or modified) anti-IL-2 antibody, wherein the antibody comprises a light chain variable region having the sequence of one of SEQ ID NOs:11, 13, 15, 17, 19, 21, 23, 25, 27, or 37. In one embodiment, the engineered antibody can be an IgG, IgA, IgM, IgE, IgD, a Fv, a scFv, a Fab, or a F(ab′)₂. The IgG can be of the subclass of IgG1, IgG2, IgG3, or IgG4. In another embodiment, the engineered antibody can be part of a minibody, a diabody, or a triabody antibody.

In one embodiment, the present disclosure provides an engineered (or modified) anti-IL-2 antibody, wherein the antibody comprises a heavy chain variable region and a light chain variable region having the sequences of one of SEQ ID NOs:10 and 11; SEQ ID NOs:12 and 13; SEQ ID NOs:14 and 15; SEQ ID NOs:16 and 17; SEQ ID NOs:18 and 19; SEQ ID NOs:20 and 21; SEQ ID NOs:22 and 23; SEQ ID NOs:24 and 25; SEQ ID NOs:26 and 27, or SEQ ID NOs: 36 and 37. In one embodiment, the engineered anti-IL-2 antibody comprises the sequences of SEQ ID NOs:10 and 11. In one embodiment, the engineered anti-IL-2 antibody comprises the sequences of SEQ ID NOs:12 and 13. In one embodiment, the engineered anti-IL-2 antibody comprises the sequences of SEQ ID NOs:14 and 15. In one embodiment, the engineered anti-IL-2 antibody comprises the sequences of SEQ ID NOs:16 and 17. In one embodiment, the engineered anti-IL-2 antibody comprises the sequences of SEQ ID NOs:18 and 19. In one embodiment, the engineered anti-IL-2 antibody comprises the sequences of SEQ ID NOs:20 and 21. In one embodiment, the engineered anti-IL-2 antibody comprises the sequences of SEQ ID NOs:22 and 23. In one embodiment, the engineered anti-IL-2 antibody comprises the sequences of SEQ ID NOs:24 and 25. In one embodiment, the engineered anti-IL-2 antibody comprises the sequences of SEQ ID NOs:26 and 27. In one embodiment, the engineered anti-IL-2 antibody comprises the sequences of SEQ ID NOs:36 and 37.

In some embodiments, an isolated anti-IL-2 antibody comprising a heavy chain variable region (VH) and a light chain variable region (VL), wherein said VH comprises heavy chain complementarity determining regions (HCDRs) HCDR1, HCDR2 and HCDR3, said VL comprises light chain complementarity determining regions (LCDRs) LCDR1, LCDR2 and LCDR3, wherein said CDRs have the amino acid sequences of
(a) the HCDR1 comprises the amino acid sequence of SEQ ID NO:38, the HCDR2 comprises the amino acid sequence of SEQ ID NO:39, the HCDR3 comprises the amino acid sequence of SEQ ID NO:40, the LCDR1 comprises the amino acid sequence of SEQ ID NO:41, the LCDR2 comprises the amino acid sequence of YAS, the LCDR3 comprises the amino acid sequence of SEQ ID NO:43;
(b) the HCDR1 comprises the amino acid sequence of SEQ ID NO:44, the HCDR2 comprises the amino acid sequence of SEQ ID NO:45, the HCDR3 comprises the amino acid sequence of SEQ ID NO:46, the LCDR1 comprises the amino acid sequence of SEQ ID NO:47, the LCDR2 comprises the amino acid sequence of YAS, the LCDR3 comprises the amino acid sequence of SEQ ID NO:49;
(c) the HCDR1 comprises the amino acid sequence of SEQ ID NO:50, the HCDR2 comprises the amino acid sequence of SEQ ID NO:51, the HCDR3 comprises the amino acid sequence of SEQ ID NO:52, the LCDR1 comprises the amino acid sequence of SEQ ID NO:53, the LCDR2 comprises the amino acid sequence of YAS, the LCDR3 comprises the amino acid sequence of SEQ ID NO:55;
(d) the HCDR1 comprises the amino acid sequence of SEQ ID NO:56, the HCDR2 comprises the amino acid sequence of SEQ ID NO:57, the HCDR3 comprises the amino acid sequence of SEQ ID NO:58, the LCDR1 comprises the amino acid sequence of SEQ ID NO:59, the LCDR2 comprises the amino acid sequence of YAS, the LCDR3 comprises the amino acid sequence of SEQ ID NO:61; or
(e) the HCDR1 comprises the amino acid sequence of SEQ ID NO:62, the HCDR2 comprises the amino acid sequence of SEQ ID NO:63, the HCDR3 comprises the amino acid sequence of SEQ ID NO:64, the LCDR1 comprises the amino acid sequence of SEQ ID NO:65, the LCDR2 comprises the amino acid sequence of DAS, the LCDR3 comprises the amino acid sequence of SEQ ID NO:67.

In some embodiments, an isolated anti-IL-2 antibody comprising a heavy chain variable region (VH) and a light chain variable region (VL), wherein said VH comprises heavy chain complementarity determining regions (HCDRs) HCDR1, HCDR2 and HCDR3, said VL comprises light chain complementarity determining regions (LCDRs) LCDR1, LCDR2 and LCDR3, wherein said CDRs have the amino acid sequences of
(a) the HCDR1 comprises the amino acid sequence of SEQ ID NO:38, the HCDR2 comprises the amino acid sequence of SEQ ID NO:39, the HCDR3 comprises the amino acid sequence of SEQ ID NO:40, the LCDR1 comprises the amino acid sequence of SEQ ID NO:41, the LCDR2 comprises the amino acid sequence of YAS, the LCDR3 comprises the amino acid sequence of SEQ ID NO:43.

In some embodiments, the VH and VL have the amino acid sequences wherein the VH comprises the amino acid sequence of SEQ ID NO:10, the VL comprises the amino acid sequence of SEQ ID NO:11; the VH comprises the amino acid sequence of SEQ ID NO:12, the VL comprises the amino acid sequence of SEQ ID NO: 13; the VH comprises the amino acid sequence of SEQ ID NO:14, the VL comprises the amino acid sequence of SEQ ID NO:15; the VH comprises the amino acid sequence of SEQ ID NO:16, the VL comprises the amino acid sequence of SEQ ID NO:17; the VH comprises the amino acid sequence of SEQ ID NO:18, the VL comprises the amino acid sequence of SEQ ID NO:19; the VH comprises the amino acid sequence of SEQ ID NO:20, the VL comprises the amino acid sequence of SEQ ID NO:21; the VH comprises the amino acid sequence of SEQ ID NO:22, the VL comprises the amino acid sequence of SEQ ID NO:23; the VH comprises the amino acid sequence of SEQ ID NO:24, the VL comprises the amino acid sequence of SEQ ID NO:25; the VH comprises the amino acid sequence of SEQ ID NO:26, the VL comprises the amino acid sequence of SEQ ID NO:27; or the VH comprises the amino acid sequence of SEQ ID NO:36, the VL comprises the amino acid sequence of SEQ ID NO:37. In some embodiments, the VH and VL have the amino acid sequences wherein the VH comprises the amino acid sequence of SEQ ID NO:26, the VL comprises the amino acid sequence of SEQ ID NO:27.

In some embodiments, an antibody comprising a heavy chain sequence and a light chain sequence, said heavy chain sequence set forth in SEQ ID NO: 68 and said light chain sequence set forth in SEQ ID NO: 69; said heavy chain sequence set forth in SEQ ID NO: 70 and said light chain sequence set forth in SEQ ID NO: 71; or said heavy chain sequence set forth in SEQ ID NO: 72 and said light chain sequence set forth in SEQ ID NO: 73. In some embodiments, an antibody comprising a heavy chain sequence and a light chain sequence, said heavy chain sequence set forth in SEQ ID NO: 68 and said light chain sequence set forth in SEQ ID NO: 69. In some embodiments, an antibody comprising a heavy chain sequence and a light chain sequence, said heavy chain sequence set forth in SEQ ID NO: 70 and said light chain sequence set forth in SEQ ID NO: 71. In some embodiments, an antibody comprising a heavy chain sequence and a light chain sequence, said heavy chain sequence set forth in SEQ ID NO: 72 and said light chain sequence set forth in SEQ ID NO: 73.

In one embodiment, the engineered antibody can be an IgG, IgA, IgM, IgE, IgD, a Fv, a scFv, a Fab, or a F(ab′)₂. The IgG can be of the subclass of IgG1, IgG2, IgG3, or IgG4. In another embodiment, the engineered antibody can be part of a minibody, a diabody, or a triabody antibody.

In one embodiment, the present disclosure also provides isolated polynucleotide sequence encoding a heavy chain variable region of an anti-IL-2 antibody, wherein the heavy chain variable region comprises the amino acid sequence of one of SEQ ID NOs:10, 12, 14, 16, 18, 20, 22, 24, 26, or 36. In another embodiment, the present disclosure also provides a vector comprising the above-mentioned polynucleotide sequences. In view of the amino acid sequences disclosed herein, one of ordinary skill in the art would readily construct a vector or plasmid to encode for the amino acid sequences. In another embodiment, the present disclosure also provides a host cell comprising the vector provided herein. Depending on the uses and experimental conditions, one of skill in the art would readily employ a suitable host cell to carry and/or express the above-mentioned polynucleotide sequences.

In one embodiment, the present disclosure also provides isolated polynucleotide sequence encoding alight chain variable region of an anti-IL-2 antibody, wherein the light chain variable region comprises the amino acid sequence of one of SEQ ID NOs:11, 13, 15, 17, 19, 21, 23, 25, 27, or 37. In another embodiment, the present disclosure also provides a vector comprising the above-mentioned polynucleotide sequences. In view of the amino acid sequences disclosed herein, one of ordinary skill in the art would readily construct a vector or plasmid to encode for the amino acid sequences. In another embodiment, the present disclosure also provides a host cell comprising the vector provided herein. Depending on the uses and experimental conditions, one of skill in the art would readily employ a suitable host cell to carry and/or express the above-mentioned polynucleotide sequences.

In view of the sequences for the heavy chain variable regions and light chain variable regions disclosed herein, one of ordinary skill in the art would readily employ standard techniques known in the art to construct an anti-IL-2 scFv. In one embodiment, polynucleotide sequences encoding for such anti-IL-2 scFv could have the sequence of one of SEQ ID NOs:1-5 or one of SEQ ID NO: 31-35.

In certain embodiments, an isolated polynucleotide sequence disclosed herein, encoding the heavy chain variable region of an anti-IL-2 antibody, comprises a VH amino acid sequence set forth in the amino acid sequence of any of SEQ ID NO:10, 12, 14, 16, 18, 20, 22, 24, 26, or 36. In some embodiments, a vector comprises the polynucleotide sequence of any of SEQ ID NO:10, 12, 14, 16, 18, 20, 22, 24, 26, or 36. In some embodiments, a host cell comprising the vector comprising the polynucleotide sequence of any of SEQ ID NO:10, 12, 14, 16, 18, 20, 22, 24, 26, or 36.

In certain embodiments, an isolated polynucleotide sequence disclosed herein, encoding the light chain variable region of an anti-IL-2 antibody, comprises a VL amino acid sequence as set forth in the amino acid sequence of any of SEQ ID NO: 11, 13, 15, 17, 19, 21, 23, 25, 27, or 37. In some embodiments, a vector comprises the polynucleotide sequence comprising the amino acid sequence of any of SEQ ID NO: 11, 13, 15, 17, 19, 21, 23, 25, 27, or 37. In some embodiments, a host cell comprises a vector comprising the polynucleotide sequence encoding the amino acid sequence of any of SEQ ID NO: 11, 13, 15, 17, 19, 21, 23, 25, 27, or 37. In some embodiments, an isolated polynucleotide sequence encodes an anti-IL-2 scFv, wherein the polynucleotide sequence is set forth in SEQ ID NO: 1, 2, 3, 4, 5, 31, 32, 33, 34, or 35. In some embodiments, a vector comprises an isolated polynucleotide sequence encodes an anti-IL-2 scFv, wherein the polynucleotide sequence is set forth in SEQ ID NO: 1, 2, 3, 4, 5, 31, 32, 33, 34, or 35. In some embodiments, a host cell comprises a vector comprising an isolated polynucleotide sequence encoding an anti-IL-2 scFv, wherein the polynucleotide sequence is set forth in SEQ ID NO: 1, 2, 3, 4, 5, 31, 32, 33, 34, or 35.

In another embodiment, the present disclosure also provides an isolated anti-IL-2 antibody, wherein the antibody comprises a heavy chain variable region having complementarity determining region (CDR) 1, CDR2 and CDR3. In one embodiment, the CDR1, CDR2 and CDR3 comprise amino acid sequences of SEQ ID NOs:38-40 respectively; SEQ ID NOs:44-46 respectively; SEQ ID NOs:50-52 respectively; SEQ ID NOs:56-58 respectively; or SEQ ID NOs:62-64 respectively. In one embodiment, the antibody can be an IgG, IgA, IgM, IgE, IgD, a Fv, a scFv, a Fab, a F(ab′)₂, a minibody, a diabody, or a triabody antibody. The IgG can be IgG1, IgG2, IgG3, or an IgG4. In one embodiment, the present disclosure also encompasses a composition comprising the above-mentioned antibody and a pharmaceutically acceptable carrier.

In another embodiment, the present disclosure also provides an isolated anti-IL-2 antibody, wherein the antibody comprises a light chain variable region having complementarity determining region (CDR) 1, CDR2 and CDR3. In one embodiment, the CDR1, CDR2 and CDR3 comprise amino acid sequences of SEQ ID NOs:41-43 respectively; SEQ ID NOs:47-49 respectively; SEQ ID NOs:53-55 respectively; SEQ ID NOs:59-61 respectively; or SEQ ID NOs:65-67 respectively. In one embodiment, the antibody can be an IgG, IgA, IgM, IgE, IgD, a Fv, a scFv, a Fab, a F(ab′)₂, a minibody, a diabody, or a triabody antibody. The IgG can be IgG1, IgG2, IgG3, or an IgG4. In one embodiment, the present disclosure also encompasses a composition comprising the above-mentioned antibody and a pharmaceutically acceptable carrier.

In another embodiment, the present disclosure also provides an isolated anti-IL-2 antibody, wherein the antibody comprises a heavy chain variable region having complementarity determining region (CDR) 1, CDR2 and CDR3, and a light chain variable region having CDR1, CDR2 and CDR3. In one embodiment, the heavy chain CDR1, CDR2 and CDR3 comprise amino acid sequences of SEQ ID NOs:38-40 respectively; SEQ ID NOs:44-46 respectively; SEQ ID NOs:50-52 respectively; SEQ ID NOs:56-58 respectively; or SEQ ID NOs:62-64 respectively. In one embodiment, the light chain CDR1, CDR2 and CDR3 comprise amino acid sequences of SEQ ID NOs:41-43 respectively; SEQ ID NOs:47-49 respectively; SEQ ID NOs:53-55 respectively; SEQ ID NOs:59-61 respectively; or SEQ ID NOs:65-67 respectively. In one embodiment, the antibody can be an IgG, IgA, IgM, IgE, IgD, a Fv, a scFv, a Fab, a F(ab′)₂, a minibody, a diabody, or a triabody antibody. The IgG can be IgG1, IgG2, IgG3, or an IgG4. In one embodiment, the present disclosure also encompasses a composition comprising the above-mentioned antibody and a pharmaceutically acceptable carrier.

### Pharmaceutical Compositions

In some embodiments, disclosed herein are compositions for therapeutic use. In some embodiments, a composition described herein comprises an anti-IL-2 antibody as disclosed herein and a pharmaceutically acceptable carrier.

As used herein, the terms “composition” and pharmaceutical composition” may in some embodiments, be used interchangeably having all the same qualities and meanings. In some embodiments, disclosed herein is a pharmaceutical composition for the treatment of a condition or disease as described herein.

In some embodiments, disclosed herein are pharmaceutical compositions for use in a combination therapy.

In another embodiment, disclosed herein are compositions for use treating a disease or condition in a subject. In some embodiments, the disease comprises a viral infection, a bacterial infection, or a cancer. In some embodiments the condition comprises an IL-2 induced condition. In some embodiments, the IL-2 induced condition comprises pulmonary edema or vascular leakage.

### Administration

The VH and/or VL polypeptides disclosed herein can be administered to a subject (e.g., a human or an animal) alone, or in combination with a carrier, i.e., a pharmaceutically acceptable carrier. By pharmaceutically acceptable is meant a material that is not biologically or otherwise undesirable, i.e., the material can be administered to a subject without causing any undesirable biological effects or interacting in a deleterious manner with any of the other components of the pharmaceutical composition in which it is contained. As would be well-known to one of ordinary skill in the art, the carrier is selected to minimize any degradation of the polypeptides disclosed herein and to minimize any adverse side effects in the subject. The pharmaceutical compositions may be prepared by methodology well known in the pharmaceutical art.

The above pharmaceutical compositions comprising the polypeptides disclosed herein can be administered (e.g., to a mammal, a cell, or a tissue) in any suitable manner depending on whether local or systemic treatment is desired. For example, the composition can be administered topically (e.g., ophthalmically, vaginally, rectally, intranasally, transdermally, and the like), orally, by inhalation, or parenterally (including by intravenous drip or subcutaneous, intracavity, intraperitoneal, intradermal, or intramuscular injection). Topical intranasal administration refers to delivery of the compositions into the nose and nasal passages through one or both of the nares. The composition can be delivered by a spraying mechanism or droplet mechanism, or through aerosolization. Delivery can also be directed to any area of the respiratory system (e.g., lungs) via intubation. Alternatively, administration can be intratumoral, e.g., local or intravenous injection.

If the composition is to be administered parenterally, the administration is generally by injection. Injectables can be prepared in conventional forms, either as liquid solutions or suspensions, solid forms suitable for suspension in liquid prior to injection, or as emulsions. Additionally, parental administration can involve preparation of a slow-release or sustained-release system so as to maintain a constant dosage.

In some embodiments, a composition comprises an anti-IL-2 antibody comprising a heavy chain variable region having the sequence of one of SEQ ID NOs:10, 12, 14, 16, 18, 20, 22, 24, 26, or 36. In some embodiments, a composition comprises an anti-IL-2 antibody comprising a light chain variable region having the sequence of one of SEQ ID NOs:11, 13, 15, 17, 19, 21, 23, 25, 27, or 37. In some embodiments, a composition comprises an anti-IL-2 antibody comprising a heavy chain variable region and a light chain variable region having the sequences of one of: SEQ ID NOs:10 and 11; SEQ ID NOs:12 and 13; SEQ ID NOs:14 and 15; SEQ ID NOs:16 and 17; SEQ ID NOs:18 and 19; SEQ ID NOs:20 and 21; SEQ ID NOs:22 and 23; SEQ ID NOs:24 and 25; SEQ ID NOs:26 and 27, or SEQ ID NOs: 36 and 37. In some embodiments, a composition comprises an anti-IL-2 antibody comprising a heavy chain variable domain comprising CDR1, CDR2 and CDR3 regions comprising amino acid sequences of SEQ ID NOs:38-40 respectively; SEQ ID NOs:44-46 respectively; SEQ ID NOs:50-52 respectively; SEQ ID NOs:56-58 respectively; or SEQ ID NOs:62-64 respectively. In some embodiments, a composition comprises an anti-IL-2 antibody comprising a light chain variable domain comprising CDR1, CDR2 and CDR3 regions comprising amino acid sequences of SEQ ID NOs:41-43 respectively; SEQ ID NOs:47-49 respectively; SEQ ID NOs:53-55 respectively; SEQ ID NOs:59-61 respectively; or SEQ ID NOs:65-67, respectively. In some embodiments, a composition comprises an anti-IL-2 antibody comprising a heavy chain variable domain comprising CDR1, CDR2 and CDR3 regions comprising amino acid sequences of SEQ ID NOs:38-40 respectively; SEQ ID NOs:44-46 respectively; SEQ ID NOs:50-52 respectively; SEQ ID NOs:56-58 respectively; or SEQ ID NOs:62-64, respectively, and a light chain variable domain comprising CDR1, CDR2, and CDR3 regions comprising amino acid sequences of SEQ ID NOs:41-43 respectively; SEQ ID NOs:47-49 respectively; SEQ ID NOs:53-55 respectively; SEQ ID NOs:59-61 respectively; or SEQ ID NOs:65-67, respectively.

In some embodiments, a composition comprises an anti-IL-2 antibody comprising any of clones BDG 17.014, BDG 17.023, BDG 17.038, BDG 17.043, BDG 17.053, BDG 17.054, BDG 17.066, BDG 17.067, and BDG 17.069.

In one embodiment, methods disclosed herein administer compositions comprising an anti-IL-2 antibody as disclosed herein, as a monotherapy. In another embodiment, methods disclosed herein administer compositions comprising an anti-IL-2 antibody as disclosed herein, as disclosed herein, in conjunction with a one-time dosage of IL-2. In another embodiment, methods disclosed herein administer a combination therapy comprising compositions comprising an anti-IL-2 antibody as disclosed herein and IL-2.

In some embodiments, where an anti-IL2 antibody and IL-2 are administered, they may be administered in the same composition. In some embodiments, where an anti-IL2 antibody and IL-2 are administered, they may be administered in separate compositions.

In some embodiments, IL-2 may be administered prior to, concurrent with, or following the step of administering the anti-IL2 antibody. In some embodiments, IL-2 administration is prior to administering the anti-IL2 antibody. In some embodiments, IL-2 administration is concurrent with administering the anti-IL2 antibody. In some embodiments, IL-2 administration follows the step of administering the anti-IL2 antibody.

In some embodiments, the route of IL-2 administration is subcutaneous. In some embodiments, IL-2 subcutaneous administration is at much lower doses and much less frequently than the approved regimen of intravenously administered aldesleukin. In some embodiments, the route of anit-IL-2 administration is by intravenous injection. In some embodiments, wherein a subject receives both an anti-IL-2 antibody and IL-2, the route of administration of the anti-IL2 antibody is by intravenous injection and the route of administration of the IL-2 is by subcutaneous injection.

In some embodiments, administration of an anti-IL-2 antibody comprises a monotherapy. In some embodiments, administration of an anti-IL-2 antibody comprises including a loading dose of IL-2 with the anti-IL2 antibody monotherapy. In some embodiments, administration of an anti-IL-2 antibody comprises a combination therapy, wherein anti-IL-2 antibody and IL-2 are administered to a subject at regular intervals.

In some embodiments, multiple doses of an anti-IL2 antibody are administered over a given time period. In some embodiments, doses of an anti-IL2 antibody are administered weekly. In some embodiments, doses of an anti-IL2 antibody are administered bi-weekly (once every two weeks). In some embodiments, doses of an anti-IL2 antibody are administered once every three weeks. In some embodiments, a one-time dose of IL-2 may be administered prior to, concurrent with, or following a first dose of an anti-IL antibody as described herein. In some embodiments, multiple doses of an anti-IL2 antibody and IL-2 are administered over a given time period. In some embodiments, doses of an anti-IL2 antibody and IL-2 are administered weekly. In some embodiments, doses of an anti-IL2 antibody and IL-2 are administered bi-weekly (once every two weeks). In some embodiments, doses of an anti-IL2 antibody and IL-2 are administered once every three weeks.

In some embodiments, an anti-IL2 antibody is administered weekly, bi-weekly, or every three weeks, wherein IL-2 is administered as a one-time dose, weekly, bi-weekly, or once every three weeks, wherein the administration of the anti-IL-2 antibody and the IL-2 are administered concurrently. In some embodiments, an anti-IL2 antibody is administered weekly, bi-weekly, or every three weeks, wherein IL-2 is administered as a one-time dose, weekly, bi-weekly, or once every three weeks, wherein the administration of the anti-IL-2 antibody and the IL-2 are administered independent of each other, for example but not limited to an anti-IL2 antibody is administered weekly, bi-weekly, or every three weeks, wherein IL-2 is administered in a one-time dose or anti-IL2 antibody is administered weekly, bi-weekly, or every three weeks, wherein IL-2 is administered every week or anti-IL2 antibody is administered weekly, bi-weekly, or every three weeks, wherein IL-2 is administered bi-weekly or anti-IL2 antibody is administered weekly, bi-weekly, or every three weeks, wherein IL-2 is administered every three weeks.

In some embodiments, therapeutic dosages of anti-IL-2 are administered over a period of months. In some embodiments, therapeutic dosages of anti-IL-2 are administered for up to 3 months. In some embodiments, therapeutic dosages of anti-IL-2 are administered for at least 3 months. In some embodiments, therapeutic dosages of anti-IL-2 are administered for up to 6 months. In some embodiments, therapeutic dosages of anti-IL-2 are administered for at least 6 months. In some embodiments, therapeutic dosages of anti-IL-2 are administered for up to 9 months. In some embodiments, therapeutic dosages of anti-IL-2 are administered for at least 9 months.

In some embodiments, therapeutic dosages of anti-IL-2 are administered over a period of up to a year. In some embodiments, therapeutic dosages of anti-IL-2 are administered over a period of at least a year.

In some embodiments, an anti-IL-2 antibody disclosed herein, is administered in combination with IL-2, wherein the dose of IL-2 is considered low dose of IL-2. In some embodiments, IL-2 is administered in a single dose (loading dose). In some embodiments, IL-2 is administered over the same period of time as an anti-IL-2 antibody. In some embodiments, IL-2 is administered as multiple doses prior to, concurrent with, or following administration of an anti-IL-2 antibody.

In some embodiments, the dose of an anti-IL-2 antibody disclosed herein is between about 0.5 mg/kg and 12 mg/kg. In some embodiments, the dose of an anti-IL-2 antibody disclosed herein is about 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 9.5 mg/kg, 10.0 mg/kg, 10.5 mg/kg, 11.0 mg/kg, 11.5 mg/kg, and 12 mg/kg.

In some embodiments, the dose of an anti-IL-2 antibody disclosed herein is 0.5 mg/kg. In some embodiments, the dose of an anti-IL-2 antibody disclosed herein is 1.0 mg/kg. In some embodiments, the dose of an anti-IL-2 antibody disclosed herein is 1.5 mg/kg. In some embodiments, the dose of an anti-IL-2 antibody disclosed herein is 2.0 mg/kg. In some embodiments, the dose of an anti-IL-2 antibody disclosed herein is 2.5 mg/kg. In some embodiments, the dose of an anti-IL-2 antibody disclosed herein is 3.0 mg/kg. In some embodiments, the dose of an anti-IL-2 antibody disclosed herein is 3.5 mg/kg. In some embodiments, the dose of an anti-IL-2 antibody disclosed herein is 4.0 mg/kg. In some embodiments, the dose of an anti-IL-2 antibody disclosed herein is 4.5 mg/kg. In some embodiments, the dose of an anti-IL-2 antibody disclosed herein is 5.0 mg/kg. In some embodiments, the dose of an anti-IL-2 antibody disclosed herein is 5.5 mg/kg. In some embodiments, the dose of an anti-IL-2 antibody disclosed herein is 6.0 mg/kg. In some embodiments, the dose of an anti-IL-2 antibody disclosed herein is 6.5 mg/kg. In some embodiments, the dose of an anti-IL-2 antibody disclosed herein is 7.0 mg/kg. In some embodiments, the dose of an anti-IL-2 antibody disclosed herein is 7.5 mg/kg. In some embodiments, the dose of an anti-IL-2 antibody disclosed herein is 8.0 mg/kg. In some embodiments, the dose of an anti-IL-2 antibody disclosed herein is 8.5 mg/kg. In some embodiments, the dose of an anti-IL-2 antibody disclosed herein is 9.0 mg/kg. In some embodiments, the dose of an anti-IL-2 antibody disclosed herein is 9.5 mg/kg. In some embodiments, the dose of an anti-IL-2 antibody disclosed herein is 10.0 mg/kg. In some embodiments, the dose of an anti-IL-2 antibody disclosed herein is 10.5 mg/kg. In some embodiments, the dose of an anti-IL-2 antibody disclosed herein is 11.0 mg/kg. In some embodiments, the dose of an anti-IL-2 antibody disclosed herein is 11.5 mg/kg. In some embodiments, the dose of an anti-IL-2 antibody disclosed herein is 12.0 mg/kg. In some embodiments, the dose of an anti-IL-2 antibody disclosed herein is 12.5 mg/kg.

In some embodiments, the dose of IL-2 comprises a low dose. One skilled in the art would appreciate that a low dose of IL-2 may encompass dose level below those provided in studies currently known in the art. In certain embodiments, an IL-2 dose between about 10×10³ IU/kg-300×10³ IU/kg encompasses a low dose of IL-2. In certain embodiments, an IL-2 dose between about 10×10³ IU/kg-500×10³ IU/kg encompasses a low dose of IL-2.

In some embodiments, the dose of IL-2 is between about 10×10³ IU/kg-500×10³ IU/kg. In some embodiments, the dose of IL-2 is between about 10×10³ IU/kg-300×10³ IU/kg. In some embodiments, the dose of IL-2 is between about 15×10³ IU/kg-270×10³ IU/kg. In some embodiments, the dose of IL-2 is about 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, or 500×10³ IU/kg. In some embodiments, the dose of IL-2 is about 15×10³ IU/kg. In some embodiments, the dose of IL-2 is about 45×10³IU/kg. In some embodiments, the dose of IL-2 is about 135×10³ IU/kg. In some embodiments, the dose of IL-2 is about 270×10³ IU/kg. In some embodiments, the dose of IL-2 is about 300×10³ IU/kg. In some embodiments, the dose of IL-2 is about 400×10³ IU/kg. In some embodiments, the dose of IL-2 is about 500×10³ IU/kg. In some embodiments, the dose of IL-2 is 15×10³ IU/kg. In some embodiments, the dose of IL-2 is 45×10³ IU/kg. In some embodiments, the dose of IL-2 is 135×10³ IU/kg. In some embodiments, the dose of IL-2 is 270×10³ IU/kg. In some embodiments, the dose of IL-2 is 300×10³ IU/kg. In some embodiments, the dose of IL-2 is 400×10³ IU/kg. In some embodiments, the dose of IL-2 is 500×10³ IU/kg.

In some embodiments when administering an anti-IL-2 antibody disclosed herein and a single loading dose of IL-2, the dose of anti-IL2 antibody is between about 0.1 mg/kg-12 mg/kg anti-IL-2 antibody and the dose of IL-2 is between about 10×10³ IU/kg-300×10³ IU/kg. In some embodiments when administering an anti-IL-2 antibody disclosed herein and a single loading dose of IL-2, the dose of anti-IL2 antibody is between about 0.1 mg/kg-12 mg/kg anti-IL-2 antibody and the dose of IL-2 is between about 10×10³ IU/kg-500×10³ IU/kg. In some embodiments when administering an anti-IL-2 antibody disclosed herein and a single loading dose of IL-2, the dose of anti-IL2 antibody is 0.5 mg/kg and the dose of IL-2 is 15×10³ IU/kg. In some embodiments when administering an anti-IL-2 antibody disclosed herein and a single loading dose of IL-2, the dose of anti-IL2 antibody is 0.5 mg/kg and the dose of IL-2 is 45×10³ IU/kg. In some embodiments when administering an anti-IL-2 antibody disclosed herein and a single loading dose of IL-2, the dose of anti-IL2 antibody is 0.5 mg/kg and the dose of IL-2 is 135×10³ IU/kg. In some embodiments when administering an anti-IL-2 antibody disclosed herein and a single loading dose of IL-2, the dose of anti-IL2 antibody is 0.5 mg/kg and the dose of IL-2 is 270×10³ IU/kg. In some embodiments when administering an anti-IL-2 antibody disclosed herein and a single loading dose of IL-2, the dose of anti-IL2 antibody is 0.5 mg/kg and the dose of IL-2 is 300×10³ IU/kg. In some embodiments when administering an anti-IL-2 antibody disclosed herein and a single loading dose of IL-2, the dose of anti-IL2 antibody is 0.5 mg/kg and the dose of IL-2 is 400×10³ IU/kg. In some embodiments when administering an anti-IL-2 antibody disclosed herein and a single loading dose of IL-2, the dose of anti-IL2 antibody is 0.5 mg/kg and the dose of IL-2 is 500×10³ IU/kg.

In some embodiments when administering an anti-IL-2 antibody disclosed herein and a single loading dose of IL-2, the dose of anti-IL2 antibody is 1.5 mg/kg and the dose of IL-2 is 15×10³ IU/kg. In some embodiments when administering an anti-IL-2 antibody disclosed herein and a single loading dose of IL-2, the dose of anti-IL2 antibody is 1.5 mg/kg and the dose of IL-2 is 45×10³ IU/kg. In some embodiments when administering an anti-IL-2 antibody disclosed herein and a single loading dose of IL-2, the dose of anti-IL2 antibody is 1.5 mg/kg and the dose of IL-2 is 135×10³ IU/kg. In some embodiments when administering an anti-IL-2 antibody disclosed herein and a single loading dose of IL-2, the dose of anti-IL2 antibody is 1.5 mg/kg and the dose of IL-2 is 270×10³ IU/kg. In some embodiments when administering an anti-IL-2 antibody disclosed herein and a single loading dose of IL-2, the dose of anti-IL2 antibody is 1.5 mg/kg and the dose of IL-2 is 300×10³ IU/kg. In some embodiments when administering an anti-IL-2 antibody disclosed herein and a single loading dose of IL-2, the dose of anti-IL2 antibody is 1.5 mg/kg and the dose of IL-2 is 400×10³ IU/kg. In some embodiments when administering an anti-IL-2 antibody disclosed herein and a single loading dose of IL-2, the dose of anti-IL2 antibody is 1.5 mg/kg and the dose of IL-2 is 500×10³ IU/kg.

In some embodiments when administering an anti-IL-2 antibody disclosed herein and a single loading dose of IL-2, the dose of anti-IL2 antibody is 4.5 mg/kg and the dose of IL-2 is 15×10³ IU/kg. In some embodiments when administering an anti-IL-2 antibody disclosed herein and a single loading dose of IL-2, the dose of anti-IL2 antibody is 4.5 mg/kg and the dose of IL-2 is 45×10³ IU/kg. In some embodiments when administering an anti-IL-2 antibody disclosed herein and a single loading dose of IL-2, the dose of anti-IL2 antibody is 4.5 mg/kg and the dose of IL-2 is 135×10³ IU/kg. In some embodiments when administering an anti-IL-2 antibody disclosed herein and a single loading dose of IL-2, the dose of anti-IL2 antibody is 4.5 mg/kg and the dose of IL-2 is 270×10³ IU/kg. In some embodiments when administering an anti-IL-2 antibody disclosed herein and a single loading dose of IL-2, the dose of anti-IL2 antibody is 4.5 mg/kg and the dose of IL-2 is 300×10³ IU/kg. In some embodiments when administering an anti-IL-2 antibody disclosed herein and a single loading dose of IL-2, the dose of anti-IL2 antibody is 4.5 mg/kg and the dose of IL-2 is 400×10³ IU/kg. In some embodiments when administering an anti-IL-2 antibody disclosed herein and a single loading dose of IL-2, the dose of anti-IL2 antibody is 4.5 mg/kg and the dose of IL-2 is 500×10³ IU/kg.

In some embodiments when administering an anti-IL-2 antibody disclosed herein and a single loading dose of IL-2, the dose of anti-IL2 antibody is 9.0 mg/kg and the dose of IL-2 is 15×10³ IU/kg. In some embodiments when administering an anti-IL-2 antibody disclosed herein and a single loading dose of IL-2, the dose of anti-IL2 antibody is 9.0 mg/kg and the dose of IL-2 is 45×10³ IU/kg. In some embodiments when administering an anti-IL-2 antibody disclosed herein and a single loading dose of IL-2, the dose of anti-IL2 antibody is 9.0 mg/kg and the dose of IL-2 is 135×10³ IU/kg. In some embodiments when administering an anti-IL-2 antibody disclosed herein and a single loading dose of IL-2, the dose of anti-IL2 antibody is 9.0 mg/kg and the dose of IL-2 is 270×10³ IU/kg. In some embodiments when administering an anti-IL-2 antibody disclosed herein and a single loading dose of IL-2, the dose of anti-IL2 antibody is 9.0 mg/kg and the dose of IL-2 is 300×10³ IU/kg. In some embodiments when administering an anti-IL-2 antibody disclosed herein and a single loading dose of IL-2, the dose of anti-IL2 antibody is 9.0 mg/kg and the dose of IL-2 is 400×10³ IU/kg. In some embodiments when administering an anti-IL-2 antibody disclosed herein and a single loading dose of IL-2, the dose of anti-IL2 antibody is 9.0 mg/kg and the dose of IL-2 is 500×10³ IU/kg.

In some embodiments when administering an anti-IL-2 antibody disclosed herein and a single loading dose of IL-2, the dose of anti-IL2 antibody is 12 mg/kg and the dose of IL-2 is 15×10³ IU/kg. In some embodiments when administering an anti-IL-2 antibody disclosed herein and a single loading dose of IL-2, the dose of anti-IL2 antibody is 12 mg/kg and the dose of IL-2 is 45×10³ IU/kg. In some embodiments when administering an anti-IL-2 antibody disclosed herein and a single loading dose of IL-2, the dose of anti-IL2 antibody is 12 mg/kg and the dose of IL-2 is 135×10³ IU/kg. In some embodiments when administering an anti-IL-2 antibody disclosed herein and a single loading dose of IL-2, the dose of anti-IL2 antibody is 12 mg/kg and the dose of IL-2 is 270×10³ IU/kg. In some embodiments when administering an anti-IL-2 antibody disclosed herein and a single loading dose of IL-2, the dose of anti-IL2 antibody is 12 mg/kg and the dose of IL-2 is 300×10³ IU/kg. In some embodiments when administering an anti-IL-2 antibody disclosed herein and a single loading dose of IL-2, the dose of anti-IL2 antibody is 12 mg/kg and the dose of IL-2 is 400×10³ IU/kg. In some embodiments when administering an anti-IL-2 antibody disclosed herein and a single loading dose of IL-2, the dose of anti-IL2 antibody is 12 mg/kg and the dose of IL-2 is 250×10³ IU/kg.

In some embodiments when administering a combination therapy comprising an anti-IL-2 antibody disclosed herein and IL-2, wherein the dose of anti-IL2 antibody is between about 0.1 mg/kg and 12 mg/kg anti-IL-2 antibody and the dose of IL-2 is between about 10×10³ IU/kg-300×10³ IU/kg. In some embodiments when administering a combination therapy comprising an anti-IL-2 antibody disclosed herein and IL-2, wherein the dose of anti-IL2 antibody is between about 0.1 mg/kg and 12 mg/kg anti-IL-2 antibody and the dose of IL-2 is between about 10×10³ IU/kg-500×10³ IU/kg. In some embodiments when administering a combination therapy comprising an anti-IL-2 antibody disclosed herein and IL-2, wherein the dose of anti-IL2 antibody is between about 0.5 mg/kg and 12 mg/kg anti-IL-2 antibody and the dose of IL-2 is between about 10×10³ IU/kg-300×10³ IU/kg. In some embodiments when administering a combination therapy comprising an anti-IL-2 antibody disclosed herein and IL-2, wherein the dose of anti-IL2 antibody is between about 0.5 mg/kg and 12 mg/kg anti-IL-2 antibody and the dose of IL-2 is between about 10×10³ IU/kg-500×10³ IU/kg.

In some embodiments when administering a combination therapy comprising an anti-IL-2 antibody disclosed herein and IL-2, the dose of anti-IL2 antibody is 0.5 mg/kg and the dose of IL-2 is 15×10³ IU/kg. In some embodiments when administering a combination therapy comprising an anti-IL-2 antibody disclosed herein and IL-2, the dose of anti-IL2 antibody is 0.5 mg/kg and the dose of IL-2 is 45×10³ IU/kg. In some embodiments when administering a combination therapy comprising an anti-IL-2 antibody disclosed herein and IL-2, the dose of anti-IL2 antibody is 0.5 mg/kg and the dose of IL-2 is 135×10³ IU/kg. In some embodiments when administering a combination therapy comprising an anti-IL-2 antibody disclosed herein and IL-2, the dose of anti-IL2 antibody is 0.5 mg/kg and the dose of IL-2 is 270×10³ IU/kg. In some embodiments when administering a combination therapy comprising an anti-IL-2 antibody disclosed herein and IL-2, the dose of anti-IL2 antibody is 0.5 mg/kg and the dose of IL-2 is 300×10³ IU/kg. In some embodiments when administering a combination therapy comprising an anti-IL-2 antibody disclosed herein and IL-2, the dose of anti-IL2 antibody is 0.5 mg/kg and the dose of IL-2 is 400×10³ IU/kg. In some embodiments when administering a combination therapy comprising an anti-IL-2 antibody disclosed herein and IL-2, the dose of anti-IL2 antibody is 0.5 mg/kg and the dose of IL-2 is 500×10³ IU/kg.

In some embodiments when administering a combination therapy comprising an anti-IL-2 antibody disclosed herein and IL-2, the dose of anti-IL2 antibody is 1.5 mg/kg and the dose of IL-2 is 15×10³ IU/kg. In some embodiments when administering a combination therapy comprising an anti-IL-2 antibody disclosed herein and IL-2, the dose of anti-IL2 antibody is 1.5 mg/kg and the dose of IL-2 is 45×10³ IU/kg. In some embodiments when administering a combination therapy comprising an anti-IL-2 antibody disclosed herein and IL-2, the dose of anti-IL2 antibody is 1.5 mg/kg and the dose of IL-2 is 135×10³ IU/kg. In some embodiments when administering a combination therapy comprising an anti-IL-2 antibody disclosed herein and IL-2, the dose of anti-IL2 antibody is 1.5 mg/kg and the dose of IL-2 is 270×10³ IU/kg. In some embodiments when administering a combination therapy comprising an anti-IL-2 antibody disclosed herein and IL-2, the dose of anti-IL2 antibody is 1.5 mg/kg and the dose of IL-2 is 300×10³ IU/kg. In some embodiments when administering a combination therapy comprising an anti-IL-2 antibody disclosed herein and IL-2, the dose of anti-IL2 antibody is 1.5 mg/kg and the dose of IL-2 is 400×10³ IU/kg. In some embodiments when administering a combination therapy comprising an anti-IL-2 antibody disclosed herein and IL-2, the dose of anti-IL2 antibody is 1.5 mg/kg and the dose of IL-2 is 500×10³ IU/kg.

In some embodiments when administering a combination therapy comprising an anti-IL-2 antibody disclosed herein and IL-2, the dose of anti-IL2 antibody is 4.5 mg/kg and the dose of IL-2 is about 10×10³ IU/kg-300 IU/kg. In some embodiments when administering a combination therapy comprising an anti-IL-2 antibody disclosed herein and IL-2, the dose of anti-IL2 antibody is 4.5 mg/kg and the dose of IL-2 is about 10×10³ IU/kg-500 IU/kg.

In some embodiments when administering a combination therapy comprising an anti-IL-2 antibody disclosed herein and IL-2, the dose of anti-IL2 antibody is 4.5 mg/kg and the dose of IL-2 is 15×10³ IU/kg. In some embodiments when administering a combination therapy comprising an anti-IL-2 antibody disclosed herein and IL-2, the dose of anti-IL2 antibody is 4.5 mg/kg and the dose of IL-2 is 45×10³ IU/kg. In some embodiments when administering a combination therapy comprising an anti-IL-2 antibody disclosed herein and IL-2, the dose of anti-IL2 antibody is 4.5 mg/kg and the dose of IL-2 is 135×10³ IU/kg. In some embodiments when administering a combination therapy comprising an anti-IL-2 antibody disclosed herein and IL-2, the dose of anti-IL2 antibody is 4.5 mg/kg and the dose of IL-2 is 270×10³ IU/kg. In some embodiments when administering a combination therapy comprising an anti-IL-2 antibody disclosed herein and IL-2, the dose of anti-IL2 antibody is 4.5 mg/kg and the dose of IL-2 is 300×10³ IU/kg. In some embodiments when administering a combination therapy comprising an anti-IL-2 antibody disclosed herein and IL-2, the dose of anti-IL2 antibody is 4.5 mg/kg and the dose of IL-2 is 400×10³ IU/kg. In some embodiments when administering a combination therapy comprising an anti-IL-2 antibody disclosed herein and IL-2, the dose of anti-IL2 antibody is 4.5 mg/kg and the dose of IL-2 is 500×10³ IU/kg. In some embodiments when administering a combination therapy comprising an anti-IL-2 antibody disclosed herein and IL-2, the dose of anti-IL2 antibody is 9.0 mg/kg and the dose of IL-2 is about 10×10³ IU/kg-300 IU/kg. In some embodiments when administering a combination therapy comprising an anti-IL-2 antibody disclosed herein and IL-2, the dose of anti-IL2 antibody is 9.0 mg/kg and the dose of IL-2 is about 10×10³ IU/kg-500 IU/kg.

In some embodiments when administering a combination therapy comprising an anti-IL-2 antibody disclosed herein and IL-2, the dose of anti-IL2 antibody is 9.0 mg/kg and the dose of IL-2 is 15×10³ IU/kg. In some embodiments when administering a combination therapy comprising an anti-IL-2 antibody disclosed herein and IL-2, the dose of anti-IL2 antibody is 9.0 mg/kg and the dose of IL-2 is 45×10³ IU/kg. In some embodiments when administering a combination therapy comprising an anti-IL-2 antibody disclosed herein and IL-2, the dose of anti-IL2 antibody is 9.0 mg/kg and the dose of IL-2 is 135×10³ IU/kg. In some embodiments when administering a combination therapy comprising an anti-IL-2 antibody disclosed herein and IL-2, the dose of anti-IL2 antibody is 9.0 mg/kg and the dose of IL-2 is 270×10³ IU/kg. In some embodiments when administering a combination therapy comprising an anti-IL-2 antibody disclosed herein and IL-2, the dose of anti-IL2 antibody is 9.0 mg/kg and the dose of IL-2 is 300×10³ IU/kg. In some embodiments when administering a combination therapy comprising an anti-IL-2 antibody disclosed herein and IL-2, the dose of anti-IL2 antibody is 9.0 mg/kg and the dose of IL-2 is 400×10³ IU/kg. In some embodiments when administering a combination therapy comprising an anti-IL-2 antibody disclosed herein and IL-2, the dose of anti-IL2 antibody is 9.0 mg/kg and the dose of IL-2 is 500×10³ IU/kg.

In some embodiments when administering a combination therapy comprising an anti-IL-2 antibody disclosed herein and IL-2, the dose of anti-IL2 antibody is 12 mg/kg and the dose of IL-2 is 15×10³ IU/kg. In some embodiments when administering a combination therapy comprising an anti-IL-2 antibody disclosed herein and IL-2, the dose of anti-IL2 antibody is 12 mg/kg and the dose of IL-2 is 45×10³ IU/kg. In some embodiments when administering a combination therapy comprising an anti-IL-2 antibody disclosed herein and IL-2, the dose of anti-IL2 antibody is 12 mg/kg and the dose of IL-2 is 135×10³ IU/kg. In some embodiments when administering a combination therapy comprising an anti-IL-2 antibody disclosed herein and IL-2, the dose of anti-IL2 antibody is 12 mg/kg and the dose of IL-2 is 270×10³ IU/kg. In some embodiments when administering a combination therapy comprising an anti-IL-2 antibody disclosed herein and IL-2, the dose of anti-IL2 antibody is 12 mg/kg and the dose of IL-2 is 300×10³ IU/kg. In some embodiments when administering a combination therapy comprising an anti-IL-2 antibody disclosed herein and IL-2, the dose of anti-IL2 antibody is 12 mg/kg and the dose of IL-2 is 400×10³ IU/kg. In some embodiments when administering a combination therapy comprising an anti-IL-2 antibody disclosed herein and IL-2, the dose of anti-IL2 antibody is 12 mg/kg and the dose of IL-2 is 500×10³ IU/kg.

In some embodiments, the dose of IL-2 is considered low when compared with other therapies. In some embodiments, a low dose of IL-2 is between about 10×10³ IU/kg-500×10³ IU/kg. In some embodiments, a low dose of IL-2 is between about 10×10³ IU/kg-500×10³ IU/kg. In some embodiments, a low dose of IL-2 is between about 15×10³ IU/kg-500×10³ IU/kg. In some embodiments, a low dose of IL-2 is between about 45×10³ IU/kg-500×10³ IU/kg. In some embodiments, a low dose of IL-2 is equal to or less than about 500×10³IU/kg. In some embodiments, a composition comprises an anti-IL-2 antibody comprising anti-IL-2 clone BDG 17.014. In some embodiments, a composition comprises an anti-IL-2 antibody comprising anti-IL-2 clone BDG 17.023. In some embodiments, a composition comprises an anti-IL-2 antibody comprising anti-IL-2 clone BDG 17.038. In some embodiments, a composition comprises an anti-IL-2 antibody comprising anti-IL-2 clone BDG 17.043. In some embodiments, a composition comprises an anti-IL-2 antibody comprising anti-IL-2 clone BDG 17.053. In some embodiments, a composition comprises an anti-IL-2 antibody comprising anti-IL-2 clone BDG 17.054. In some embodiments, a composition comprises an anti-IL-2 antibody comprising anti-IL-2 clone BDG 17.066. In some embodiments, a composition comprises an anti-IL-2 antibody comprising anti-IL-2 clone BDG 17.067. In some embodiments, a composition comprises an anti-IL-2 antibody comprising anti-IL-2 clone BDG 17.069.

In some embodiments, compositions comprise an anti-IL2 antibody and a pharmaceutically acceptable carrier. In some embodiments, compositions comprise an anti-IL2 antibody and IL-2, and a pharmaceutically acceptable carrier. In some embodiments, compositions comprise an anti-IL2 antibody complexed with IL-2, and a pharmaceutically acceptable carrier.

In some embodiments, an anti-IL-2 antibody and IL-2 are comprised in the same composition. In some embodiments, an anti-IL-2 antibody and IL-2 are comprised in different compositions. In some embodiments, administration of a combination of an anti-IL-2 antibody and IL-2, or composition(s) thereof are concurrent. In some embodiments, administration of a combination of an anti-IL-2 antibody and IL-2, or composition(s) thereof comprises administration of an anti-IL-2 antibody or a composition thereof, prior to the IL-2 or a composition thereof. In some embodiments, administration of a combination of an anti-IL-2 antibody and IL-2, or composition(s) thereof comprises administration of an anti-IL-2 antibody or a composition thereof, following administration of the IL-2 or a composition thereof.

A skilled artisan would appreciate that a “pharmaceutical composition” may encompass a preparation of one or more of the active ingredients described herein with other chemical components such as physiologically suitable carriers and excipients. The purpose of a pharmaceutical composition is to facilitate administration of an active agent, for example but not limited to an antibody or a compound, to an organism.

In some embodiments, disclosed herein is a pharmaceutical composition for a therapy use treating a subject with a weakened immune system. In some embodiments, disclosed herein is a pharmaceutical composition for a therapy use treating a subject suffering from a viral infection, a bacterial infection, or a cancer. In some embodiments, disclosed herein is a pharmaceutical composition for use as part of a combination therapy for treating a subject with a weakened immune system. In some embodiments, disclosed herein is a pharmaceutical composition for use as part of a combination therapy for use treating a subject suffering from a viral infection, a bacterial infection, or a cancer.

A skilled artisan would appreciate that the phrases “physiologically acceptable carrier”, “pharmaceutically acceptable carrier”, “physiologically acceptable excipient”, and “pharmaceutically acceptable excipient”, may be used interchangeably may encompass a carrier, excipient, or a diluent that does not cause significant irritation to an organism and does not abrogate the biological activity and properties of the administered active ingredient.

A skilled artisan would appreciate that an “excipient” may encompass an inert substance added to a pharmaceutical composition to further facilitate administration of an active ingredient. In some embodiments, excipients include calcium carbonate, calcium phosphate, various sugars and types of starch, cellulose derivatives, gelatin, vegetable oils and polyethylene glycols.

Techniques for formulation and administration of drugs are found in “Remington's Pharmaceutical Sciences,” Mack Publishing Co., Easton, PA, latest edition, which is incorporated herein by reference.

In some embodiments, the composition as disclosed herein comprises a therapeutic composition. In some embodiments, the composition as disclosed herein comprises a therapeutic efficacy.

### Combination Therapies

In some embodiments, an anti-IL-2 antibody or composition thereof as disclosed herein, is used as part of a combination therapy. In some embodiments, an anti-IL-2 antibody or composition thereof as disclosed herein, is used in combination with IL-2. In some embodiments, an anti-IL-2 antibody or composition thereof as disclosed herein, is used in combination with IL-2 and with an immune checkpoint inhibitor. In some embodiments, an anti-IL-2 antibody or composition thereof as disclosed herein, is used in combination with an immune checkpoint inhibitor.

In some embodiments, an anti-IL-2 antibody or a composition thereof, is used in combination with an immune checkpoint inhibitor. In some embodiments, the term “immune checkpoint inhibitor” may encompass any compound or molecule capable of inhibiting the function of a checkpoint protein. In some embodiments, the term “immune checkpoint inhibitor” may encompass any compound or molecule which targets immune checkpoint proteins. An artisan would appreciate that “immune checkpoints” are key regulators of the immune system that when stimulated can dampen the immune response to an immunologic stimulus. Checkpoint inhibitors can block inhibitory checkpoints, and thereby restore immune system function. In some embodiments, the one or more checkpoint inhibitors comprise immune checkpoint inhibitors.

A skilled artisan would appreciate that the terms “immune checkpoint inhibitors” (ICIs), “checkpoint inhibitors,” and the like may be used interchangeably herein having all the same qualities and meanings, wherein an immune checkpoint inhibitor encompasses compounds that inhibit the activity or control mechanism(s) of the immune system. Immune system checkpoints, or immune checkpoints, are inhibitory pathways in the immune system that generally act to maintain self-tolerance or modulate the duration and amplitude of physiological immune responses to minimize collateral tissue damage. Checkpoint inhibitors can inhibit an immune system checkpoint by inhibiting the activity of a protein in the pathway.

Immune checkpoint inhibitor targets include, but are not limited to PD-1, PD-L1, CTLA-4, TIGIT, TIM-3, B7-H3, CD73, LAG3, CD27, CD70, 4-1BB, GITR, OX40, SIRP-alpha (CD47), CD39, ILDR2, VISTA, BTLA, and VTCN-1. In some embodiments, an anti-IL-2 antibody therapy is used in combination with an immune checkpoint inhibitor, wherein the target of the immune checkpoint inhibitor comprises PD-1, PD-L1, CTLA-4, TIGIT, TIM-3, B7-H3, CD73, LAG3, CD27, CD70, 4-1BB, GITR, OX40, SIRP-alpha (CD47), CD39, ILDR2, VISTA, BTLA, or VTCN-1, or any combination thereof.

Checkpoint inhibitors may include antibodies, or antigen binding fragments thereof, other binding proteins, biologic therapeutics, or small molecules, that bind to and block or inhibit the activity of one or more of PD-1, PD-L1, CTLA-4, TIGIT, TIM-3, B7-H3, CD73, LAG3, CD27, CD70, 4-1BB, GITR, OX40, SIRP-alpha (CD47), CD39, ILDR2, VISTA, BTLA, or VTCN-1. Illustrative checkpoint inhibitors include but are not limited to those listed in Table 1 below.

TABLE 1
Non-Limiting Examples of Checkpoint Inhibitors
and the Immune Checkpoint Inhibitor Target.

| Drug name | ICI Target |
|---|---|
| Avelumab | anti-PD-L1 |
| Atezolizumab | anti-PD-L1 |
| Durvalumab | anti-PD-L1 |
| Sugemalimab | anti-PD-L1 |
| Envafolimab | anti-PD-L1 |
| Nivolumab (Opdivo)-BMS | anti-PD-1 |
| Pembrolizumab(keytruda) | anti-PD-1 |
| Cemiplimab | anti-PD-1 |
| Camrelizumab | anti-PD-1 |
| Zimberelimab | anti-PD-1 |
| Tislelizumab | anti-PD-1 |
| Sintilimab | anti-PD-1 |
| Teriprizumab | anti-PD-1 |
| Prolgolimab | anti-PD-1 |
| Penpulimab | anti-PD-1 |
| Dostarlimab | anti-PD-1 |
| Genolimzumab | anti-PD-1 |
| Retifanlimab | anti-PD-1 |
| Ipilimumab (Yervoy) | anti-CTLA4 |
| Tiragolumab | anti-TIGIT |
| Domvanalimab | anti-TIGIT |
| Vibostolimab | anti-TIGIT |
| BMS-986207 | anti-TIGIT |
| EOS-448 | anti-TIGIT |
| COM-902 | anti-TIGIT |
| Sabatolimab | anti-TIM3 |
| Cobolimab | anti-TIM3 |
| BMS-986258 | anti-TIM3 |
| INCAGN-02390 | anti-TIM3 |
| S-95018 | anti-TIM3 |
| Omburtamab | anti-B7-H3 |
| MGC-018 | anti-B7-H3 |
| Enoblituzumab | anti-B7-H3 |
| Oleclumab | anti-CD73 |
| BMS-986179 | anti-CD73 |
| NZV-930 | anti-CD73 |
| CPX-006 | anti-CD73 |
| MK-4280 | anti-LAG3 |
| Sym-022 | anti-LAG3 |
| Ieramilimab | anti-LAG3 |
| BI-754111 | anti-LAG3 |
| MK-5890 | anti-CD27 |
| Varlilumab | anti-CD27 |
| Cusatuzumab | anti-CD70 |
| Vorsetuzumab | anti-CD70 |
| Urelumab | anti-4-1BB (agonist) |
| Utomilumab | anti-4-1BB (agonist) |
| ATOR-1017 | anti-4-1BB (agonist) |
| RO-7122290 | anti-4-1BB (agonist) |
| INCAGN-01876 | anti-GITR (agonist) |
| BMS-986156 | anti-GITR (agonist) |
| TRX-518 | anti-GITR (agonist) |
| GWN-323 | anti-GITR (agonist) |
| BMS-986178 | anti-OX40 (agonist) |
| INCAGN-1949 | anti-OX40 (agonist) |
| GSK-3174998 | anti-OX40 (agonist) |
| BGB-A-445 | anti-OX40 (agonist) |
| BI-765063 | anti- SIRP-alpha (CD47) |
| ALX-148 | SIRP-alpha (CD47) |
| IPH-52 | anti-CD39 |
| TTX-030 | anti-CD39 |
| BAY-1905254 | anti-ILDR2 |
| Onvatilimab | anti-VISTA |
| K01401-020 | anti-VISTA |
| JS-004 | anti-BTLA |
| FPA-150 | anti-VTCN1 |

In some embodiments, the checkpoint inhibitor comprises a PD-1 inhibitor. In some embodiments, the checkpoint inhibitor comprises a PD-L1 inhibitor. In some embodiments, the checkpoint inhibitor comprises a CTLA-4 inhibitor. In some embodiments, the checkpoint inhibitor comprises a TIGIT inhibitor. In some embodiments, the checkpoint inhibitor comprises a TIM-3 inhibitor. In some embodiments, the checkpoint inhibitor comprises a B7-H3 inhibitor. In some embodiments, the checkpoint inhibitor comprises a CD73 inhibitor. In some embodiments, the checkpoint inhibitor comprises a LAG3 inhibitor. In some embodiments, the checkpoint inhibitor comprises a CD27 inhibitor. In some embodiments, the checkpoint inhibitor comprises a CD70 inhibitor. In some embodiments, the checkpoint inhibitor comprises a 4-1BB agonist binder. In some embodiments, the checkpoint inhibitor comprises a GITR agonist binder. In some embodiments, the checkpoint inhibitor comprises a OX40 agonist binder. In some embodiments, the checkpoint inhibitor comprises a SIRP-alpha (CD47) inhibitor. In some embodiments, the checkpoint inhibitor comprises a CD39 inhibitor. In some embodiments, the checkpoint inhibitor comprises a ILDR2 inhibitor. In some embodiments, the checkpoint inhibitor comprises a VISTA inhibitor. In some embodiments, the checkpoint inhibitor comprises a BTLA inhibitor. In some embodiments, the checkpoint inhibitor comprises a VTCN-1 inhibitor.

In some embodiments, the checkpoint inhibitor comprises a combination of a PD-1 inhibitor, a PD-L1 inhibitor, a CTLA-4 inhibitor, a TIGIT inhibitor, a TIM-3 inhibitor, a B7-H3 inhibitor, a CD73 inhibitor, a LAG3 inhibitor, a CD27 inhibitor, a CD70 inhibitor, a 4-1BB inhibitor, a GITR inhibitor, a OX40 inhibitor, a SIRP-alpha (CD47) inhibitor, a CD39 inhibitor, a ILDR2 inhibitor, a VISTA inhibitor, a BTLA inhibitor, a VTCN-1 inhibitor. In some embodiments, the checkpoint inhibitor comprises at least two checkpoint inhibitors selected from of a PD-1 inhibitor, a PD-L1 inhibitor, a CTLA-4 inhibitor, a TIGIT inhibitor, a TIM-3 inhibitor, a B7-H3 inhibitor, a CD73 inhibitor, a LAG3 inhibitor, a CD27 inhibitor, a CD70 inhibitor, a 4-1BB inhibitor, a GITR inhibitor, a OX40 inhibitor, a SIRP-alpha (CD47) inhibitor, a CD39 inhibitor, a ILDR2 inhibitor, a VISTA inhibitor, a BTLA inhibitor, and a VTCN-1 inhibitor.

In some embodiments, a pharmaceutical composition for use in a combination therapy, as described herein, comprises an effective amount of a checkpoint inhibitor, as described herein, and a pharmaceutically acceptable carrier.

In some embodiments, a composition disclosed herein comprises a checkpoint inhibitor and a pharmaceutically acceptable carrier. In some embodiments, a composition disclosed herein comprises a combination of checkpoint inhibitors, and a pharmaceutically acceptable carrier. In some embodiments, a composition comprises a checkpoint inhibitor comprising a PD-1 inhibitor, a PD-L1 inhibitor, a CTLA-4 inhibitor, a TIGIT inhibitor, a TIM-3 inhibitor, a B7-H3 inhibitor, a CD73 inhibitor, a LAG3 inhibitor, a CD27 inhibitor, a CD70 inhibitor, a 4-1BB inhibitor, a GITR inhibitor, a OX40 inhibitor, a SIRP-alpha (CD47) inhibitor, a CD39 inhibitor, a ILDR2 inhibitor, a VISTA inhibitor, a BTLA inhibitor, a VTCN-1 inhibitor. In some embodiments, the checkpoint inhibitor comprises at least two checkpoint inhibitors selected from of a PD-1 inhibitor, a PD-L1 inhibitor, a CTLA-4 inhibitor, a TIGIT inhibitor, a TIM-3 inhibitor, a B7-H3 inhibitor, a CD73 inhibitor, a LAG3 inhibitor, a CD27 inhibitor, a CD70 inhibitor, a 4-1BB inhibitor, a GITR inhibitor, a OX40 inhibitor, a SIRP-alpha (CD47) inhibitor, a CD39 inhibitor, a ILDR2 inhibitor, a VISTA inhibitor, a BTLA inhibitor, or a VTCN-1 inhibitor, and a pharmaceutically acceptable carrier. In some embodiments, a composition comprises at least two checkpoint inhibitors selected from a PD-1 inhibitor, a PD-L1 inhibitor, a CTLA-4 inhibitor, a TIGIT inhibitor, a TIM-3 inhibitor, a B7-H3 inhibitor, a CD73 inhibitor, a LAG3 inhibitor, a CD27 inhibitor, a CD70 inhibitor, a 4-1BB inhibitor, a GITR inhibitor, a OX40 inhibitor, a SIRP-alpha (CD47) inhibitor, a CD39 inhibitor, a ILDR2 inhibitor, a VISTA inhibitor, a BTLA inhibitor, a VTCN-1 inhibitor. In some embodiments, the checkpoint inhibitor comprises at least two checkpoint inhibitors selected from of a PD-1 inhibitor, a PD-L1 inhibitor, a CTLA-4 inhibitor, a TIGIT inhibitor, a TIM-3 inhibitor, a B7-H3 inhibitor, a CD73 inhibitor, a LAG3 inhibitor, a CD27 inhibitor, a CD70 inhibitor, a 4-1BB inhibitor, a GITR inhibitor, a OX40 inhibitor, a SIRP-alpha (CD47) inhibitor, a CD39 inhibitor, a ILDR2 inhibitor, a VISTA inhibitor, a BTLA inhibitor, and a VTCN-1 inhibitor, and a pharmaceutically acceptable carrier.

In certain embodiments, when more than one checkpoint inhibitor is used in a therapeutic method described herein, each checkpoint inhibitor is comprised within a separate composition. In certain embodiments, when more than one checkpoint inhibitor is used in a therapeutic method described herein, checkpoint inhibitor may be comprised within the same composition.

In some embodiments, a combination therapy comprises use of an anti-IL-2 antibody or composition thereof as described herein, and a checkpoint inhibitor or a composition thereof. In some embodiments, a combination therapy comprises use of an anti-IL-2 antibody or composition thereof and IL-2 as described herein, and a checkpoint inhibitor or a composition thereof. In some embodiments, a combination therapy comprises use of an anti-IL-2 antibody or composition thereof complexed with IL-2 as described herein, and a checkpoint inhibitor or a composition thereof.

In some embodiments, a combination therapy comprises use of an anti-IL-2 antibody or composition thereof as described herein, and at least two checkpoint inhibitors or a composition thereof. In some embodiments, a combination therapy comprises use of an anti-IL-2 antibody or composition thereof and IL-2 as described herein, and at least two checkpoint inhibitors or a composition thereof. In some embodiments, a combination therapy comprises use of an anti-IL-2 antibody or composition thereof complexed with IL-2 as described herein, and at least two checkpoint inhibitors or a composition thereof.

In some embodiments, a combination therapy comprises a second composition comprising one or more checkpoint inhibitors, as described herein.

In some embodiments, a combination therapy comprises use of anti-IL-2 antibody BDG17.069 or composition thereof, and IL-2 as described herein; and a checkpoint inhibitor or a composition thereof as described herein. In some embodiments, a combination therapy comprises use of BDG17.069 or composition thereof, and a low dose of IL-2 as described herein; and a checkpoint inhibitor or a composition thereof as described herein. In some embodiments, a combination therapy comprises use of BDG17.069 or composition thereof, and a low dose of IL-2 as described herein; and a checkpoint inhibitor or a composition thereof, wherein said checkpoint inhibitor is selected from a PD-1 inhibitor, a PDL-1PD-L1 inhibitor, a CTLA-4 inhibitor, a TIGIT inhibitor, a TIM-3 inhibitor, a B7-H3 inhibitor, a CD73 inhibitor, a LAG3 inhibitor, a CD27 inhibitor, a CD70 inhibitor, a 4-1BB inhibitor, a GITR inhibitor, a OX40 inhibitor, a SIRP-alpha (CD47) inhibitor, a CD39 inhibitor, a ILDR2 inhibitor, a VISTA inhibitor, a BTLA inhibitor, and a VTCN-1 inhibitor.

In some embodiments, a combination therapy comprises use of BDG17.069 or composition thereof, and a low dose of IL-2 as described herein; and a checkpoint inhibitor or a composition thereof, wherein said checkpoint comprises PD-L1. In some embodiments, a combination therapy comprises use of BDG17.069 or composition thereof, and a low dose of IL-2 (aldesleukin); and avelumab or a composition thereof. In certain embodiments of a combination therapy comprising BDG17.069 or composition thereof, and a low dose of IL-2 (aldesleukin); and avelumab or a composition thereof, the IL-2 is administered by subcutaneous injection.

In some embodiments of a combination therapy, the IL-2 administered comprises a low dose of IL-2. In some embodiments of a combination therapy, the IL-2 administered is by subcutaneous administration. In some embodiments of a combination therapy, the IL-2 administered comprises a low dose of IL-2 administered by subcutaneous administration.

In some embodiments of a combination therapy, an anti-IL-2 antibody and IL-2 are comprised in the same composition as a checkpoint inhibitor. In some embodiments, an anti-IL-2 antibody and IL-2 are comprised in different compositions from each other and from a checkpoint inhibitor. In some embodiments, an anti-IL-2 antibody, IL-2, and a checkpoint inhibitor are comprised in the same composition. In some embodiments, an anti-IL-2 antibody and IL-2 are comprised in a composition, and a checkpoint inhibitor is comprised in a different composition. In some embodiments, an anti-IL-2 antibody a checkpoint inhibitor are comprised in a composition, and IL-2 is comprised in a different composition.

In some embodiments of a combination therapy, BDG17.069 and aldesleukin are comprised in the same composition as a PD-L1 checkpoint inhibitor. In some embodiments of a combination therapy, BDG17.069 and aldesleukin are comprised in the same composition as avelumab. In some embodiments, BDG17.069 and aldesleukin are comprised in different compositions from each other and from avelumab. In some embodiments, BDG17.069 and aldesleukin and avelumab are comprised in the same composition. In some embodiments, BDG17.069 and aldesleukin are comprised in a composition, and avelumab is comprised in a different composition. In some embodiments, BDG17.069 and avelumab are comprised in a composition, and aldesleukin is comprised in a different composition. In some embodiments of a combination therapy, the order of administration of an anti-IL-2 antibody or a composition thereof and a checkpoint inhibitor or a composition thereof, may be in any order. In some embodiments of a combination therapy, the order of administration of BDG17.069 or a composition thereof or a composition thereof and avelumab or a composition thereof, may be in any order. In some embodiments of a combination therapy, the order of administration of an anti-IL-2 antibody or a composition thereof, IL-2 or a composition thereof, and a checkpoint inhibitor or a composition thereof, may be in any order. In some embodiments of a combination therapy, the order of administration of BDG17.069 or a composition thereof, aldesleukin or a composition thereof, and avelumab or a composition thereof, may be in any order. For example, but not limited to the anti-IL-2 antibody may be administered prior to, concurrent with, or following administration of the checkpoint inhibitor. Similarly, a combination of an anti-IL-2 antibody and IL-2 may be administered prior to, concurrent with, or following administration of the checkpoint inhibitor. For example, but not limited to the BDG17.069 may be administered prior to, concurrent with, or following administration of the avelumab. Similarly, a combination of BDG17.069 and aldesleukin may be administered prior to, concurrent with, or following administration of the avelumab. In some embodiments, the anti-IL-2 antibody may be administered prior to, concurrent with, or following administration of the at least two checkpoint inhibitors. Similarly, a combination of an anti-IL-2 antibody and IL-2 may be administered prior to, concurrent with, or following administration of the at least two checkpoint inhibitors.

In some embodiments, administration of a combination therapy with a checkpoint inhibitor comprises concurrent administration of an anti-IL-2 antibody or a composition thereof and the checkpoint inhibitor. In some embodiments, administration of a combination therapy with a checkpoint inhibitor comprises concurrent administration of BDG17.069 or a composition thereof and avelumab. In some embodiments, administration of a combination therapy with a checkpoint inhibitor comprises concurrent administration of an anti-IL-2 antibody and IL-2, or composition(s) thereof and the checkpoint inhibitor. In some embodiments, administration of a combination therapy with a checkpoint inhibitor comprises concurrent administration of BDG17.069 and aldesleukin, or composition(s) thereof and avelumab. In some embodiments, administration of a combination therapy with a checkpoint inhibitor comprises prior administration of an anti-IL-2 antibody or a composition thereof before the checkpoint inhibitor. In some embodiments, administration of a combination therapy with a checkpoint inhibitor comprises prior administration of BDG17.069 or a composition thereof before the avelumab. In some embodiments, administration of a combination therapy with a checkpoint inhibitor comprises prior administration of an anti-IL-2 antibody and IL-2, or composition(s) thereof before the checkpoint inhibitor. In some embodiments, administration of a combination therapy with a checkpoint inhibitor comprises prior administration of BDG17.069 and aldesleukin, or composition(s) thereof before the avelumab. In some embodiments, administration of a combination therapy with a checkpoint inhibitor comprises later administration of an anti-IL-2 antibody or a composition thereof following administration of the checkpoint inhibitor. In some embodiments, administration of a combination therapy with a checkpoint inhibitor comprises later administration of BDG17.069 or a composition thereof following administration of the avelumab. In some embodiments, administration of a combination therapy with a checkpoint inhibitor comprises later administration of an anti-IL-2 antibody and IL-2, or composition(s) thereof following the administration of the checkpoint inhibitor. In some embodiments, administration of a combination therapy with a checkpoint inhibitor comprises later administration of BDG17.069 and aldesleukin, or composition(s) thereof following the administration of the avelumab.

In some embodiments, a combination therapy comprises use of a checkpoint inhibitor and an anti-IL-2 antibody comprising a heavy chain variable region having the sequence of one of SEQ ID NOs:10, 12, 14, 16, 18, 20, 22, 24, 26, or 36. In some embodiments, a combination therapy comprises use of a checkpoint inhibitor and an anti-IL-2 antibody comprising alight chain variable region having the sequence of one of SEQ ID NOs:11, 13, 15, 17, 19, 21, 23, 25, 27, or 37. In some embodiments, a combination therapy comprises use of a checkpoint inhibitor and an anti-IL-2 antibody comprising a heavy chain variable region and a light chain variable region having the sequences of one of SEQ ID NOs:10 and 11; SEQ ID NOs:12 and 13; SEQ ID NOs:14 and 15; SEQ ID NOs:16 and 17; SEQ ID NOs:18 and 19; SEQ ID NOs:20 and 21; SEQ ID NOs:22 and 23; SEQ ID NOs:24 and 25; SEQ ID NOs:26 and 27, or SEQ ID NOs: 36 and 37. In some embodiments, a combination therapy comprises use of a checkpoint inhibitor and an anti-IL-2 antibody comprising a heavy chain variable domain comprising CDR1, CDR2 and CDR3 regions comprising amino acid sequences of SEQ ID NOs:38-40 respectively; SEQ ID NOs:44-46 respectively; SEQ ID NOs:50-52 respectively; SEQ ID NOs:56-58 respectively; or SEQ ID NOs:62-64 respectively. In some embodiments, a combination therapy comprises use of a checkpoint inhibitor and an anti-IL-2 antibody comprising a light chain variable domain comprising CDR1, CDR2 and CDR3 regions comprising amino acid sequences of SEQ ID NOs:41-43 respectively; SEQ ID NOs:47-49 respectively; SEQ ID NOs:53-55 respectively; SEQ ID NOs:59-61 respectively; or SEQ ID NOs:65-67, respectively. In some embodiments, a combination therapy comprises use of a checkpoint inhibitor and an anti-IL-2 antibody comprising a heavy chain variable domain comprising CDR1, CDR2 and CDR3 regions comprising amino acid sequences of SEQ ID NOs:38-40 respectively; SEQ ID NOs:44-46 respectively; SEQ ID NOs:50-52 respectively; SEQ ID NOs:56-58 respectively; or SEQ ID NOs:62-64, respectively, and alight chain variable domain comprising CDR1, CDR2, and CDR3 regions comprising amino acid sequences of SEQ ID NOs:41-43 respectively; SEQ ID NOs:47-49 respectively; SEQ ID NOs:53-55 respectively; SEQ ID NOs:59-61 respectively; or SEQ ID NOs:65-67, respectively.

In some embodiments, a combination therapy comprises use of a checkpoint inhibitor and an anti-IL-2 antibody comprising any of clones BDG 17.014, BDG 17.023, BDG 17.038, BDG 17.043, BDG 17.053, BDG 17.054, BDG 17.066, BDG 17.067, and BDG 17.069. In some embodiments, a combination therapy comprises use of a checkpoint inhibitor and an anti-IL-2 antibody comprising anti-IL-2 clone BDG 17.014. In some embodiments, a combination therapy comprises use of a checkpoint inhibitor and an anti-IL-2 antibody comprising anti-IL-2 clone BDG 17.023. In some embodiments, a combination therapy comprises use of a checkpoint inhibitor and an anti-IL-2 antibody comprising anti-IL-2 clone BDG 17.038. In some embodiments, a combination therapy comprises use of a checkpoint inhibitor and an anti-IL-2 antibody comprising anti-IL-2 clone BDG 17.043. In some embodiments, a combination therapy comprises use of a checkpoint inhibitor and an anti-IL-2 antibody comprising anti-IL-2 clone BDG 17.053. In some embodiments, a combination therapy comprises use of a checkpoint inhibitor and an anti-IL-2 antibody comprising anti-IL-2 clone BDG 17.054. In some embodiments, a combination therapy comprises use of a checkpoint inhibitor and an anti-IL-2 antibody comprising anti-IL-2 clone BDG 17.066. In some embodiments, a combination therapy comprises use of a checkpoint inhibitor and an anti-IL-2 antibody comprising anti-IL-2 clone BDG 17.067. In some embodiments, a combination therapy comprises use of a checkpoint inhibitor and an anti-IL-2 antibody comprising anti-IL-2 clone BDG 17.069.

In some embodiments, a combination therapy comprises use of a checkpoint inhibitor; and an anti-IL-2 antibody comprising any of clones BDG 17.014, BDG 17.023, BDG 17.038, BDG 17.043, BDG 17.053, BDG 17.054, BDG 17.066, BDG 17.067, and BDG 17.069; and IL-2. In some embodiments, a combination therapy comprises use of a checkpoint inhibitor and an anti-IL-2 antibody comprising anti-IL-2 clone BDG 17.014, and IL-2. In some embodiments, a combination therapy comprises use of a checkpoint inhibitor and an anti-IL-2 antibody comprising anti-IL-2 clone BDG 17.023, and IL-2. In some embodiments, a combination therapy comprises use of a checkpoint inhibitor and an anti-IL-2 antibody comprising anti-IL-2 clone BDG 17.038, and IL-2. In some embodiments, a combination therapy comprises use of a checkpoint inhibitor and an anti-IL-2 antibody comprising anti-IL-2 clone BDG 17.043, and IL-2. In some embodiments, a combination therapy comprises use of a checkpoint inhibitor and an anti-IL-2 antibody comprising anti-IL-2 clone BDG 17.053, and IL-2. In some embodiments, a combination therapy comprises use of a checkpoint inhibitor and an anti-IL-2 antibody comprising anti-IL-2 clone BDG 17.054, and IL-2. In some embodiments, a combination therapy comprises use of a checkpoint inhibitor and an anti-IL-2 antibody comprising anti-IL-2 clone BDG 17.066, and IL-2. In some embodiments, a combination therapy comprises use of a checkpoint inhibitor and an anti-IL-2 antibody comprising anti-IL-2 clone BDG 17.067, and IL-2. In some embodiments, a combination therapy comprises use of a checkpoint inhibitor and an anti-IL-2 antibody comprising anti-IL-2 clone BDG 17.069, and IL-2.

In certain embodiments, use of a combination therapy is for treating a cancer or tumor. In some embodiments, use of a combination therapy is for treating a solid cancer or solid tumor as described herein.

In some embodiments of a combination therapy disclosed herein, treating a solid tumor comprises treating the primary tumor and secondary metastasis of the tumor. In some embodiments of a combination therapy disclosed herein, treating a solid tumor comprises a first line treatment of the tumor. In some embodiments of a combination therapy disclosed herein, treating a solid tumor comprises treating the secondary metastasis of the tumor. In some embodiments of a combination therapy disclosed herein, treating a solid tumor comprises second line treatment of the tumor. In some embodiments of a combination therapy disclosed herein, treating a solid tumor comprises third line treatment of the tumor. In some embodiments of a combination therapy disclosed herein, treating a solid tumor comprises a first line, a second, or a third line treatment, or a combination thereof, of the tumor.

In some embodiments of a combination therapy disclosed herein, a solid tumor being treated comprises a metastatic cancer. In some embodiments of a combination therapy disclosed herein, a solid tumor being treated comprises an unresectable locally advanced cancer or tumor. In some embodiments of a combination therapy disclosed herein, a solid tumor being treated comprises a metastasis. In some embodiments of a combination therapy disclosed herein, a solid tumor being treated comprises an unresectable locally advanced cancer or tumor.

In some embodiments of a combination therapy disclosed herein, a solid tumor being treated comprises ahead and neck cancer, ahead and neck squamous cell carcinoma (HNSCC), a pancreatic cancer, a lung cancer, a thyroid cancer, a non-small cell lung cancer (NSCLC), a nasopharyngeal carcinoma, a melanoma, an acral melanoma, a uveal melanoma, a colorectal cancer (CRC), a bladder cancer, cholangiocarcinoma (bile duct cancer), a uterine cancer, a cervical cancer, a gallbladder cancer, or a renal cell carcinoma (RCC). In some embodiments of a combination therapy disclosed herein, a solid tumor being treated comprises a melanoma, a metastatic melanoma, a primary melanoma and metastatic melanoma, a renal cell carcinoma (RCC), a non-small cell lung cancer (NSCLC), anasopharyngeal carcinoma, aurothelial cancer, an adrenal cortical carcinoma, a clear cell renal cell carcinoma (ccRCC), a triple-negative breast cancer, a head and neck cancer, a head and neck squamous cell carcinoma (HNSCC), a gastric or gastro-esophageal cancer, an esophageal squamous cell carcinoma, a cutaneous squamous cell carcinoma (cSCC), a pancreatic cancer, a pancreatic adenocarcinoma, a cholangiocarcinoma (bile duct cancer), a hepato-cellular carcinoma (HCC), a colorectal cancer (CRC), an epithelial ovarian cancer, a cervical cancer, an endometrial cancer, a thyroid cancer (follicular or papillary histology), a lung cancer, a bladder cancer, a uterine cancer, a gallbladder cancer, or a Merkel cell carcinomaln some embodiments of a combination therapy disclosed herein a solid tumor being treated comprises a urothelial cancer, an adrenal cortical carcinoma, a clear cell renal cell carcinoma (ccRCC), a melanoma, a triple-negative breast cancer, a head and neck squamous cell carcinoma (NSCC), a gastric or gastro-esophageal cancer, a esophageal squamous cell carcinoma, a cutaneous squamous cell carcinoma (cSCC), a pancreatic adenocarcinoma, a cholangiocarcinoma, a hepato-cellular carcinoma (HCC), a colorectal cancer (CRC), an epithelial ovarian cancer, a cervical cancer, an endometrial cancer, a thyroid cancer (follicular or papillary histology), anon-small cell lung cancer (NSCLC), or a Merkel Cell Carcinoma. In some embodiments, a urothelial cancer arises in the bladder, renal pelvis, ureter, or urethra, or any combination thereof. In certain embodiments, the cancer has progressed during or following an anti-PDx therapy and, if eligible, a platinum-containing regimen. In some embodiments, a urothelial cancer arises in the bladder, renal pelvis, ureter, or urethra, or any combination thereof, wherein the cancer has progressed during or following an anti-PDx therapy and, if eligible, a platinum-containing regimen. In some embodiments, an adrenocortical carcinoma comprises a cancer that is unresectable, locally advanced, or metastatic. In some embodiments, a clear cell renal cell carcinoma (ccRCC) comprises a cancer that has progressed during or following at least 2 approved therapeutic regimens (e.g., small molecule inhibitors, anti-PDx therapy). In some embodiments, a melanoma comprises a cancer that is either locally unresectable or metastatic, wherein said locally unresectable or metastatic cancers may encompass (a) BRAF wt: wherein the cancer progressed after receiving anti-PD-1 containing therapy with or without an anti-CTLA-4; or (b) BRAF mut: wherein the cancer progressed after a BRAF+MEK inhibitor. In some embodiments, triple-negative breast cancer comprises a cancer that is unresectable, locally advanced, or metastatic, and that is refractory to standard 1^{st} line therapy, which may include for example but not limited to cytotoxic chemotherapy alone and/or poly ADP ribose polymerase (PARP) inhibitors for breast cancer gene (BRCA), 1 or 2 mutations, and/or anti-PDx therapy in MSI-H/dMMR positive tumors. In some embodiments, head and neck squamous cell carcinoma (HNSCC) comprises a cancer that has progressed during or following treatment with for example but not limited to, an anti-PDx (unless ineligible, e.g., patients failing chemotherapy and PD-L1 combined positive score (CPS)<1) and platinum-based chemotherapy (unless ineligible for platinum chemotherapy) for metastatic or recurrent disease. In some embodiments, a gastric or gastro-esophageal cancer comprises a cancer progressing during or after cytotoxic chemotherapy (for example but not limited to paclitaxel, fluoropyrimidine, platinum agents) with or without trastuzumab (for HER2 overexpressing adenocarcinoma) and with or without anti PD-1 inhibitor therapy. Patients with a CPS >1 may have received an anti PD-1 containing regimen (unless intolerant or therapy unavailable). In some embodiments, esophageal squamous cell carcinoma comprises a cancer progressing during or after cytotoxic chemotherapy (for example but not limited to paclitaxel, fluoropyrimidine, platinum agents) with an anti PD-1 therapy. Patients with a CPS >10 may have received an anti PD-1 containing regimen (unless intolerant or therapy unavailable). In some embodiments, a cutaneous squamous cell carcinoma (cSCC) comprises a recurrent or metastatic cSCC that is not curable by surgery or radiation. In some embodiments, a pancreatic adenocarcinoma comprises a cancer that is unresectable, locally advanced, or metastatic, and received at least one line of chemotherapy (for example but not limited to FOLFIRINOX; unless ineligible or not feasible). In some embodiments, a cholangiocarcinoma comprises a cancer that is unresectable, locally advanced, or metastatic, in patients who may have had ≥1 line of systemic chemotherapy, unless the patient is ineligible for chemotherapy. In some embodiments, a hepato-cellular carcinoma (HCC) comprises a cancer progressing during or following an approved therapeutic regimen (unless ineligible). In some embodiments, a colorectal cancer (CRC) comprises (a) K-Ras wild type: wherein the patients who have progressed during or after, or are ineligible for, both irinotecan-based and oxaliplatin-based chemotherapy and who are relapsed or refractory to at least 1 prior systemic therapy that included an anti-epidermal growth factor receptor (EGFR) antibody, such as cetuximab or panitumumab; or (b) K-Ras mutant: wherein the patients who have progressed during or after, or are ineligible for, both irinotecan and oxaliplatin based chemotherapy (bevacizumab). In some embodiments, an epithelial ovarian cancer comprises a cancer progressing during or following at least one prior cytotoxic chemotherapeutic regimen (unless ineligible), and subsequent poly ADP ribose polymerase (PARP) inhibitor therapy in BRCA mutation positive patients (unless ineligible). In some embodiments, a cervical cancer comprises a cancer progressing during or following first-line cytotoxic chemotherapy and second-line cytotoxic chemotherapy or anti-PDx therapy in PD-L1 positive (CPS ≥1) or MSI-H/dMMR positive tumors (unless ineligible). In some embodiments, an endometrial cancer in patients comprises a cancer progressing on or following either cytotoxic chemotherapy (for example but not limited to ±trastuzumab) or hormone therapy, and an anti-PDx therapy in MSI-H/dMMR positive tumors. In some embodiments, a thyroid cancer (follicular or papillary histology) comprises a cancer that is iodine refractory. In some embodiments, a non-small cell lung cancer (NSCLC) comprises a cancer that has progressed during or following treatment with platinum-based chemotherapy and an anti-PDx therapy for unresectable, locally advanced, or metastatic disease. In some embodiments, NSCLC harboring an activating EGFR mutation (excluding Exon 20 insertion mutations) or anaplastic lymphoma kinase (ALK) rearrangement must have progressed following available EGFR or ALK-targeted therapy in addition to treatment with platinum-based chemotherapy (unless ineligible for platinum therapy). In some embodiments, a Merkel Cell Carcinoma comprises a metastatic Merkel cell carcinoma that is not curable by surgery or radiation.

In certain embodiments of a combination therapy disclosed herein a solid tumor being treated comprises an unresectable locally advanced or metastatic cancer. In some embodiments, an unresectable locally advanced or metastatic cancer comprises a melanoma, a renal cell carcinoma (RCC), a non-small cell lung cancer (NSCLC), a head and neck squamous cell carcinoma (HNSCC), gastric or gastro-esophageal cancer, esophageal squamous cell carcinoma, cutaneous squamous cell carcinoma (cSCC), pancreatic adenocarcinoma, cholangiocarcinoma (bile duct cancer), hepato-cellular carcinoma (HCC), colorectal cancer (CRC), epithelial ovarian cancer, cervical cancer, endometrial cancer, thyroid cancer having follicular or papillary histology, urothelial cancer, bladder cancer, uterine cancer, gallbladder cancer, or Merkel cell carcinoma.

In some embodiments of a combination therapy, the solid cancer comprises a melanoma, a metastatic melanoma, a primary melanoma and metastatic melanoma, a renal cell carcinoma (RCC), a non-small cell lung cancer (NSCLC), a bladder cancer, a head and neck cancer, a head and neck squamous cell carcinoma (HNSCC), a nasopharyngeal carcinoma, a urothelial cancer, an adrenal cortical carcinoma, a clear cell renal cell carcinoma (ccRCC), a triple-negative breast cancer, a gastric or gastro-esophageal cancer, an esophageal squamous cell carcinoma, a cutaneous squamous cell carcinoma (cSCC), a pancreatic cancer, a pancreatic adenocarcinoma, a cholangiocarcinoma (bile duct cancer), a hepato-cellular carcinoma (HCC), a colorectal cancer (CRC), an epithelial ovarian cancer, a cervical cancer, an endometrial cancer, a thyroid cancer (follicular or papillary histology), a lung cancer, a uterine cancer, a gallbladder cancer, or a Merkel cell carcinoma, or any tumors that are microsatellite instabilities (MSI)-high tumors. In yet another related aspect of a method disclosed herein, the solid cancer comprises a melanoma, a metastatic melanoma, a primary melanoma and metastatic melanoma, a renal cell carcinoma (RCC), a non-small cell lung cancer (NSCLC), a head and neck cancer, a head and neck squamous cell carcinoma (HNSCC), a pancreatic cancer, a lung cancer, a thyroid cancer, a bladder cancer, a nasopharyngeal carcinoma, a colorectal cancer (CRC), cholangiocarcinoma (bile duct cancer), a uterine cancer, a cervical cancer, a gallbladder cancer, a cutaneous squamous carcinoma. In another related aspect, the solid cancer comprises an immune sensitive cancer.

In some embodiments of a combination therapy disclosed herein, a solid tumor being treated comprises, a solid tumor comprises a head and neck cancer, a pancreatic cancer, or a non-small cell lung cancer. In some embodiments of a combination therapy disclosed herein, a solid tumor being treated comprises a non-small cell lung cancer (NSCLC), a melanoma, a metastatic melanoma, a primary melanoma and metastatic melanoma, cutaneous squamous carcinoma, or a renal cell carcinoma (RCC). In some embodiments of a combination therapy disclosed herein, a melanoma comprises an acral melanoma or a uveal melanoma. In some embodiments of a combination therapy disclosed herein, a solid tumor being treated comprises, a melanoma, a metastatic melanoma, a primary melanoma and metastatic melanoma, a renal cell carcinoma (RCC), a non-small cell lung cancer (NSCLC), a nasopharyngeal carcinoma, a urothelial cancer, an adrenal cortical carcinoma, a clear cell renal cell carcinoma (ccRCC), a triple-negative breast cancer, a head and neck cancer, a head and neck squamous cell carcinoma (HNSCC), a gastric or gastro-esophageal cancer, an esophageal squamous cell carcinoma, a cutaneous squamous cell carcinoma (cSCC), a pancreatic cancer, a pancreatic adenocarcinoma, a cholangiocarcinoma (bile duct cancer), a hepato-cellular carcinoma (HCC), a colorectal cancer (CRC), an epithelial ovarian cancer, a cervical cancer, an endometrial cancer, a thyroid cancer (follicular or papillary histology), a lung cancer, a bladder cancer, a uterine cancer, a gallbladder cancer, or a Merkel cell carcinoma. In some embodiments of a combination therapy disclosed herein, a solid tumor being treated comprises ahead and neck cancer. In some embodiments, a solid tumor comprises a pancreatic cancer. In some embodiments of a combination therapy disclosed herein, a solid tumor being treated comprises a lung cancer. In some embodiments of a combination therapy disclosed herein, a solid tumor being treated comprises a thyroid cancer. In some embodiments of a combination therapy disclosed herein, a solid tumor being treated comprises a non-small cell lung cancer (NSCLC). In some embodiments of a combination therapy disclosed herein, a solid tumor being treated comprises a nasopharyngeal carcinoma. In some embodiments of a combination therapy disclosed herein, a solid tumor being treated comprises a melanoma. In some embodiments of a combination therapy disclosed herein, a solid tumor being treated comprises an acral melanoma. In some embodiments of a combination therapy disclosed herein, a solid tumor being treated comprises a uveal melanoma. In some embodiments of a combination therapy disclosed herein, a solid tumor being treated comprises a colorectal cancer (CRC). In some embodiments of a combination therapy disclosed herein, a solid tumor being treated comprises a bladder cancer. In some embodiments of a combination therapy disclosed herein, a solid tumor being treated comprises cholangiocarcinoma (bile duct cancer). In some embodiments of a combination therapy disclosed herein, a solid tumor being treated comprises a uterine cancer. In some embodiments of a combination therapy disclosed herein, a solid tumor being treated comprises a cervical cancer. In some embodiments of a combination therapy disclosed herein, a solid tumor being treated comprises a gallbladder cancer. In some embodiments of a combination therapy disclosed herein, a solid tumor being treated comprises a renal cell carcinoma (RCC). In some embodiments of a combination therapy disclosed herein, a solid tumor being treated comprises a head and neck cancer wherein said head and neck cancer is a head and neck squamous carcinoma (HNSCC1). In some embodiments of a combination therapy disclosed herein, a solid tumor being treated comprises a CRC, wherein the CRC has high MSI. In some embodiments of a combination therapy disclosed herein, a solid tumor being treated comprises a melanoma, wherein the melanoma has a wild-type BRAF gene or has a mutant BRAF gene. In some embodiments of a combination therapy disclosed herein, a solid tumor being treated comprises a pancreatic cancer, wherein the pancreatic cancer is an adenocarcinoma. In some embodiments of a combination therapy disclosed herein, a solid tumor being treated comprises a non-small cell lung cancer (NSCLC), wherein the NSCLC is a squamous cancer. In some embodiments of a combination therapy disclosed herein, a solid tumor being treated comprises a NSCLC, wherein the NSCLC has a mutated epidermal growth factor receptor (EGFRm). In some embodiments of a combination therapy disclosed herein, a solid tumor being treated comprises a cutaneous squamous carcinoma.

### Formulations

Pharmaceutical compositions disclosed herein comprising anti-IL-2 antibodies, or a combination of anti-IL-2 antibodies and IL-2, or checkpoint inhibitors, can be conveniently provided as sterile liquid preparations, e.g., isotonic aqueous solutions, suspensions, emulsions, dispersions, or viscous compositions, which may be buffered to a selected pH, Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the anti-IL-2 antibodies, or a combination of anti-IL-2 antibodies and IL-2, or checkpoint inhibitors, described herein and utilized in practicing the methods disclosed herein, in the required amount of the appropriate solvent with various amounts of the other ingredients, as desired. Such formulations may be in admixture with a suitable carrier, diluent, or excipient such as sterile water, physiological saline, glucose, dextrose, or the like. The formulations can also be lyophilized. The formulations can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the formulations, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

In certain embodiments, the terms “pharmaceutical composition”, “composition”, and “formulation” may be used interchangeably having the same meanings and qualities.

The compositions or formulations described herein can be isotonic, i.e., they can have the same osmotic pressure as blood and lacrimal fluid. The desired isotonicity of the compositions as disclosed herein may be accomplished using sodium chloride, or other pharmaceutically acceptable agents such as dextrose, boric acid, sodium tartrate, propylene glycol or other inorganic or organic solutes. Sodium chloride may be preferred particularly for buffers containing sodium ions.

Viscosity of the compositions, if desired, can be maintained at the selected level using a pharmaceutically acceptable thickening agent. Methylcellulose may be preferred because it is readily and economically available and is easy to work with.

Other suitable thickening agents include, for example, xanthan gum, carboxymethyl cellulose, hydroxypropyl cellulose, carborner, and the like. The preferred concentration of the thickener will depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

In some embodiments, a composition is formulated to be at a pH between about pH 5.0-6.0. In some embodiments, a composition is formulated to be at a pH between about pH 5.0-7.0. In some embodiments, a composition is formulated to be at a pH between about pH 5.0-6.5. In some embodiments, a composition is formulated to be at a pH between about pH 5.0-5.5. In some embodiments, a composition is formulated to be at a pH between about pH 5.5-6.0. In some embodiments, a composition is formulated to be at a pH between about pH 5.5-6.5. In some embodiments, a composition is formulated to be at a pH between about pH 5.0. In some embodiments, a composition is formulated to be at a pH between about pH 5.5. In some embodiments, a composition is formulated to be at a pH between about pH 6.0. In some embodiments, a composition is formulated to be at a pH between about pH 6.5.

In some embodiments, a composition is formulated to be at a pH between about pH 5.0-6.0 and comprises a buffer. In some embodiments, the buffer comprises a pharmaceutically acceptable buffer. In some embodiments, the buffer comprises a histidine buffer or a citrate buffer. In some embodiments, the buffer comprises a histidine buffer. In some embodiments, the buffer comprises a citrate buffer. I

In some embodiments, a composition is formulated to be at a pH between about pH 5.0-6.0 and comprises a buffer selected from a histidine buffer and a citrate buffer. In some embodiments, a composition is formulated to be at a pH between about pH 5.0-6.0 and comprises a histidine buffer. In some embodiments, a composition is formulated to be at a pH between about pH 5.0-6.0 and comprises a citrate buffer.

In some embodiments, a composition further comprises at least one of sucrose, methionine, or PS80, or any combination thereof. In some embodiments, a composition further comprises sucrose. In some embodiments, a composition further comprises methionine. In some embodiments, a composition further comprises PS80.

In some embodiments, a composition comprises an anti-IL-2 antibody as disclosed herein and is formulated to be at a pH between about pH 5.0-6.0 and comprises a buffer selected from a histidine buffer and a citrate buffer. In some embodiments, the composition further comprises IL-2.

Those skilled in the art will recognize that the components of the compositions or formulations should be selected to be chemically inert and will not affect the viability or efficacy of the early apoptotic cell populations as described herein, for use in the methods disclosed herein. This will present no problem to those skilled in chemical and pharmaceutical principles, or problems can be readily avoided by reference to standard texts or by simple experiments (not involving undue experimentation), from this disclosure and the documents cited herein.

### Methods of Use

In one embodiment, the present disclosure provides a method of producing a heavy chain variable region of an anti-IL-2 antibody, the method comprises the step of culturing host cells under conditions conducive to expressing a vector encoding for the heavy chain variable region, thereby producing the heavy chain variable region of the anti-IL-2 antibody.

In one embodiment, the present disclosure provides a method of producing a light chain variable region of an anti-IL-2 antibody, the method comprises the step of culturing host cells under conditions conducive to expressing a vector encoding for the light chain variable region, thereby producing the light chain variable region of the anti-IL-2 antibody.

The VH and/or VL polypeptides disclosed herein may be used in therapeutic methods. In one embodiment, the polypeptides of the present disclosure can be used as immunotherapeutic agents, for example, for differential activation of immune cells as described herein. The present polypeptides can be administered to a subject directly, or by administering to the subject a nucleic acid sequence encoding the polypeptides, such nucleic acid sequence may be carried by a vector.

The exact amount of the present polypeptides or compositions thereof required to elicit the desired effects will vary from subject to subject, depending on the species, age, gender, weight, and general condition of the subject, the particular polypeptides, the route of administration, and whether other drugs are included in the regimen. Thus, it is not possible to specify an exact amount for every composition. However, an appropriate amount can be determined by one of ordinary skill in the art using routine experimentation. Dosages can vary, and the polypeptides can be administered in one or more (e.g., two or more, three or more, four or more, or five or more) doses daily, for one or more days. Guidance in selecting appropriate doses for antibodies can be readily found in the literature.

In some embodiments of a methods of use of an anti-IL-2 antibody described herein, a subject comprises a mammalian subject. In some embodiments, a subject comprises a human subject. In some embodiments, a subject suffers from immune deficiency problems. Treatment of an immune deficient subject would in some embodiments, comprise a prophylactic treatment.

In one embodiment, the present disclosure provides a method of promoting differential growth of immune cells in a subject, comprising the step of preparing a composition comprising an anti-IL-2 antibody disclosed herein, and administering the composition to the subject, thereby promoting differential growth of immune cells in the subject. In one embodiment, the present disclosure provides a method of promoting differential growth of immune cells in a subject, comprising the step of preparing a composition comprising IL-2 and the anti-IL-2 antibody disclosed herein, and administering the composition to the subject, thereby promoting differential growth of immune cells in the subject. In one embodiment, the subject can be an animal or a human. In one embodiment, the immune cells can be CD8+ cells or NK cells.

In some embodiments, disclosed herein is a method of treating a disease or a condition in a subject, comprising the step of administering to the subject a composition comprising an anti-IL-2 antibody as disclosed herein, wherein said antibody promotes differential growth of subsets of immune cells and decreases undesirable effects caused by IL-2, thereby treating said disease or condition in said subject. In some embodiments, a method of treating a disease disclosed here comprises use of a composition comprising an anti-IL-2 antibody and IL-2, or an anti-IL-2 antibody complexed with IL-2. In some embodiments, a method of treating a disease comprises treating a viral infection, a bacterial infection, or a cancer. In some embodiments, a method of treating a condition comprises treating a weak immune system and the treatment prophylactically boosts the immune system.

In some embodiments of a method of treating a disease or a condition, the condition comprises a genetic predisposition that increases likelihood of cancer in said subject. In some embodiments, the genetic predisposition comprises a change in expression or activity of a gene product. In some embodiments, a genetic predisposition that increases the likelihood of cancer comprises a mutation in a tumor suppressor gene or a mismatch repair (MMR) gene, or a combination thereof.

Many hereditary cancers are known in the art non-limiting examples include but are not limited to Hereditary Breast and Ovarian Cancer (HBOC) syndrome, Lynch syndrome (hereditary non-polyposis colorectal cancer), and Li-Fraumeni syndrome.

In some embodiments, the genetic predisposition increases the likelihood of HBOC. HBOC is associated with mutations in the BRAC1 and BRAC2 genes. HBOC is associated with a number of different cancers not just breast cancer, including but not limited to fallopian tube cancer, primary peritoneal cancer, male breast cancer, pancreatic cancer, and prostate cancer. In some embodiments, the genetic predisposition increases the likelihood of any of breast cancer, ovarian cancer, fallopian tube cancer, primary peritoneal cancer, male breast cancer, pancreatic cancer, or prostate cancer, or a combination thereof.

In some embodiments, the genetic predisposition increases the likelihood of hereditary non-polyposis colorectal cancer (HNPCC). HNPCC is associated with mutation in genes including but not limited to MLH1, MSH2, MSH6, PMS1, and PMS2. HNPCC is associated with high risk of developing endometrial cancer, as well as cancers of the ovary, stomach, small intestine, pancreas, kidney, brain, ureters, and bile duct. In some embodiments, the genetic predisposition increases the likelihood of any of hereditary non-polyposis colorectal cancer, cancer of the ovary, stomach cancer, cancer of the small intestine, pancreatic cancer, kidney cancer, brain cancer, cancer of ureters, and cancer of a bile duct.

In some embodiments, the genetic predisposition increases the likelihood of Li-Fraumeni syndrome. Li-Fraumeni syndrome in genes including but not limited to TP53 and CHEK2, or a combination thereof. Li-Fraumeni syndrome is associated with cancers, including sarcoma, osteosarcoma, soft-tissue sarcomas, leukemia, brain (central nervous system) cancers, cancer of the adrenal cortex and breast cancer, or combinations thereof. In some embodiments, the genetic predisposition increases the likelihood of any of a sarcoma, osteosarcoma, soft-tissue sarcomas, leukemia, brain (central nervous system) cancers, cancer of the adrenal cortex and breast cancer, or combinations thereof.

The anti-IL2 antibodies described and exemplified herein, bind the portion of IL-2 that interacts with the alpha (CD25) receptor subunit that is a component of the IL-2 trimeric receptor (CD25/CD132/CD122, sometimes represented as α/β/γ) found on Treg cells, eosinophils, and pulmonary and vascular endothelial cells. In some embodiments, the anti-IL2 antibodies disclosed herein prevent activation of the trimeric IL-2 receptor found on Tregs, eosinophils, and pulmonary and vascular endothelial cells. In some embodiments, the anti-IL2 antibodies disclosed herein bound to IL-2, activate signaling through the IL-2 dimer receptor (CD132/CD122, sometimes represented as β/γ) found on Naïve Teff cells, NK cells, and Natural killer T (NKT) cells.

In some embodiments, a condition being treated in a subject, comprises treating a subject with a genetic predisposition comprising a change in expression or activity of a gene product, said gene comprising BRCA1, BRAC2, MLH1, MSH2, MSH6, PMS1, PMS2, TP53, or CHEK2, or a combination thereof.

As described herein, a complex of IL-2 and the anti-IL-2 antibodies disclosed exhibited pronounced effect in inducing proliferation of memory phenotype effector T cells (MP) CD8+ cells and NK cells, while there was much smaller effect on CD4+Tregs. Thus, the engineered anti-IL-2 antibodies disclosed herein would be useful in adjusting immune cell populations and inducing differential expansion of certain immune effector cells. In one embodiment, such differential expansion of immune effect cells would result in robust activation of the immune system and could be useful for treatment of tumors.

In some embodiments, treatment comprises treating a solid tumor. In some embodiments, treatment comprises treating a non-solid tumor. In some embodiments, treating comprises treating solid and or non-solid tumors, such as but not limited to melanoma, renal cell carcinoma, small cell lung cancer or other cancer conditions. In another embodiment, the method disclosed herein would be useful for treatment of viral infection or bacterial infection. In certain embodiments, a method of treatment comprises treating a solid tumor comprising a melanoma, a metastatic melanoma, a primary melanoma and metastatic melanoma, a renal cell carcinoma (RCC), a non-small cell lung cancer (NSCLC), a nasopharyngeal carcinoma, a urothelial cancer, an adrenal cortical carcinoma, a clear cell renal cell carcinoma (ccRCC), a triple-negative breast cancer, a head and neck cancer, a head and neck squamous cell carcinoma (HNSCC), a gastric or gastro-esophageal cancer, an esophageal squamous cell carcinoma, a cutaneous squamous cell carcinoma (cSCC), a pancreatic cancer, a pancreatic adenocarcinoma, a cholangiocarcinoma (bile duct cancer), a hepato-cellular carcinoma (HCC), a colorectal cancer (CRC), an epithelial ovarian cancer, a cervical cancer, an endometrial cancer, a thyroid cancer (follicular or papillary histology), a lung cancer, a bladder cancer, a uterine cancer, a gallbladder cancer, or a Merkel cell carcinoma. In another embodiment, the method disclosed herein would be useful for treating or preventing a condition caused by IL-2 binding to endothelial CD25 expressing cells, e.g., pulmonary edema, or IL-2-induced vascular leakage.

In some embodiments, a method of use described treating a disease or condition, treats a solid tumor. In some embodiments, a solid tumor comprises a head and neck cancer, a head and neck squamous cell carcinoma (HNSCC), a pancreatic cancer, a lung cancer, a thyroid cancer, a non-small cell lung cancer (NSCLC), a nasopharyngeal carcinoma, a melanoma, an acral melanoma, a uveal melanoma, a colorectal cancer (CRC), a bladder cancer, cholangiocarcinoma (bile duct cancer), a uterine cancer, a cervical cancer, a gallbladder cancer, a cutaneous squamous carcinoma, or a renal cell carcinoma (RCC). In some embodiments, a solid tumor comprises a head and neck cancer, a pancreatic cancer, or a non-small cell lung cancer. In some embodiments, a solid tumor comprises a non-small cell lung cancer (NSCLC), a melanoma, a metastatic melanoma, a primary melanoma and metastatic melanoma, or a renal cell carcinoma (RCC). In some embodiments, a solid tumor comprises a melanoma, a metastatic melanoma, a primary melanoma and metastatic melanoma, a renal cell carcinoma (RCC), a non-small cell lung cancer (NSCLC), a nasopharyngeal carcinoma, a urothelial cancer, an adrenal cortical carcinoma, a clear cell renal cell carcinoma (ccRCC), a triple-negative breast cancer, a head and neck cancer, a head and neck squamous cell carcinoma (HNSCC), a gastric or gastro-esophageal cancer, an esophageal squamous cell carcinoma, a cutaneous squamous cell carcinoma (cSCC), a pancreatic cancer, a pancreatic adenocarcinoma, a cholangiocarcinoma (bile duct cancer), a hepato-cellular carcinoma (HCC), a colorectal cancer (CRC), an epithelial ovarian cancer, a cervical cancer, an endometrial cancer, a thyroid cancer (follicular or papillary histology), a lung cancer, a bladder cancer, a uterine cancer, a gallbladder cancer, or a Merkel cell carcinoma. In some embodiments, a solid tumor comprises a head and neck cancer. In some embodiments, a solid tumor comprises a pancreatic cancer. In some embodiments, a solid tumor comprises a lung cancer. In some embodiments, a solid tumor comprises a thyroid cancer. In some embodiments, a solid tumor comprises a non-small cell lung cancer (NSCLC). In some embodiments, a solid tumor comprises a nasopharyngeal carcinoma. In some embodiments, a solid tumor comprises a melanoma. In some embodiments, a melanoma comprises an acral melanoma or a uveal melanoma. In some embodiments, a solid tumor comprises a colorectal cancer (CRC). In some embodiments, a solid tumor comprises a bladder cancer. In some embodiments, a solid tumor comprises cholangiocarcinoma (bile duct cancer). In some embodiments, a solid tumor comprises a uterine cancer. In some embodiments, a solid tumor comprises a cervical cancer. In some embodiments, a solid tumor comprises a gallbladder cancer. In some embodiments, a solid tumor comprises a renal cell carcinoma (RCC). In some embodiments, a head and neck cancer is a head and neck squamous carcinoma (HNSCC1). In some embodiments, a CRC has high MSI. In some embodiments, a melanoma has a wild-type BRAF gene. In some embodiments, a melanoma has a mutant BRAF gene. In some embodiments, a pancreatic cancer is an adenocarcinoma. In some embodiments, a non-small cell lung cancer (NSCLC) is a squamous cancer. In some embodiments, a NSCLC has a mutated epidermal growth factor receptor (EGFRm). In some embodiments, a solid tumor comprises a cutaneous squamous carcinoma.

In some embodiments, a method of use for treating cancer comprises treating a solid tumor. In certain embodiments, a solid tumor being treated comprises a urothelial cancer, an adrenal cortical carcinoma, a clear cell renal cell carcinoma (ccRCC), a melanoma, a triple-negative breast cancer, a head and neck squamous cell carcinoma (NSCC), a gastric or gastro-esophageal cancer, a esophageal squamous cell carcinoma, a cutaneous squamous cell carcinoma (cSCC), a pancreatic adenocarcinoma, a cholangiocarcinoma, a hepato-cellular carcinoma (HCC), a colorectal cancer (CRC), an epithelial ovarian cancer, a cervical cancer, an endometrial cancer, a thyroid cancer (follicular or papillary histology), anon-small cell lung cancer (NSCLC), or a Merkel Cell Carcinoma. In some embodiments, a solid tumor being treated comprises a melanoma, a metastatic melanoma, a primary melanoma and metastatic melanoma, a renal cell carcinoma (RCC), a non-small cell lung cancer (NSCLC), a nasopharyngeal carcinoma, a urothelial cancer, an adrenal cortical carcinoma, a clear cell renal cell carcinoma (ccRCC), a triple-negative breast cancer, a head and neck cancer, a head and neck squamous cell carcinoma (HNSCC), a gastric or gastro-esophageal cancer, an esophageal squamous cell carcinoma, a cutaneous squamous cell carcinoma (cSCC), a pancreatic cancer, a pancreatic adenocarcinoma, a cholangiocarcinoma (bile duct cancer), a hepato-cellular carcinoma (HCC), a colorectal cancer (CRC), an epithelial ovarian cancer, a cervical cancer, an endometrial cancer, a thyroid cancer (follicular or papillary histology), a lung cancer, a bladder cancer, a uterine cancer, a gallbladder cancer, or a Merkel cell carcinoma. In some embodiments, a urothelial cancer arises in the bladder, renal pelvis, ureter, or urethra, or any combination thereof. In certain embodiments, the cancer has progressed during or following an anti-PDx therapy and, if eligible, a platinum containing regimen. In some embodiments, a urothelial cancer arises in the bladder, renal pelvis, ureter, or urethra, or any combination thereof, wherein the cancer has progressed during or following an anti-PDx therapy and, if eligible, a platinum containing regimen. In some embodiments, an adrenocortical carcinoma comprises a cancer that is unresectable, locally advanced, or metastatic. In some embodiments, a clear cell renal cell carcinoma (ccRCC) comprises a cancer that has progressed during or following at least 2 approved therapeutic regimens (e.g., small molecule inhibitors, anti-PDx therapy). In some embodiments, a melanoma comprises a cancer that is either locally unresectable or metastatic, wherein said locally unresectable or metastatic cancers may encompass (a) BRAF wt: wherein the cancer progressed after receiving anti-PD-1 containing therapy with or without an anti-CTLA-4; or (b) BRAF mut: wherein the cancer progressed after a BRAF+MEK inhibitor. In some embodiments, triple-negative breast cancer comprises a cancer that is unresectable, locally advanced, or metastatic, and that is refractory to standard 1st line therapy, which may include for example but not limited to cytotoxic chemotherapy alone and/or poly ADP ribose polymerase (PARP) inhibitors for breast cancer gene (BRCA), 1 or 2 mutations, and/or anti-PDx therapy in MSI-H/dMMR positive tumors. In some embodiments, head and neck squamous cell carcinoma (HNSCC) comprises a cancer that has progressed during or following treatment with for example but not limited to, an anti-PDx (unless ineligible, e.g., patients failing chemotherapy and PD-L1 combined positive score (CPS)<1) and platinum-based chemotherapy (unless ineligible for platinum chemotherapy) for metastatic or recurrent disease. In some embodiments, a gastric or gastro-esophageal cancer comprises a cancer progressing during or after cytotoxic chemotherapy (for example but not limited to paclitaxel, fluoropyrimidine, platinum agents) with or without trastuzumab (for HER2 overexpressing adenocarcinoma) and with or without anti PD-1 inhibitor therapy. Patients with a CPS ≥1 may have received an anti PD-1 containing regimen (unless intolerant or therapy unavailable). In some embodiments, esophageal squamous cell carcinoma comprises a cancer progressing during or after cytotoxic chemotherapy (for example but not limited to paclitaxel, fluoropyrimidine, platinum agents) with an anti PD-1 therapy. Patients with a CPS ≥10 may have received an anti PD-1 containing regimen (unless intolerant or therapy unavailable). In some embodiments, a cutaneous squamous cell carcinoma (cSCC) comprises a recurrent or metastatic cSCC that is not curable by surgery or radiation. In some embodiments, a pancreatic adenocarcinoma comprises a cancer that is unresectable, locally advanced, or metastatic, and received at least one line of chemotherapy (for example but not limited to FOLFIRINOX; unless ineligible or not feasible). In some embodiments, a cholangiocarcinoma comprises a cancer that is unresectable, locally advanced, or metastatic, in patients who may have had ≥1 line of systemic chemotherapy, unless the patient is ineligible for chemotherapy. In some embodiments, a hepato-cellular carcinoma (HCC) comprises a cancer progressing during or following an approved therapeutic regimen (unless ineligible). In some embodiments, a colorectal cancer (CRC) comprises (a) K-Ras wild type: wherein the patients who have progressed during or after, or are ineligible for, both irinotecan-based and oxaliplatin-based chemotherapy and who are relapsed or refractory to at least 1 prior systemic therapy that included an anti-epidermal growth factor receptor (EGFR) antibody, such as cetuximab or panitumumab; or (b) K-Ras mutant: wherein the patients who have progressed during or after, or are ineligible for, both irinotecan and oxaliplatin based chemotherapy (+bevacizumab). In some embodiments, an epithelial ovarian cancer comprises a cancer progressing during or following at least one prior cytotoxic chemotherapeutic regimen (unless ineligible), and subsequent poly ADP ribose polymerase (PARP) inhibitor therapy in BRCA mutation positive patients (unless ineligible). In some embodiments, a cervical cancer comprises a cancer progressing during or following first-line cytotoxic chemotherapy and second-line cytotoxic chemotherapy or anti-PDx therapy in PD-L1 positive (CPS ≥1) or MSI-H/dMMR positive tumors (unless ineligible). In some embodiments, an endometrial cancer in patients comprises a cancer progressing on or following either cytotoxic chemotherapy (for example but not limited to ±trastuzumab) or hormone therapy, and an anti-PDx therapy in MSI-H/dMMR positive tumors. In some embodiments, a thyroid cancer (follicular or papillary histology) comprises a cancer that is iodine refractory. In some embodiments, a non-small cell lung cancer (NSCLC) comprises a cancer that has progressed during or following treatment with platinum-based chemotherapy and an anti-PDx therapy for unresectable, locally advanced, or metastatic disease. In some embodiments, NSCLC harboring an activating EGFR mutation (excluding Exon 20 insertion mutations) or anaplastic lymphoma kinase (ALK) rearrangement must have progressed following available EGFR or ALK-targeted therapy in addition to treatment with platinum-based chemotherapy (unless ineligible for platinum therapy). In some embodiments, a Merkel Cell Carcinoma comprises a metastatic Merkel cell carcinoma that is not curable by surgery or radiation.

In certain embodiments of method of treating a solid tumor, the solid tumor comprises an unresectable locally advanced or metastatic cancer. In certain embodiments of method of treating a solid tumor, an unresectable locally advanced or metastatic cancer comprises a melanoma, a renal cell carcinoma (RCC), a non-small cell lung cancer (NSCLC), a head and neck squamous cell carcinoma (HNSCC), gastric or gastro-esophageal cancer, esophageal squamous cell carcinoma, cutaneous squamous cell carcinoma (cSCC), pancreatic adenocarcinoma, cholangiocarcinoma (bile duct cancer), hepato-cellular carcinoma (HCC), colorectal cancer (CRC), epithelial ovarian cancer, cervical cancer, endometrial cancer, thyroid cancer having follicular or papillary histology, urothelial cancer, bladder cancer, uterine cancer, gallbladder cancer, or Merkel cell carcinoma.

In certain embodiments of method of treating a solid tumor, a solid tumor comprises a melanoma, a metastatic melanoma, a primary melanoma and metastatic melanoma, a renal cell carcinoma (RCC), anon-small cell lung cancer (NSCLC), a bladder cancer, ahead and neck cancer, a head and neck squamous cell carcinoma (HNSCC), a nasopharyngeal carcinoma, a urothelial cancer, an adrenal cortical carcinoma, a clear cell renal cell carcinoma (ccRCC), a triple-negative breast cancer, a gastric or gastro-esophageal cancer, an esophageal squamous cell carcinoma, a cutaneous squamous cell carcinoma (cSCC), a pancreatic cancer, a pancreatic adenocarcinoma, a cholangiocarcinoma (bile duct cancer), a hepato-cellular carcinoma (HCC), a colorectal cancer (CRC), an epithelial ovarian cancer, a cervical cancer, an endometrial cancer, a thyroid cancer (follicular or papillary histology), a lung cancer, a uterine cancer, a gallbladder cancer, or a Merkel cell carcinoma, or any tumors that are microsatellite instabilities (MSI)-high tumors. In yet another related aspect of a method disclosed herein, the solid cancer comprises a melanoma, a metastatic melanoma, a primary melanoma and metastatic melanoma, a renal cell carcinoma (RCC), a non-small cell lung cancer (NSCLC), a head and neck cancer, a head and neck squamous cell carcinoma (HNSCC), a pancreatic cancer, a lung cancer, a thyroid cancer, a bladder cancer, a nasopharyngeal carcinoma, a colorectal cancer (CRC), cholangiocarcinoma (bile duct cancer), a uterine cancer, a cervical cancer, a gallbladder cancer, a cutaneous squamous carcinoma. In another related aspect, the solid cancer comprises an immune sensitive cancer.

In some embodiments, treating a solid tumor comprises treating the primary tumor and secondary metastasis of the tumor. In some embodiments, treating a solid tumor comprises treating the secondary metastasis of the tumor. In some embodiments, treating a solid tumor comprises a first line treatment of the tumor. In some embodiments, treating a solid tumor comprises second line treatment of the tumor. In some embodiments, treating a solid tumor comprises third line treatment of the tumor. In some embodiments, treating a solid tumor comprises second and third line treatments of the tumor.

As used throughout, the terms “cancer” and “tumor” may in some embodiments be used interchangeably having the same meanings and qualities.

In some embodiments, a solid tumor being treated comprises a metastatic cancer. In some embodiments, a solid tumor being treated comprises an unresectable locally advanced cancer or tumor. In some embodiments, a solid tumor being treated comprises a metastasis. In some embodiments, a solid tumor being treated comprises an unresectable locally advanced cancer or tumor.

In some embodiments, a subject being treating by a method disclosed here has any of 19 solid tumors. In some embodiments, the solid tumor comprises an irresectable locally advanced or metastatic cancer. In some embodiments, the solid tumor comprises an irresectable locally advanced cancer. In some embodiments, the solid tumor comprises a metastatic cancer. In some embodiments, a subject being treated by a method disclosed herein is not eligible for treatment with standard and or approved therapies. In some embodiments, a subject being treated is a human.

In some embodiments, subject being treated by a method disclosed herein has or is suffering from a urothelial cancer, an adrenal cortical carcinoma, a clear cell renal cell carcinoma (ccRCC), a melanoma, a triple-negative breast cancer, a head and neck squamous cell carcinoma (NSCC), a gastric or gastro-esophageal cancer, a esophageal squamous cell carcinoma, a cutaneous squamous cell carcinoma (cSCC), a pancreatic adenocarcinoma, a cholangiocarcinoma, a hepato-cellular carcinoma (HCC), a colorectal cancer (CRC), an epithelial ovarian cancer, a cervical cancer, an endometrial cancer, a thyroid cancer (follicular or papillary histology), a non-small cell lung cancer (NSCLC), or a Merkel Cell Carcinoma. In some embodiments, a subject being treated by a method disclosed herein has or is suffering from a melanoma, a metastatic melanoma, a primary melanoma and metastatic melanoma, a renal cell carcinoma (RCC), a non-small cell lung cancer (NSCLC), a nasopharyngeal carcinoma, a urothelial cancer, an adrenal cortical carcinoma, a clear cell renal cell carcinoma (ccRCC), a triple-negative breast cancer, a head and neck cancer, a head and neck squamous cell carcinoma (HNSCC), a gastric or gastro-esophageal cancer, an esophageal squamous cell carcinoma, a cutaneous squamous cell carcinoma (cSCC), a pancreatic cancer, a pancreatic adenocarcinoma, a cholangiocarcinoma (bile duct cancer), a hepato-cellular carcinoma (HCC), a colorectal cancer (CRC), an epithelial ovarian cancer, a cervical cancer, an endometrial cancer, a thyroid cancer (follicular or papillary histology), a lung cancer, a bladder cancer, a uterine cancer, a gallbladder cancer, or a Merkel cell carcinoma. In some embodiments, a urothelial cancer arises in the bladder, renal pelvis, ureter, or urethra, or any combination thereof. In certain embodiments, the cancer has progressed during or following an anti-PDx therapy and, if eligible, a platinum containing regimen. In some embodiments, a urothelial cancer arises in the bladder, renal pelvis, ureter, or urethra, or any combination thereof, wherein the cancer has progressed during or following an anti-PDx therapy and, if eligible, a platinum containing regimen. In some embodiments, an adrenocortical carcinoma comprises a cancer that is unresectable, locally advanced, or metastatic. In some embodiments, a clear cell renal cell carcinoma (ccRCC) comprises a cancer that has progressed during or following at least 2 approved therapeutic regimens (e.g., small molecule inhibitors, anti-PDx therapy). In some embodiments, a melanoma comprises a cancer that is either locally unresectable or metastatic, wherein said locally unresectable or metastatic cancers may encompass (a) BRAF wt: wherein the cancer progressed after receiving anti-PD-1 containing therapy with or without an anti-CTLA-4; or (b) BRAF mut: wherein the cancer progressed after a BRAF+MEK inhibitor. In some embodiments, triple-negative breast cancer comprises a cancer that is unresectable, locally advanced, or metastatic, and that is refractory to standard 1st line therapy, which may include for example but not limited to cytotoxic chemotherapy alone and/or poly ADP ribose polymerase (PARP) inhibitors for breast cancer gene (BRCA), 1 or 2 mutations, and/or anti-PDx therapy in MSI-H/dMMR positive tumors. In some embodiments, head and neck squamous cell carcinoma (HNSCC) comprises a cancer that has progressed during or following treatment with for example but not limited to, an anti-PDx (unless ineligible, e.g., patients failing chemotherapy and PD-L1 combined positive score (CPS)<1) and platinum-based chemotherapy (unless ineligible for platinum chemotherapy) for metastatic or recurrent disease. In some embodiments, a gastric or gastro-esophageal cancer comprises a cancer progressing during or after cytotoxic chemotherapy (for example but not limited to paclitaxel, fluoropyrimidine, platinum agents) with or without trastuzumab (for HER2 overexpressing adenocarcinoma) and with or without anti PD-1 inhibitor therapy. Patients with a CPS ≥1 may have received an anti PD-1 containing regimen (unless intolerant or therapy unavailable). In some embodiments, esophageal squamous cell carcinoma comprises a cancer progressing during or after cytotoxic chemotherapy (for example but not limited to paclitaxel, fluoropyrimidine, platinum agents) with an anti PD-1 therapy. Patients with a CPS ≥10 may have received an anti PD-1 containing regimen (unless intolerant or therapy unavailable). In some embodiments, a cutaneous squamous cell carcinoma (cSCC) comprises a recurrent or metastatic cSCC that is not curable by surgery or radiation. In some embodiments, a pancreatic adenocarcinoma comprises a cancer that is unresectable, locally advanced, or metastatic, and received at least one line of chemotherapy (for example but not limited to FOLFIRINOX; unless ineligible or not feasible). In some embodiments, a cholangiocarcinoma comprises a cancer that is unresectable, locally advanced, or metastatic, in patients who may have had ≥1 line of systemic chemotherapy, unless the patient is ineligible for chemotherapy. In some embodiments, a hepato-cellular carcinoma (HCC) comprises a cancer progressing during or following an approved therapeutic regimen (unless ineligible). In some embodiments, a colorectal cancer (CRC) comprises (a) K-Ras wild type: wherein the patients who have progressed during or after, or are ineligible for, both irinotecan-based and oxaliplatin-based chemotherapy and who are relapsed or refractory to at least 1 prior systemic therapy that included an anti-epidermal growth factor receptor (EGFR) antibody, such as cetuximab or panitumumab; or (b) K-Ras mutant: wherein the patients who have progressed during or after, or are ineligible for, both irinotecan and oxaliplatin based chemotherapy (±bevacizumab). In some embodiments, an epithelial ovarian cancer comprises a cancer progressing during or following at least one prior cytotoxic chemotherapeutic regimen (unless ineligible), and subsequent poly ADP ribose polymerase (PARP) inhibitor therapy in BRCA mutation positive patients (unless ineligible). In some embodiments, a cervical cancer comprises a cancer progressing during or following first-line cytotoxic chemotherapy and second-line cytotoxic chemotherapy or anti-PDx therapy in PD-L1 positive (CPS ≥1) or MSI-H/dMMR positive tumors (unless ineligible). In some embodiments, an endometrial cancer in patients comprises a cancer progressing on or following either cytotoxic chemotherapy (for example but not limited to ±trastuzumab) or hormone therapy, and an anti-PDx therapy in MSI-H/dMMR positive tumors. In some embodiments, a thyroid cancer (follicular or papillary histology) comprises a cancer that is iodine refractory. In some embodiments, a non-small cell lung cancer (NSCLC) comprises a cancer that has progressed during or following treatment with platinum-based chemotherapy and an anti-PDx therapy for unresectable, locally advanced, or metastatic disease. In some embodiments, NSCLC harboring an activating EGFR mutation (excluding Exon 20 insertion mutations) or anaplastic lymphoma kinase (ALK) rearrangement must have progressed following available EGFR or ALK-targeted therapy in addition to treatment with platinum-based chemotherapy (unless ineligible for platinum therapy). In some embodiments, a Merkel Cell Carcinoma comprises a metastatic Merkel cell carcinoma that is not curable by surgery or radiation.

In some embodiments, methods of treating a solid cancer or in combination methods of treating a solid cancer, the cancer comprises an immune sensitive cancer. In some embodiments, immune sensitive cancers comprise a melanoma, a RCC, a NSCLC, a bladder cancer, ahead and neck cancer, a nasopharyngeal cancer, a Merkel cell carcinoma, or any tumors that are microsatellite instabilities (MSI)-high tumors.

In some embodiments of a methods of treating a disease or condition, the immune cells showing differential growth comprise one or more of naïve T cells, memory T cells, CD8+ T cells, NK cells, or Natural Killer T cells. In some embodiments of a methods of treating a disease or condition, the undesirable effect caused by IL-2 comprises one or more of activation of regulatory T cells, apoptosis of CD25+T effector cells, IL-2 induced pulmonary edema, IL-2 induced pneumonia, or IL-2-induced vascular leakage. In some embodiments of a method of treating a disease or condition, an anti-IL-2 antibody disclosed herein inhibits IL-2 binding to CD25.

In some embodiments, treatment of cancer comprises maintenance treatments. In some embodiments, maintenance treatments are administered to maintain the absence of a cancer or tumor. In some embodiments, maintenance treatments are administered to maintain lack of metastasis of a cancer or tumor. In some embodiments, maintenance treatments are administered to inhibit metastasis of a cancer or tumor. In some embodiments, maintenance treatments are administered to maintain lack of growth of a cancer or tumor. In some embodiments, maintenance treatments are administered to inhibit growth of a cancer or tumor.

In some embodiments, treatment of a solid cancer in a subject reduces the size of the tumor, inhibits or reduces growth of the tumor, or inhibits or reduces metastases of said tumor, or any combination thereof.

In some embodiments, treatment of cancer comprises prophylactic treatment, for example but not limited to a subject harboring a genetic marker or markers with a high risk of developing cancer. In some embodiments, the genetic marker comprises a mutation in the BRCA1 gene.

In some embodiments of methods of promoting differential growth of immune cells in a subject, comprising the step of preparing and administering a composition comprising an anti-IL-2 antibody disclosed herein.

In some embodiments of methods of promoting differential growth of immune cells in a subject, comprising the step of preparing and administering a composition comprising IL-2 and an anti-IL-2 antibody disclosed herein, the administration of a combination of an anti-IL-2 antibody and IL-2, or composition(s) thereof are concurrent. In some embodiments of methods of promoting differential growth of immune cells in a subject, comprising the step of preparing and administering a composition comprising IL-2 and an anti-IL-2 antibody, the administration of an anti-IL-2 antibody and IL-2, or composition(s) thereof comprises administration of an anti-IL-2 antibody or a composition thereof, prior to the IL-2 or a composition thereof. In some embodiments of methods of promoting differential growth of immune cells in a subject, comprising the step of preparing and administering a composition comprising IL-2 and an anti-IL-2 antibody, the administration of an anti-IL-2 antibody and IL-2, or composition(s) thereof comprises administration of an anti-IL-2 antibody or a composition thereof, following administration of the IL-2 or a composition thereof.

In some embodiments, the present disclosure provides a method of treating a subject with a disease or a condition through induction of differential growth of immune cells. In one embodiment, the disease can be viral infection, bacterial infection, or cancer. In one embodiment, the condition can be IL-2 induced pulmonary edema, or IL-2-induced vascular leakage. The method comprises the step of
(a) preparing a composition comprising an anti-IL-2 antibody as disclosed herein; and
(b) administering the composition from (a) to the subject, thereby treating the subject through differential growth of immune cells in the subject. In certain embodiments, any of the engineered anti-IL-2 antibodies disclosed herein may be used in the method of treatment as described.

In some embodiments, the present disclosure provides a method of treating a subject with a disease or a condition through induction of differential growth of immune cells. In one embodiment, the disease can be viral infection, bacterial infection, or cancer. In one embodiment, the condition can be IL-2 induced pulmonary edema, or IL-2-induced vascular leakage. The method comprises the step of
(a) preparing a composition comprising IL-2 and an anti-IL-2 antibody as disclosed herein; and
(b) administering the composition from (a) to the subject, thereby treating the subject through differential growth of immune cells in the subject. In addition to facilitate expansion of subsets of immune effector cells, the antibody/IL-2 complex would also decrease undesirable effects caused by IL-2 (e.g., IL-2 induced pulmonary edema, or IL-2-induced vascular leakage). In one embodiment, the subject can be an animal or a human. In certain embodiments, any of the engineered anti-IL-2 antibodies disclosed herein may be used in the method of treatment as described.

In one embodiment, the present disclosure provides a method of treating a disease or a condition in a subject (e.g., an animal or a human), comprising the step of administering to the subject a composition comprising anti-IL-2 antibodies, wherein the antibodies facilitate expansion of subsets of immune cells and decrease undesirable effects caused by IL-2, thereby treating the disease or condition in the subject. In some embodiments of a method of treating a disease or a condition in a subject, comprising the step of preparing and administering a composition comprising an anti-IL-2 antibody disclosed herein, and administering the composition comprising the anti-IL-2 antibody. In one embodiment, the composition comprises IL-2 and the anti-IL-2 antibodies as disclosed herein, or the composition comprises anti-IL-2 antibodies that are complexed with IL-2.

In some embodiments of a method of treating a disease or a condition in a subject, comprising the step of preparing and administering a composition comprising IL-2 and an anti-IL-2 antibody disclosed herein, the administration of a combination of an anti-IL-2 antibody and IL-2, or composition(s) thereof are concurrent. In some embodiments of a method of treating a disease or a condition in a subject, comprising the step of preparing and administering a composition comprising IL-2 and an anti-IL-2 antibody, the administration of an anti-IL-2 antibody and IL-2, or composition(s) thereof comprises administration of an anti-IL-2 antibody or a composition thereof, prior to the IL-2 or a composition thereof. In some embodiments of a method of treating a disease or a condition in a subject, comprising the step of preparing and administering a composition comprising IL-2 and an anti-IL-2 antibody, the administration of an anti-IL-2 antibody and IL-2, or composition(s) thereof comprises administration of an anti-IL-2 antibody or a composition thereof, following administration of the IL-2 or a composition thereof.

In one embodiment, the method of treatment would be effective for treating conditions such as IL-2 induced pulmonary edema, or IL-2-induced vascular leakage. In another embodiment, the method of treatment would be effective for treating pulmonary edema (mild or chronic) resulting from viral or bacterial infections.

In one embodiment, the disease can be viral infection, bacterial infection, cancer, autoimmune disease or immune disorder. In one embodiment, the disease can be upper respiratory viral infections, early-stage lung infections, or late stage lung infections. A number of diseases and cancers are known to be caused by viruses. Examples of disease-causing viruses include, but are not limited to, norovirus; rotavirus; hepatitis virus A, B, C, D, or E; rabies virus, West Nile virus, enterovirus, echovirus, coxsackievirus, herpes simplex virus (HSV), HSV-2, varicella-zoster virus, mosquito-borne viruses, arbovirus, St. Louis encephalitis virus, California encephalitis virus, lymphocytic choriomeningitis virus, human immunodeficiency virus (HIV), poliovirus, zika virus, rubella virus, cytomegalovirus, human papillomavirus (HPV), enterovirus D68, severe acute respiratory syndrome (SARS) coronavirus, Middle East respiratory syndrome coronavirus, SARS coronavirus 2, Epstein-Barr virus, influenza virus, respiratory syncytial virus, polyoma viruses (such as JC virus, BK virus), Ebola virus, Dengue virus, or any combination thereof. In one embodiment, the viral infection is caused by SARS CoV-2. In another embodiment, the cancer can be, but is not limited to, melanoma or renal cell carcinoma.

In one embodiment, the immune cells that are expanded by treatment with the anti-IL-2 antibodies comprise one or more of naïve T cells, memory T cells, CD8+ T cells, NK cells, and Natural Killer T cells. In one embodiment, treatment with the anti-IL-2 antibodies would decrease one or more undesirable effects caused by IL-2 such as activation of regulatory T cells, apoptosis of CD25+T effector cells, pulmonary edema, pneumonia, and IL-2-induced vascular leakage.

In one embodiment, the anti-IL-2 antibodies administered in the above method are engineered or modified anti-IL-2 antibodies that can inhibit IL-2 binding to CD25. In some embodiments, the engineered or modified anti-IL-2 antibodies comprise a heavy chain variable region having the sequence of one of SEQ ID NOs:10, 12, 14, 16, 18, 20, 22, 24, 26, or 36. In some embodiments, the engineered or modified anti-IL-2 antibodies comprise a light chain variable region having the sequence of one of SEQ ID NOs:11, 13, 15, 17, 19, 21, 23, 25, 27, or 37. In some embodiments, the engineered or modified anti-IL-2 antibodies comprise a heavy chain variable region and a light chain variable region having the sequences of one of SEQ ID NOs:10 and 11; SEQ ID NOs:12 and 13; SEQ ID NOs:14 and 15; SEQ ID NOs:16 and 17; SEQ ID NOs:18 and 19; SEQ ID NOs:20 and 21; SEQ ID NOs:22 and 23; SEQ ID NOs:24 and 25; SEQ ID NOs:26 and 27; or SEQ ID NOs: 36 and 37.

In another embodiment, the engineered or modified anti-IL-2 antibodies comprise a heavy chain variable region having complementarity determining region (CDR) 1, CDR2 and CDR3. In one embodiment, the heavy chain CDR1, CDR2 and CDR3 comprise amino acid sequences of SEQ ID NOs:38-40 respectively; SEQ ID NOs:44-46 respectively; SEQ ID NOs:50-52 respectively; SEQ ID NOs:56-58 respectively; or SEQ ID NOs:62-64 respectively.

In another embodiment, the engineered or modified anti-IL-2 antibodies comprise a light chain variable region having complementarity determining region (CDR) 1, CDR2 and CDR3. In one embodiment, the light chain CDR1, CDR2 and CDR3 comprise amino acid sequences of SEQ ID NOs:41-43 respectively; SEQ ID NOs:47-49 respectively; SEQ ID NOs:53-55 respectively; SEQ ID NOs:59-61 respectively; or SEQ ID NOs:65-67 respectively.

In some embodiments, the engineered anti-IL-2 antibody can be an IgG, IgA, IgM, IgE, IgD, a Fv, a scFv, a Fab, or a F(ab′)₂. The IgG can be of the subclass of IgG1, IgG2, IgG3, or IgG4. In some embodiments, the engineered antibody can be part of a minibody, a diabody, or a triabody antibody.

In some embodiments, a polynucleotide sequence encoding an engineered anti-IL-2 antibody is used in a method of treating a subject with a disease or condition as described herein, wherein the polynucleotide encodes an antibody comprising a heavy chain variable region having the amino acid sequence of one of SEQ ID NOs:10, 12, 14, 16, 18, 20, 22, 24, 26, or 36 In some embodiments, a polynucleotide sequence encoding an engineered anti-IL-2 antibody is used in a method of treating a subject with a disease or condition as described herein, wherein the polynucleotide encodes an antibody comprising alight chain variable region having the amino acid sequence of one of SEQ ID NOs:11, 13, 15, 17, 19, 21, 23, 25, 27, or 37. In some embodiments, a polynucleotide sequence encoding an engineered anti-IL-2 antibody is used in a method of treating a subject with a disease or condition as described herein, wherein the polynucleotide encodes an antibody comprising a heavy chain variable region and a light chain variable region having the amino acid sequences of one of SEQ ID NOs:10 and 11; SEQ ID NOs:12 and 13; SEQ ID NOs:14 and 15; SEQ ID NOs:16 and 17; SEQ ID NOs:18 and 19; SEQ ID NOs:20 and 21; SEQ ID NOs:22 and 23; SEQ ID NOs:24 and 25; SEQ ID NOs:26 and 27; or SEQ ID NOs: 36 and 37.

In some embodiments of a method of using a polynucleotide to treat a disease or condition as described above, the polynucleotide encodes an engineered anti-IL-2 antibody that can be an IgG, IgA, IgM, IgE, IgD, a Fv, a scFv, a Fab, or a F(ab′)₂. The IgG can be of the subclass of IgG1, IgG2, IgG3, or IgG4. In some embodiments, the polynucleotide encodes an engineered antibody which is part of a minibody, a diabody, or a triabody antibody.

In some embodiments a polynucleotide sequence encoding an engineered anti-IL-2 antibody is used in a method of treating a subject with a disease or condition as described herein, wherein the polynucleotide sequence comprises the sequence of one of SEQ ID NOs: 1, 2, 3, 4, 5, 31, 32, 33, 34, or 35.

In some embodiments of a method of treating a disease or condition as described herein, the immune effector cells that are activated by the treatment are CD8+ cells or NK cells. In one embodiment, the anti-IL-2 antibodies disclosed herein, or a complex of IL-2 and the anti-IL-2 antibodies disclosed herein, exhibits pronounced effect in inducing proliferation of MP CD8⁺ cells and NK cells, while there was much smaller effect on CD4⁺ Tregs. In certain embodiments, there is no effect on CD4⁺ Tregs.

In certain embodiments, methods of use of an anti-IL-2 antibody disclosed herein provide a pro-stimulatory effect. A skilled artisan would appreciate that use of the anti-IL-2 antibodies described and exemplified herein e.g., Example 1, clearly demonstrates a pro-stimulatory effect as opposed to an anti-stimulatory or pro-regulatory effect.

In some embodiments, use of an engineered or modified anti-IL-2 antibody comprising a heavy chain variable region having the sequence of one of SEQ ID NOs:10, 12, 14, 16, 18, 20, 22, 24, 26, or 36, provides a pro-stimulatory immune effect in a subject in need thereof. In some embodiments, use of an engineered or modified anti-IL-2 antibody comprising a light chain variable region having the sequence of one of SEQ ID NOs:11, 13, 15, 17, 19, 21, 23, 25, 27, or 37, provides a pro-stimulatory immune effect in a subject in need thereof. In some embodiments, use of an engineered or modified anti-IL-2 antibody comprising a heavy chain variable region and a light chain variable region having the sequences of one of SEQ ID NOs:10 and 11; SEQ ID NOs:12 and 13; SEQ ID NOs:14 and 15; SEQ ID NOs:16 and 17; SEQ ID NOs:18 and 19; SEQ ID NOs:20 and 21; SEQ ID NOs:22 and 23; SEQ ID NOs:24 and 25; SEQ ID NOs:26 and 27; or SEQ ID NOs: 36 and 37, provides a pro-stimulatory immune effect in a subject in need thereof. In some embodiments, said use comprises the anti-IL-2 antibody. In some embodiments, said use comprises the anti-IL-2 antibody and an IL-2. In some embodiments, use comprises a complex of an anti-IL-2 antibody with an IL-2.

In some embodiments, use of an engineered or modified anti-IL-2 antibody comprising a heavy chain variable region having the sequence of one of SEQ ID NOs:10, 12, 14, 16, 18, 20, 22, 24, 26, or 36, provides a pro-stimulatory immune effect in a subject in need thereof as opposed to an anti-stimulatory or pro-regulatory effect. In some embodiments, use of an engineered or modified anti-IL-2 antibody comprising a light chain variable region having the sequence of one of SEQ ID NOs:11, 13, 15, 17, 19, 21, 23, 25, 27, or 37, provides a pro-stimulatory immune effect in a subject in need thereof as opposed to an anti-stimulatory or pro-regulatory effect. In some embodiments, use of an engineered or modified anti-IL-2 antibody comprising a heavy chain variable region and a light chain variable region having the sequences of one of SEQ ID NOs:10 and 11; SEQ ID NOs:12 and 13; SEQ ID NOs:14 and 15; SEQ ID NOs:16 and 17; SEQ ID NOs:18 and 19; SEQ ID NOs:20 and 21; SEQ ID NOs:22 and 23; SEQ ID NOs:24 and 25; SEQ ID NOs:26 and 27; or SEQ ID NOs: 36 and 37, provides a pro-stimulatory immune effect in a subject in need thereof as opposed to an anti-stimulatory or pro-regulatory effect. In some embodiments, said use comprises the anti-IL-2 antibody. In some embodiments, said use comprises the anti-IL-2 antibody and an IL-2. In some embodiments, use comprises a complex of an anti-IL-2 antibody with an IL-2.

In some embodiments, use of an anti-IL-2 antibody comprising a heavy chain variable region comprising heavy chain CDR1, CDR2 and CDR3 as set forth in amino acid sequences SEQ ID NOs:38-40 respectively; SEQ ID NOs:44-46 respectively, SEQ ID NOs:50-52 respectively; SEQ ID NOs:56-58 respectively; or SEQ ID NOs:62-64 respectively, provides a pro-stimulatory immune effect in a subject in need thereof. In some embodiments, use of an anti-IL-2 antibody comprising a light chain comprising light chain CDR1, CDR2 and CDR3 as set forth in amino acid sequences SEQ ID NOs:41-43 respectively; SEQ ID NOs:47-49 respectively; SEQ ID NOs:53-55 respectively; SEQ ID NOs:59-61 respectively; or SEQ ID NOs:65-67 respectively, provides a pro-stimulatory immune effect in a subject in need thereof. In some embodiments, use of an anti-IL-2 antibody comprising a heavy chain variable region comprising heavy chain CDR1, CDR2 and CDR3 as set forth in amino acid sequences SEQ ID NOs:38-40 respectively; SEQ ID NOs:44-46 respectively, SEQ ID NOs:50-52 respectively; SEQ ID NOs:56-58 respectively; or SEQ ID NOs:62-64 respectively, and a light chain comprising light chain CDR1, CDR2 and CDR3 as set forth in amino acid sequences SEQ ID NOs:41-43 respectively; SEQ ID NOs:47-49 respectively; SEQ ID NOs:53-55 respectively; SEQ ID NOs:59-61 respectively; or SEQ ID NOs:65-67 respectively, provides a pro-stimulatory immune effect in a subject in need thereof. In some embodiments, said use comprises the anti-IL-2 antibody. In some embodiments, said use comprises the anti-IL-2 antibody and an IL-2. In some embodiments, use comprises a complex of an anti-IL-2 antibody with an IL-2.

In some embodiments, use of an anti-IL-2 antibody comprising a heavy chain variable region comprising heavy chain CDR1, CDR2 and CDR3 as set forth in amino acid sequences SEQ ID NOs:38-40 respectively; SEQ ID NOs:44-46 respectively, SEQ ID NOs:50-52 respectively; SEQ ID NOs:56-58 respectively; or SEQ ID NOs:62-64 respectively, provides a pro-stimulatory immune effect in a subject in need thereof as opposed to an anti-stimulatory or pro-regulatory effect. In some embodiments, use of an anti-IL-2 antibody comprising a light chain comprising light chain CDR1, CDR2 and CDR3 as set forth in amino acid sequences SEQ ID NOs:41-43 respectively; SEQ ID NOs:47-49 respectively; SEQ ID NOs:53-55 respectively; SEQ ID NOs:59-61 respectively; or SEQ ID NOs:65-67 respectively, provides a pro-stimulatory immune effect in a subject in need thereof as opposed to an anti-stimulatory or pro-regulatory effect. In some embodiments, use of an anti-IL-2 antibody comprising a heavy chain variable region comprising heavy chain CDR1, CDR2 and CDR3 as set forth in amino acid sequences SEQ ID NOs:38-40 respectively; SEQ ID NOs:44-46 respectively, SEQ ID NOs:50-52 respectively; SEQ ID NOs:56-58 respectively; or SEQ ID NOs:62-64 respectively, and a light chain comprising light chain CDR1, CDR2 and CDR3 as set forth in amino acid sequences SEQ ID NOs:41-43 respectively; SEQ ID NOs:47-49 respectively; SEQ ID NOs:53-55 respectively; SEQ ID NOs:59-61 respectively; or SEQ ID NOs:65-67 respectively, provides a pro-stimulatory immune effect in a subject in need thereof, as opposed to an anti-stimulatory or pro-regulatory effect. In some embodiments, said use comprises the anti-IL-2 antibody. In some embodiments, said use comprises the anti-IL-2 antibody and an IL-2. In some embodiments, use comprises a complex of an anti-IL-2 antibody with an IL-2.

Thus, the engineered anti-IL-2 antibodies disclosed herein would be useful in adjusting immune cell populations and inducing differential expansion of certain immune effector cells in a method of treating a disease such as viral infection, bacterial infection, or cancer, or treating a condition such as IL-2 induced pulmonary edema, or IL-2-induced vascular leakage.

In some embodiments, disclosed herein is a method of immunizing of a subject, wherein said immunization comprises administration of a vaccine comprising an adjuvant, said adjuvant comprising an IL-2 antibody adjuvant. In some embodiments, an IL-2 antibody adjuvant comprises the anti-IL-2 antibody and IL-2, or comprises an anti-IL-2 antibody complexed with IL-2. In some embodiments, an IL-2 antibody adjuvant comprises the anti-IL-2 antibody and IL-2. In some embodiments, an IL-2 antibody adjuvant comprises an anti-IL-2 antibody complexed with IL-2. In some embodiments, an IL-2 antibody adjuvant comprises an anti-IL-2 antibody.

In some embodiments, the subject being immunized is a mammalian subject. In some embodiments, the subject being immunized is a human. In some embodiments, the subject being immunized has a weakened immune system.

In some embodiments of a method of immunization, the anti-IL-2 antibody comprise a heavy chain variable region having the sequence of one of SEQ ID NOs: 10, 12, 14, 16, 18, 20, 22, 24, 26, or 36. In some embodiments of a method of immunization, the anti-IL-2 antibody comprise a light chain variable region having the sequence of one of SEQ ID NOs: 11, 13, 15, 17, 19, 21, 23, 25, 27, or 37. In some embodiments of a method of immunization, the anti-IL-2 antibody comprise an anti-IL-2 antibody comprising a heavy chain variable region and a light chain variable region having the sequences of one of SEQ ID NOs:10 and 11; SEQ ID NOs:12 and 13; SEQ ID NOs:14 and 15; SEQ ID NOs:16 and 17; SEQ ID NOs:18 and 19; SEQ ID NOs:20 and 21; SEQ ID NOs:22 and 23; SEQ ID NOs:24 and 25; SEQ ID NOs:26 and 27; or SEQ ID NOs:36 and 37.

In some embodiments of a method of immunization, the anti-IL-2 antibody comprises an anti-IL-2 antibody comprising a heavy chain variable region comprising complementarity determining region (CDR) 1, CDR2 and CDR3, said CDR1, CDR2 and CDR3 comprise amino acid sequences of SEQ ID NOs:38-40 respectively; SEQ ID NOs:44-46 respectively; SEQ ID NOs:50-52 respectively; SEQ ID NOs:56-58 respectively; or SEQ ID NOs:62-64 respectively. In some embodiments of a method of immunization, the anti-IL-2 antibody comprises and anti-IL-2 antibody comprising a light chain variable region comprising complementarity determining region (CDR) 1, CDR2 and CDR3, said CDR1, CDR2 and CDR3 comprise amino acid sequences of SEQ ID NOs:41-43 respectively; SEQ ID NOs:47-49 respectively; SEQ ID NOs:53-55 respectively; SEQ ID NOs:59-61 respectively; or SEQ ID NOs:65-67 respectively. In some embodiments of a method of immunization, the anti-IL-2 antibody comprises an anti-IL-2 antibody comprise a heavy chain variable region and a light chain variable region, each of said heavy chain variable region and light chain variable region comprises complementarity determining region (CDR) 1, CDR2 and CDR3, wherein said heavy chain CDR1, CDR2 and CDR3 comprise amino acid sequences of SEQ ID NOs:38-40 respectively; SEQ ID NOs:44-46 respectively; SEQ ID NOs:50-52 respectively; SEQ ID NOs:56-58 respectively; or SEQ ID NOs:62-64 respectively, wherein said light chain CDR1, CDR2 and CDR3 comprise amino acid sequences of SEQ ID NOs:41-43 respectively; SEQ ID NOs:47-49 respectively; SEQ ID NOs:53-55 respectively; SEQ ID NOs:59-61 respectively; or SEQ ID NOs:65-67 respectively.

In some embodiments of a method of immunizing a subject, an immunization comprises administration of a vaccine comprising an adjuvant, said adjuvant comprising an IL-2 antibody adjuvant, said anti-IL-2 antibody comprising an anti-IL-2 antibody as disclosed herein. In certain embodiments, an IL-2 antibody adjuvant comprises the anti-IL-2 antibody and IL-2, or comprises an anti-IL-2 antibody complexed with IL-2. In some embodiments, of a method of immunizing a subject, a subject has a weakened immune system.

In some embodiments, a subject for immunization with a vaccine comprising an IL-2 antibody adjuvant comprises a subject suffering from a condition comprising a genetic predisposition that increases likelihood of cancer in said subject. In some embodiments, the genetic predisposition comprises a change in expression or activity of a gene product. In some embodiments, a genetic predisposition that increases the likelihood of cancer comprises a mutation in a tumor suppressor gene or a mismatch repair (MMR) gene, or a combination thereof. Many hereditary cancers are known in the art non-limiting examples include but are not limited to Hereditary Breast and Ovarian Cancer (HBOC) syndrome, Lynch syndrome (hereditary non-polyposis colorectal cancer), and Li-Fraumeni syndrome.

In some embodiments, a subject treated by a method disclosed herein for treating a disease or condition is further treated with one or more immune checkpoint inhibitors targeting one or more immune checkpoints. In some embodiments, a subject is treated with said immune checkpoint inhibitors concurrently, before, or after treatment with said anti-IL-2 antibody. In some embodiments of a method of treating disclosed herein, an immune checkpoint comprises PD-1, PD-L1, CTLA-4, TIGIT, TIM-3, B7-H3, CD73, LAG3, CD27, CD70, 4-1BB, GITR, OX40, SIRP-alpha (CD47), CD39, ILDR2, VISTA, BTLA, or VTCN-1, or a combination thereof.

As discussed above, in some embodiments, a therapeutic method of treatment as disclosed herein, further comprises an additional active agent comprising a checkpoint inhibitor. One skilled in the art would appreciate that a combination therapy comprising an anti-IL-2 antibody therapy in the presence or absence of IL-2, and additionally comprising a checkpoint inhibitor may utilize any of the therapeutic compositions or formulations comprising an anti-IL-2 antibody +/−IL-2, and a checkpoint inhibitor as provided herein. In some embodiments, at least two checkpoint inhibitors are used in a combination therapy.

Embodiments of this application include:

An isolated anti-IL-2 antibody, wherein the antibody comprises a heavy chain variable region comprising the sequence of one of SEQ ID NOs:10, 12, 14, 16, 18, 20, 22, 24, 26, or 36.

An antibody including an antibody comprising an IgG, IgA, IgM, IgE, IgD, a Fv, a scFv, a Fab, a F(ab′)₂, a minibody, a diabody, or a triabody antibody.

An IgG comprising.
(a) an IgG1, IgG2, IgG3, or an IgG4;
(b) a heavy chain comprising a mutation that that reduces binding to a Fcγ receptor (FcγRs); or
(c), a lambda or kappa light chain; or
(d) any combination of (a)-(c) thereof.

A composition comprising the isolated anti-IL-2 antibody and a pharmaceutically acceptable carrier.

An isolated anti-IL-2 antibody, wherein the antibody comprises a light chain variable region comprising the sequence of one of SEQ ID NOs:11, 13, 15, 17, 19, 21, 23, 25, 27, or 37.

An isolated anti-IL-2 antibody, wherein the antibody comprises a heavy chain variable region and a light chain variable region having the sequences of one of SEQ ID NOs:10 and 11; SEQ ID NOs:12 and 13; SEQ ID NOs:14 and 15; SEQ ID NOs:16 and 17; SEQ ID NOs:18 and 19; SEQ ID NOs:20 and 21; SEQ ID NOs:22 and 23; SEQ ID NOs:24 and 25; SEQ ID NOs:26 and 27; or SEQ ID NOs:36 and 37.

An isolated anti-IL-2 antibody, wherein the antibody comprises a heavy chain variable region having complementarity determining region 1 (CDR1), CDR2 and CDR3, said CDR1, CDR2 and CDR3 comprise amino acid sequences of SEQ ID NOs:38-40 respectively; SEQ ID NOs:44-46 respectively; SEQ ID NOs:50-52 respectively; SEQ ID NOs:56-58 respectively; or SEQ ID NOs:62-64 respectively.

An isolated anti-IL-2 antibody, wherein the antibody comprises a light chain variable region having complementarity determining region 1 (CDR1), CDR2 and CDR3, said CDR1, CDR2 and CDR3 comprise amino acid sequences of SEQ ID NOs:41-43 respectively; SEQ ID NOs:47-49 respectively; SEQ ID NOs:53-55 respectively; SEQ ID NOs:59-61 respectively; or SEQ ID NOs:65-67 respectively.

An isolated anti-IL-2 antibody, wherein the antibody comprises a heavy chain variable region comprising complementarity determining region 1 (CDR1), CDR2 and CDR3, said CDR1, CDR2 and CDR3 comprise amino acid sequences of SEQ ID NOs:38-40 respectively; SEQ ID NOs:44-46 respectively; SEQ ID NOs:50-52 respectively; SEQ ID NOs:56-58 respectively; or SEQ ID NOs:62-64 respectively; and a light chain variable region having complementarity determining region 1 (CDR1), CDR2 and CDR3, said CDR1, CDR2 and CDR3 comprise amino acid sequences of SEQ ID NOs:41-43 respectively; SEQ ID NOs:47-49 respectively; SEQ ID NOs:53-55 respectively; SEQ ID NOs:59-61 respectively; or SEQ ID NOs:65-67 respectively.

An isolated polynucleotide sequence encoding a heavy chain variable region of an anti-IL-2 antibody, wherein the heavy chain variable region comprises the amino acid sequence of one of SEQ ID NOs:10, 12, 14, 16, 18, 20, 22, 24, 26, or 36.

A vector comprising a polynucleotide sequence described herein. A host cell comprising a vector described herein.

An isolated polynucleotide sequence encoding a light chain variable region of an anti-IL-2 antibody, wherein the light chain variable region comprises the amino acid sequence of one of SEQ ID NOs:11, 13, 15, 17, 19, 21, 23, 25, 27, or 37.

An isolated polynucleotide sequence encoding a heavy chain variable region of an anti-IL-2 antibody, wherein the heavy chain variable region comprises the amino acid sequence of one of SEQ ID NOs:10, 12, 14, 16, 18, 20, 22, 24, 26, or 36, and encoding a light chain variable region of an anti-IL-2 antibody, wherein the light chain variable region comprises the amino acid sequence of one of SEQ ID NOs:11, 13, 15, 17, 19, 21, 23, 25, 27, or 37.

An isolated polynucleotide sequence encoding a scFv, said polynucleotide sequence comprises the sequence of one of SEQ ID NOs: 1, 2, 3, 4, 5, 31, 32, 33, 34, or 35.

A method of producing a heavy chain variable region of an anti-IL-2 antibody, said method comprises the step of culturing a host cell comprising a vector disclosed herein, under conditions conducive to expressing said vector in said host cell, thereby producing the heavy chain variable region of the anti-IL-2 antibody.

A method of producing a light chain variable region of an anti-IL-2 antibody, said method comprises the step of culturing a host cell under conditions conducive to expressing said vector in said host cell, thereby producing the light chain variable region of the anti-IL-2 antibody.

A method of producing an anti-IL-2 antibody comprising a heavy chain variable region and a light chain variable region of an anti-IL-2 antibody, said method comprises the step of culturing a host cell under conditions conducive to expressing said vector in said host cell, thereby producing the light chain variable region of the anti-IL-2 antibody.

A method of promoting differential growth of immune cells in a subject, comprising the step of administering a composition comprising an anti-IL-2 antibody, thereby promoting differential growth of immune cells in the subject. In some embodiments, the composition comprises the anti-IL-2 antibody and IL-2, or the anti-IL-2 antibody complexed with IL-2.

A method of treating a subject with cancer through induction of differential growth of immune cells, comprising the step of administering a composition comprising an anti-IL-2 antibody, thereby treating a subject with cancer.

A method of treating a disease or a condition in a subject, comprising the step of administering to the subject a composition comprising an anti-IL-2 antibody wherein said antibody facilitates expansion of subsets of immune cells and decreases undesirable effects caused by IL-2, thereby treating said disease or condition in said subject.

In some embodiments, the disease comprises a viral infection, a bacterial infection, or a cancer. In some embodiments, the viral infection is caused by SARS CoV-2; norovirus; rotavirus; hepatitis virus A, B, C, D, or E; rabies virus; West Nile virus; enterovirus; echovirus; coxsackievirus; herpes simplex virus (HSV); HSV-2; varicella-zoster virus; mosquito-borne viruses; arbovirus; St. Louis encephalitis virus; California encephalitis virus; lymphocytic choriomeningitis virus; human immunodeficiency virus (HIV); poliovirus; zika virus; rubella virus; cytomegalovirus; human papillomavirus (HPV); enterovirus D68; severe acute respiratory syndrome (SARS) coronavirus; Middle East respiratory syndrome coronavirus; Epstein-Barr virus; influenza virus; respiratory syncytial virus; polyoma viruses including JC virus; BK virus); Ebola virus; Dengue virus; or any combination thereof. In some embodiments, the condition comprises a weak immune system and said treatment prophylactically boosts the immune system.

In some embodiments, the condition comprises IL-2 induced pulmonary edema.

In some embodiments of a method disclosed herein, the immune cells comprise one or more of naïve T cells, memory T cells, CD8+ T cells, NK cells, or Natural Killer T cells.

In some embodiments, the undesirable effect caused by IL-2 comprises one or more of activation of regulatory T cells, apoptosis of CD25+ T effector cells, IL-2 induced pulmonary edema, pneumonia, or IL-2-induced vascular leakage.

In some embodiments, an anti-IL-2 antibody disclosed herein inhibits IL-2 binding to CD25.

A method of immunizing of a subject, wherein said immunization comprises administration of a vaccine comprising an adjuvant, said adjuvant comprising an IL-2 antibody adjuvant.

In some embodiments, the IL-2 antibody adjuvant comprises the anti-IL-2 antibody and IL-2, or comprises an anti-IL-2 antibody complexed with IL-2.

In some embodiments, a subject is an animal or a human. In some embodiments, the subject has a weakened immune system.

In some embodiments of a method disclosed herein, the immune cells are CD8+ cells or NK cells.

In some embodiments of a method disclosed herein, said anti-IL-2 antibody comprise a heavy chain variable region having the sequence of one of SEQ ID NOs: 10, 12, 14, 16, 18, 20, 22, 24, 26, or 36.

In some embodiments of a method disclosed herein, the anti-IL-2 antibody comprise a light chain variable region having the sequence of one of SEQ ID NOs: 11, 13, 15, 17, 19, 21, 23, 25, 27, or 37.

In some embodiments of a method disclosed herein, the anti-IL-2 antibody comprise a heavy chain variable region and a light chain variable region having the sequences of one of: SEQ ID NOs:10 and 11; SEQ ID NOs:12 and 13; SEQ ID NOs:14 and 15; SEQ ID NOs:16 and 17; SEQ ID NOs:18 and 19; SEQ ID NOs:20 and 21; SEQ ID NOs:22 and 23; SEQ ID NOs:24 and 25; SEQ ID NOs:26 and 27; or SEQ ID NOs:36 and 37.

In some embodiments of a method disclosed herein, the anti-IL-2 antibody comprise a heavy chain variable region comprising complementarity determining region (CDR) 1, CDR2 and CDR3, said CDR1, CDR2 and CDR3 comprise amino acid sequences of SEQ ID NOs:38-40 respectively; SEQ ID NOs:44-46 respectively; SEQ ID NOs:50-52 respectively; SEQ ID NOs:56-58 respectively; or SEQ ID NOs:62-64 respectively.

In some embodiments of a method disclosed herein, the anti-IL-2 antibody comprise a light chain variable region comprising complementarity determining region (CDR) 1, CDR2 and CDR3, said CDR1, CDR2 and CDR3 comprise amino acid sequences of SEQ ID NOs:41-43 respectively; SEQ ID NOs:47-49 respectively; SEQ ID NOs:53-55 respectively; SEQ ID NOs:59-61 respectively; or SEQ ID NOs:65-67 respectively.

In some embodiments of a method disclosed herein, the anti-IL-2 antibody comprise a heavy chain variable region and a light chain variable region, each of said heavy chain variable region and light chain variable region comprises complementarity determining region (CDR) 1, CDR2 and CDR3, wherein said heavy chain CDR1, CDR2 and CDR3 comprise amino acid sequences of SEQ ID NOs:38-40 respectively; SEQ ID NOs:44-46 respectively; SEQ ID NOs:50-52 respectively; SEQ ID NOs:56-58 respectively; or SEQ ID NOs:62-64 respectively, wherein said light chain CDR1, CDR2 and CDR3 comprise amino acid sequences of SEQ ID NOs:41-43 respectively; SEQ ID NOs:47-49 respectively; SEQ ID NOs:53-55 respectively; SEQ ID NOs:59-61 respectively; or SEQ ID NOs:65-67 respectively.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

A skilled artisan would appreciate that the term “about”, may encompass a deviance of between 0.0001-5% from the indicated number or range of numbers. In some instances, the term “about”, may encompass a deviance of between 1-10% from the indicated number or range of numbers. In some instances, the term “about”, encompasses a deviance of up to 25% from the indicated number or range of numbers.

### EXAMPLES

### Example 1

This example describes generation of modified anti-IL-2 antibodies based on embodiments of the antibodies generated. The exemplification of generating modified anti-IL-2 antibodies is based on a sub-set of the antibodies disclosed herein. The description and results presented in Example 1 are exemplary and do not limit the generation of modified anti-IL-2 antibodies disclosed throughout this application.

### Library Design

A library was designed to introduce variation into the sequence of JES6.1. Amino acid sequences for the heavy chain variable region and light chain variable region of JES6.1 are shown in SEQ ID NO:6 and SEQ ID NO:7 respectively. Briefly, three positions were varied with a codon encoding all amino acids (codon NNS). The design of the library allowed for one mutation in both CDRs L3 and H3, as well as a one mutation in one of the following CDRs: H1, H2, or L2. CDRs were defined by meeting either the IMGT or ABR (Kunik et al., 2012) definitions. CDR residues that are conserved (based on Blast search against the PDB database) or don't form specific interactions with mouse IL-2 (mIL-2) in the crystal structure of the mIL2-JES6.1 complex (PDB 4YQX), were excluded from variation. The theoretical size of the library was 1.38E+7 variants.

### Library Selection

### Screening and Selection Using Yeast Surface Display

Yeast-displayed scFv libraries were grown in a SDCAA selective medium and induced for expression with 2% w/v galactose at 30° C. overnight according to established protocols. The library was incubated with 100 nM of recombinant human IL-2 with a 6xhis tag (hIL-2-His) (Reprokine, Israel) in PBS 0.1% BSA for 1 hour, then washed three times with PBS 0.1% BSA and labeled with fluorescent labeled antibodies mouse anti Myc-FITC (Santa Cruze, USA) and mouse monoclonal anti-His APC (Miltenyi Biotec, Germany. cat 0020130-119-782). Post labeling the library was sorted on BioRad S3e Fluorescence Activated Cell Sorter for high affinity binders to recombinant human IL-2. Isolated clones from the final sort were sequenced by extraction of plasmid DNA from the yeast clones using a Zymoprep kit (Zymo Research, USA) and the DNA was sequenced.

### Koff Selection

To select for binders with improved off rate the clones of round 2 of selection were incubated for 15 min with 10 nM 6xhis tag (hIL-2), then the yeast were washed 3× with 1 ml PBS 0.1% BSA and incubated for 5 minutes, 4 h, 6 h, and 24 h with 100 nM unlabeled IL-2. At the indicated time points the yeast were washed and labeled with Myc-FITC (Santa Cruze, USA) and monoclonal anti-His APC (Miltenyi Biotec, Germany. cat 0020130-119-782), and sorted on a Se3 as described above.

### IgG Production

JES6.1w.t. was bought from Thermo Fisher (cat: 16-7022-81). JES6.1.RMC was cloned as a rat Fv with a mouse IgG2a constant region, and produced by GeneScript antibody production services (Genscript NJ, USA). BDG17.0014 was cloned into human IgG1 constant region and produced by GeneScript antibody production services. Amino acid sequences for the heavy chain variable region and light chain variable region of JES6.1.RMC are shown in SEQ ID NO:8 and SEQ ID NO:9 respectively. All other antibodies were generated as described below.

### Reformatting

Selected scFv clone was reformatted to human IgG1 format. The sequences of the light chain (LC) and heavy chain (HC) variable regions were optimized to mammalian codon usage and ordered as genblocks (GB) from IDT (Integrated DNA Technologies. Coralville, Iowa USA). The GB were cloned using standard cloning techniques into pSF-CMV-HuIgG1 HC (HC plasmid) and pSF-CMV-HuLambda_LC (LC plasmid) (Oxford genetics, Oxford UK). When indicated, the variable Heavy chain was cloned into pSF-CMV-HuIgG1 HC_LALA (HC plasmid) in which the DNA coding for L234 and L235 of the heavy chain was mutated to alanine codons (L234A, L235A)

### IgG Expression

Expi-CHO cells (Thermo Fisher Scientific, USA) were transfected with LC and HC plasmids at a ratio of 2:1 and expression was done according to the manufacturer's instructions. Briefly: 50 ml Expi-CHO cells were cultured at 37° C., 120 rpm, 8% CO₂ to a density of 6×10⁶ cells/ml. Then, 50 pg of heavy chain and light chain expression plasmids at a ratio of 1:2 were transfected into the CHO cells. Post transfections, a booster enhancer and feed were added to the culture, and growth conditions were changed to 32° C., 120 rpm, 5% CO₂. The cells were harvested 10 days after transfection. The IgGs were purified from the supernatant using protein A beads (Tosoh Bioscience GmbH, Germany), followed by size exclusion chromatography (SEC) purification on superdex 200 10/300 increase column, with PBS as mobile phase (GE healthcare, USA).

### Sequences

DNA sequences encoding scFv of clone 1 (17.021) is shown in SEQ ID NO:1. DNA sequences encoding scFv of clone 2 (17.022) is shown in SEQ ID NO:2. DNA sequences encoding scFv of clone 4 (17.023) is shown in SEQ ID NO:3. DNA sequences encoding scFv of clone 5 (17.030) is shown in SEQ ID NO:4. DNA sequences encoding scFv of clone 6 (17.035) is shown in SEQ ID NO:5.

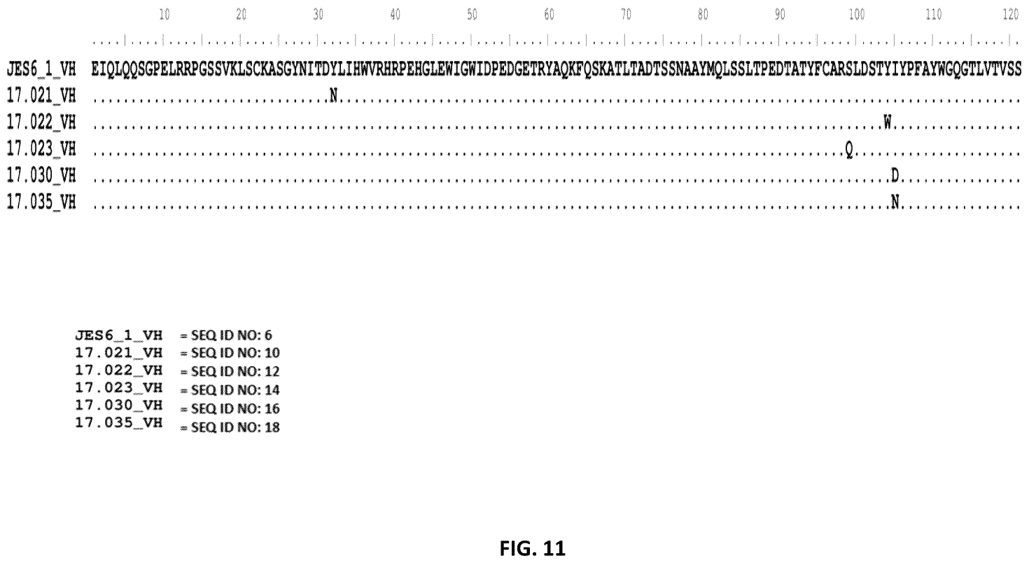

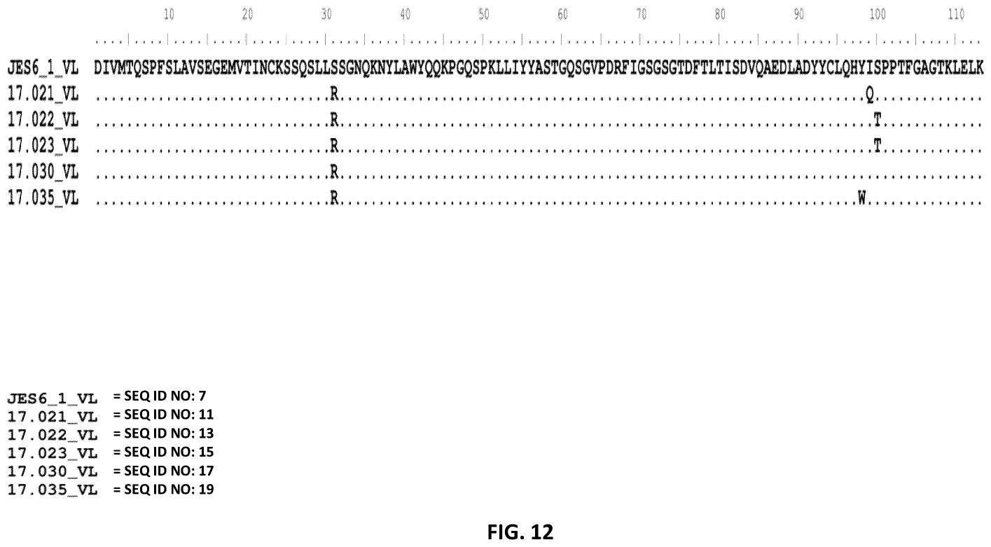

Amino acid sequences for the heavy chain variable region and light chain variable region of original JES6_1 starting sequence and the various anti-IL-2 clones are shown in the table below and in FIGS. 11 and 12.

TABLE 2
VH and VL Amino Acid Sequences of
a Sub-Set of Anti-IL-2 Clones.

| | Heavy Chain | Light Chain |
|---|---|---|
| BDG Clone | Variable Region | Variable Region |
| 17.021 | SEQ ID NO: 10 | SEQ ID NO: 11 |
| 17.022 | SEQ ID NO: 12 | SEQ ID NO: 13 |
| 17.023 | SEQ ID NO: 14 | SEQ ID NO: 15 |
| 17.030 | SEQ ID NO: 16 | SEQ ID NO: 17 |
| 17.035 | SEQ ID NO: 18 | SEQ ID NO: 19 |

### Measurements of IgG Binding to Human IL-2

The SPR analysis was done on Biacore 200 (GE healthcare, USA) on CM5 chips (cat:br10005-30, GE healthcare, USA). The chip was crosslinked with primary capture Ab against human IgG (Cat: br-1008-39, GE healthcare, USA) or primary capture antibody against mouse IgG (Cat: BR-1008-38, GE healthcare, USA) to a target of 8000RU. After cross-linking of the primary Ab, the mouse and human tested antibodies were immobilized on the primary Ab to a target of approximately additional 500RU. JES6.1 was cross-linked directly to the CM5 chip. Human IL-2 (Cat: 60568, Reprokine, Israel) analyte was streamed in HEB-EP or PBS 0.05% tween-20 (PBS-T) buffer at concentrations ranging from 128 nM to 0.03 nM in a series of two-fold or three-fold dilutions, one concentration for each cycle. Mouse IL-2 (Cat: RKP04351, Reprokine, Israel) was streamed in HEB-EP or PBS-T buffer at concentrations ranging from 0.5 nM to 40 nM. At the end of each cycle the analyte and tested antibody were stripped from the chip using 3M MgCl₂ and new tested Ab was loaded on the chip as described above. When indicated, instead of stripping the antibodies, kinetics was determined by injecting series of analyte concentrations in one cycle by the Single-cycle kinetics method. Binding kinetics were determined by the 1:1 Binding model using the Biacore T200 evaluation software.

### Binding of IgGs to Cynomolgus Monkey IL-2

The SPR analysis was done on Biacore 200 (GE healthcare, USA) on CM5 chips (cat:br10005-30, GE healthcare, USA). The chip was crosslinked with primary capture Ab against human IgG (Cat: br-1008-39, GE healthcare, USA) to a target of 5000RU and cynomolgus monkey IL-2 (cIL-2) was tested by the multi-cycle method in the same conditions described above.

### Sec Analysis.

To analyze the IgGs, 100 mg samples were loaded on a Superdex 200 10/300 increase column (GE healthcare, USA) at a flow rate of 0.8 ml/min on a GE AKTA Explorer chromatography system (GE healthcare, USA). Monitoring of antibody retention time was done at 280 nm.

### Testing for Specific Binding to CD25 and CD122

To test specific binding to CD25, BDG17.023 was immobilized to the CM5 chip to a target RU of approximately 300RU as described above. Subsequently, 50 nM IL-2 was injected till the BDG17.023 or control antibody were saturated. Then the Ab-IL-2 complex was washed with PBS-T buffer for 10 sec and 1000 nM of CD25 was injected and monitored for response.

To test specific binding to CD122, BDG17.023 was immobilized to the CM5 chip to a target RU of approximately 300RU-500RU as described above. Subsequently, 50 nM hIL-2 was injected till the BDG17.023 antibody was saturated with hIL-2. Next the Ab-IL-2 complex was washed with PBS-T buffer for 10 sec and 1000 nM of CD122 was injected and response was monitored.

To test specific binding of the humanized antibodies IL-2 complex to CD122 and CD25, antibodies BDG17.038, BDG17.043, BDG17.053, BDG17.054, BDG17.067, BDG17.069 (See, Tables 6 and 7 of Example 2 for sequence information for these clones) were immobilized to a capture antibody attached to CM5 chip channel to a target RU of approximately 300RU as described above. Subsequently, 50 nM IL-2 was injected till the respective antibody was saturated with hIL-2. Then the Ab-IL-2 complex was washed with PBS-T buffer for 60 sec and 1000 nM of CD25 was injected and monitored for response. Subsequently, running buffer was injected for 60 seconds to reach a steady baseline, and then 1000 nM of CD122 was injected for 30 seconds at a flow rate of 30 ul/min. To test CD122 binding, the same experiment was repeated in reverse order, with CD122 injected first flowed by injection of CD25.

### DSF Analysis of IgG Tm

To determine the T-onset and Tm of the humanized anti-hIL-2 antibodies, antibodies were diluted to 0.5 mg/ml in PBS and analyzed using NanoDSF Prometheus NT.48 (Nanotemper, Germany) in a temperature elevation rate of 1° C./min.

### In Vivo Experiments

Treatment of Mice with the IL-2/Ab Complex

Groups of six male C57BL/6 mice, 7-8 weeks old, were injected intraperitoneally (i.p.) with BDG17.023/hIL-2 or JES6.1/mIL-2 immune complex daily, for four consecutive days. PBS and free hIL-2 or mIL-2 served as controls. At the end of the fourth day the mice were sacrificed, spleens were harvested and homogenized into a single cell suspension. The cells were filtered, centrifuged (400 g for 5 minutes) resuspended in 5 ml PBS to a final concentration of 5×10⁶ lymphocytes/ml. The experiment was done in accordance with the guidelines of the national council for Institutional Animal Care and Use Committee (IACUC) in Israel.

Groups of six male C57BL/6 mice, 7-8 weeks old, were injected intraperitoneally (IP.) with BDG17.038/hIL-2, BDG17.043/hIL-2, BDG17.054/hIL-2, BDG17.038/hIL-2 or Isotype control/hIL-2 immune complex daily, for four consecutive days. To form the complex, 10 mg of the antibody was pre-incubated with 0.5 mg of hIL-2 for 30 minutes at 37° C. before the injection. At the end of the fourth day the mice were sacrificed, spleens were harvested and homogenized into a single cell suspension. The cells were filtered, centrifuged (400 g for 5 minutes) resuspended in 5 ml PBS to a final concentration of 5×10⁶ lymphocytes/ml. The experiment was done in accordance with the guidelines of the national council for Institutional Animal Care and Use Committee (IACUC) in Israel.

### B16F10 Murine Melanoma Tumor Xenograft Model

Female C57BL/6 mice were inoculated subcutaneously in the right rear flank region with (2 ×105) B16-F10 tumor cells. Five days post inoculation when the tumor volume reached ˜30-50 mm³, the mice were randomized into experimental groups (n=10 per group) and injected intraperitoneally daily with single doses of 10 mg anti-IL-2 antibody/1 mg hIL-2 complex of the indicated antibodies or with PBS control for four consecutive days. The mice were monitored for tumor volume growth, body weight loss and for non-specific clinical signs throughout the experiments.

### Determination of Immune Cell Population By FACS

In order to identify immune cell populations, spleen lymphocytes were labeled with the antibodies described below according to the manufacturer's instructions. Regulatory T cells (Tregs) were designated as cells labeled as CD45⁺/CD3⁺/CD4⁺/CD25⁺/FoxP3⁺. Memory phenotype effector T cells (MP CD8⁺): CD45⁺/CD3⁺/CD8⁺/CD44⁺/IL-2RB(CD122)+. Natural killer cells (NK): CD45⁺/CD3−/ CD49b+/ NK1.1 (CD161). Natural killer T cells (NKT): CD45⁺/CD3⁺/CD49b⁺/NK1.1 (CD161). Positive cells frequency and number were calculated from the raw data acquired on the flow cytometer.

TABLE 3
Marker/Labeled Antibodies

| | Catalog No. | | | | | |
|---|---|---|---|---|---|---|
| | Thermo | | | | | |
| | Fisher | | Excitation | Emission | Concertation | Dilution |
| Reagent | Scientific | Channel | Filter | Filter | (mg/ml) | (μg)/test |
| Anti-MO/RT | 11-5773-82 | FL1 | 488 | 525/50 | 0.5 | 1 |
| FOXP3 | | | | | | |
| FJK-16S | | | | | | |
| FITC | | | | | | |
| 100 UG | | | | | | |
| CD25 | 12-0251-82 | FL2 | 488 | 575/30 | 0.2 | 0.125 |
| Monoclonal | | | | | | |
| Antibody | | | | | | |
| (CD25-4E3), | | | | | | |
| PE 100T | | | | | | |
| CD49b | 61-5971-82 | FL3 | 488 | 620/30 | 0.2 | 0.5 |
| (Integrin | | | | | | |
| alpha 2) | | | | | | |
| Monoclonal | | | | | | |
| Antibody | | | | | | |
| (DX5), PE- | | | | | | |
| eFluor | | | | | | |
| 610/100 μg | | | | | | |
| CD4 anti | BLG | FL4 | 488 | 695/30 | 0.2 | 0.25 |
| mouse, | 100540 | | | | | |
| PerCP/Cyanine5.5 | | | | | | |
| 100 μg | | | | | | |
| CD122 (IL- | BLG | FL5 | 488 | 755LP | 0.2 | 0.25 |
| 2Rβ) anti | 123216 | | | | | |
| mouse, PE- | | | | | | |
| Cyanine7/ | | | | | | |
| 100 ug | | | | | | |
| NK1.1 | 17-5941-82 | FL6 | 638 | 660/20 | 0.2 | 0.125 |
| Monoclonal | | | | | | |
| Antibody | | | | | | |
| (PK136), | | | | | | |
| APC/ | | | | | | |
| 100 μg | | | | | | |
| CD3 | 56-0032-82 | FL7 | 638 | 725/20 | 0.2 | 0.25 |
| Monoclonal | | | | | | |
| Antibody | | | | | | |
| (17A2), | | | | | | |
| Alexa Fluor | | | | | | |
| 700, | | | | | | |
| eBioscience ™/ | | | | | | |
| 100 μg | | | | | | |
| CD44 | 47-0441-82 | FL8 | 638 | 755LP | 0.2 | 0.25 |
| Monoclonal | | | | | | |
| Antibody | | | | | | |
| (IM7), | | | | | | |
| APC- | | | | | | |
| eFluor 780/ | | | | | | |
| 100 μg | | | | | | |
| CD8a | 62-0081-82 | FL9 | 405 | 450/50 | 0.2 | 0.25 |
| Monoclonal | | | | | | |
| Antibody | | | | | | |
| (53-6.7), | | | | | | |
| Super | | | | | | |
| Bright 436, | | | | | | |
| eBioscience ™/ | | | | | | |
| 100 μg | | | | | | |
| CD45 | 69-0451-82 | FL10 | 405 | 525/50 | 0.2 | 0.5 |
| Monoclonal | | | | | | |
| Antibody | | | | | | |
| (30-F11), | | | | | | |
| eFluor 506, | | | | | | |
| eBioscience ™/ | | | | | | |
| 100 μg | | | | | | |

### Results

JES6.1 Binds Mouse Strongly but does not Bind the Human IL-2

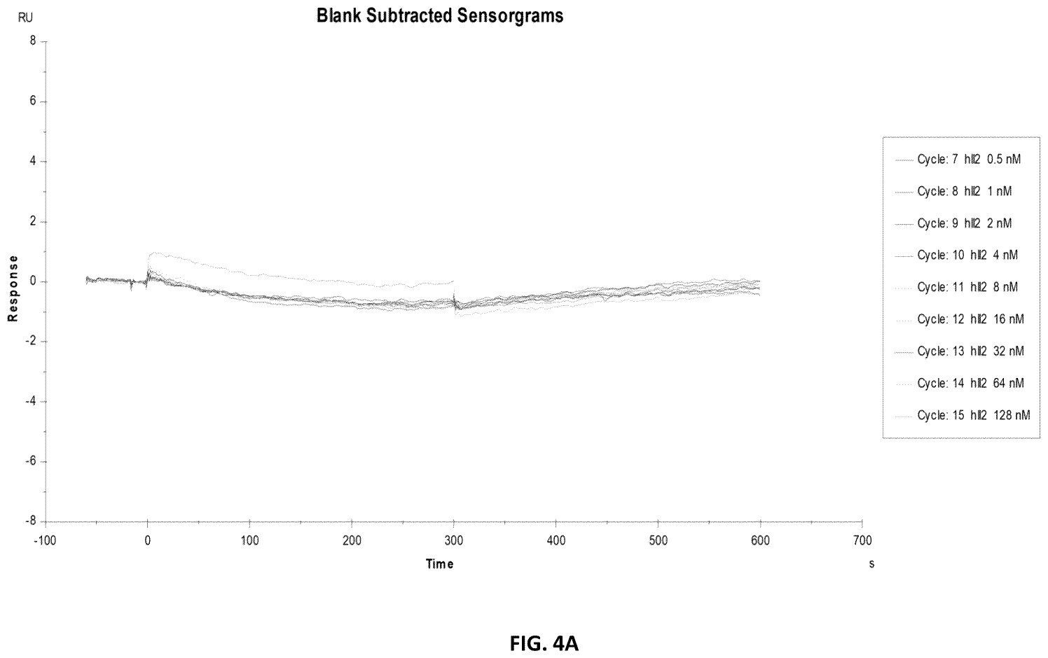

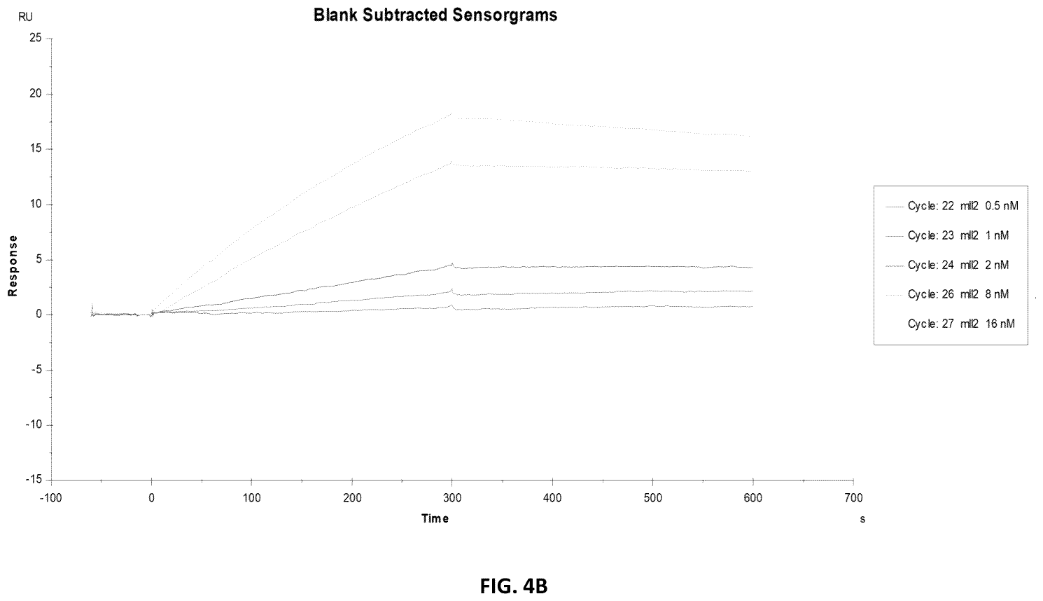

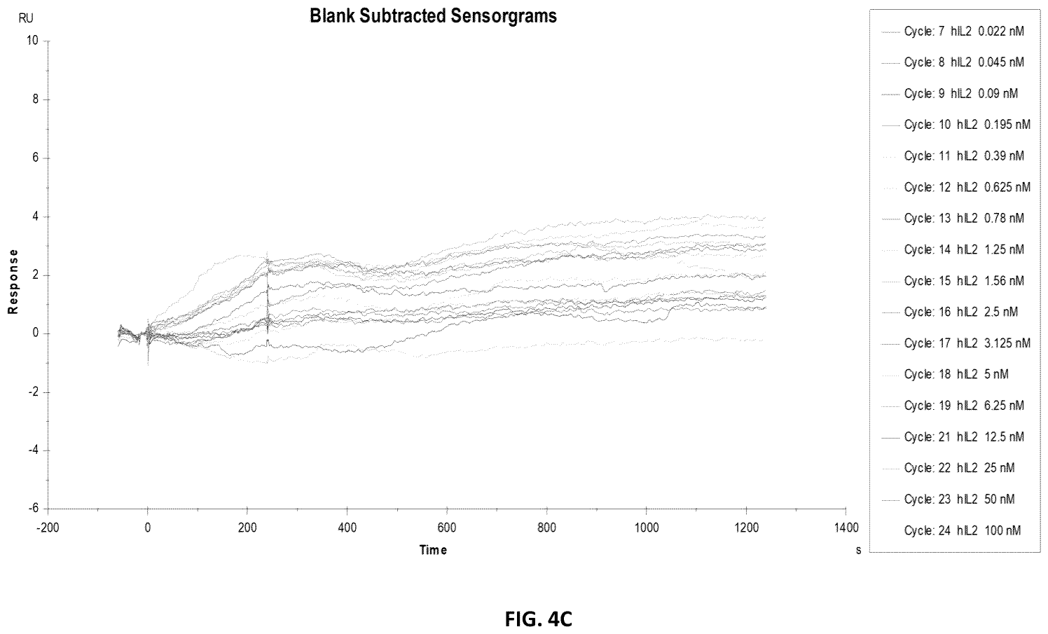

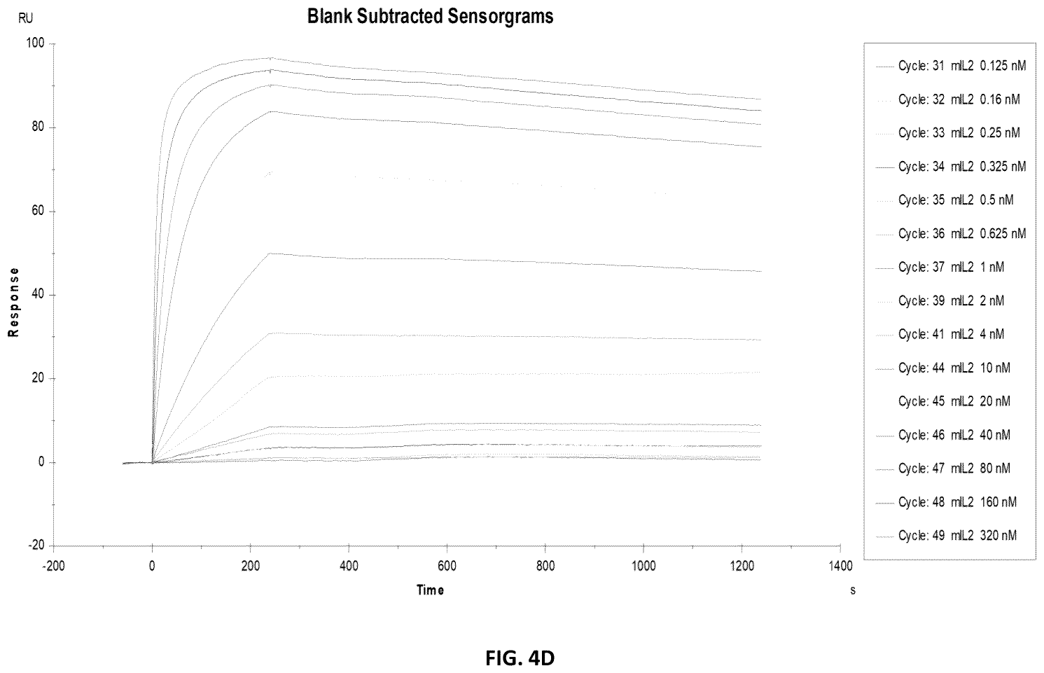

JES6.1 has been reported to bind mouse IL-2 (mIL-2) at a K_{D} of 5.6 nM. To test if JES6.1 could bind human IL-2 (hIL-2), JES6.1 antibody was tested by SPR on BiacoreT200. JES6.1 was cross-linked directly to the CM5 chip, then human IL-2 or mouse IL-2 analytes were streamed at concentrations ranging from 0.5 nM to 128 nM or 0.5 to 16 nM respectively. As can be seen in FIG. 4A, when tested with human IL-2, the JES6.1 showed no apparent change in response unit (RU). On the other hand, when mouse IL-2 served as analyte, a robust response was apparent (FIG. 4B), indicating that JES6.1 binds mouse IL-2 strongly but does not bind human IL-2. The experiment was repeated with the JES6.1RMC antibody chimera which was expressed as a JES6.1 rat FV with a mouse constant region as described herein. The JES6.1RMC was immobilized on the CM5 chip using the GE antibody capture kit. Streaming hIL-2 at a concentration of up to 100 nM resulted in no change in RU, indicating no binding to the human IL-2 (FIG. 4C). To test whether the JES6.1RMC chimera retained its mIL-2 binding properties like the JES6.1 above, it was tested for binding to mouse IL-2. Streaming of mIL-2 at a concentration of 0.5 nM to 320 nM resulted in large change in RU, indicating robust binding (FIG. 4D). These results indicate that JES6.1 and JES6.1RMC bind mouse IL-2 strongly but show no apparent binding to human IL-2. Analysis of the JES6.1RMC binding kinetics to both hIL-2 and mIL-2 are shown below.

TABLE 4
Binding Kinetics of JES6.1RMC.

| | Mouse IL-2 | | | Human IL-2 | | |
|---|---|---|---|---|---|---|
| Antibody | K_{D (M)} | ka (1/Ms) | kd (1/s) | K_{D (M)} | ka (1/Ms) | kd (1/s) |
| BDG JES6.1RMC | 1.3*10\hat{ }−10 | 4.8*10\hat{ }5 | 6.3*10−5 | N.D. | N.D | N.D. |
| (17.006) | | | | | | |

Changing Binding Specificity from Mouse IL-2 to Human IL-2

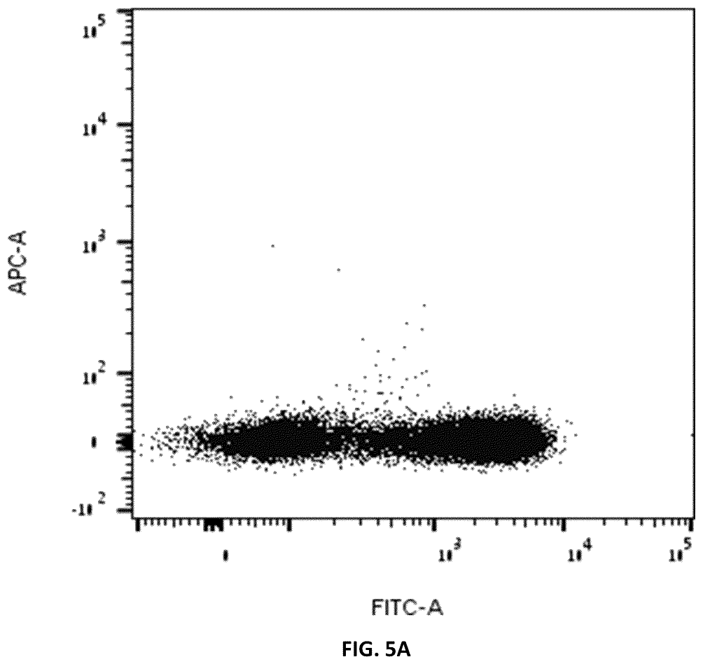

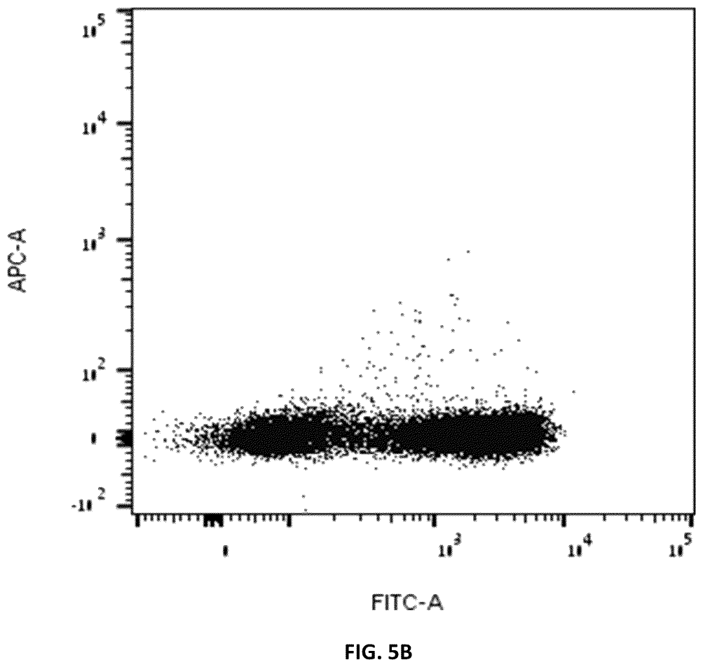

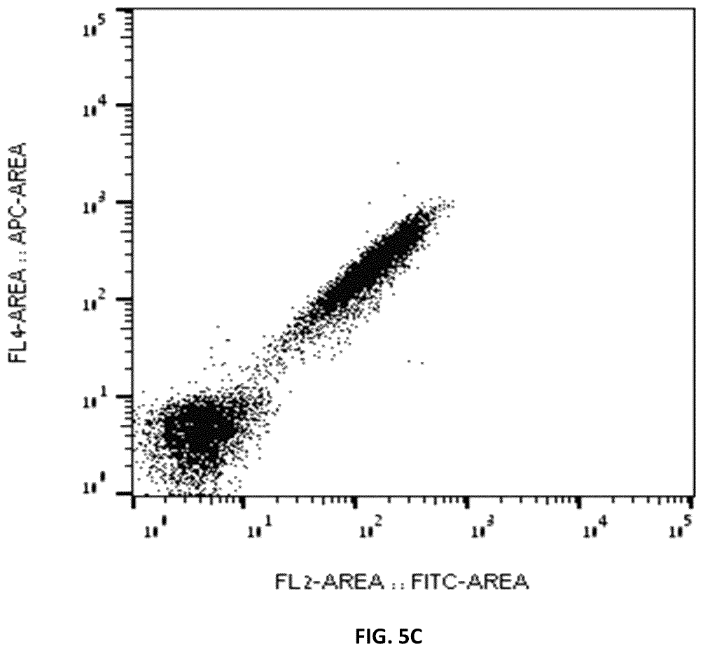

To change binding specificity from mouse IL-2 to human IL-2, JES6.1 was cloned as a scFv into a yeast display vector. The scFv format of JES6.1 expressed well on the yeast surface as indicated from the carboxy terminal myc tag labeling (FIGS. 5A-5C). Incubating 100 nM JES6.1 in IgG format with YSD clones expressing mouse IL-2 resulted in strong binding (FIGS. 5A-5C). However, in correlation with the SPR results shown above, incubation of JES6.1 YSD clones with up to 1 uM labeled human IL-2 showed no increase in fluorescence, indicating that the JES6.1 scFv does not bind hIL-2.

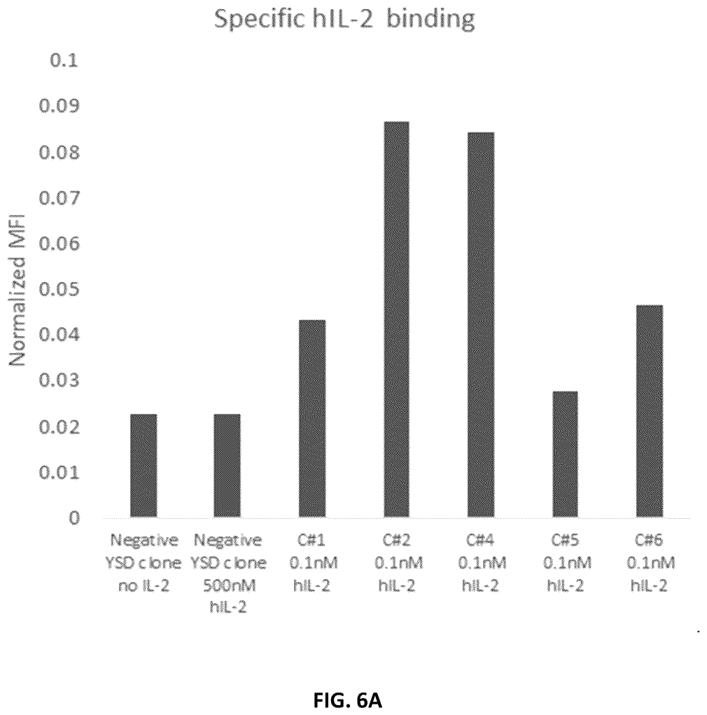

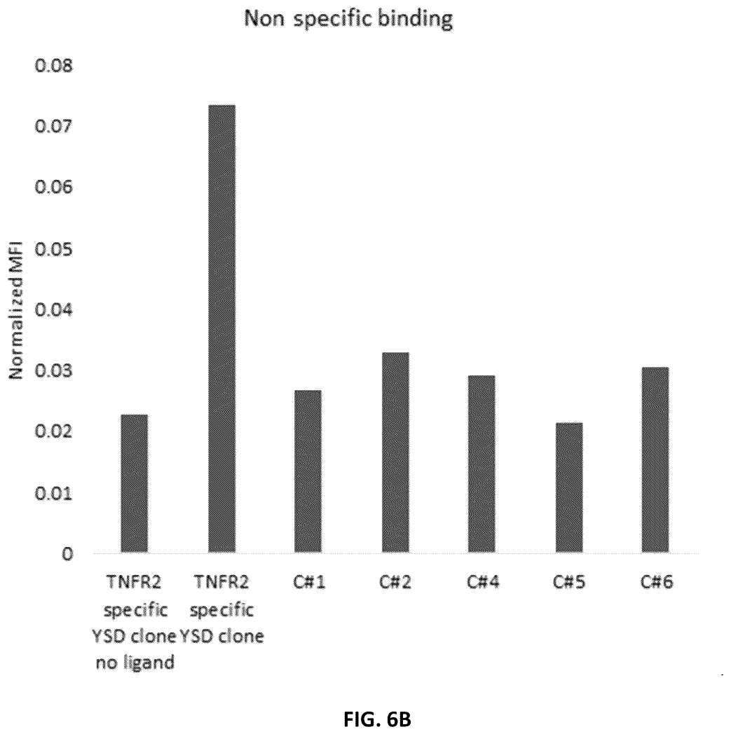

Based on the JES6.1 scFv, a mutagenesis library was generated as described above. Briefly, the YSD library was selected against recombinant human IL-2 as described above. The mutant library went through one round of MACS selection against 1 uM of human IL-2 and additional round of FACS selection against 1 uM of human IL-2. The top 0.2% clones were selected. Subsequently, the library underwent two additional rounds of selection specifically aimed at improving the koff properties of the selected clones as described above. For 3^{rd} round of selection the yeast were incubated with 10 nM His-tagged hIL-2 for 15 minutes at room temperature, then the yeast were washed of the hIL-2 and incubated for 5 minutes with 100 nM unlabeled IL-2 at room temperature. The 4^{th} round was done in a similar fashion but post labeling and wash, the yeast were incubated in fold of initial volume in PBS for 24 hours. In the 5^{th} round, the yeast were labeled and washed, and then incubated with 100 nM unlabeled IL-2 at room temperature for six hours. After five rounds of selection the clones were isolated. Five YSD clones that gained binding to hIL-2 (FIGS. 6A-6B) were sequenced. In addition, these clones were tested for specificity by labeling with a mixture of 500 nM soluble TNFR2, and 500 nM OX40 and 500 nM PD1. As can be seen in FIGS. 6A-6B, these clones are specific to hIL-2 and do not bind any of the other proteins.

### Expression of BDG17.023

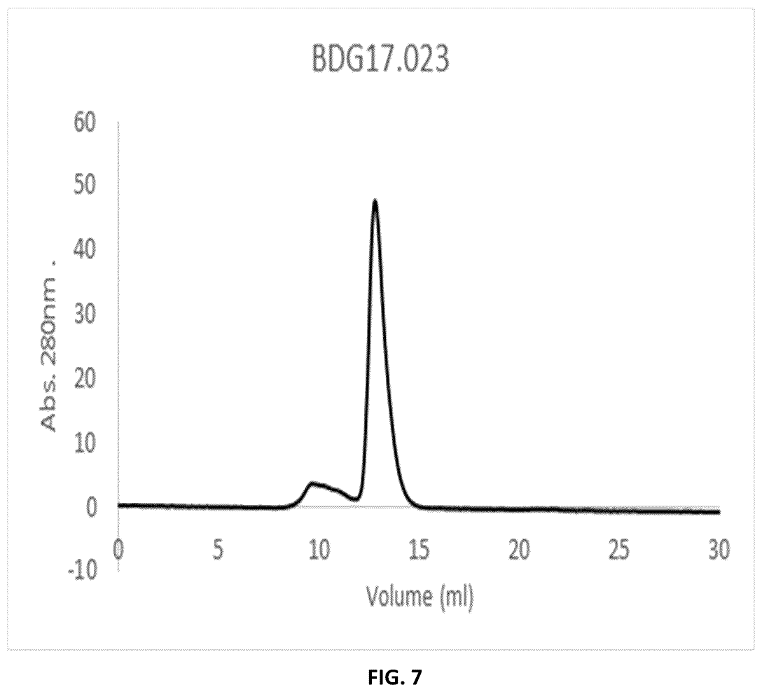

Subsequent to YSD characterization, clone #4, which showed significant binding to hIL-2, was reformatted to human chimeric IgG1 (BDG17.023) with rat FV and human Fc chimera. The rat variable domain was subcloned into two separate expression vectors, pSF-CMV-HuIgG1_HC and pSF-CMV-HuLambda_LC as described above. The IgG was expressed in ExpiCHO cells as described above. The purified IgGs were >95% pure as evident from a SDS PAGE analysis. Size exclusion chromatography of BDG17.023 on superdex200 10/300 showed two main peaks: the first peak with a retention time of ˜9.2 ml (0.36CV) was typical of large aggregate and the second peak with retention of approximately ˜12.6 ml (0.528CV) was typical of an ordinary human hIgG1. Peak integration of these SEC runs showed 110% and 89% respectively (FIG. 7).

### Binding Kinetics of BDG17.023

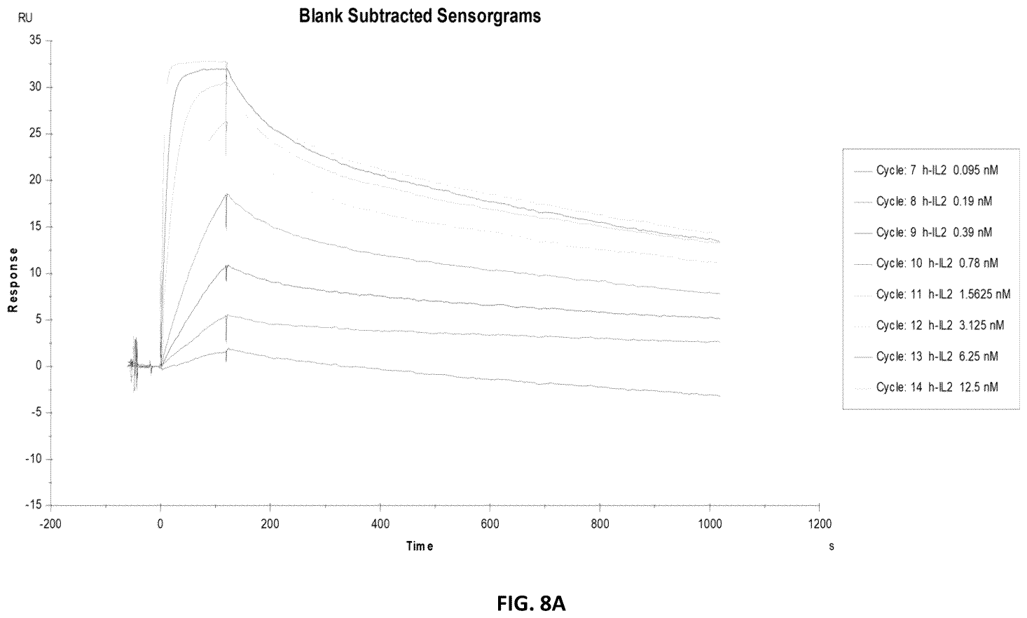

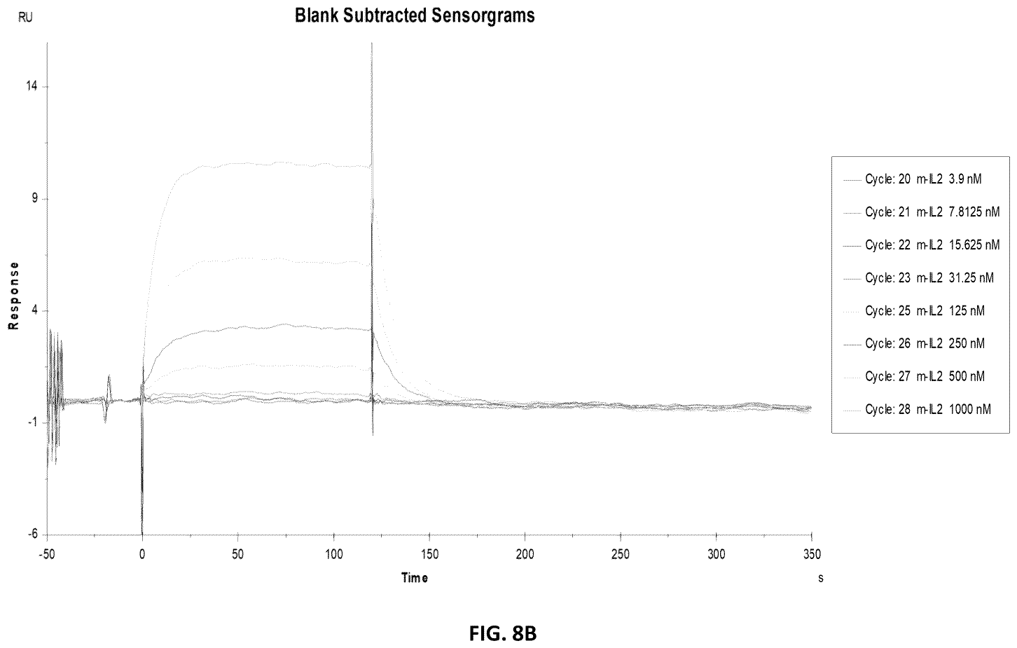

To determine BDG17.023 binding kinetics and affinity to mIL-2 and hIL-2, the IgG was analyzed by SPR on a BIAcore T200 using the GE capture antibody kit as described above. As shown in FIGS. 8A-8B, BDG17.023 binds hIL-2 with an affinity of approximately 8×10\hat{ }−11, with a on rate of 1.3*10\hat{ }7 and off rate of 1*10\hat{ }−3. BDG17.023 also showed binding to mIL-2 with a much lower affinity of approximately 2.5×10\hat{ }−6.

TABLE 5
Kinetic parameters of BDG17.023.

| | Mouse IL-12 | | | Human IL-12 | | |
|---|---|---|---|---|---|---|
| Antibody | K_{D (M)} | ka (1/Ms) | kd (1/s) | K_{D (M)} | ka (1/Ms) | kd (1/s) |
| BDG17.023 | 2.46E−06 | 3.98E+04 | 0.09795 | 8.18E−11 | 1.31E+07 | 0.00107 |

Receptor Discrimination of the BDG17.023-hIL-2 Complex

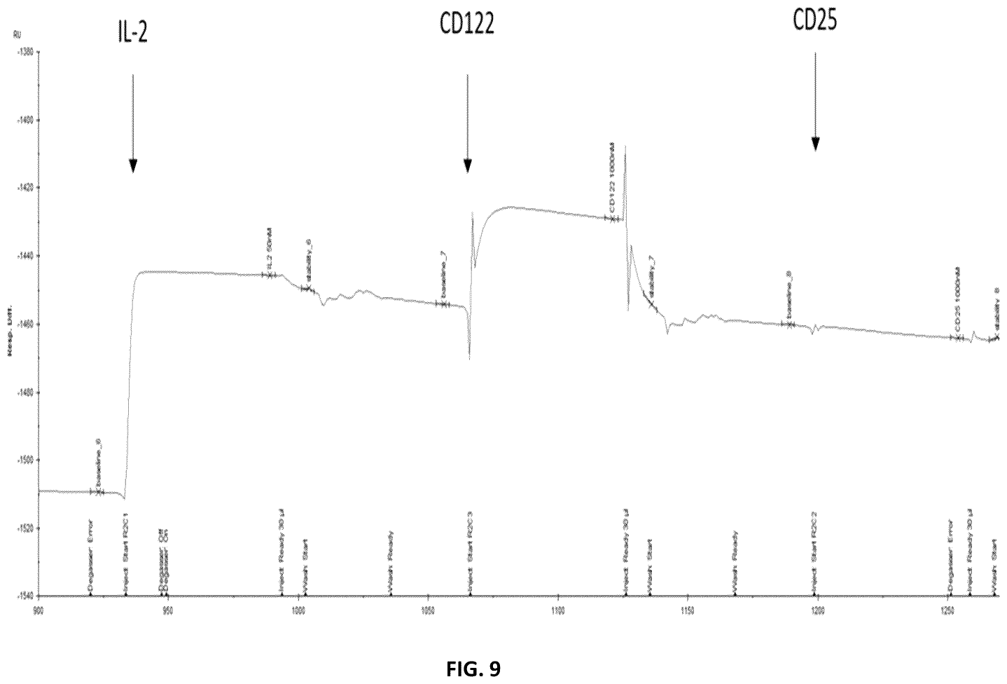

JES61-mIL-2 complex was reported to bind specifically to CD25 but not CD122. It was shown that the JES6.1-mIL-2 complex bound to a SPR chip could bind CD25 but not CD122. To test whether BDG17.023-hIL-2 complex can discriminate between binding to CD25 and CD122, a similar experiment was performed as described above. As shown in FIG. 9, a control antibody complexed with hIL-2 binds human CD25 but could not bind CD122. In contrast, the BDG17.023-hIL-2 complex was found to bind CD122 but not CD25. This result indicates that although BDG17.023 is derived from JES6.1, the JES6.1-mIL-2 complex and the BDG17.023-hIL-2 complex show a very different IL-2 receptor preference, possibly through binding of different epitope of the mIL-2 and the hIL-2 respectively. Alternatively, different allosteric effects may be induced on the mIL-2 and hIL-2 that affects binding preference to the IL-2 receptors.

### In Vivo Characterization of BDG17.023

In vivo administration of JES6.1 complexed with mIL-2 resulted in robust proliferation of regulatory T cells and much smaller proliferation of effector T cells, thereby shifting the MP CD8⁺/Tregs ratio towards immune suppression. Since the BDG17.023-hIL-2 complex showed preference to binding CD122 and excluded CD25 from binding in an SPR biochemical assay, it is predicted that the BDG17.023-hIL-2 complex would enhance proliferation of CD8⁺ effector cells and NK cells in vivo. Human IL-2 can cross react with the mouse IL-2 receptors, thus the BDG17.023-hIL-2 complex was administered to C57BL/6 mice to test its effect in vivo as described above. Briefly, the 17.023 antibody-hIL-2 complex was incubated with hIL-2 at a 1:1 molar ratio and injected intraperitoneally to C57BL/6 mice daily, for four consecutive days. As a control. JES6.1-mIL-2 complex, hIL-2 alone, or mIL-2 alone was also administered in a similar fashion. On the fifth day the mouse spleens were harvested, cells were labeled and analyzed by FACS as described above.

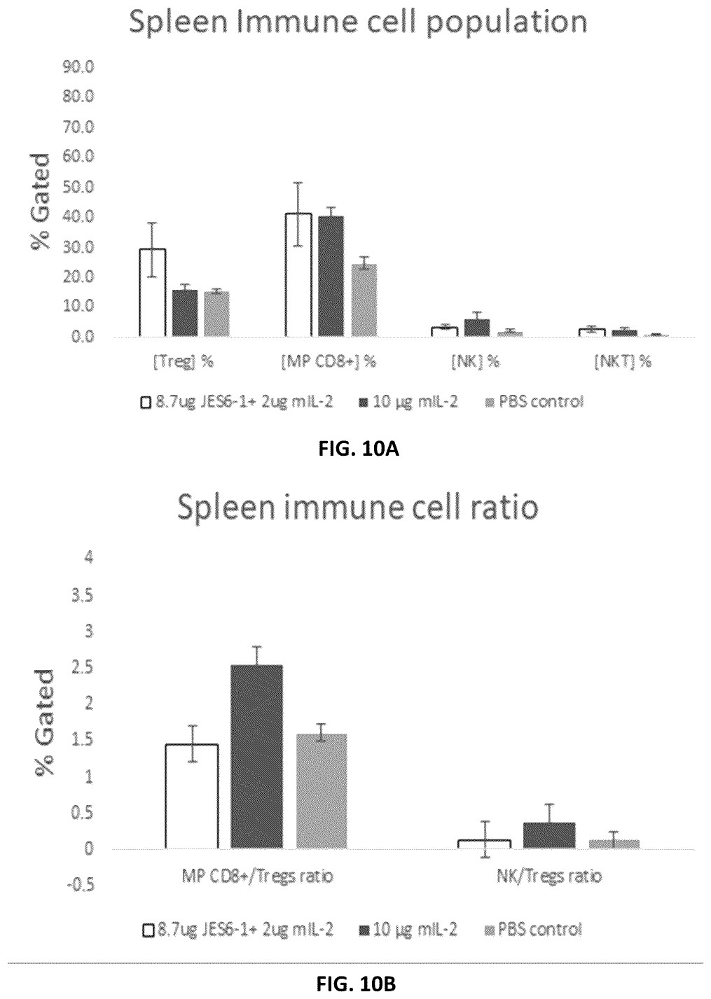

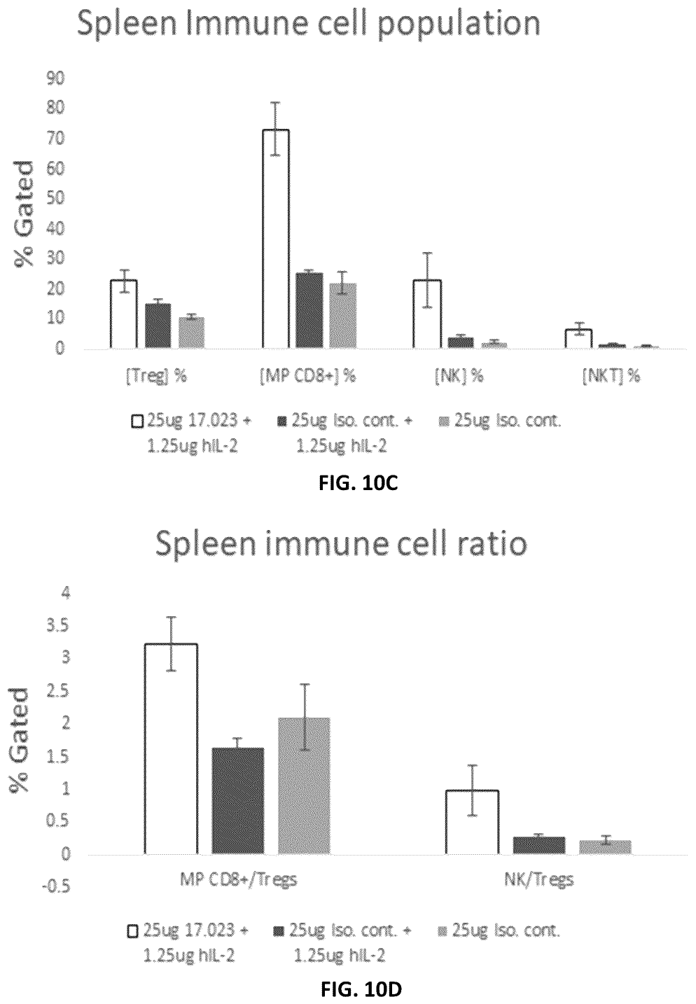

As can be seen in FIGS. 10A-10D, BDG17.023 shows pronounced effect in inducing proliferation of MP CD8⁺ cells and NK cells, while there was much smaller effect on CD4⁺ Tregs. On the other hand, JES6.1 showed a much different effect in accordance with its reported anti-inflammatory effect. These results demonstrate that in agreement with the binding data and in contrast to JES6.1, BDG17.023-IL-2 complex has a strong stimulatory effect on the immune system in vivo, as opposed to an anti-stimulatory or pro-regulatory effect.

### Example 2

This example presents results on further selection of human IL-2 binder and generation of humanized antibodies.

Additional selection strategy was used to select human IL-2 binder under alternative selection pressure. Briefly: the library went through one round of MACS selection against 1 uM of human IL-2 and additional four rounds of FACS selection against 100 nM of human IL-2. After the first round of FACS selection, all binders were selected, followed by selection of the top 0.5%, top 0.5% and top 0.1% of the binders. After five rounds of selection, clone C #7 (173R5C1-17.002) (SEQ ID NO:28) was isolated, verified for binding and sequenced.

### Antibody Humanization

Clone C #7 (173R5C1-17.002) was selected as template for humanization. A human template was chosen using the Schrodinger BioLuminate ‘Antibody Humanization: CDR Grafting’ CDR tool (Kai Zhu, Tyler Day et al., Antibody structure determination using a combination of homology modeling, energy-based refinement, and loop prediction. Proteins: Structure, Function and Bioinformatics, 82, 8, 8 2014), and the PDB entry of JES6-1 was used as a query (4YQX; Jamie B. Spangler, Jakub Tomala et al., Antibodies to Interleukin-2 Elicit Selective T Cell Subset Potentiation through Distinct Conformational Mechanisms. Immunity, 42, 5, 5 2015). PDB entry 5118 (Alexey Teplyakov, Galina Obmolova et al., Structural diversity in a human antibody germline library. mAbs, 8, 6, 8 2016) was chosen as it had the best score with regard to a combination of framework identity of the L and H chains and stem geometry. Mutations were introduced to positions that either interact (within 5A radius) with the CDR regions (according to IMGT numbering scheme), or the antigen in 4YQX. The variability on these positions was selected to include amino acids of the human template (5118) as well as the mouse query (4YQX). In addition, due to significant structural changes between H1 of the query and the template, the option of complete transition between H1 of 4YQX and 5118 was introduced. This library had approximately 1300 different variants.

The library underwent selection by FACS for two rounds. In the first round, the library was labeled with 5 nM hIL-2 and the top 5% binding clones were sorted. In the second round, the library was labeled with 1 nM hIL-2 and the top 5% of the clones were sorted. The clones were sequenced after selection and clone C #8 (173.2A.C6-17.014) (SEQ ID NO:31) was used as a template for affinity maturation.

### Humanized Antibodies Affinity Maturation

CDR positions (IMGT/ABR definition) that were predicted to have a high rate of somatic hypermutation were selected for variation. Additional positions in L3 that are presumed to interact with the antigen based on JES6-1, as well as all of H3, were also targeted for variation. The variation was based on sequence conservation of all positions except for H3 which was the DHY codon. The theoretical diversity of this library was 3.21×10¹².

Based on the clone C #8 (173.2A.C6-17.014) another library was constructed, in which all CDR positions were explored using the NNS degenerate codon that encodes all amino acids. Variants of 2-3 mutations, up to one on each CDR were screened. The theoretical size of such a library is 2×10⁶ double mutants and 1×10⁹ triple mutants.

The humanization affinity maturation libraries generated above were pooled together for selection. Briefly, in the first round the pooled YSD libraries were labeled with 10 nM hIL-2 and selected on MACS. In the second round the yeast were labeled with 0.1 nM hIL-2 and selected using MACS. In the third round of selection, the yeast were labeled with 0.1 nM hIL-2 and all binders were selected on FACS. For the fourth and fifth round of selections, the yeast were labeled with 10 nM hIL-2 and competed with 100 nM unlabeled hIL-2 for 24 hours and 48 hours respectively. Subsequently, the yeast were sorted on FACS to select all the binders. The final round of selection was done in a similar fashion to the fourth and fifth rounds, but post labeling and wash, the yeast were incubated in 1000 fold of initial volume in PBS at room temperature for a week.

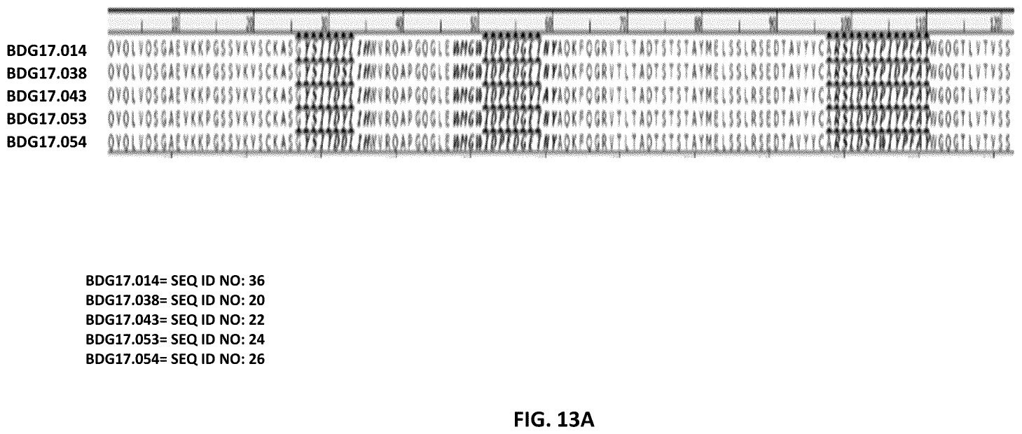

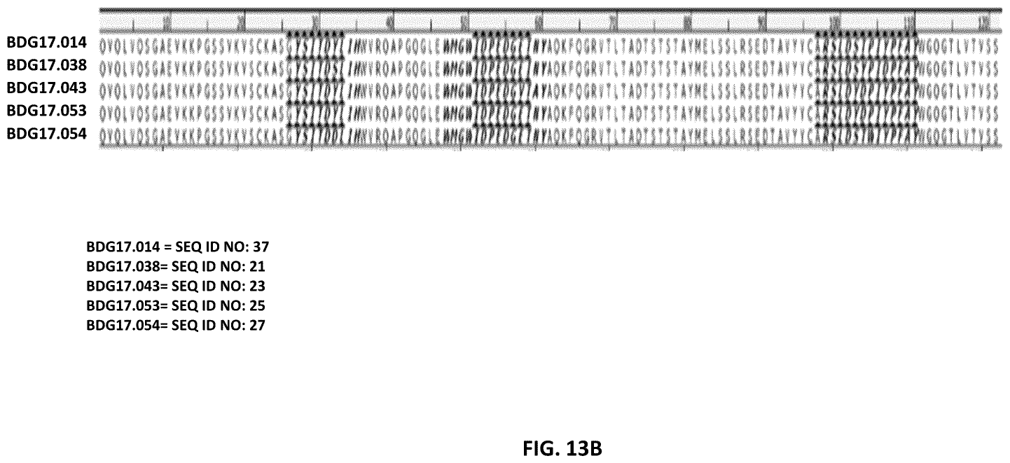

After selection, the clones were isolated, verified for binding and sequenced. Amino acid sequences for several clones are shown below. Alignment of Heavy Chain and Light Chain variable regions, and CDR regions of a subset of clones is presented in FIGS. 13A and 13B.

TABLE 6
VH and VL Amino Acid Sequences of
a Sub-Set of Anti-IL-2 Clones.

| | Heavy Chain | Light Chain |
|---|---|---|
| Clone | Variable Region | Variable Region |
| 17.038 | SEQ ID NO: 20 | SEQ ID NO: 21 |
| 17.043 | SEQ ID NO: 22 | SEQ ID NO: 23 |
| 17.053 | SEQ ID NO: 24 | SEQ ID NO: 25 |
| 17.054 | SEQ ID NO: 26 | SEQ ID NO: 27 |
| 17.014 | SEQ ID NO: 36 | SEQ ID NO: 37 |
| 17.066 | SEQ ID NO: 20 | SEQ ID NO: 21 |
| 17.067 | SEQ ID NO: 22 | SEQ ID NO: 23 |
| 17.069 | SEQ ID NO: 26 | SEQ ID NO: 27 |

It should be noted that clones 17.066, 17.067, and 17.069 comprise the LALA mutation (L234A, L235A mutations).

CDR sequences for certain selected clones are shown below.

TABLE 7
CDR Amino Acid Sequences of Anti-IL-2 Clones.

| | Heavy Chain | | | Light Chain | | |
|---|---|---|---|---|---|---|
| Clones | CDR1 | CDR2 | CDR3 | CDR1 | CDR2 | CDR3 |
| 17.014 | SEQ ID | SEQ ID | SEQ ID | SEQ ID | YAS | SEQ ID |
| | NO: 38 | NO: 39 | NO: 40 | NO: 41 | | NO: 43 |
| 17.038 | SEQ ID | SEQ ID | SEQ ID | SEQ ID | YAS | SEQ ID |
| | NO: 44 | NO: 45 | NO: 46 | NO: 47 | | NO: 49 |
| 17.043 | SEQ ID | SEQ ID | SEQ ID | SEQ ID | YAS | SEQ ID |
| | NO: 50 | NO: 51 | NO: 52 | NO: 53 | | NO: 55 |
| 17.053 | SEQ ID | SEQ ID | SEQ ID | SEQ ID | YAS | SEQ ID |
| | NO: 56 | NO: 57 | NO: 58 | NO: 59 | | NO: 61 |
| 17.054 | SEQ ID | SEQ ID | SEQ ID | SEQ ID | DAS | SEQ ID |
| | NO: 62 | NO: 63 | NO: 64 | NO: 65 | | NO: 67 |
| 17.066 | SEQ ID | SEQ ID | SEQ ID | SEQ ID | YAS | SEQ ID |
| | NO: 44 | NO: 45 | NO: 46 | NO: 47 | | NO: 49 |
| 17.067 | SEQ ID | SEQ ID | SEQ ID | SEQ ID | YAS | SEQ ID |
| | NO: 50 | NO: 51 | NO: 52 | NO: 53 | | NO: 55 |
| 17.069 | SEQ ID | SEQ ID | SEQ ID | SEQ ID | DAS | SEQ ID |
| | NO: 62 | NO: 63 | NO: 64 | NO: 65 | | NO: 67 |

TABLE 8
Full-length Amino Acid Sequences
of a Subset of Anti-IL-2 Clones.

| BGD-Clone | Heavy Chain (LALA) | Light Chain |
|---|---|---|
| BDG17.066 | SEQ ID NO: 68 | SEQ ID NO: 69 |
| BDG17.067 | SEQ ID NO: 70 | SEQ ID NO: 71 |
| BDG17.069 | SEQ ID NO: 72 | SEQ ID NO: 73 |

### Binding Kinetics of Humanized Antibodies

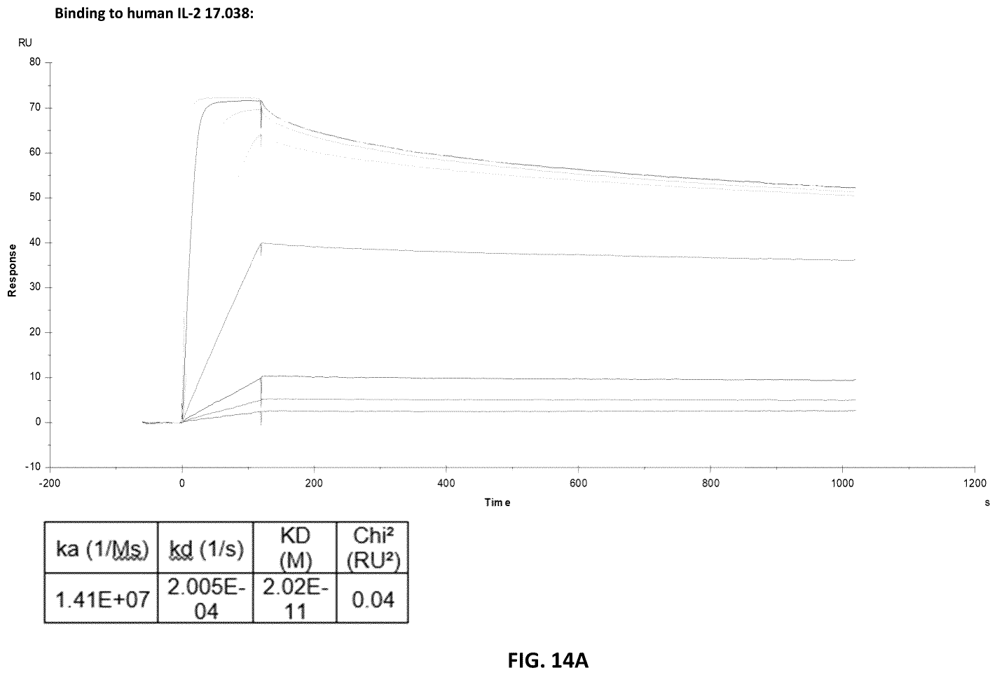

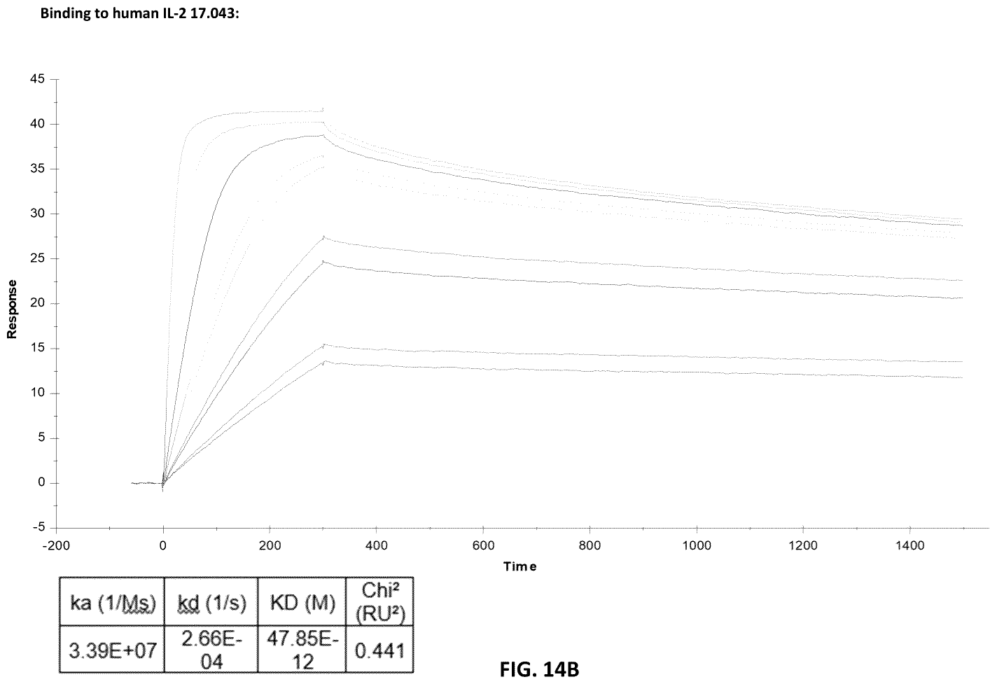

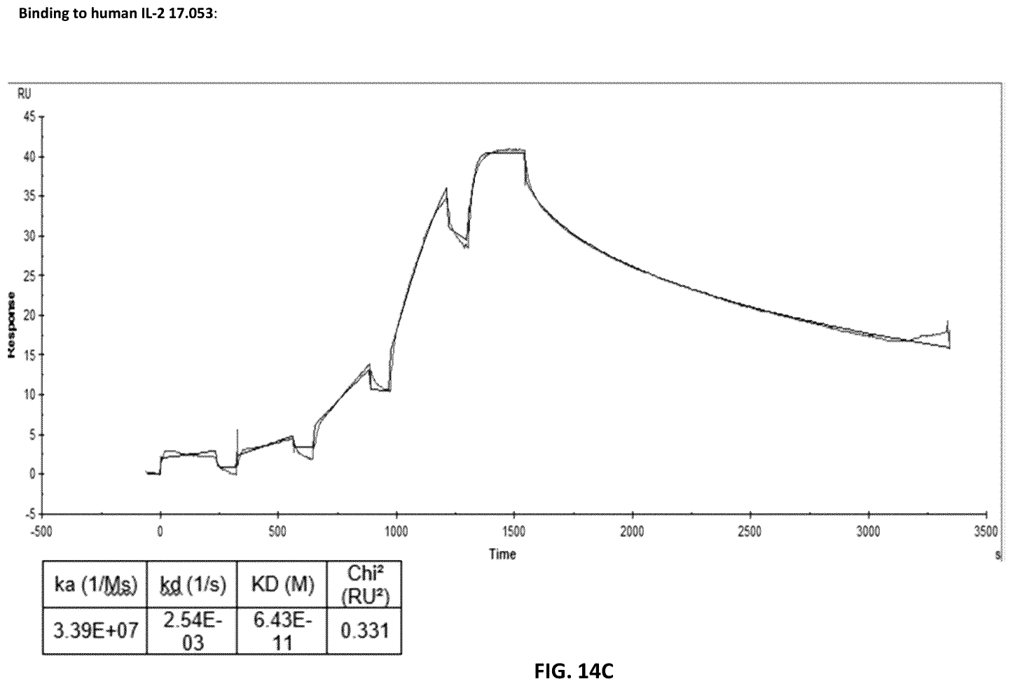

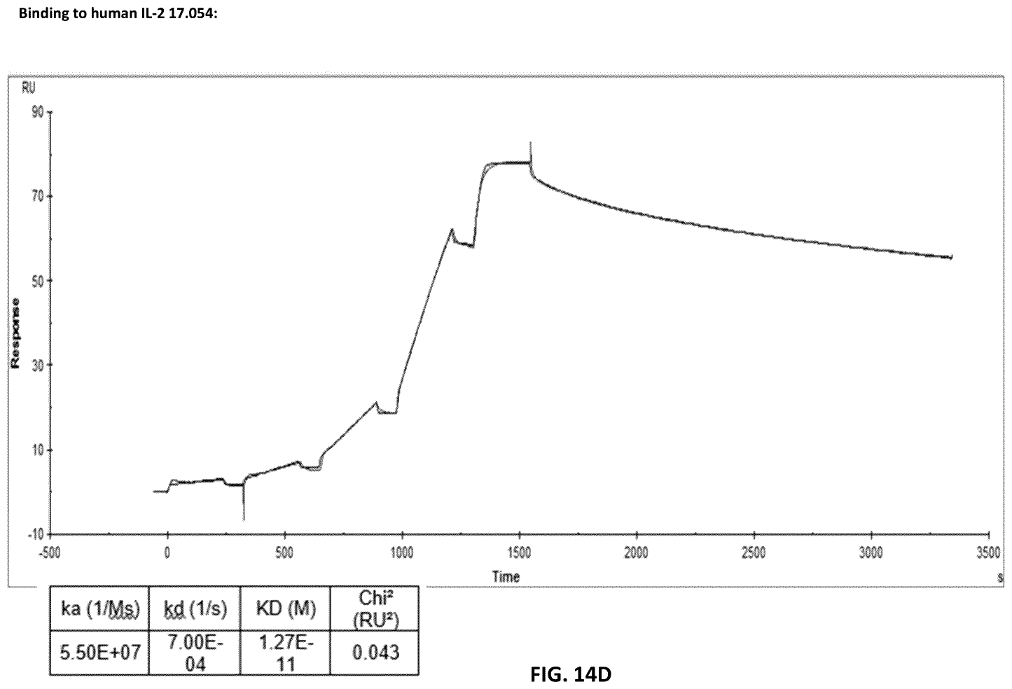

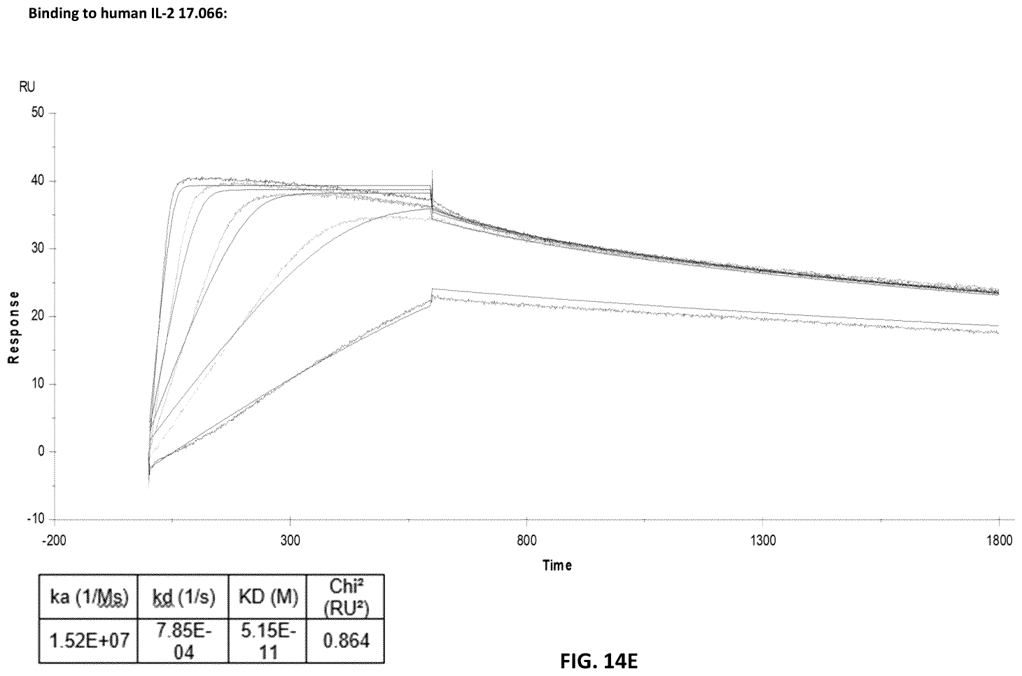

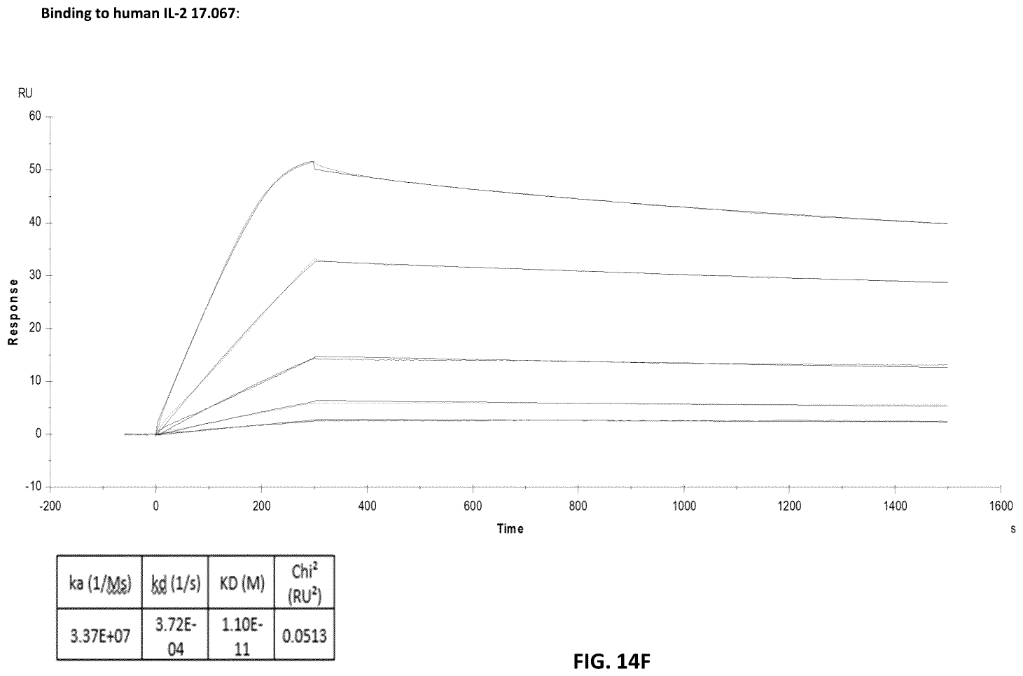

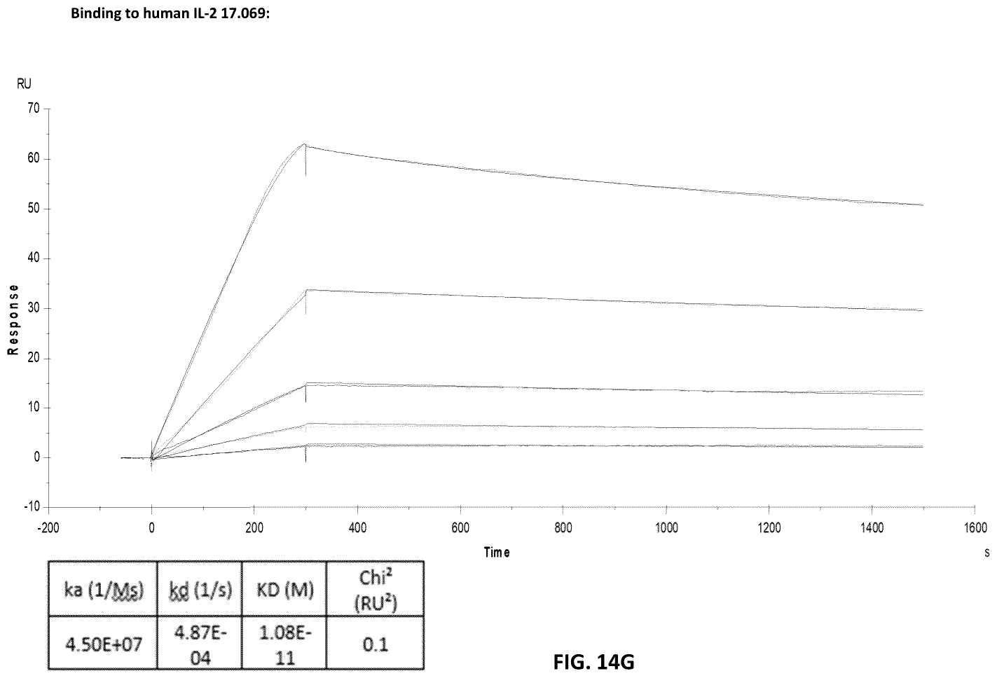

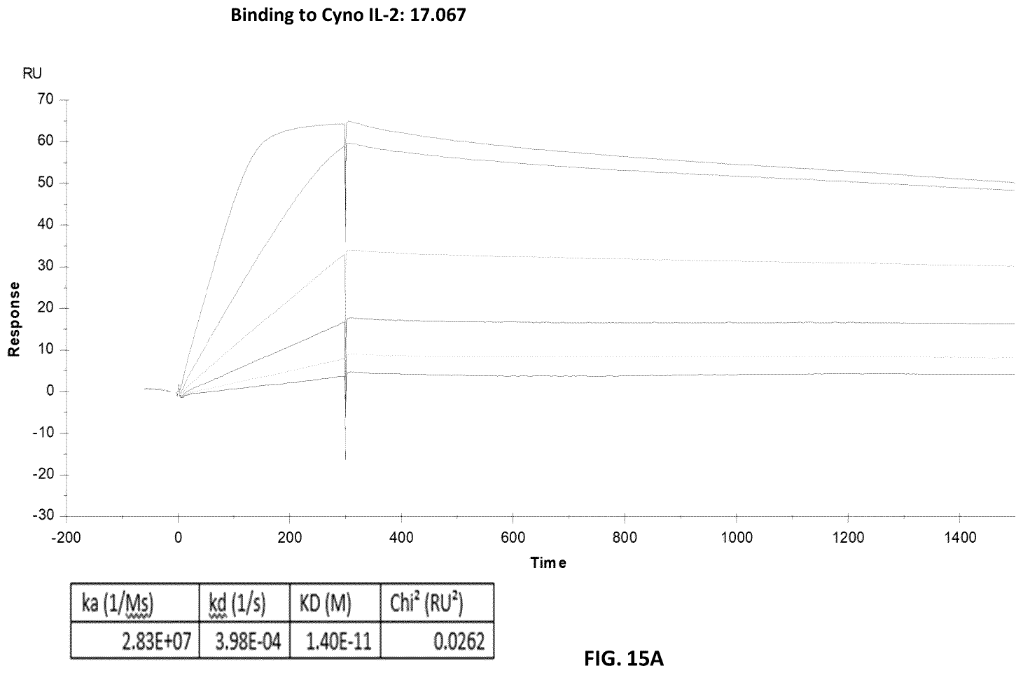

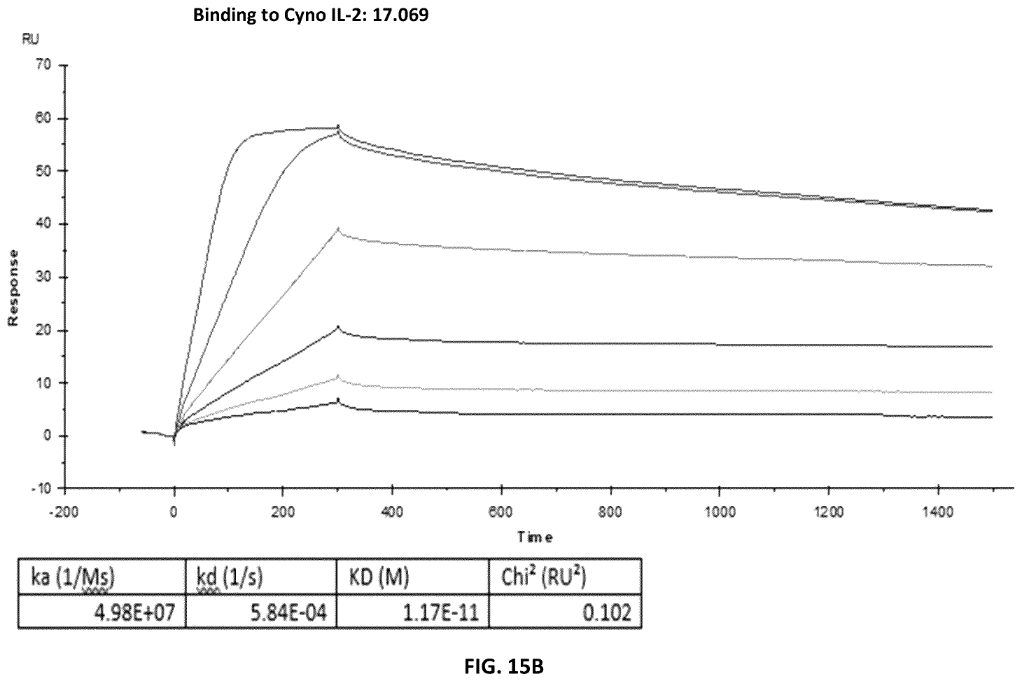

To determine BDG17.038, BDG17.043, BDG17.053, BDG17.054, BDG17.067, BDG17.066 and BDG 17.069 binding kinetics and affinity to hIL-2 and cynomolgus monkey IL-2 (cIL-2), the clones were reformatted to IgG expressed and purified; Subsequently the antibodies were analyzed by SPR on a BIAcore T200 using the GE capture antibody kit as described herein. As shown in Table 9, FIGS. 14A-14G, and FIGS. 15A-15B, the antibodies bind tightly to both human and cynomolgus monkey IL-2 in the low double digit pM range.

### Size Exclusion Chromatography Profile and Thermal Stability of the Humanized Antibodies.

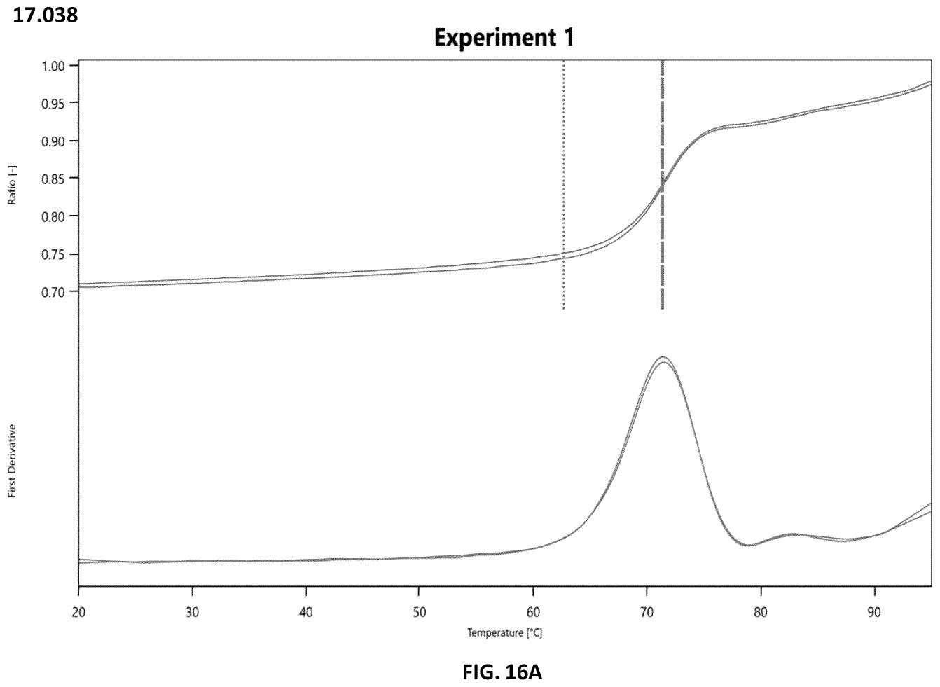

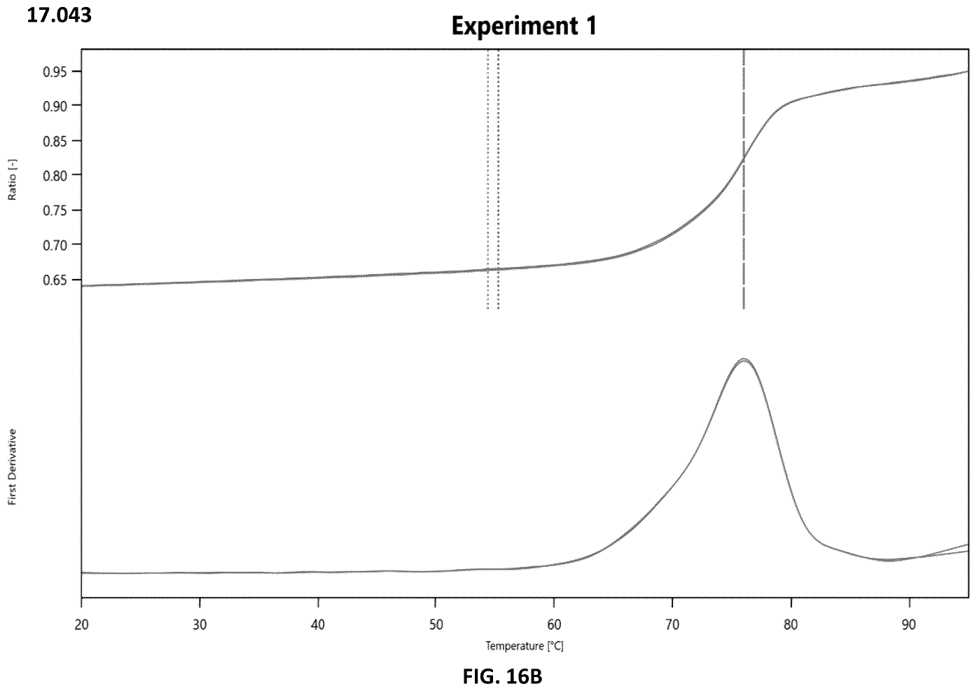

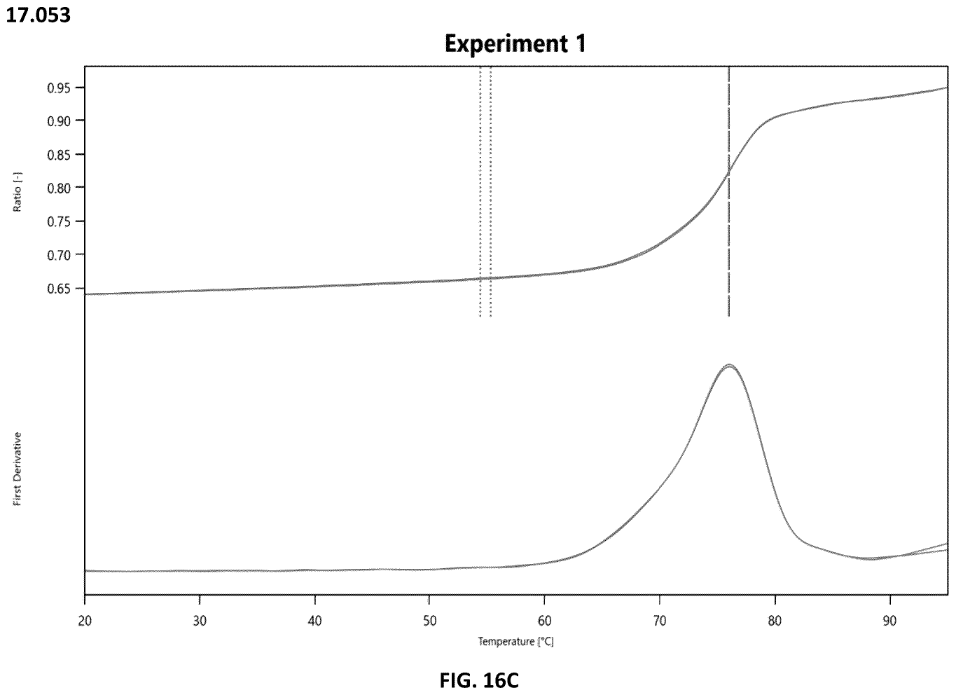

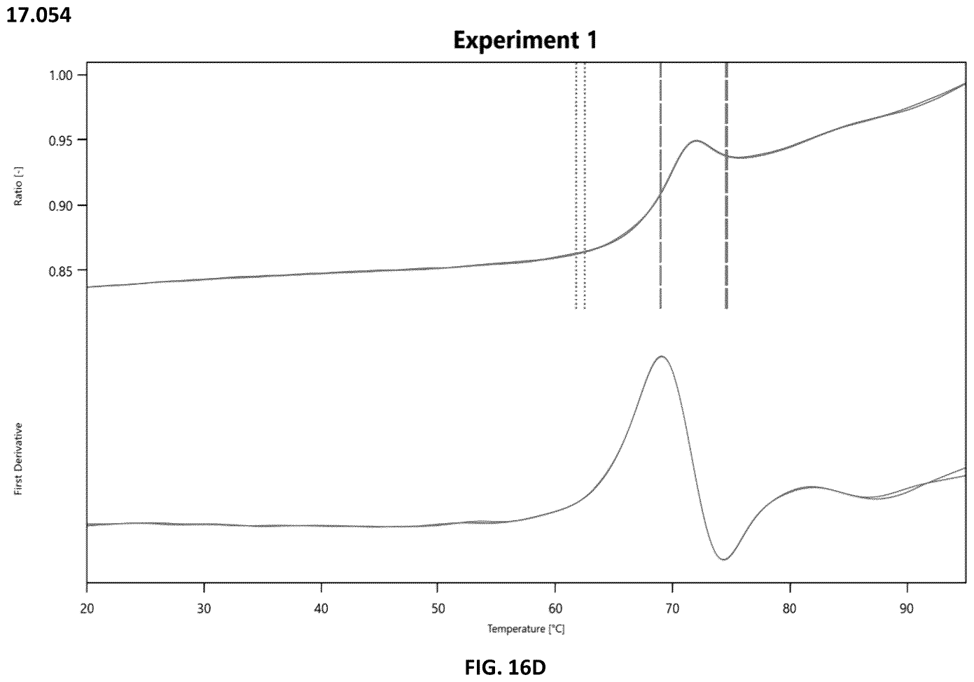

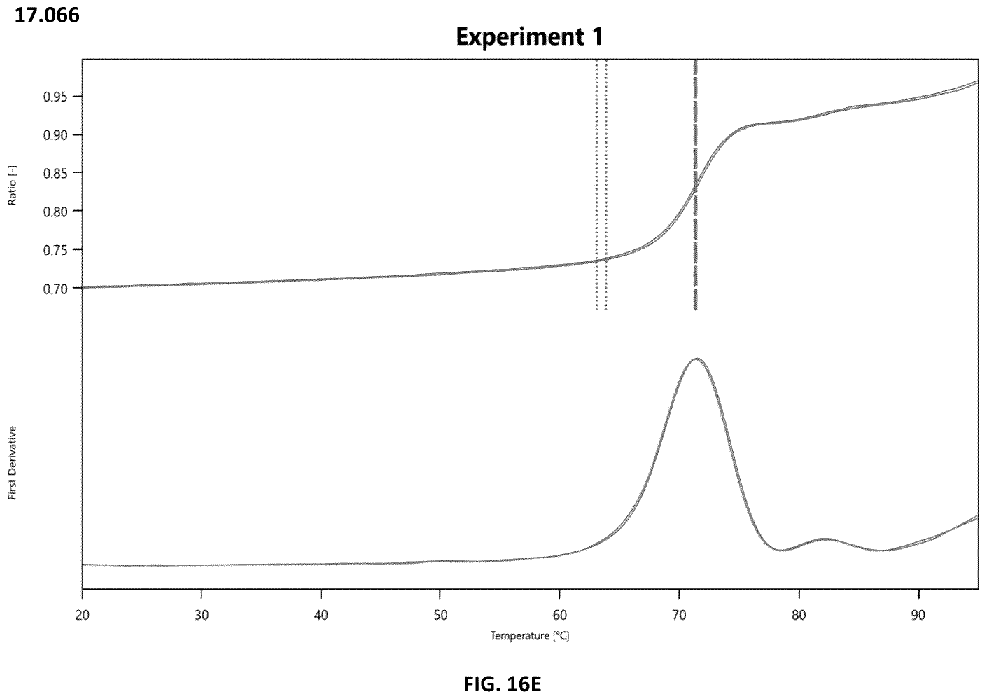

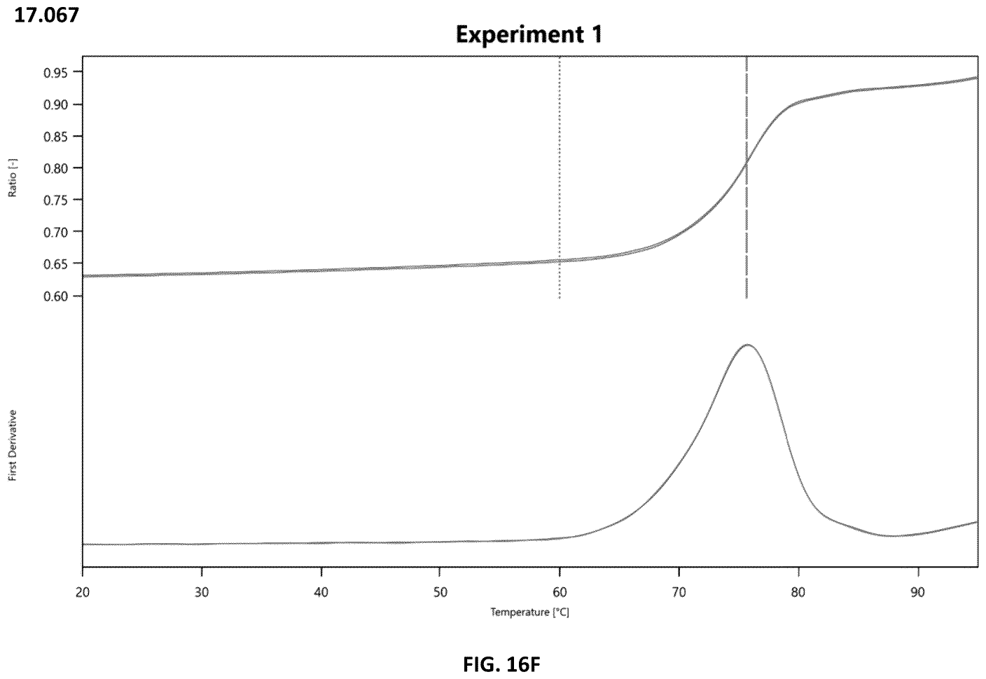

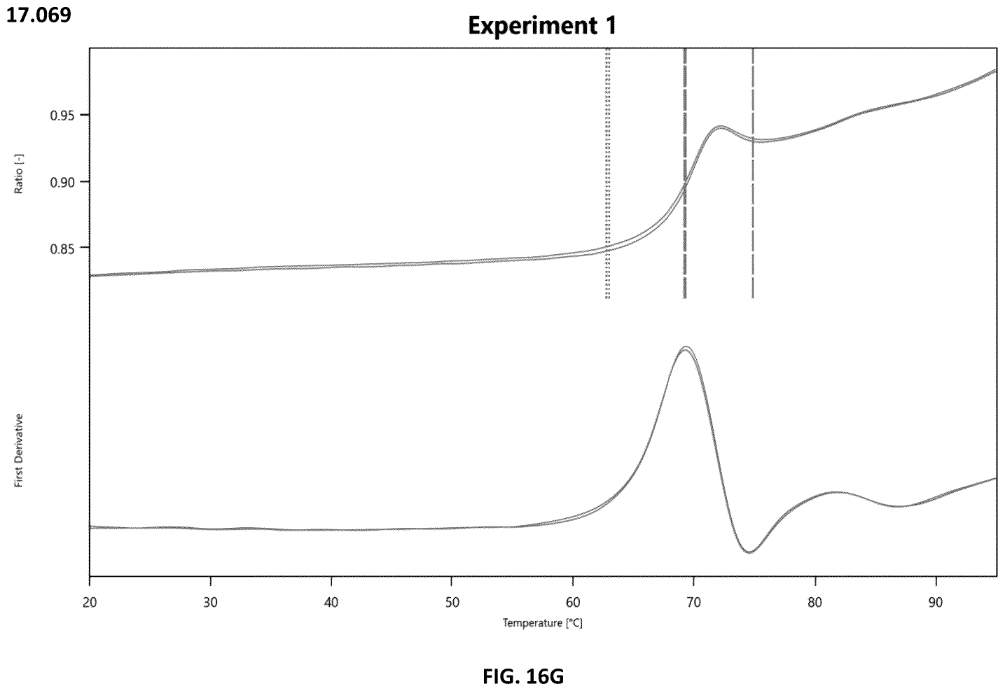

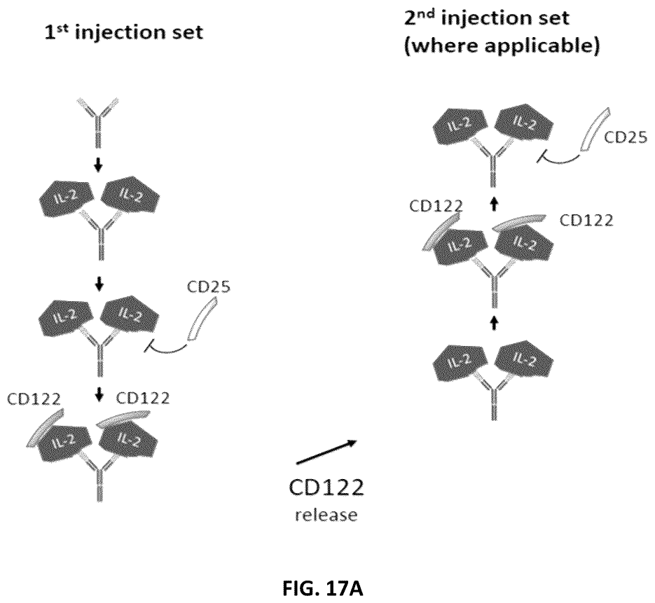

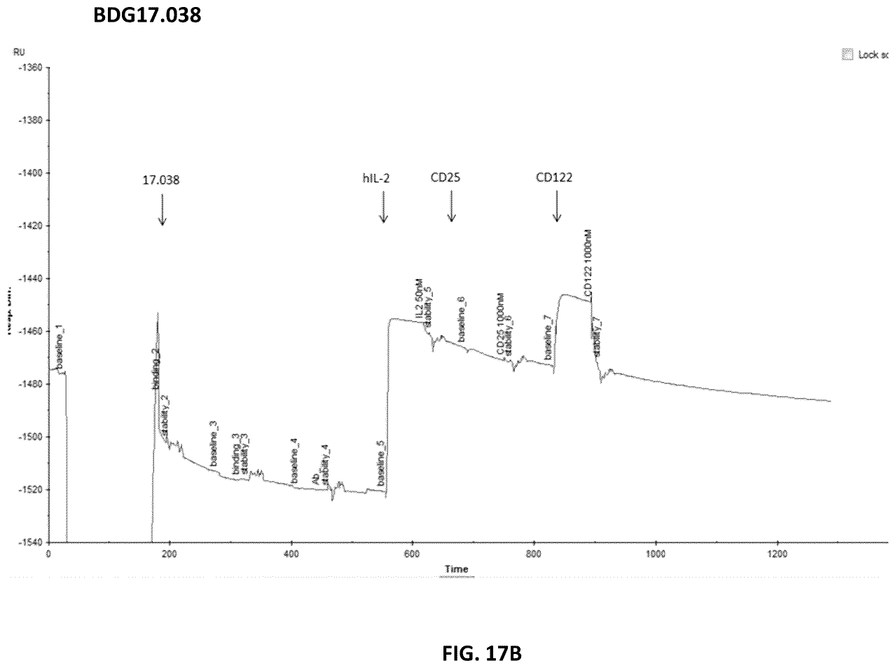

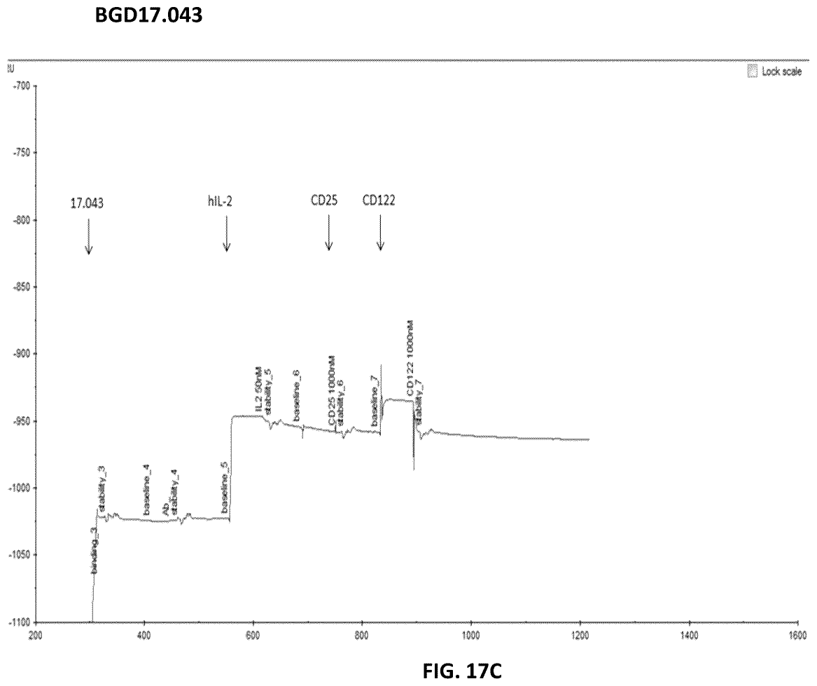

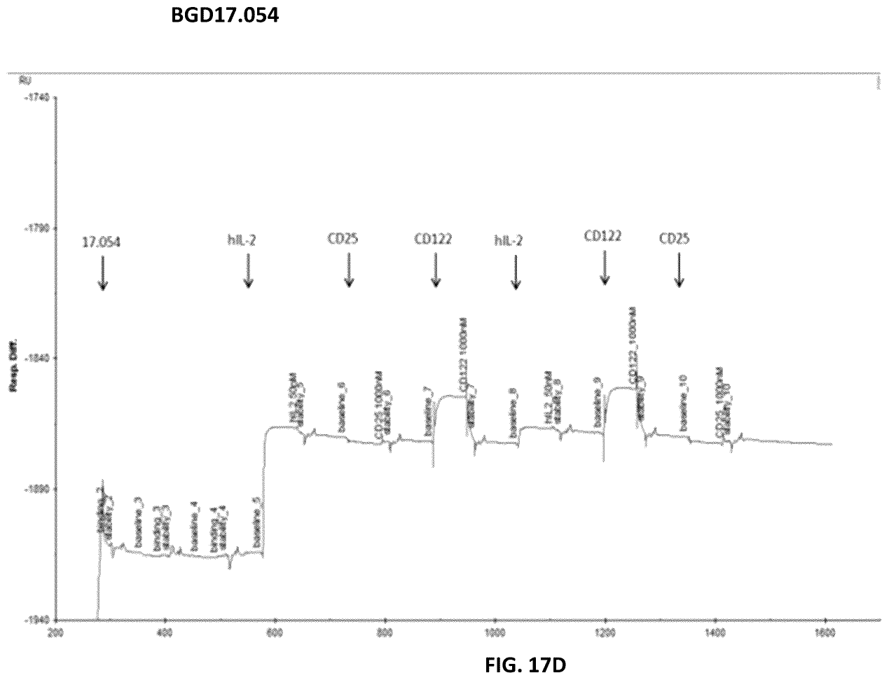

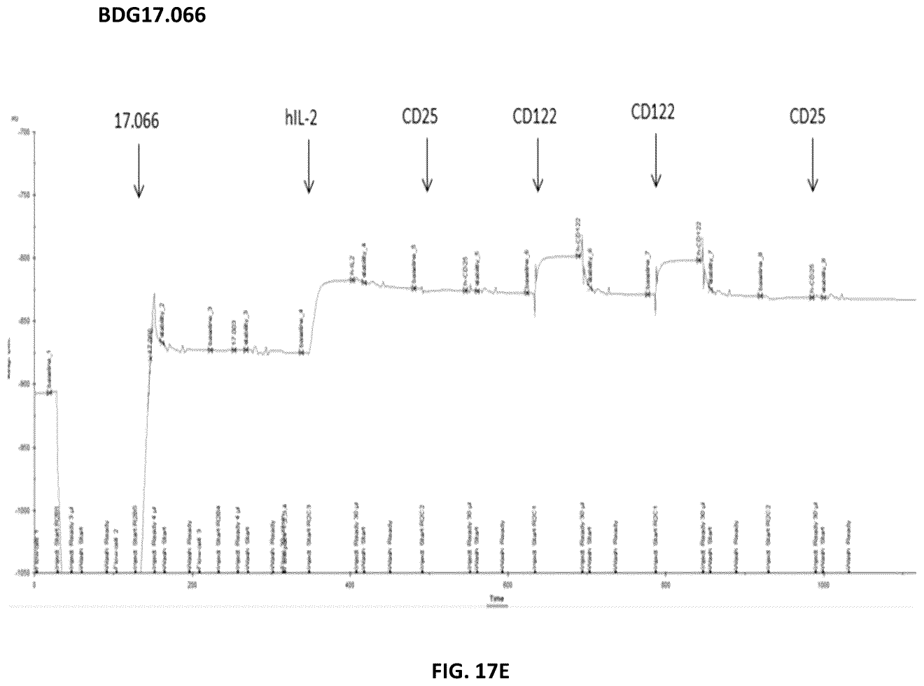

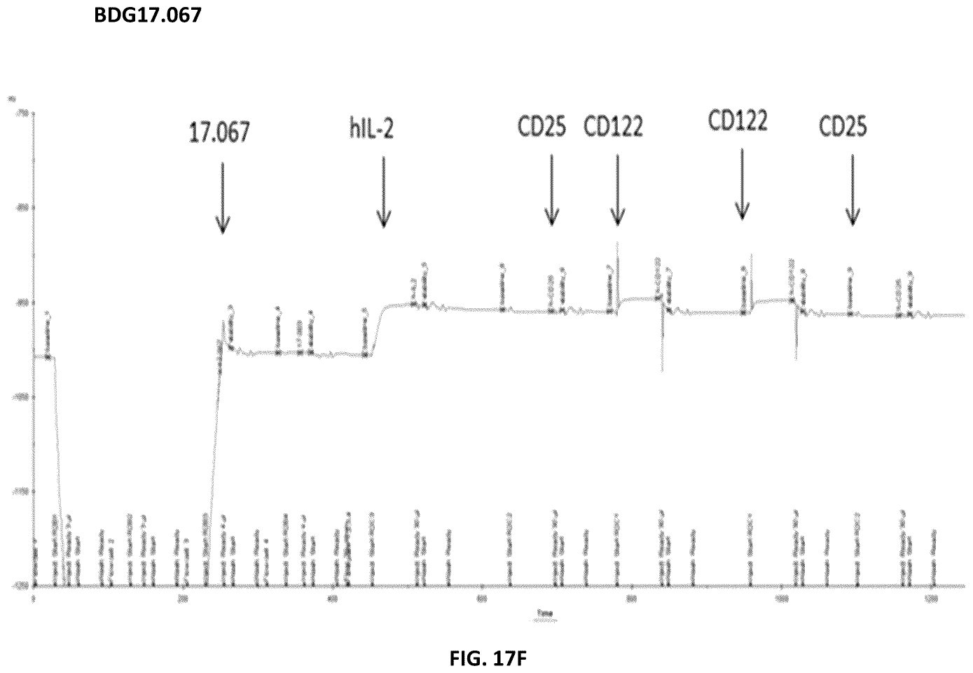

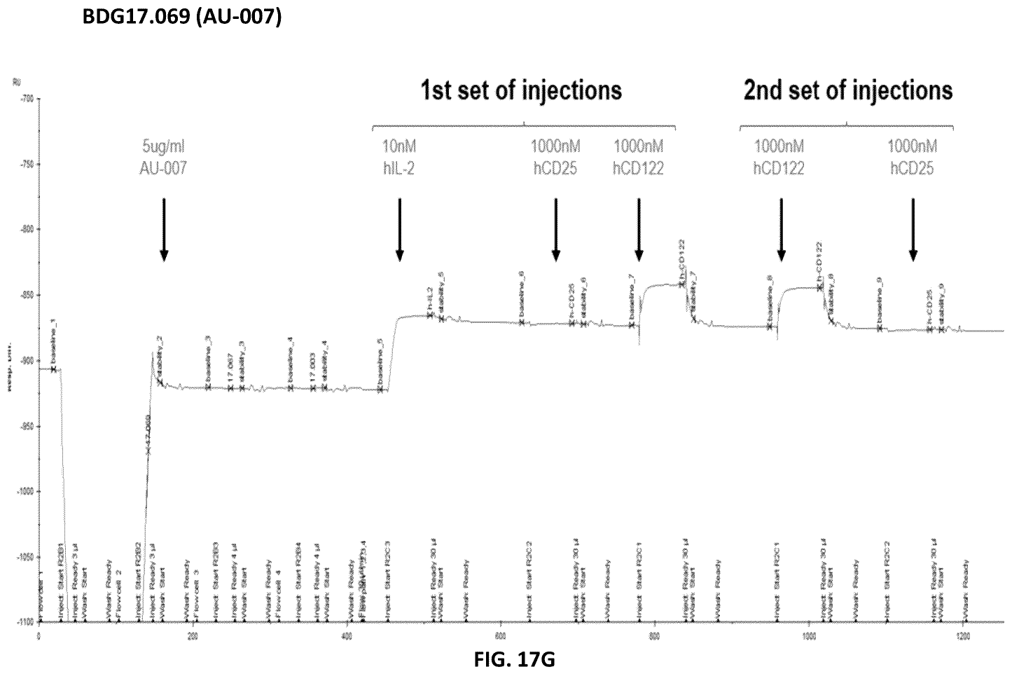

To test if the humanized IgGs BDG17.038, BDG17.043, BDG17.053, BDG17.054, BDG17.066, BDG17.067 and BDG 17.069 are folded correctly and stable, the antibodies were subjected to size exclusion chromatography and Differential Scanning Fluorimetry (DSF) analysis as described above. The results are summarized in FIGS. 16A-16G and Table 8, suggest that these IgGs are produced as >95% non-aggregated species, have a SEC retention profile typical of a human IgG1 and thermal denaturation profile with Tonset of >=54.4° C. and Tm1 >=69° C. An illustration of the receptor discrimination assay is presented in FIG. 17A, and the SPR results for the different clones are presented in FIGS. 17B-17G. To conclude the SPR, SEC and DSF experiments indicate that the humanized antibodies bind human and cynomolgus money IL-2 tightly, are folded correctly and are highly stable.

TABLE 9
Biophysical Properties of Select Improved Humanized Clones

| | | | | | Size exclusion | | | | | SPR | | |
|---|---|---|---|---|---|---|---|---|---|---|---|---|
| | | | | | chromatography | | SPR | | | Kinetics of | | |
| | | | | | GE superdex200 | | Kinetics of | | | binding | | |
| | | | | | increase 10/300 | | binding | | | Cynomolgus | | |
| | IgG | | | | Peak | % | human IL-2 | | | monkey IL-2 | | |
| IgG | infor- | DSF | | | vol- | aggre- | Ka | Kd | KD | Ka | Kd | KD |
| ID | mation | T onset | Tm1 | Tm2 | ume | gation | (Ms⁻¹) | (s⁻¹) | (M) | (Ms⁻¹) | (s⁻¹) | (M) |
| 17.038 | Humanized | 62.6° | 71.4° | N/A | 13.33 | 0.38 | 1.41E+07 | 2.00E−04 | 2.02E−11 | Not | Not | Not |
| | IgG1 | C. | C. | | ml | | | | | tested | tested | tested |
| 17.043 | Humanized | 54.4° | 76.0° | N/A | 13.04 | 0 | N/A | 2.22E−04 | 4.82E−11 | Not | Not | Not |
| | IgG1 | C. | C. | | ml | | | | | tested | tested | tested |
| 17.053 | Humanized | 55.1° | 76.1° | N/A | 13.01 | 4.13 | 3.96E+07 | 2.54E−03 | 6.43E−11 | Not | Not | Not |
| | IgG1 | C. | C. | | ml | | | | | tested | tested | tested |
| 17.054 | Humanized | 61.8° | 69.0° | 74.6° | 13.04 | 4.16 | 5.50E+07 | 7.00E−04 | 1.27E−11 | Not | Not | Not |
| | IgG1 | C. | C. | C. | ml | | | | | tested | tested | tested |
| 17.066 | Humanized | 63.9° | 71.5° | N/A | 12.96 | 3.8 | 1.52E+07 | 7.85E−04 | 5.15E−11 | Not | Not | Not |
| | IgG1 with | C. | C. | | ml | | | | | Tested | Tested | Tested |
| | reduced | | | | | | | | | | | |
| | Fc-gamma | | | | | | | | | | | |
| | binding | | | | | | | | | | | |
| | (LALA | | | | | | | | | | | |
| | mutation) | | | | | | | | | | | |
| 17.067 | Humanized | 60.0° | 75.7° | N/A | 13.02 | 4.7 | 3.37E+07 | 3.72E−04 | 1.10E−11 | 2.83E+07 | 3.98E−04 | 1.40E−11 |
| | IgG1 with | C. | C. | | | | | | | | | |
| | reduced | | | | | | | | | | | |
| | Fc-gamma | | | | | | | | | | | |
| | binding | | | | | | | | | | | |
| | (LALA | | | | | | | | | | | |
| | mutation) | | | | | | | | | | | |
| 17.069 | Humanized | 62.8° | 69.3° | N/A | 13.07 | 0.8 | 4.50E+07 | 4.87E−04 | 1.08E−11 | 4.98E+07 | 5.84E−04 | 1.17E−11 |
| | IgG1 with | C. | C. | | ml | | | | | | | |
| | reduced | | | | | | | | | | | |
| | Fc-gamma | | | | | | | | | | | |
| | binding | | | | | | | | | | | |
| | (LALA | | | | | | | | | | | |
| | mutation) | | | | | | | | | | | |

### Example 3

This example provides disclosure on anti-IL-2 antibodies that specifically block the binding of human IL-2 to IL-2 receptor CD25 and modulate the immune system in vivo.

Anti-IL-2 antibodies that bind human IL-2 at an epitope that specifically block interaction between IL-2 and human CD25, have several implications. This allows for the binding of IL-2 antibody complex to effector T cells and NK cells but prevents the binding of human IL-2 to non-immune cells expressing high levels of CD25 (e.g., lung endothelium and vascular endothelium) or to immune cells expressing the high affinity trimeric complex (e.g., Treg cells and CD25+ short lived cytotoxic effector T cells). As a result, these anti-IL-2 antibodies are able to expand effector T cells and NK cells without significantly expanding regulatory T cells (See, FIG. 1 and FIG. 2). Additionally, it has been previously shown that lung endothelial cells express CD25 and high levels of IL-2 in their presence would lead to pulmonary edema. Similar effect was observed for IL-2 induction of vascular leaking through IL-2's interaction with CD25 expressed on vascular endothelial cells. Thus, by targeting IL-2 away from CD25, the anti-IL-2 antibodies disclosed herein are also expected to reduce any IL-2 related pulmonary and vascular toxicity (FIGS. 3A-3B).

### Receptor Discrimination of the Humanized IgG-hIL-2 Complex

BDG17.023-hIL-2 complex (anti-IL-2 antibody-human IL-2 complex) showed receptor binding discrimination, the BDG17.023-hIL-2 complex was found to bind CD122 but not CD25 which resulted in a specific immune system modulation outcome. To test if the humanized antibodies have a similar effect, they were complexed with hIL-2 and tested for binding to CD122 and CD25 by SPR. The analysis was done in a similar fashion to BDG17.023 as described herein. As can be seen by the SPR traces in FIGS. 17B-17G when the humanized antibodies BDG17.038, BDG17.043, BDG17.053, BDG17.054, BDG17.066, BDG17.0067 or BDG17.069 are complexed with hIL-2, the complex bind CD122 but cannot bind CD25, indicating that these antibodies retained the binding discrimination properties of human rat chimera BDG17.023.

### In Vivo Characterization of the Humanized Antibodies

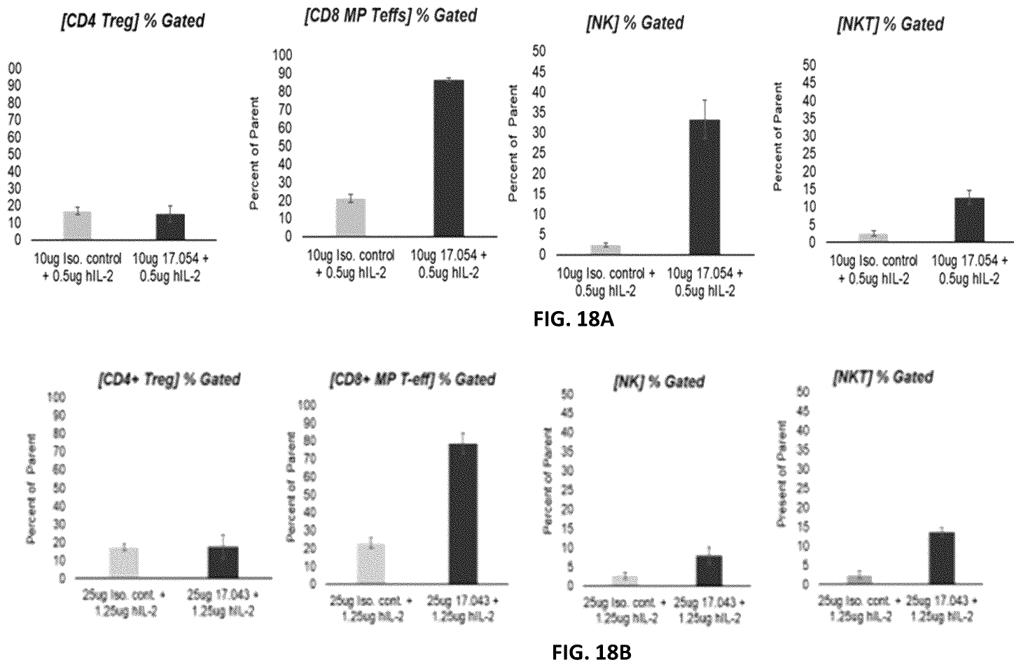

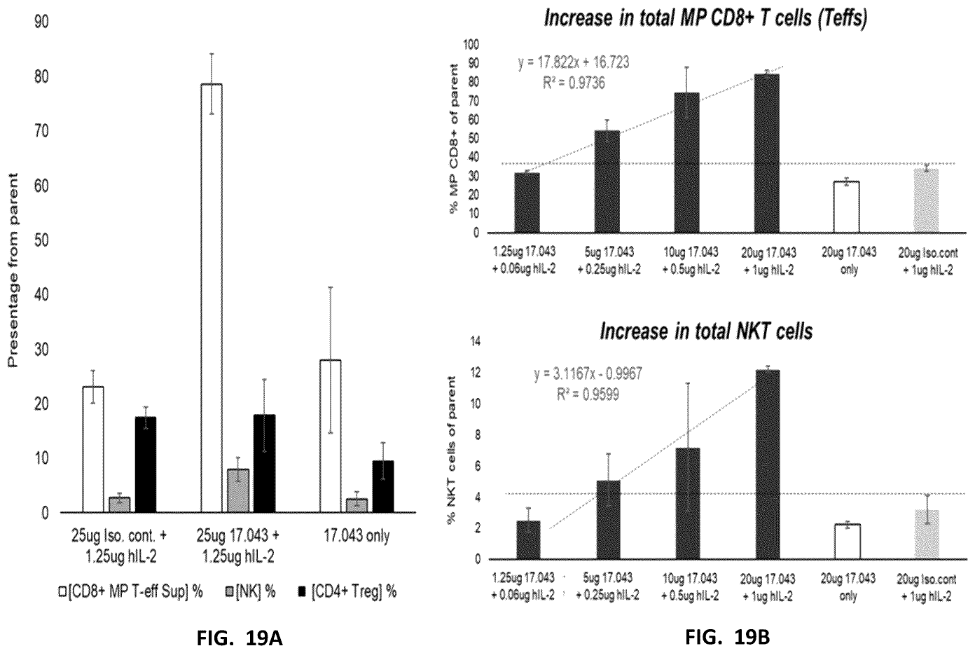

It has been hypothesized that blocking the CD25 binding epitope on IL-2 by high affinity antibodies allows for the binding of human IL-2 to effector T cells and NK cells but prevents the binding of human IL-2 to non-immune cells expressing CD25 (e.g., lung endothelium and vascular endothelium) or cells expressing the trimeric complex (e.g., Treg cells, CD25+ effector T cells). To test this hypothesis in vivo, anti-IL-2 antibodies were pre-complexed with human IL-2 and administered to healthy C57BL/6 male mice as described herein (FIGS. 18A-B). As can be seen in FIGS. 18A and 18B the anti-IL-2 antibodies BDG17.043 and BDG17.054, were able to expand effector T cells, NKT cells and NK cell populations without significantly expanding regulatory T cells (FIG. 18A-18B), likely due to their epitope specific properties. Additionally, the proliferation of MP CD8⁺ T cells and NKT is dependent on the administered dose of IgG-hIL-2 complex and is much more robust than the administered isotype control with hIL2, suggesting that BDG17.043/IL-2 and BDG17.054/IL-2 are actively promoting the CD122/CD132 dimer activation pathway while sparing the CD25/CD122/CD132 trimeric pathway (FIGS. 19A-19B).

BDG17.043 and BDG17.054 complexed with IL-2 induced proliferation of MP CD8+ effector T cells and NK cells but do not promote significant expansion of regulatory T cells, suggesting that these antibody-IL2 complexes have strong stimulatory effect on the immune system as opposed to other antibodies IL-2 complexes like JES 6.1-IL-2 (Spangler J B, Tomala J, Luca V C, Jude K M, Dong S, Ring A M, Votavova P, Pepper M, Kovar M, Garcia K C. Antibodies to Interleukin-2 Elicit Selective T Cell Subset Potentiation through Distinct Conformational Mechanisms. Immunity. 2015 May 19; 42(5):815-25.) and Pfizer's F5111.2-IL2 (Trotta E, Bessette P H, Silveria S L, et al. A human anti-IL-2 antibody that potentiates regulatory T cells by a structure-based mechanism. Nat Med. 2018; 24(7):1005-1014.) that induces immune system anti-stimulatory effect by promoting proliferation of Tregs.)

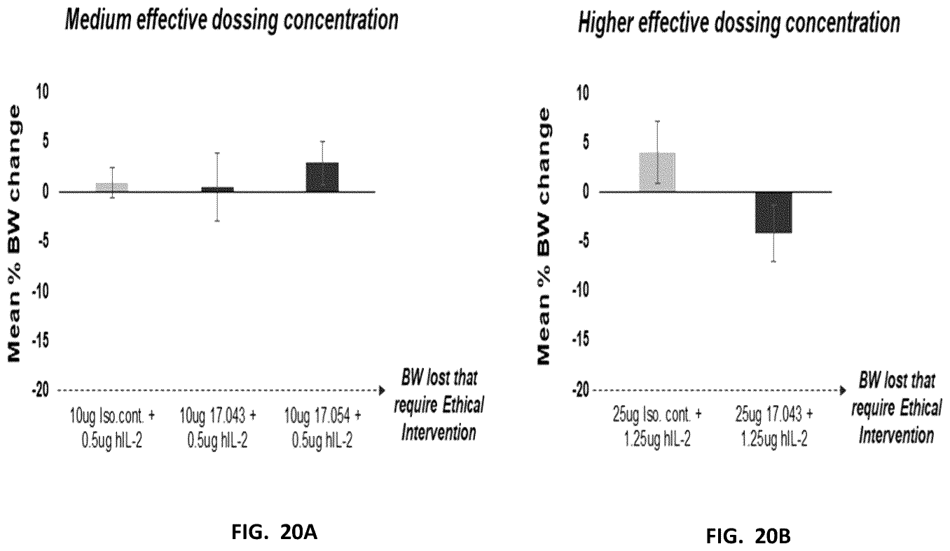

In the mouse study described above, the animals were monitored daily for body weight loss and for non-specific clinical signs. When evaluating drug compounds in mice, a 20% percent body weight loss is considered to be an actionable item that requires ethical intervention. As shown in FIGS. 20A-B, mice administered with and 17.043/IL-2 complex or 17.054/IL-2 complex showed no or less than 10% percent of body weight loss at the end of the experiments. This effect was observed in all dose cohorts including mice treated with the highest dose of 25 mg IgG/1.25 mg IL-2 complex, indicating that the administered complexes are well tolerated.

### Activity of the Humanized Antibodies in B16F10 Syngeneic Cancer Model

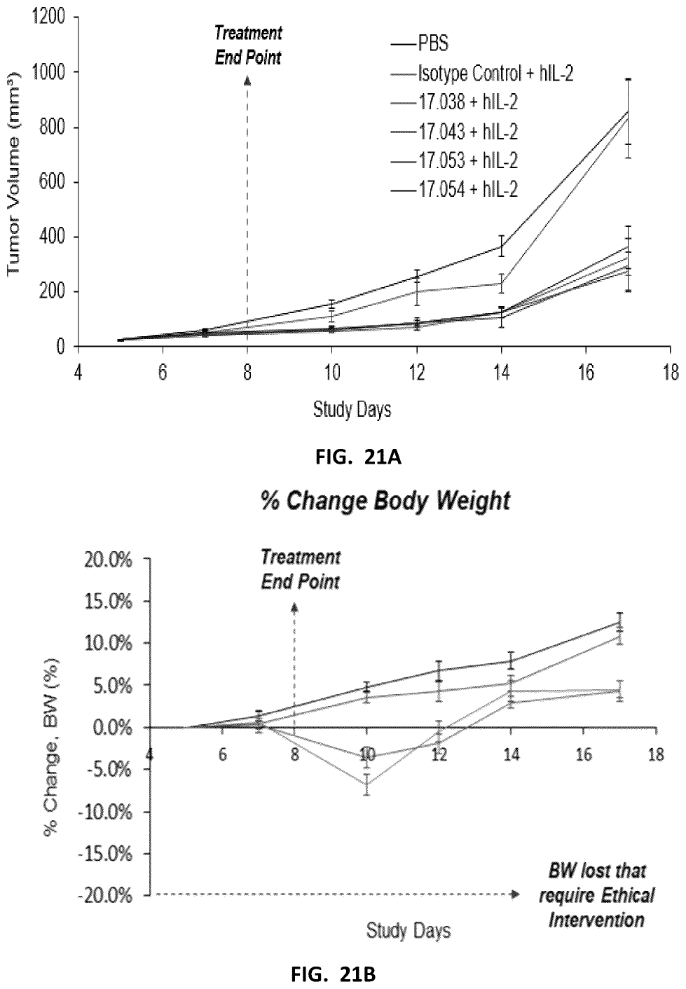

Both viral clearance and cancer therapy share the common need to expand the acquired T cell immune response for efficacy. The ability of anti-IL-2 antibodies to effectively activate immune response was tested in a B16F10 syngeneic melanoma model. C57BL/6 mice were inoculated with B16F10 melanoma and treated with 10 mg anti-IL-2 antibody/1 mg hIL-2 complex or PBS control as described herein. As shown in FIG. 21A, all mice treated with BDG17.038, BDG17.043, BDG17.053, or BDG17.054 complexed with IL-2 showed significant tumor growth inhibition 8 days post treatment that is much larger than treating the mice with Isotype control and hIL-2 (study day 17), likely due to robust and specific immune stimulation. Additionally, as can be seen in FIG. 21B the average transient weight loss in mice treated with two of the anti-IL-2 antibodies is of 6.84%+/−3.9% and 3.6%+/−5.2%, indicating that in a setting of syngeneic B16F10 tumor model the administered antibody/IL-2 complexes were well tolerated.

In summary, these studies show that anti-IL-2 antibodies (17.043 & 17.054) bind human IL-2 with high affinity on a pre-defined epitope such that the antibodies completely prevent the interaction of IL-2 with its receptor CD25. Consequently, the antibody/IL-2 complex is directed to bind and activate the dimeric form of the IL-2R (CD122/CD132). Moreover, anti-IL2 antibodies disclosed and exemplified herein, e.g., 17.038, 17.043, 17.053, and 17.054 show inhibition of tumor growth in a tumor model resistant to checkpoint inhibitors (B16F10 mice melanoma model).

The dimeric receptor complex is found on effector cells. Analysis of the immune stimulating effect in vivo demonstrated that IL-2 in the presence of anti-IL-2 clones 17.043 and 17.054, increases T-effector cell populations (IL-2 Rβγ binding and signaling) with no observed effect on regulatory T cells (IL-2 Rαβγ binding and signaling). This demonstrated that the interaction of IL-2 with the dimeric IL-2 receptor resulted in non-toxic immune stimulation. Taken together, these data support the hypothesis that an anti-human IL-2 antibody that interferes with the ability of the cytokine to bind CD25 positive cells could be used to treat oncologic patients to enhance immune responses that lead to immune response against cancer or in the case of COVID-19 infection can increased clearance of viral load. Additionally, these antibody's properties may prevent IL-2-induced pulmonary edema and possibly prevent lung tissue damage in SARS-CoV-2 infected lungs.

### Example 4

This example provides a description of studies performed examining formulations of anti-IL-2 antibodies.

Methods: Formulation analysis was done by incubating 30 mg/ml of anti-IL-2 clone BDG17.069 in four different formulations:
F1) 20 mM Histidine, 8% sucrose, 0.04% PS80, pH5.5;
F2) 20 mM Histidine, 8% sucrose, 0.04% PS80, pH6.0;
F3) 20 mM Citrate, 8% sucrose, 0.04% PS80, pH5.5; and
F4) 20 mM Histidine, 8% sucrose, 10 mM Methionine, 0.04% PS80, pH5.5.

The antibody was subjected to (1) incubation for one and two weeks at 40° C., (2) agitation of 300 rpm at 25° C. for three days, and (3) 3-5 cycles of Freeze/Thaw (F/T). At T=0 and post treatment the antibody was analyzed for appearance, size-exclusion chromatography-ultraperformance liquid chromatography (SEC-UPLC), pH, protein concentration, PI (Capillary isoelectric focusing-cIEF), subvisible particles (Micro flow imaging—MFI) and Tm (DSC).

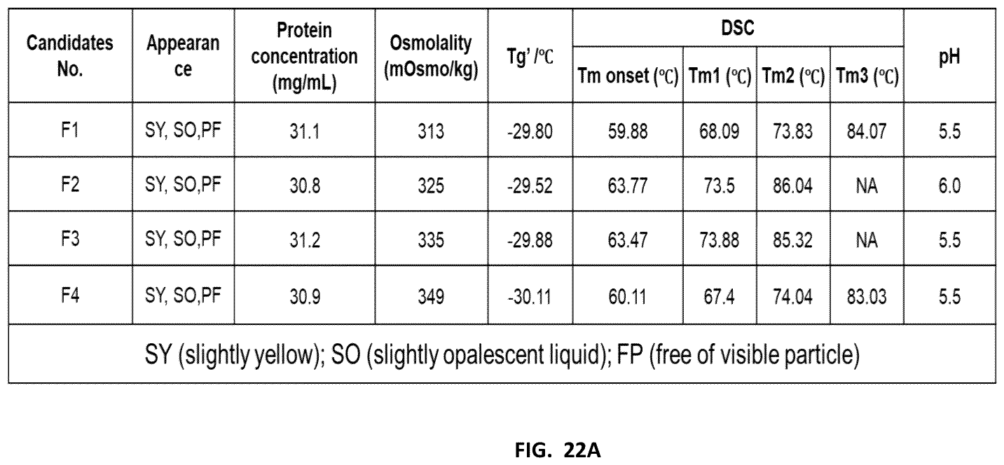

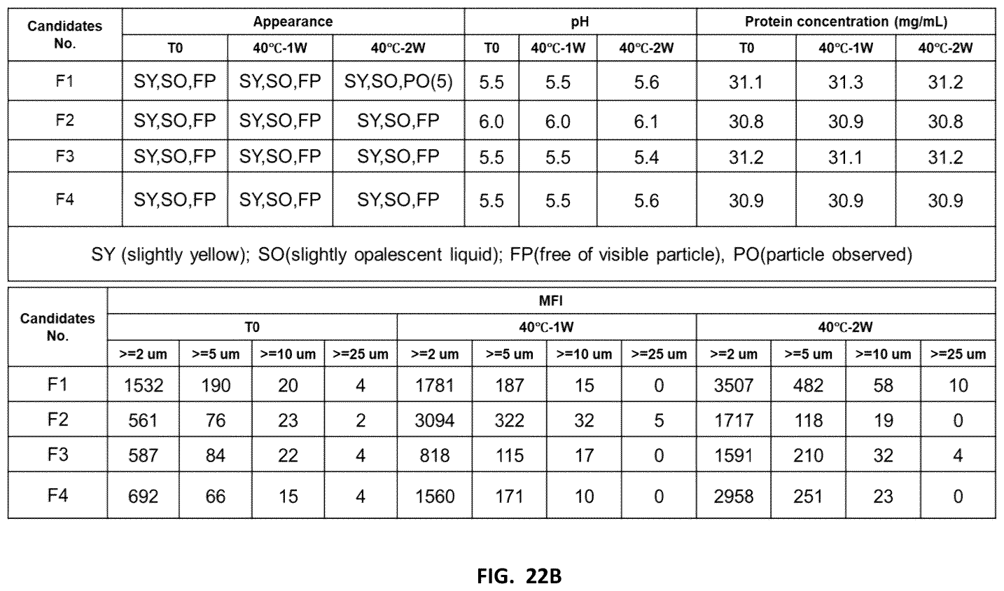

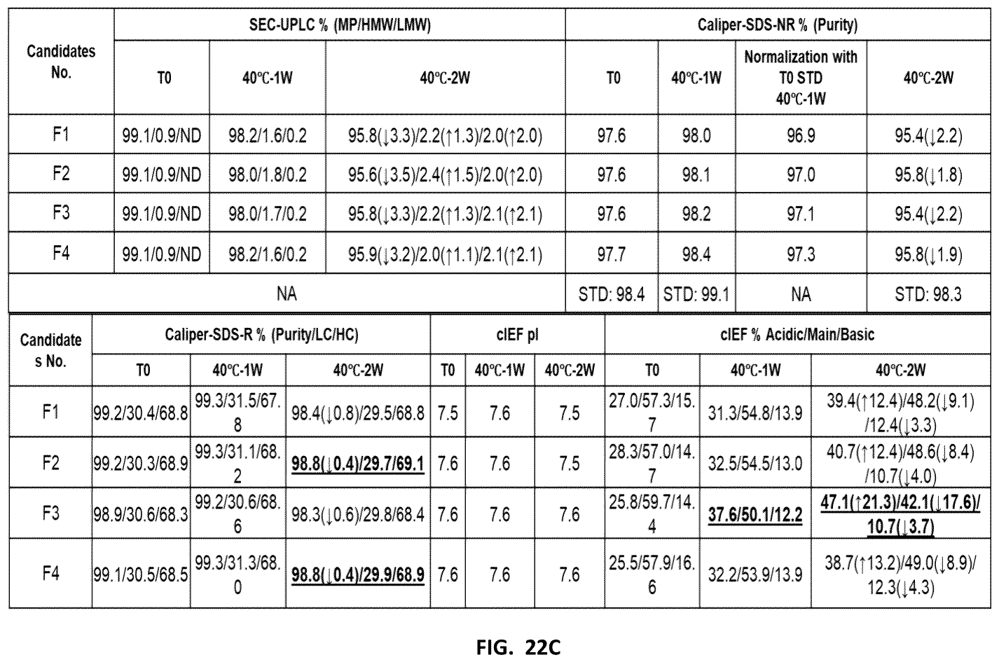

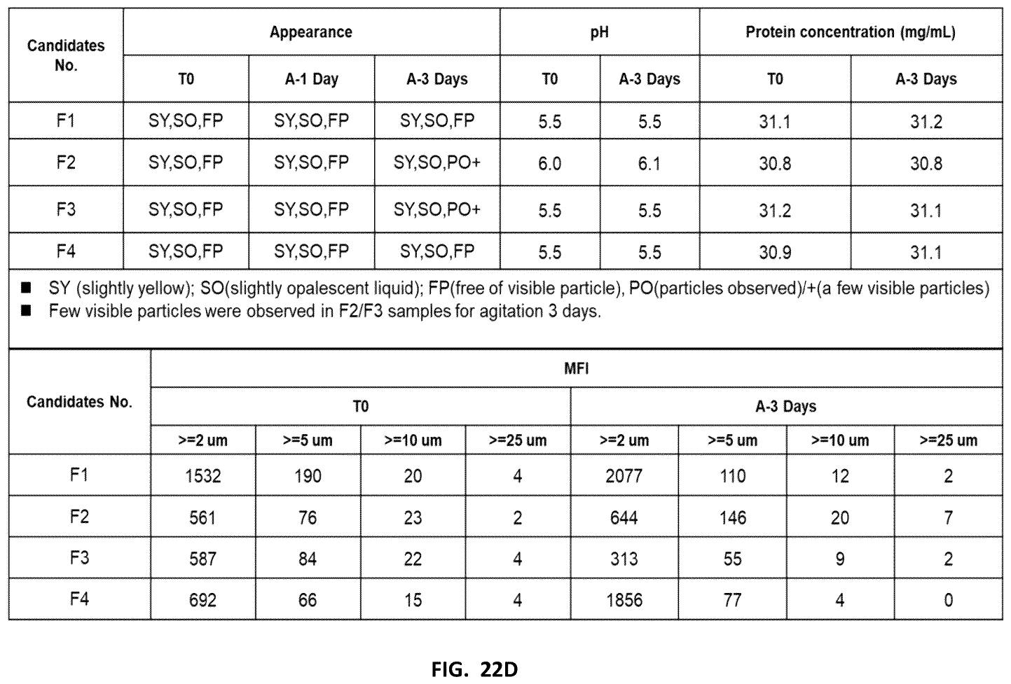

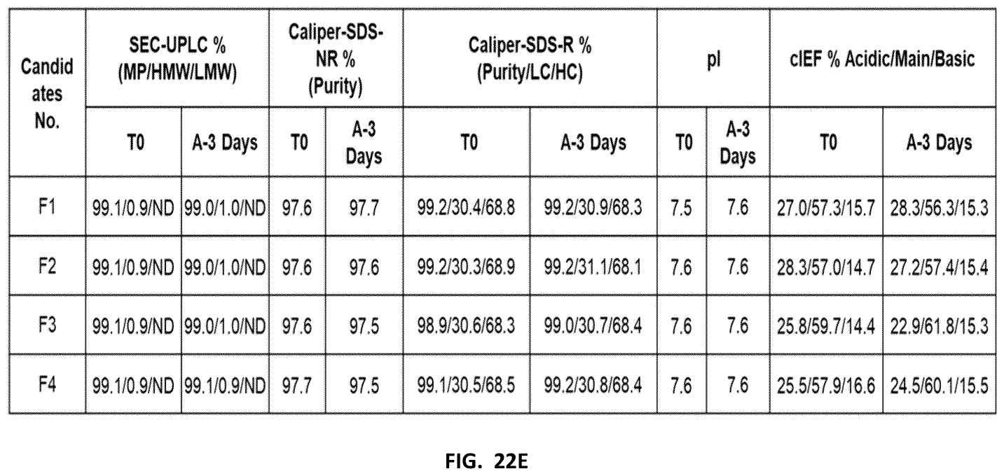

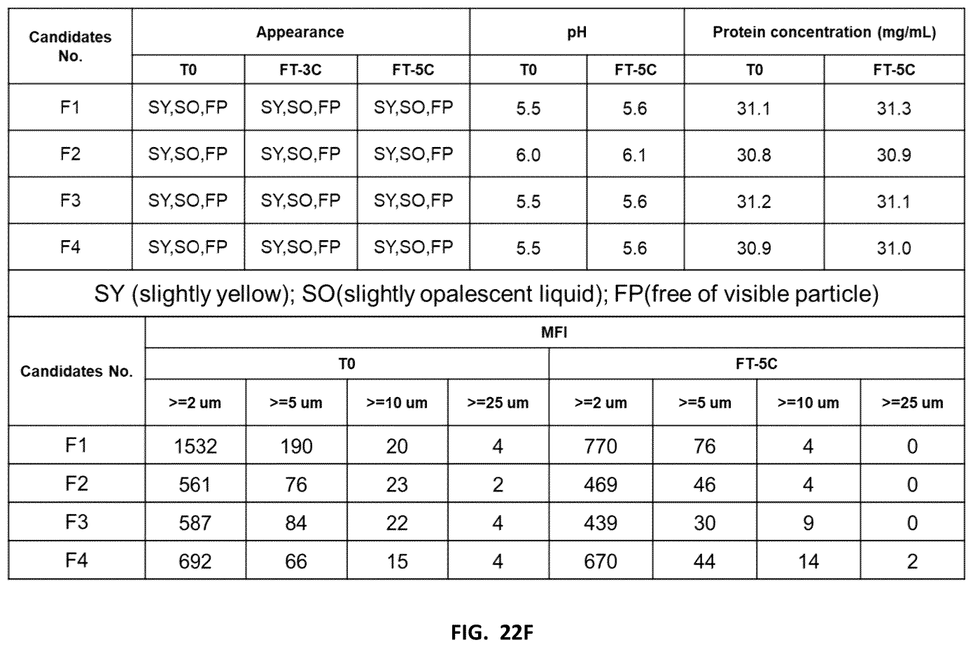

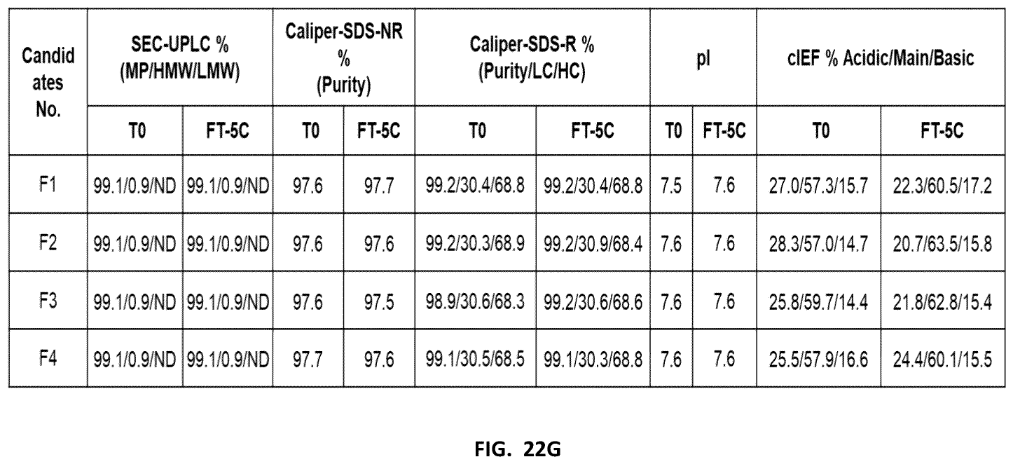

Results: The tables presented as FIGS. 22A-22G, show the results of analyzing the different antibody formulations.

As can be seen in tables provided in FIGS. 22A-22G BDG17.069 formulated in F2, F3, F4 showed no apparent change in concentration, nor change in appearance is detected after one or two weeks of incubation at 40° C. Analysis of sub-visual particles by MFI demonstrate that in BDG17.069 formulated in F1, F2, or F4 shows no formation of particles >=25 uM and only minor change in formation of particles >=10 uM (FIG. 22B). SEC-UPLC analysis revealed only minor increase in small MW species for all four formulation conditions. Additionally, caliper-SDS analysis demonstrated minimal changes for BDG 17.069 formulated in F2 and F4, and analysis by cIEF displayed relatively small changes for BDG17.069 formulated in F4. However, BDG17.069 formulated in F3 presented substantial increase in acidic percentage at 40° C. after 2 weeks of incubation (FIG. 22C).

While BDG17.069 formulated in F2 and in F3 exhibited slight particle formation post agitation (FIG. 22D), and after 5 cycles of Freeze/Thaw, BDG17.069 formulated in F4 and F1 did not show significant changes to its appearance, pH, concentration or any >5 uM sub visible particles formation (FIG. 22F).

Taken together these experiments demonstrate that in formulations F1, F2, F3 and F4, BDG17.069 has good stability performance after agitation, freeze-thaw stresses and a very moderate aggregate percentage after incubation at 40° C. for 2 weeks; taking all stress conditions into account it is apparent that BDG 17.069 is most stable when formulated in 20 mM Histidine, 8% sucrose, 10 mM Methionine, 0.04% PS80, pH5.5 (F4).

### Example 5

This example provides a description of studies demonstrating the safety and efficacy of epitope specific anti-hIL-2 antibodies that are designed to enhance the immune response to infectious agents such as SARS-CoV-2 by specifically activating effector T and NK cells while reducing the binding of IL-2 to regulatory T cells and lung endothelium.

### Pre-Clinical Studies

The following pre-clinical studies can be done in 2 phases: phase 1 is to demonstrate the safety of the compound as a single intravenous dose in healthy volunteers; phase 2 is to demonstrate safety of multidoses in a virally infected animal model.

As disclosed herein, the anti-IL-2 antibodies specifically bind to human IL-2 and do not bind to mouse IL-2. Although mouse IL-2 receptors can bind human IL-2, allowing one to load hIL-2 into a complex and use a mouse model, normal mice are not desirable as they would subsequently begin to produce high levels of mouse IL-2. Secreted mIL-2 would therefore be available to bind receptors in the lung to induce edema, thereby confounding the safety interpretation. Therefore, animal models that allow either use of human IL-2 or for which antibody cross reactivity with endogenous IL-2 does occur are preferred models. For mouse models, several options are available. First, to investigate whether the antibody or Ab/IL2 complex has any detrimental effect directly on CD25 expressing lung tissue, an IL-2 knock-out mouse is available. The primary limitation of this model is the lack of any subsequent amplification of the immune response and any additional immune elicited cytokines. A second option is to use healthy C57BL/6 or BalbC mice which have been depleted of mouse IL-2 using a neutralizing antibody. However, optimization of this model will still be needed. A third option is to use CD34+ human cord blood cells transferred into the NOD-EXL mouse to generate a mouse expressing a human immune system. The advantage is that a near full human immune system (albeit with some limitations) is expressed, particularly with all T cell and NK cell populations. This model is also conducive for use as an acute viral infection model as well as a model system in oncology research.

Based data presented herein, since 17.067 and 17.069 demonstrated robust affinity towards cynomolgus monkey IL-2 and based on the high homology between hIL-2 and cIL-2 are likely to presented Ab-IL2 complex receptor discrimination for both, primate safety models can be completed. Healthy cynomolgus monkeys can be tested in a single ascending dose test. It has been demonstrated that cynomolgus monkeys can be infected by SARS-CoV-1 and rhesus monkeys have been shown to be infected by SARS-CoV-2. In both situations, a mild case of lung edema was observed, similar to what is observed in mild human infection. As such, these monkey models would allow for examination of both the safety and efficacy of the anti-IL-2 antibodies/IL-2 complexes.

Dose escalation experiments can be done in healthy animals (e.g., humanized mice, cynomolgus monkey etc.) as follows:

### Supporting First in Man (FIM) Healthy

a. IL-2 KO mouse dose escalation; and/or
b. mIL2 antibody depleted hIL2+ antibody; and/or
c. CD34+ SCT humanized mouse (+/−IL2);
d. Healthy cynomolgus or rhesus single dose escalation;
e. Healthy cynomolgus or rhesus multi dose (done concurrently with FIM single ascending dose).

### Supporting Patient Dosing (Phase 1b or 2)

a. Mouse (b or c above and/or hACE2 transgenic) viral loaded (acute flu infection model or coronavirus respectively).
b. If available, cynomolgus (SARS-CoV) or rhesus (SARS-CoV-2) coronavirus acute infection model.

### Clinical Trials

Part 1) A dose escalation trial in healthy volunteers can be done to identify the maximum tolerated dose. Up to eight cohorts of 10 individuals (8 test, 2 placebo) can be done using an appropriate interval between doses to all for safety determinations.

Part 2) Using the doses determined in Part 1, recently symptomatic SARS-CoV-2 positive patients with mild symptoms can be treated and monitored for safety and efficacy. Efficacy can be determined by decrease in time to viral clearance and a decrease in the signs and symptoms of respiratory infection. Exploratory endpoints can include changes in peripheral blood immune cell populations and activation states.

### Chemistry, Manufacturing and Controls

Cell line, drug substance (DS), and drug product (DP) can be done in a GMP qualified manufacturer under GMP guidance (e.g., Wuxi Biologics). An accelerated material production can be done to have material in 6 months ready for an IND. All, bioburden, viral clearance, viral load and host cell proteins can be examined and reduced to specifications as determined by current manufacturing guidelines to ensure product safety. DP can be formulated for intravenous administration. Stability testing can be done concurrently to ensure product quality over time.

### Example 6

This example provides a description of studies performed evaluating anti-IL-2 antibody clone BDG17.069 (AU-007) as (1A) a monotherapy. Additional studies to be performed include those evaluating anti-IL-2 antibody clone BDG17.069 (AU-007) (1B) in combination with a single loading dose of aldesleukin (Aldesleukin is the generic name for the trade drug names Proleukin® (aldesleukin)®. Interleukin-2 and IL-2 are other names for Aldesleukin.) and a study (1C) with both BDG17.069 and aldesleukin given every 2 weeks (Q2w).

As disclosed and exemplified above, AU-007 is a human IgG1-LALA antibody that binds to human IL-2 at the CD25 binding site without impinging on the CD122 or CD132 binding motifs on IL-2. This binding leads to an inhibition of IL-2 binding to the CD25-containing trimeric receptor found on regulatory T cells (Tregs), activated effector cells (which upon binding induces reactivation cell death), eosinophils, and vascular endothelium. However, AU-007 binding to IL-2 still allows IL-2 binding to the dimeric receptor expressed on naive T cells, memory phenotype T cells (a key population to target with cancer immune therapy), NK cells, and NK T cells.

### Study Design

The BDG17.069 monotherapy evaluated doses sufficiently high to ensure enough BDG17.069 is available to bind all IL-2 molecules: both exogenously administered (aldesleukin) and endogenous IL-2.

Patient inclusion criteria specified any of 19 solid tumor histologies.

Adverse events were graded by the Common Terminology Criteria for Adverse Events (CTCAE).

Efficacy evaluation was based on PD (Pharmacodynamic) markers of immune stimulation, total IL-2 (bound to BDG17.069+ free IL-2) and objective responses.

Optional paired pre/on-treatment biopsies were performed.

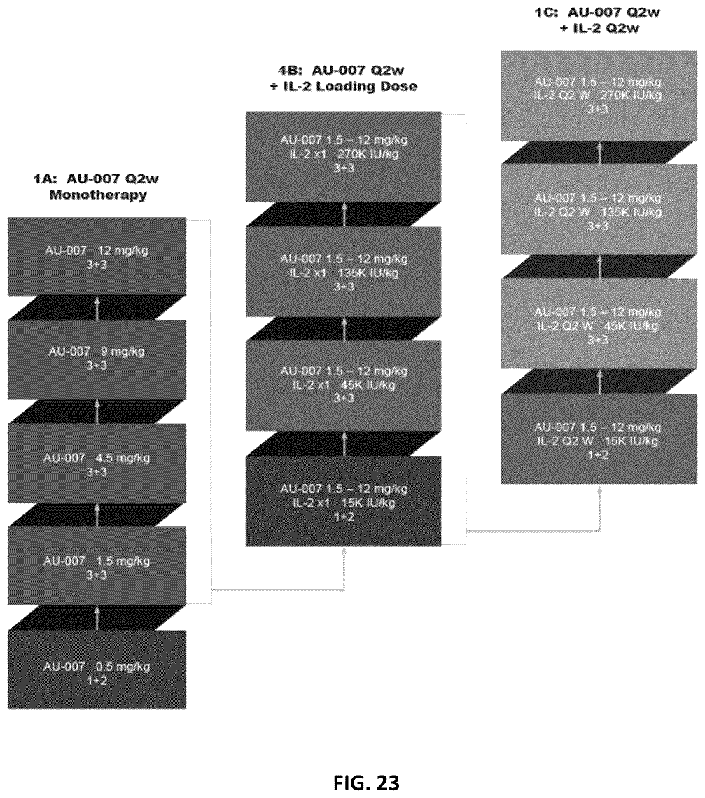

FIG. 23 graphically presents the administration and dosage schemes of BDG17.069 Q2w Monotherapy (left-1A), BDG17.069+ a single IL-2 Loading Dose (center-1B), and combination therapy BDG17.069 Q2w+IL-2 Q2w (right-1C). When administered, recombinant human IL-2 (aldesleukin) was administered subcutaneously, at much lower doses and much less frequently than the approved regimen of intravenously administered aldesleukin.

In FIG. 23, the terms “3+3” and “1+2” refer to the size of the cohort at a given dose level. It means in the case of 3+3 that the first 3 enrolled patients were or will be administered BDG 17.069 at that dose level. If no dose-limiting toxicities (DLTs) are seen, then administration may escalate to the next highest dose level. However, if any of the first 3 patients do have a dose-limiting toxicity that is drug-related, then an additional three more patients need to receive treatment at that same dose level to see if they have DLTs before escalating. 1+2 follows the same principle—start with one patient, if DLTs are observed, add two more, or if no DLTs are observed in the first single patient, then dosages can be escalated.

### Results

### Patient Demographics

Four patients have been enrolled into dose escalation, Arm 1A, BDG17.069 monotherapy.

The initial patient enrolled received one dose of BDG17.069 but discontinued study after 10 days with deteriorating clinical condition secondary to his widespread disease. The head and neck cancer patient in Cohort 1 is the replacement for the initial patient. Table 10 presents patient demographics.

TABLE 10
Dosage escalation patient characteristics.
Dose Escalation Patient Characteristics

| | | AU-007 | | Age/ | Prior Treatment/ | |
|---|---|---|---|---|---|---|
| Arm | Cohort | Dose | Histology | Sex | Best Response | ECOG |
| 1A | 1 | 0.5 | Pt 1 Head and Neck | 68 M | 1) Cisplatinum | 1 |
| | | mg/kg | Squamous Carcinoma | | (adjuvant)/NA | |
| | | | (HNSCC) | | 2) AMG2562/PR | |
| | 2 | 1.5 | Pt 2 Pancreatic | 52 M | 1) FOLFIRINOX/PD | 0 |
| | | mg/kg | (adenocarcinoma) | | 2) Gemcitabine + | |
| | | | | | paclitaxel/PD | |
| | | | Pt 3 NSCLC | 82 M | 1) Carboplatin + | 1 |
| | | | (squamous) | | paclitaxel/PD | |
| | | | | | 2) Durvalumab/PD | |

1: Head and neck cancer histology: mucoepidermoid with squamous cell component.
2: IL-21 mutein x anti-PD-1 bispecific.

### Efficacy

FIG. 24 presents Duration of Treatment and Efficacy Details. The patient in Cohort 1 received 0.5 mg/kg BDG 17.069, while the patients in Cohort 2 received 1.5 mg/kg BDG 17.069. Patients 1 (Cohort 1) and 3 (Cohort 2) continued with treatment past the data cutoff shown here.

The data in Table 11 demonstrates the efficacy of treatment wherein Patients 1 and 3 had an absence of new lesions.

TABLE 11
Efficacy Details

| | | | Target Lesion | | | | | |
|---|---|---|---|---|---|---|---|---|
| | | | Sum (mm) | | | Non- | | |
| AU-007 | | Metastatic | Base- | Cycle | Cycle | Target | New | AU-007 |
| Dose | Histology | Sites | line | 1 | 2 | Lesions | Lesions | Doses |
| 0.5 | Head and | Anterior and | 59 | 59 | 61 | P* | No | 10 |
| mg/kg | Neck | Posterior Pleura, Lung | | | | | | |
| 1.5 | Pancreatic | 1 Celiac and 2 | 86 | 82 | | P* | Yes | 3 |
| mg/kg | | Mediastinal Masses | | | | | | |
| | NSCLC | Lung, Left and | 101 | 87 | | P* | No | 6 |
| | | Right Adrenals | | | | | | |

*Present.

### Safety

Tables 12A and 12B present the Safety details, wherein adverse events associated with BDG 17.069 were limited to a single event (diarrhea).

TABLE 12A
BDG 17.069 Related Adverse Events.
AU-007 Related Adverse Events

| AU-007 Dose | Patient's Tumor | | | |
|---|---|---|---|---|
| (patient number) | Histology | Event | Grade 1/2 | Grade 3 |
| 0.5 mg/kg (n = 1) | Head and Neck | Diarrhea | 1 | |

TABLE 12B
All Adverse Events
Adverse Events Regardless of Causality

| AU-007 Dose | | | | |
|---|---|---|---|---|
| (number of patients) | Tumor Histology | Event | Grade 1/2 | Grade 3 |
| 0.5 mg/kg | | Pleuritic Chest Pain | 1 | |
| (n = 1) | Head and Neck | Hemoptysis | 1 | |
| | | Diarrhea | 1 | |
| 1.5 mg/kg | | GGT Increase | | 1 |
| (n = 2) | | Increased ALP | 1 | |
| | | Bilirubin Increase | 1 | |
| | | Pancreatic Enzyme | 1 | |
| | | Decrease | | |
| | Pancreatic | Biliary obstruction | | 1* |
| | | Cancer pain | 1 | |
| | | Anemia; upper GI | | 1 |
| | | bleeding | | |
| | | Creatinine Increase | 1 | |
| | | Fatigue | 1 | |
| | | Hyperglycaemia | 1 | |
| | NSCLC | Constipation | 1 | |

*SAE: Hospitalization for per-cutaneous drainage of biliary obstruction

In summary, 4 patients have received at least one dose of BGD 17.096. Only one drug-related Adverse Event (AE), Grade 1 diarrhea, occurred in the patient with head and neck cancer receiving 0.5 mg/kg BGD 17.096. The initial patient enrolled onto trial was a 60 y.o. man with nasopharyngeal carcinoma and widespread metastases to liver, lung and bone. He received one dose of AU-007 at 0.5 mg/kg but was discontinued from study after 10 days with deteriorating clinical status. This patient had no reported AEs. The pancreatic cancer patient experienced signs and symptoms of biliary obstruction after receiving 3 doses of BGD 17.096. He was hospitalized for endoscopic and MRI evaluation confirming biliary outflow obstruction secondary to post-Whipple procedure stricture and potential tumor obstruction that was relieved via per-cutaneous drainage.

### Pharmacokinetics (PK)

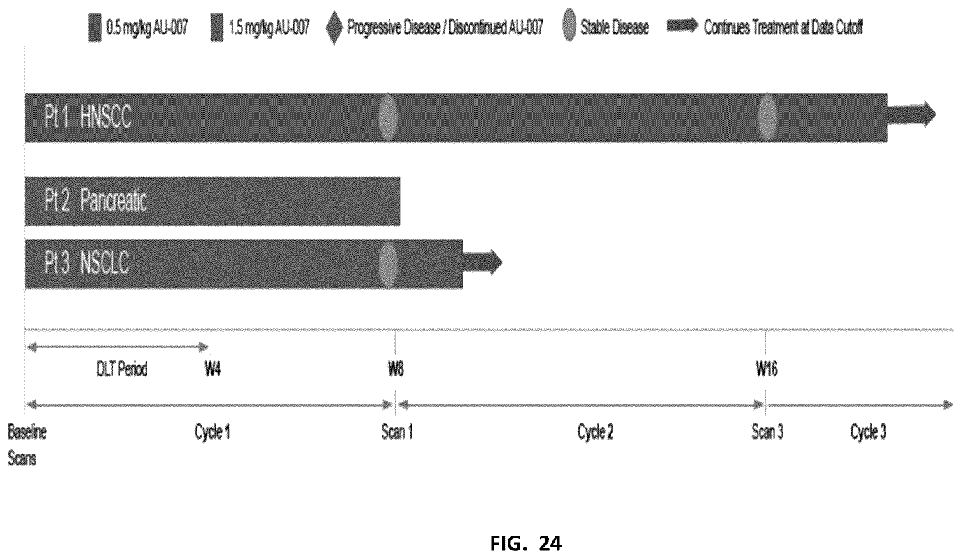

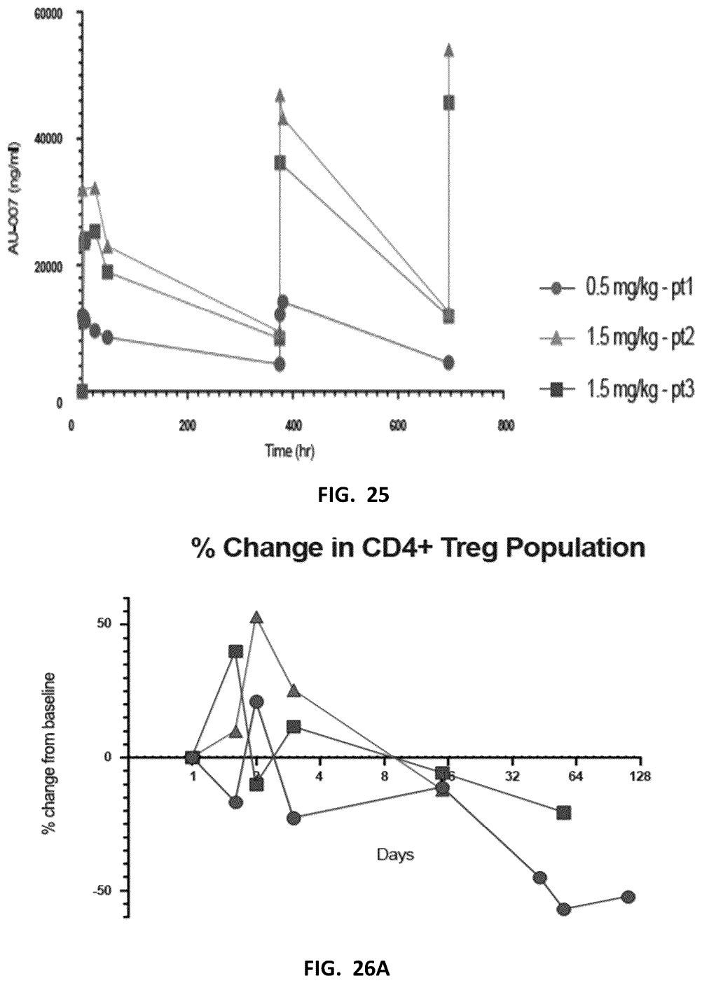

FIG. 25 presents pharmacokinetics data showing the early pharmacokinetic profile of the first 3 patients administered monotherapy BGD 17.096, 1 patient with 1.5 mg/kg and 2 patients with 1.5 mg/kg. The initial PK data demonstrates BGD 17.096 concentration is dose proportional and has PK characteristics similar to a standard IgG1 therapeutic human monoclonal antibody.

### Pharmacodynamics (PD)

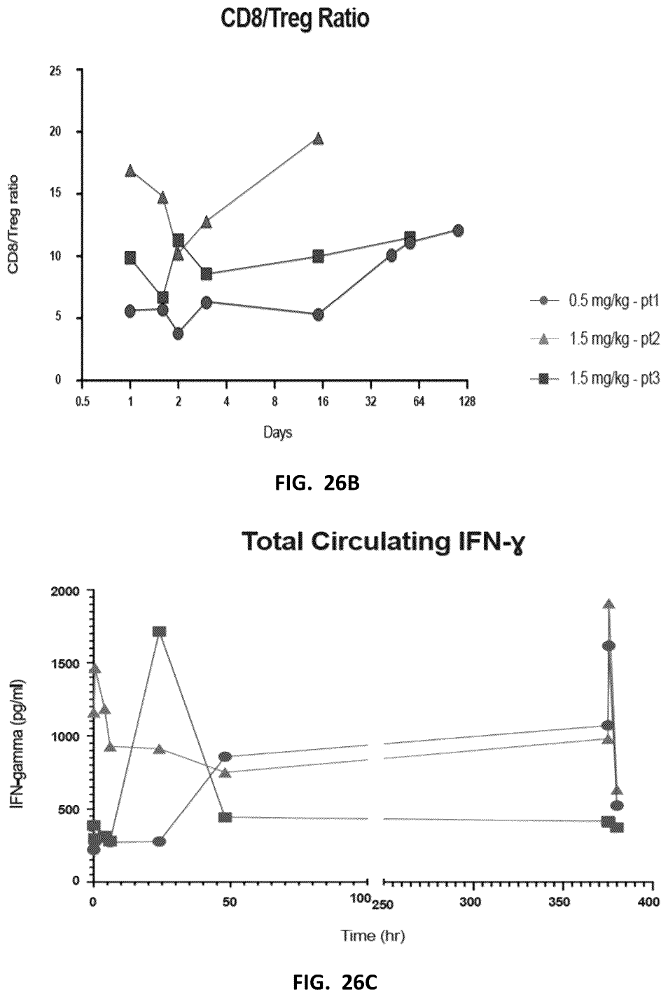

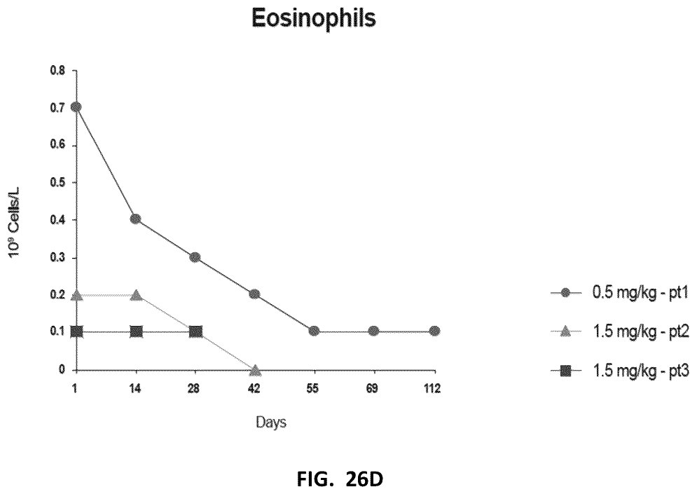

FIGS. 26A-26D graphically present PD data showing the effects of BDG 17.069 and the mechanism of action. FIG. 26A shows flow cytometry characterization of circulating Tregs (CD3+ CD4+ CD25+ CD127 dim, Foxp3+) demonstrating an overall trend toward decreasing percentage of circulating Tregs in the first 3 patients. FIG. 26B shows that the decrease in Tregs resulted in an increase in the CD8/Treg ratio. FIG. 26C shows the changes in interferon gamma (INFγ or IFN-gamma) in the peripheral blood of first 3 patients. Serum samples were taken and examined for the presence of circulating IFN-gamma using a qualified ECL technique (LLOQ 13.8 fg/ml). FIG. 26D shows that even though eosinophils express the same trimeric, CD25+ IL-2 receptor as Tregs and vascular endothelium, no increase in the absolute eosinophil counts were observed, with 2 patients having early decreasing trends.

### SUMMARY

BDG 17.069 continues to be evaluated at the second dose-level (1.5 mg/kg) of the monotherapy dose escalation cohort. In 3 evaluable patients, 1 patient at 0.5 mg/kg and 2 patients at 1.5 mg/kg, the only drug related toxicity has been Grade 1 diarrhea in a single patient. Two of the three evaluable patients have a best response of stable disease with some tumor shrinkage seen in one patient, and are continuing on with the study treatment.

The early PK profile demonstrates characteristics similar to a standard IgG1 therapeutic human monoclonal antibody.

Trends toward decreasing Treg cells with concordant increases in CD8/Treg ratio, initial IFN-γ increases and decreasing absolute eosinophils are consistent with the novel mechanism of action of BDG 17.069, and consistent with the preclinical data from mice with syngeneic tumors, human PBMCs and cynomolgus monkeys treated with BDG 17.069. In contrast, reported clinical data and preclinical data from other IL-2 therapeutics demonstrate that all other agents assessed, substantially increase Tregs, likely due to the negative feedback loop, contributing to disappointing clinical efficacy findings.

Dosing continues with similar positive results expected.

### Example 7

Anti-IL2 Antibody Control of Endogenous IL-2 and Prevention of Treg Expansion while Allowing Expansion of Teffs

Objective: Stimulating effector T-cells (Teffs) without inducing regulatory T-cells (Tregs) has been the primary goal of IL-2-based therapies for cancer. Recently, modified IL-2 designed for differential T-cell expansion for the treatment of cancer had failed in the clinic. It is proposed herein that treatments based on exogenous administrations of modified IL-2 are inherently undermined by a negative feedback loop, caused by IL-2 secreted endogenously from activated effector T-cells. This endogenous IL-2 secretion subsequentially induces Treg expansion and inhibits the immune response that is essential for cancer clearance. The objective here was to analyze treatments utilizing exogenous modified IL-2 to determine if they induce Treg expansion, and to circumvent this negative feedback, utilizing monoclonal humanized anti-IL2 antibody 17.069 (AU-007) that binds human IL-2 with pM affinity in a predefined epitope and completely blocks IL-2 binding to CD25 that is highly expressed over Tregs, without hindering IL-2 binding to CD122/CD132 dimer receptor expressed over effector cells.

Introduction: Interleukin-2 (IL-2) is a key mediator in the expansion and activation of T-cells and natural killer cells (NKs). IL-2 plays a major role in the secondary signals required for T-cell activation. It binds two forms of the IL-2 receptor: a high-affinity trimeric receptor composed of CD25, CD122, and CD132, and a low-affinity dimeric receptor composed of CD122/CD132. T effector cells (Teffs) and NKs can receive signaling via IL-2 binding to the dimeric receptor, leading to their expansion and activation. Regulatory T-cells (Tregs) rely on the high-affinity trimeric receptor complex to enhance their functions, which include immunosuppressive cytokine release, mitotic expansion, and sequestering IL-2 away from binding to memory and naive T-cells. Activated Teffs secrete IL-2 and transiently upregulate CD25 expression. The binding of IL-2 to these activated Teffs induces restimulation-induced cell death (RICD). IL-2 is produced by activated CD4+ T helper cells and activated CD8+ Teffs. Thus, effector cells that are expanded by IL-2 secrete more IL-2 upon activation. Autocrine and paracrine secretion of IL-2 from activated T-cells also reduces immune activation by increasing RICD of activated Teffs and by enhancing the expansion and fitness of Tregs. As such, immune activation by IL-2 is under tight negative feedback regulation through IL-2-induced IL-2 release.

Overall, IL-2 has a dual effect in regulating the immune response. During homeostasis, low levels of IL-2 are available to bind and stimulate the high-affinity trimeric receptor expressed over Tregs. IL-2 is also the predominant cytokine that is produced in high concentrations by TCR-activated and IL-12-stimulated Th1 cells in a primary response. These increasingly high levels of IL-2 interacting with the dimeric receptor expressed on naive and memory precursor (MP) T cells are required for the proliferation and differentiation of those cells into effector cytotoxic cells, those T cells also secrete IL-2 when activated. IL-2 is also known to have a role in shutting down the immune response and restoring homeostasis. TCR-activated and IL-2-stimulated T cells transiently upregulate CD25 expression, and those short-lived CD25+ cytotoxic T cells are prone to apoptosis in response to a second stimulation of IL-2. IL-2 mediates reactivation-induced T-cells death (AICD), also known as RICD, while CD25 blockade protects T cells from dying. Tregs do not secrete IL-2, stably express high levels of CD25, and are solely dependent on IL-2 for survival. Thus, IL-2 modulates the immune response in a negative feedback loop mechanism, T-cell TCR activation leads to IL-2 secretion to support immune stimulation, while a second response to IL-2 on these TCR-activated and IL-2 stimulated terminally differentiated T cells leads to their death, yet maintains the viability and functionality of Tregs to support immune suppression. IL-2 proinflammatory effect is crucial for cancer clearance due to the cytotoxic activity of the effector cells, while IL-2 immune inhibitory effect undermined it.

High-dose IL-2 (HD IL-2) is approved for the treatment of melanoma and renal cell carcinoma, but its therapeutic value is limited due to limited efficacy, severe toxicity, and short in vivo half-life. The half-life for free human IL-2 is 85 minutes (elimination half-life in plasma, per Proleukin® (aldesleukin) package insert). A significant improvement in IL-2 half-life was observed in toxicology studies using cynomolgus monkey. Measurements of half-life approximately 15 days following injection of AU-007 showed a greater than 250-fold difference (increase) of half-life compared with free IL-2 (Data not shown).

The major challenge in the development of IL-2 as a therapeutic antitumor agent is that IL-2 can act on both effector cells that express the dimeric receptor and Tregs that express the trimeric receptor. Exogenous IL-2 therapies may lead to production of endogenous IL-2, which in turn drives expansion of immunosuppressive T regulatory cells via a negative feedback loop. Cancer patients which were given a high dose of IL-2 showed limited efficacy due to increased Treg levels. Stimulating Teffs and NKs without inducing Tregs has been the main challenge in developing IL-2-based therapies for oncology. Modifying IL-2 to have a bias selectivity to cells that express IL-2 dimeric receptor and prevent its interactions with Tregs aims to solve traditional IL-2 therapy's limited efficacy. In recent years, several investigational therapeutics that are based on modified IL-2 have been developed with the goal of eliminating the inhibitory function of IL-2 while maintaining its activating effect. These approaches utilize exogenously administered modified IL-2 that preferentially activate Teffs relative to Tregs due to modifications that limit IL-2's ability to bind the CD25 subunit [Overwijk W W, et al., Engineering IL-2 to give new life to T cell immunotherapy. Annual Review of Medicine. 2021 Jan. 27; 72:281-311; Majidpoor J, and Mortezaee K. Interleukin-2 therapy of cancer-clinical perspectives. International Immunopharmacology. 2021 Sep. 1; 98:107836.]. However, a primary source of endogenous IL-2 is activated Teffs. Modified IL-2 with a bias selectivity to the dimeric receptor, indeed leads to the proliferation of effector T-cells but importantly, has no effect on the action of the native endogenous IL-2 that is secreted from the same activated T cells that it acts on. Thus, this newly endogenous IL-2 is free to shut down the immune response and undermined the modified IL-2 effect.

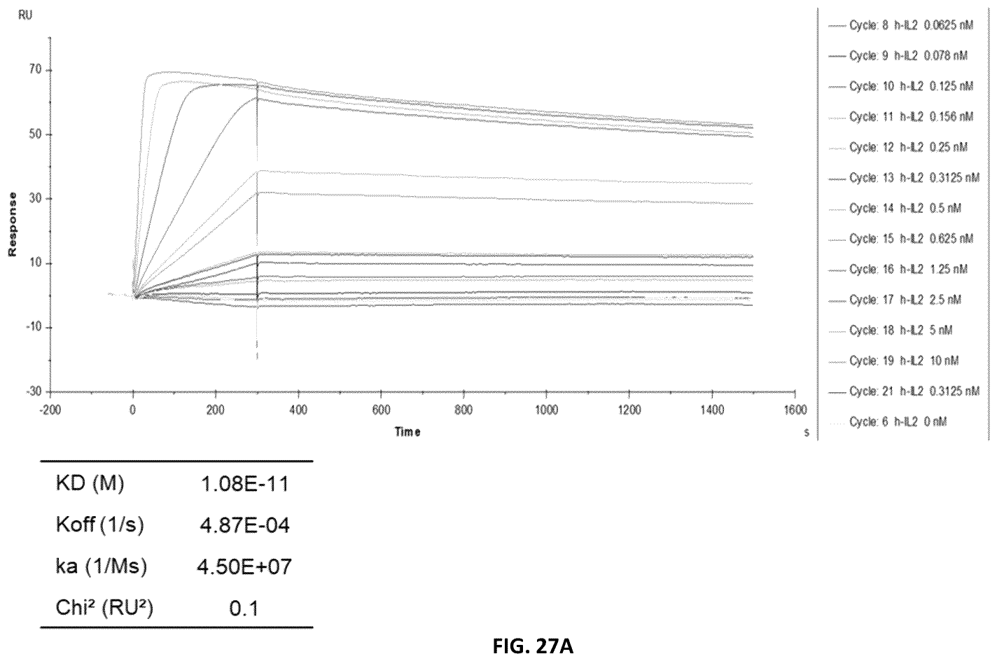

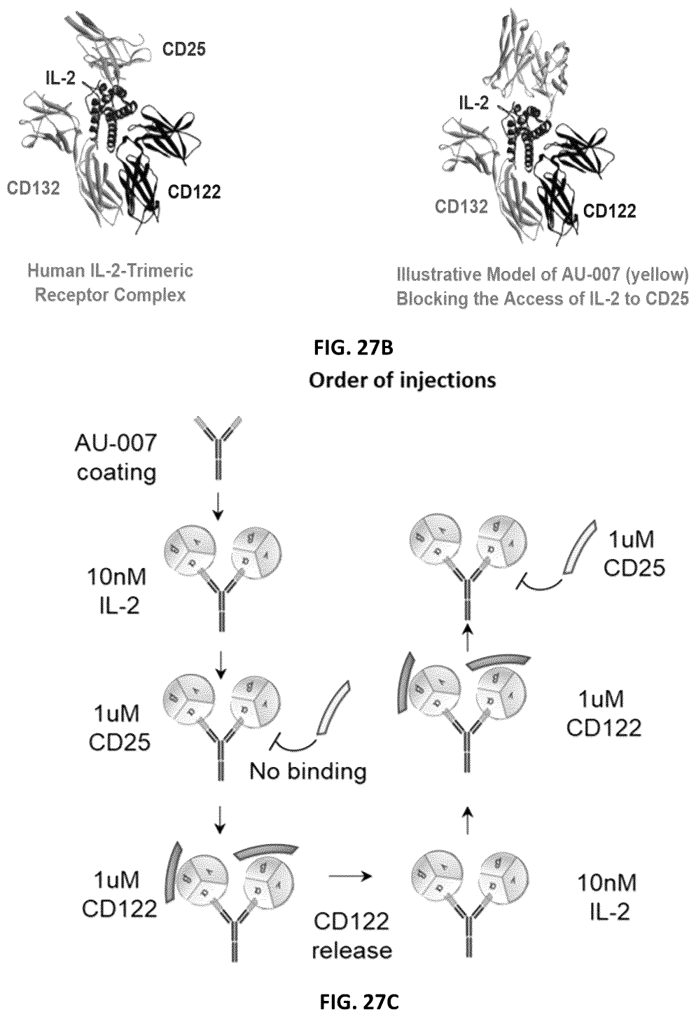

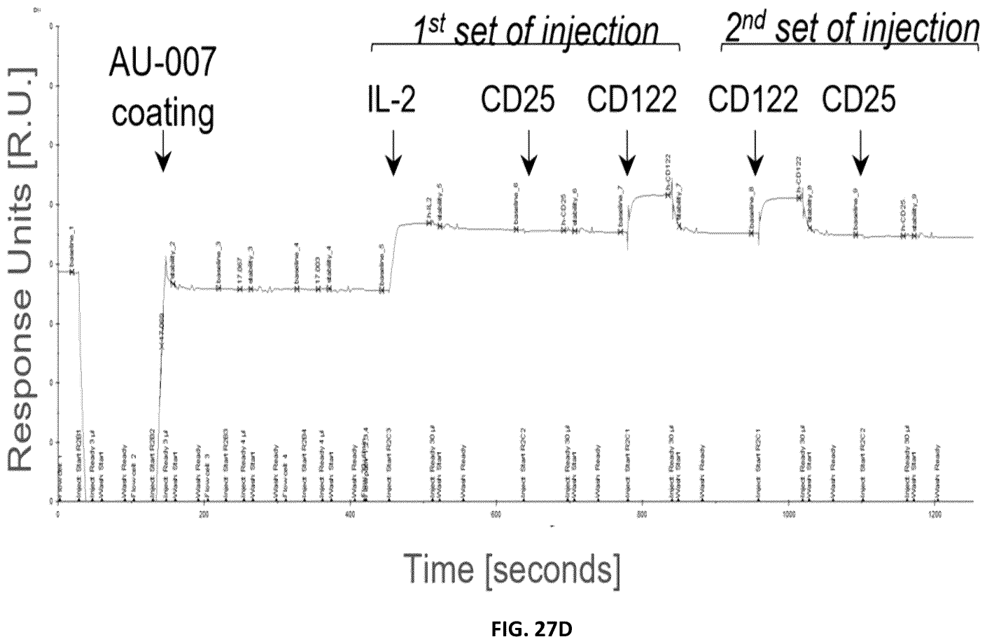

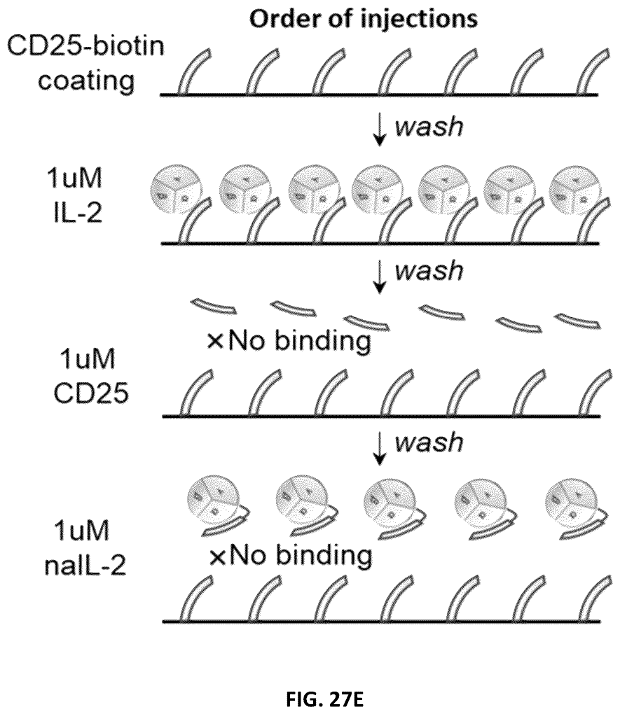

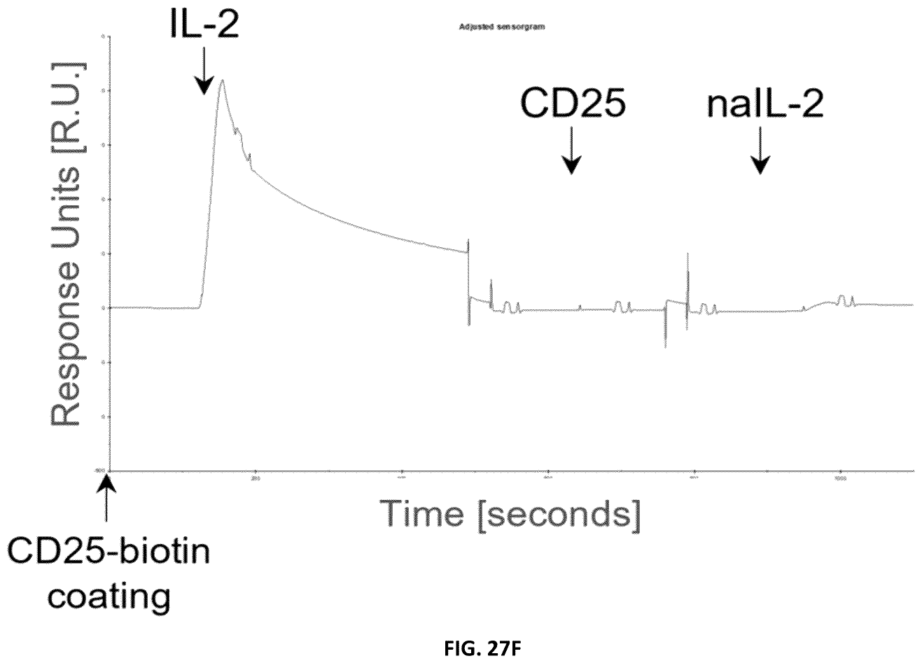

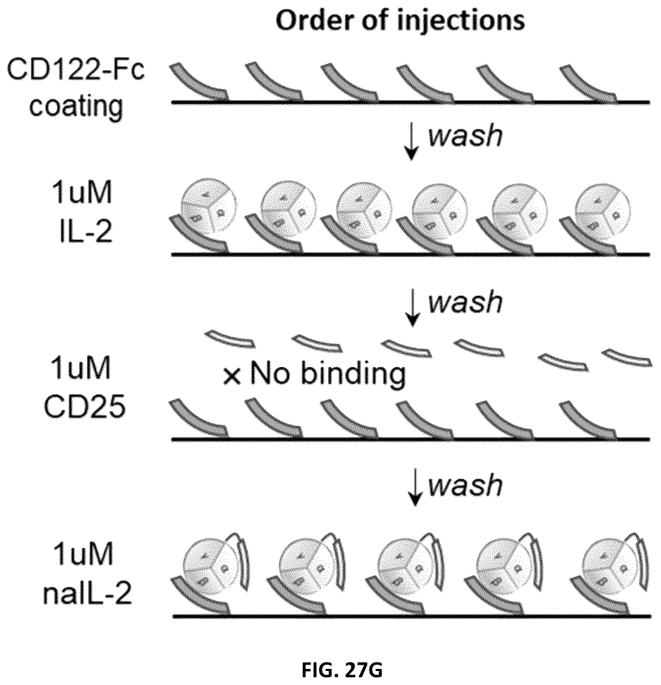

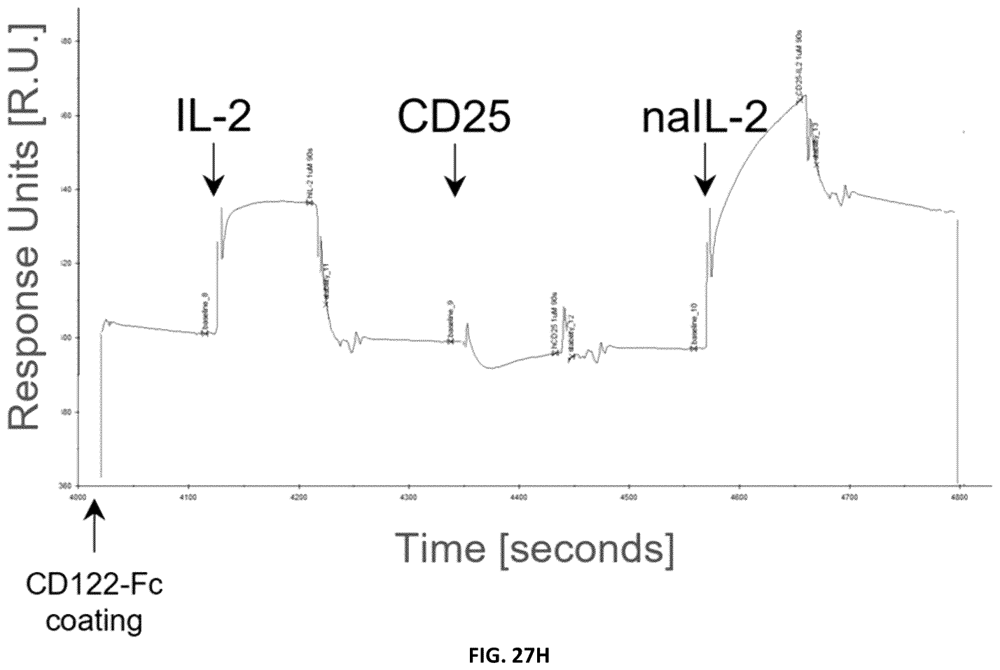

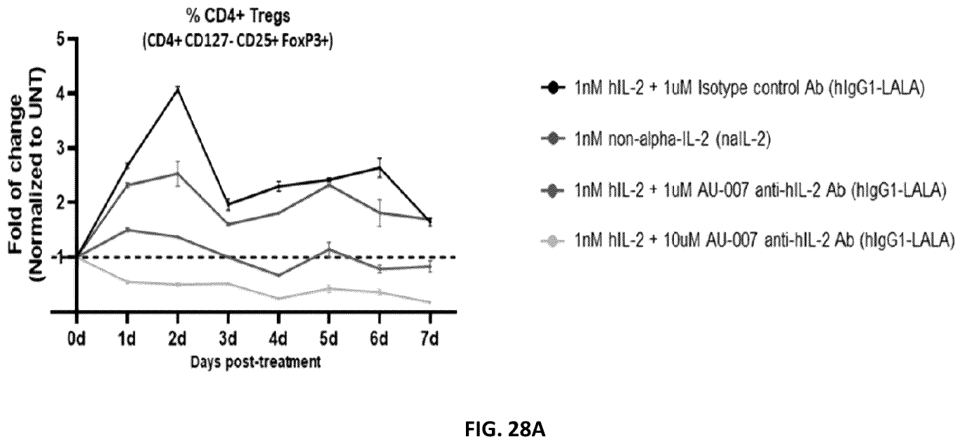

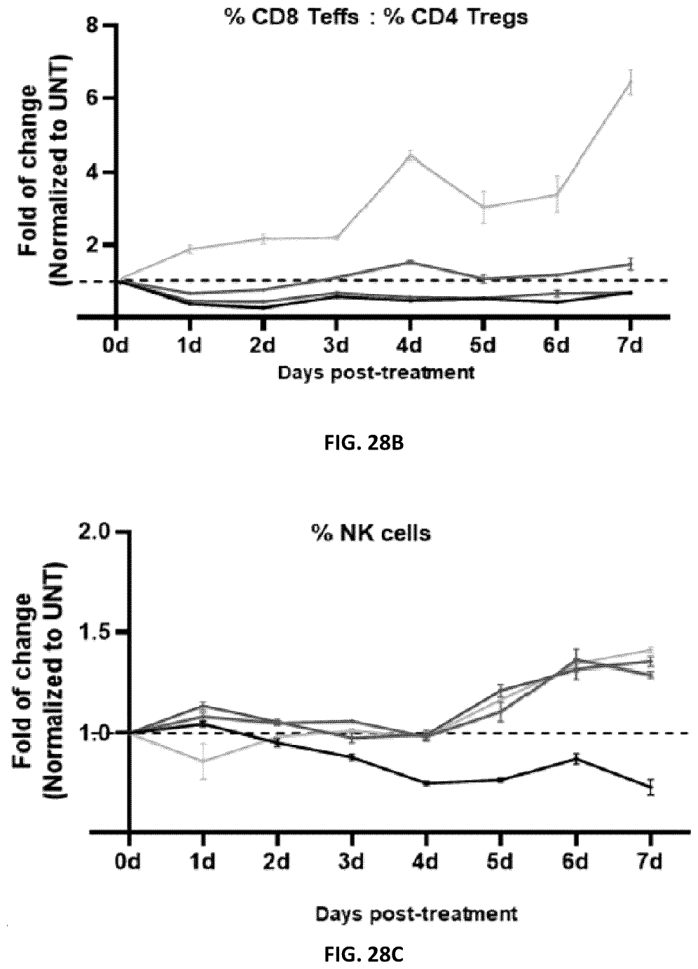

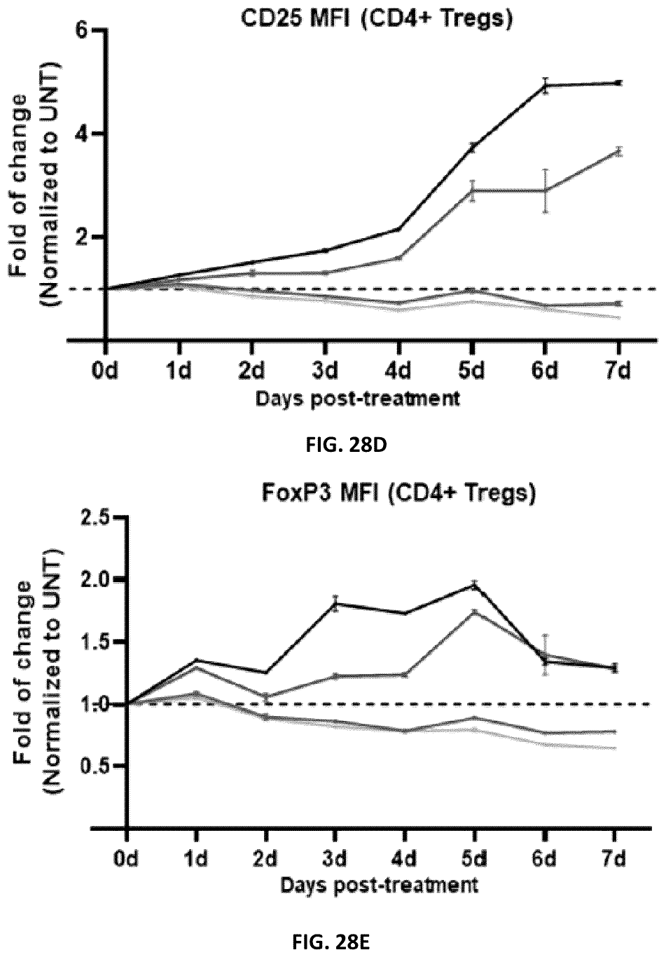

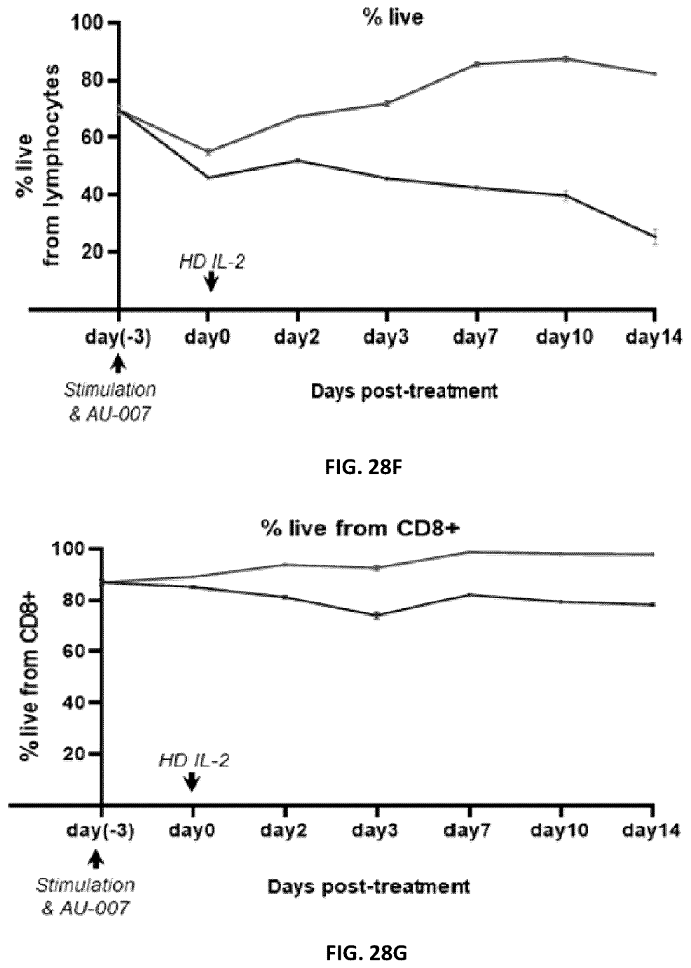

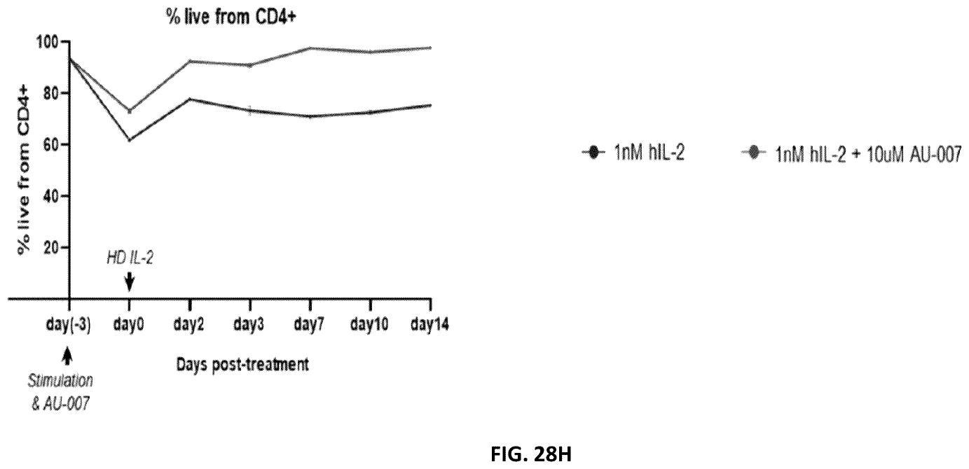

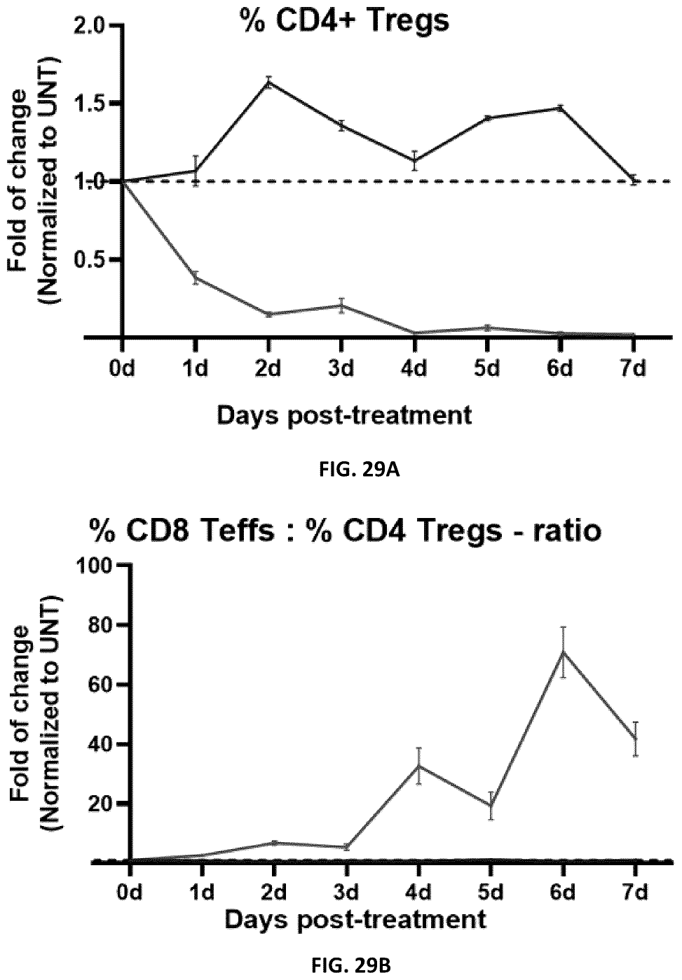

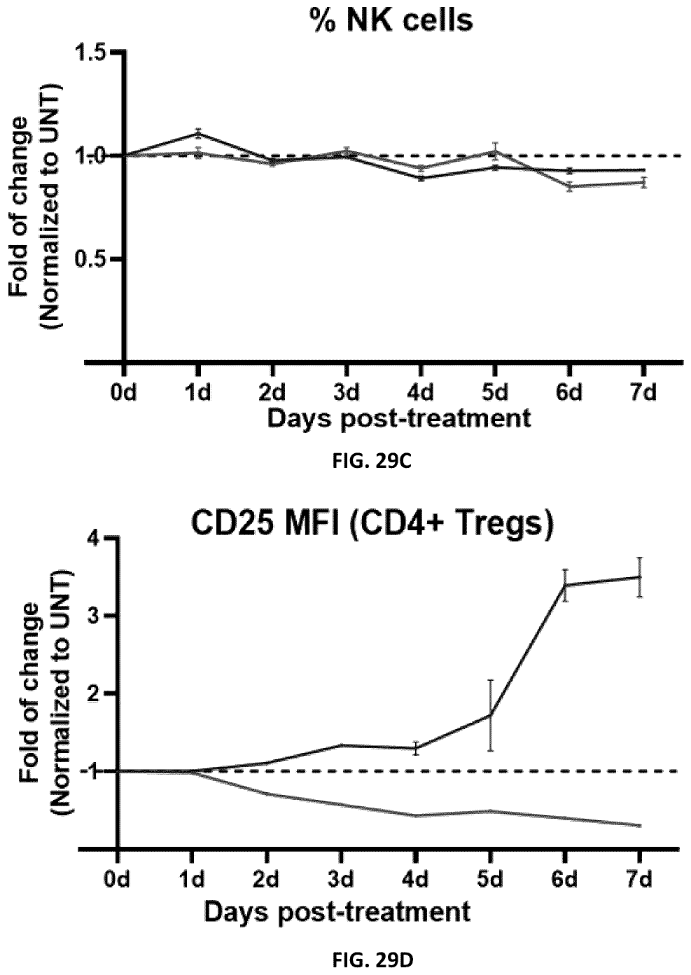

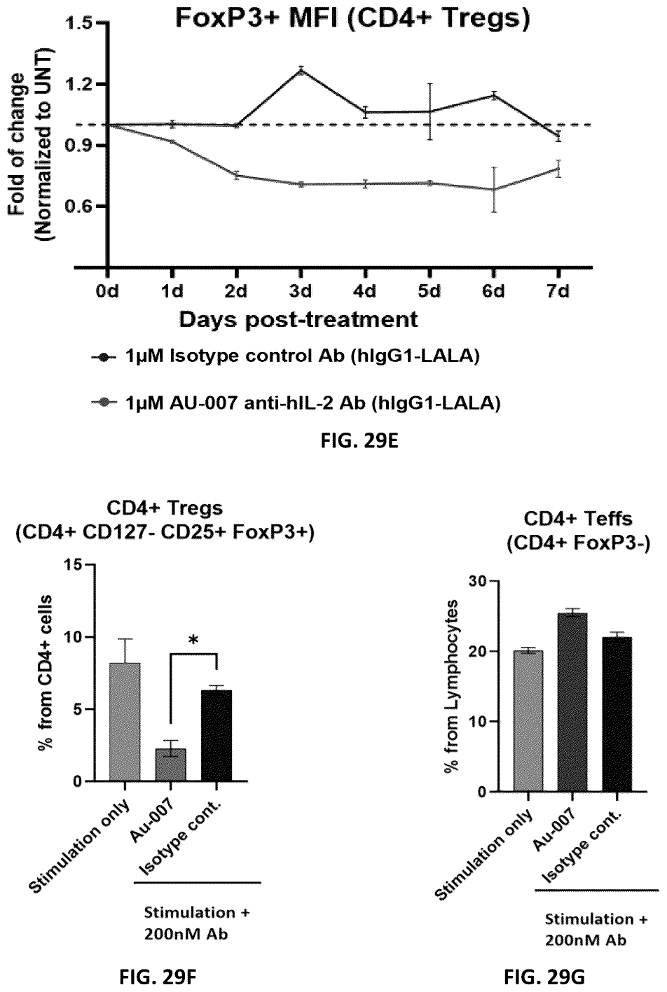

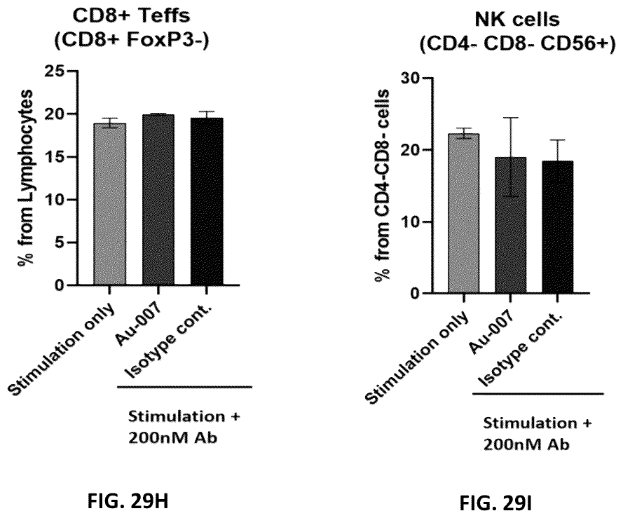

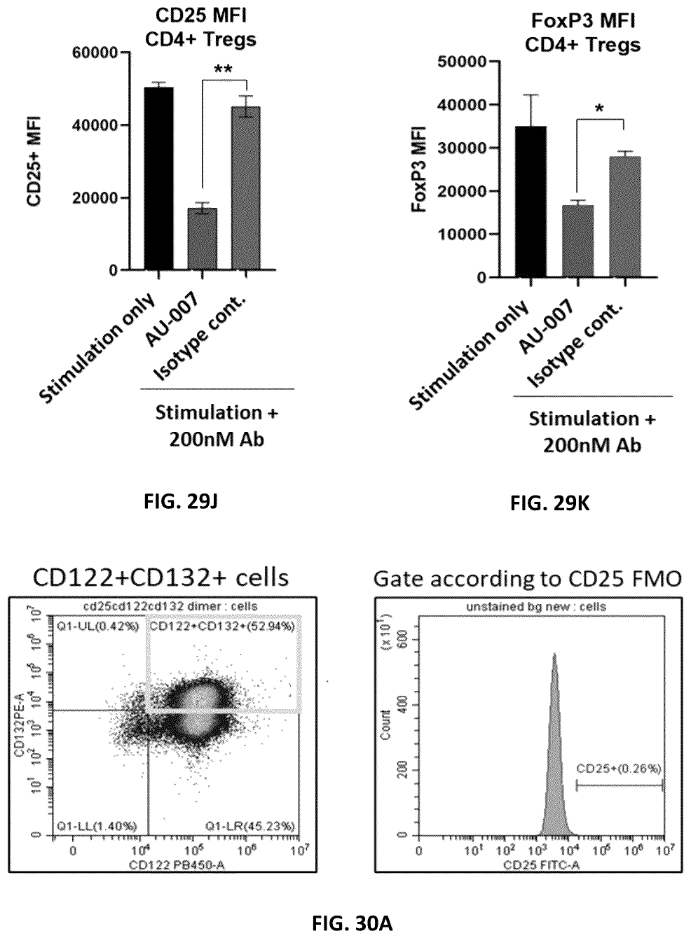

It was hypothesized herein that exogenous engineered IL-2 expands cells that secrete endogenous IL-2, creating negative feedback by expanding Tregs. Specifically, modified cytokine approaches lack the ability to control the autocrine/paracrine-secreted IL-2 and their efficacy will be limited by the expansion of Tregs and enhanced RICD triggered by endogenously released, unmodified, IL-2. It was further hypothesized that controlling endogenously secreted IL-2 will prevent this autoinhibitory feedback loop and replace it with a positive feedback loop that will improve efficacy. To test this hypothesis, the activity and effect of three compounds was compared: non-modified IL-2, a non-alpha-IL-2 (naIL-2, IL-2 conjugated to CD25), and the high-affinity anti-IL-2 monoclonal antibody 17.069 (AU-007) that completely block IL-2 interaction with CD25 while sparing its interaction with the dimeric CD122/CD132 receptor (FIGS. 27A-27H). It was found that treatment with exogenous IL-2 or naIL-2 resulted in a downstream expansion of Tregs (FIGS. 28A-28H) while controlling endogenous IL-2 using an epitope-specific blocking Ab (AU-007) resolved IL-2 immune inhibitory negative feedback loop and replaced it with a positive feedback loop that is preferred for cancer immunotherapy (FIGS. 28A-28H and 29A-29K).

### Methods:

Non-alpha-IL-2 (naIL-2) production. Human IL-2 with an 8xHis C-terminal tag conjugated with a linker (G4STRG4STG4SG4SS) to human CD25 extracellular domain at the IL-2 N-terminus was expressed in Expi293 cells (ThermoFisher, A14527) using pSF-CMV expression vector according to the manufacturer protocol. In brief, 1 ug/ml DNA was transfected to cell culture at a density of 3×10\hat{ }6 (viability of 97%) using an ExpiFectamin293 kit containing transfection reagent and enhancers (ThermoFisher, A14524). After transfection, cells were incubated at 37° C. with 80% humidity and 8% C02. Enhancers were added to the transfected cell culture 21 hours post-transfection. 7 days post-transfection, cells were harvested, and the supernatant was collected and dialyzed overnight to PBS supplemented with 25 mM imidazole and 200 mM NaCl (buffer A). The following day, the supernatant was loaded on 5 ml HisTrap (Cytiva, 17524802) at a flow rate of 2.5 ml/min. Following buffer A wash, elution was done in steps 20, 40, and 60% buffer B containing PBS, 200 mM NaCl, and 0.5M imidazole at a flow rate of 4.5 ml/min. After SDS-PAGE analysis, fractions were pooled and dialyzed overnight to PBS. The non-alpha-IL-2 protein was flash-frozen in liquid N₂ at 1 mg/ml.

SPR Kinetics for determination of AU-007 affinity to IL-2. Kinetic measurements of AU-007 for human IL-2 were done using Biacore T200 (GE Healthcare, USA) on a CM5 chip (BR10005-30, Cytiva). The chip was crosslinked with a human antibody capture kit (BR100839, GE Healthcare, USA) according to the manufacturer's protocol to target 3000-4000 RU. Kinetics determination was performed using a multi-cycle strategy, AU-007 was captured on the coated chip to reach ˜300 RU. Human IL-2 (RKP60568, Reprokine) was injected from 10 nM to 0.003 nM in two-fold dilutions. Between each cycle, all channels underwent regeneration using 3M MgCl₂. Binding kinetics were determined by the 1:1 Binding model using the Biacore T200 evaluation software, version 3.1.

SPR for determination of AU-007 binding epitope on IL-2. AU-007 was captured on an anti-human Fc (BR100839, Cytiva) coated CM5 chip (BR10005-30, Cytiva) to reach ˜300 RU. 50 nM human IL-2 (RKP60568, Reprokine) was injected over 60 seconds to saturate the antibody, and then either 1 uM CD25 (RKP01589, Reprokine) or 1 uM CD122 (RKP14784, Reprokine) was injected for 60 seconds. AU-007 binding epitope was characterized based on the ability of the IL-2 receptor subunits to bind IL-2 in the presence of AU-007.

SPR for determination of non-alpha-IL-2 available epitopes. To test whether the CD25 epitope is blocked in the non-alpha-IL-2 format, a biotinylated human CD25 (AVI10305-050, R&D systems) was captured on a streptavidin-coated chip (BR100531, Cytiva) and 200 nM of human IL-2 (RKP60568, Reprokine) or non-alpha-IL-2 (IL-2 conjugated to CD25 subunit) were injected for 60 seconds. To verify that the ability of the non-alpha-IL-2 to bind CD122 is preserved, an Fc-tagged CD122 (extracellular domain) (RKP14784F, Reprokine) was captured on an anti-human Fc-coated (BR100839, Cytiva) CM5 chip (BR10005-30, Cytiva) to reach ˜400 RU and 1 uM of human IL-2 (RKP60568, Reprokine) or the non-alpha-IL-2 were injected for 90 seconds. In both assays, CD25 and CD122 available epitopes were characterized based on the ability of the IL-2 receptor subunits to bind IL-2 or the non-alpha-IL-2.

Naïve Human PBMCs expansion assay. Human PBMCs were thawed into complete hPBMC media (RPMI, supplemented with 10% FBS, 1% pen/strep, 10% sodium pyruvate, 1% NEAA, 10% Glutamax, 0.10% 2-mercaptoethanol—0.05 mM), and rested for 4-5 h prior to assay initiation. After resting, cells were cultured in 24 well plates (1*10\hat{ }6 cells/1 ml in each well) with 1 uM of AU-007 or with 1 uM of an isotype control antibody without supplementation of exogenous hIL-2 (FIGS. 29A-29E). Alternatively, cells were incubated with 1 nM of non-alpha-IL-2 or 1 nM native hIL-2 in the presence of 1 uM or 10 uM of AU-007 or an isotype control antibody (FIGS. 28A-28E). Cells were monitored for 7 days, and immune cell subpopulation analysis was done daily using flow cytometry. At each time point, cells were stained with fixable viability dye according to manufacturer instructions, followed by staining of cell surface markers (CD3, CD4, CD8, CD25, CD56, CD127), fixation/permeabilization (Miltenyi, cat #130-093-142) followed with staining of the intracellular marker, FoxP3. Tregs were defined as CD3+ CD4+ CD25+ CD127-FoxP3+, CD8 Teffs were defined as CD3+ CD8+ FoxP3−, NK cells were defined as CD3-CD56+, NKTs were defined as CD3+ CD8+ CD56+. Gating was defined based on fluorescence minus one control (FMO); all gating originated from lymphocytes' live cells. The full antibodies list that was used for the detection of immune cell markers is detailed in Table 13.

TABLE 13
Antibodies used for flow cytometry in vitro experiments of cultured hPBMCs.

| Markers | Fluorochrome | Clone | Cat. | Isotypes | Vender |
|---|---|---|---|---|---|
| hCD3 | PE- CF594 | UCHT1 | 562280 | Mouse IgG1, κ | BD |
| hCD4 | FITC | RPA-T4 | 555346 | Mouse IgG1, κ | BD |
| hCD8 | APC-Cy7 | RPA-T8 | 557760 | Mouse IgG1, κ | BD |
| hCD25 | BV421 | BC96 | 302630 | Mouse IgG1, κ | BioLegend |
| hCD127 | BV711 | HIL-7R-M21 | 563165 | Mouse IgG1, κ | BD |
| hCD56 | BV605 | HCD56 | 318334 | Mouse IgG1, κ | BioLegend |
| hFoxP3 | PE | 150D/E4 | 12-4774-42 | Mouse IgG1, κ | eBioscience |
| pSTAT5 | AF647 | 47/Stat5(pY694) | 562076 | Mouse IgG1, κ | BD |
| Viability dye | EF660 | NA | 65-0864-18 | NA | eBioscience |

Activated Human PBMCs expansion assay. Human PBMCs were thawed into complete PBMC media and rested overnight prior to assay initiation. After resting, cells were activated with anti-CD3/anti-CD28 Abs (Stemcell, cat #10991) and cultured in 24 well plates (1*10\hat{ }6 cells/1 ml in each well) with 200 nM of either AU-007 or an isotype control antibody for 24 h. After incubation, cells were stained with fixable viability dye according to manufacturer instructions, followed by extracellular markers staining (CD4, CD8, CD25, CD56, CD127), fixation/permeabilization (Miltenyi, cat #130-093-142) and staining of the intracellular marker FoxP3 for the assessment of different cell subsets expanded after treatment. Tregs were defined as CD4+ CD25+ CD127-FoxP3+, CD8 Teffs were defined as CD8+ FoxP3−, NK cells were defined as CD8-CD4-CD56+, NKTs were defined as CD8+ CD56+. Gating was defined based on fluorescence minus one control (FMO); all gating originated from lymphocytes' live cells. The full antibodies list that was used for the detection of immune cell markers is detailed in Table 14.

TABLE 14
Antibodies used for flow cytometry experiments of hPBMCs that were
isolated from spleens of NOG-EXL mice engrafted with hPBMCs.

| Markers | Fluorochrome | Clone | Cat. | Isotypes | Vender |
|---|---|---|---|---|---|
| mCD45 | PerCP-Cy5.5 | 30-F11 | 103132 | Rat IgG2b | BioLegend |
| hCD45 | BV785 | HI30 | 304048 | Mouse IgG1, κ | Biolegend |
| hCD3 | BUV395 | SK7 | 564001 | Mouse BALB/c | BD |
| | | | | [symbol] | |
| hCD4 | FITC | OKT4 | 317408 | Mouse IgG2b, κ | BioLegend |
| hCD25 | BV711 | M-A251 | 356138 | Mouse IgG1, κ | BioLegend |
| hCD127 | PE-Cy7 | HIL-7R-M21 | 560822 | Mouse IgG1, κ | BD |
| hFoxP3* | PE | 259D/C7 | 560082 | Mouse BALB/c | BD |
| | | | | [symbol] | |
| hCD8 | PE/Dazzle594 | SKI | 344744 | Mouse IgG1, κ | BioLegend |
| hCD45RA | BV421 | HI100 | 304130 | Mouse IgG2b, κ | BioLegend |
| hCCR7 | APC | G043H7 | 353214 | Mouse IgG2a, κ | BioLegend |
| hCD56 | BV605 | HCD56 | 318334 | Mouse IgG1, κ | BioLegend |
| Viability dye | eF780 | NA | 65-0865-18 | NA | eBioscience |

[symbol]

indicates data missing or illegible when filed

Lymphocyte viability assay. Human PBMCs were thawed into complete PBMC media and rested for 4-5 h prior to assay initiation. After resting, cells were activated with anti-CD3/anti-CD28 Abs (Stemcell, cat #10991) and cultured in 24 well plates (1*10A6 cells/1 ml in each well) with or without 10 uM of AU-007. 3 days later cultures were supplemented with 1 nM hIL-2 and monitored by flow cytometry at 7- and 14-days post-treatment. At each time point, cells were stained with fixable viability dye according to manufacturer instructions, followed by extracellular markers staining (CD4, CD8) and viability die.

HEK dimer pSTAT5 activation. HER cells expressing the dimeric form of the IL-2 receptor (hkb-IL-2, Invivo-Gen) were grown in complete media (DMEM (high glucose)+100 L-glutamine) until harvesting on the day of the experiment. Upon cell count, 50,000 cells/100 ul were seeded per well in a 96-well plate. Cells were treated with serial dilutions of non-alpha-IL-2 (100 nM-1.28 pm) or with serial dilutions of hIL-2 (100 nM-1.28 pM) combined with 200 nM of either AU-007 or an antibody that inhibits IL-2 binding to the dimeric receptor (dimer inhibitor antibody) or with an isotype control antibody. Treatment was done in a final volume of 200 ul/well. The cells were then transferred to 37° C. for overnight incubation. After 24 h, 20 ul of cell supernatant was transferred to a new plate and 180 ul of Quanti-Blue (Invivo-Gen, Cat #: rep-qbs) working solution was added to the wells. The plate was incubated with Quanti-Blue for 1 hour and analyzed using a plate reader at 620-655 nm.

Mice study. Animal studies were held at Crown Bioscience, Inc. (San Diego). Mice were housed under specific pathogen-free conditions as per the national animal testing regulations. Animal welfare for this study complies with the U.S. Department of Agriculture's Animal Welfare Act in strict accordance with applicable Crown Bioscience, Inc. 10 days prior to dosing, 6-9 weeks of age female NOG-EXL non-humanized (Jackson Laboratory) were inoculated with 10 million PBMCs (isolated from 3 different donors) per mouse via i.v. injection. Before dosing, all animals were randomly allocated to the different study groups, each group contained 9 mice, 3 mice for each hPBMCs' donor. Randomization was performed in the Study Log software two days prior to dosing [Singh A V, Varma M, Laux P, Choudhary S, Datusalia A K, GuptaN, et al. Artificial intelligence and machine learning disciplines with the potential to improve the nanotoxicology and nanomedicine fields: a comprehensive review. Archives of Toxicology. 2023 Mar. 7; 97:963-79]. The average body weight (grams) for each group ±SD at randomization was as follows: Group 1—Isotype control 17.37±1.09, Group 2 AU-007 17.47±0.94. Animals were dosed once with 20 mg/Kg (5 ml/Kg) via i.p. Eight days post-treatment animals were harvested, hearts were terminally bled for serum collections, and spleens were collected for immune cell analysis.

Ab/hIL-2 complex detection from mice serums. Ab/hIL-2 complex was detected from mice serums using ELISA. High binding 96 well plate was coated with 200 ng/well polyclonal goat anti-hIL-2 and incubated overnight at 4° C. (R&D Bioscience, Kit catalog: #SEL202, part: #840606 Lot: QQ0819051). The plate was then washed 3 times with 300 ul PBS-T (0.05% Tween) followed by blocking using 1.5% Milk diluted in PBS-T at 300 ul/well and incubated for 1 hour at R.T. Plate was washed 3 times with 300 ul PBS-T (0.05% Tween) and incubated for 1 hour at room temperature with serums samples or with standard curve samples. Pre-incubation, serums were diluted 1:2 according to the MRD and compared to a standard curve of AU007 mixed with hIL-2 at 2:1 ratio respectively in serum diluted at room temp according to the MRD. The plate was washed 3 times with 300 ul PBS-T (0.05% Tween) and incubated for 30 minutes at room temperature with goat anti-human Fc-HRP diluted 1:20000 in PBS (Jackson ImmunoResearch 109-035-008). The plate was washed again 3 times with 300 ul PBS-T (0.05% Tween). Chromogenic substrate (TMB) was added for each well followed by a stop solution. The plate was analyzed using a plate reader at 450 nm.

### Results:

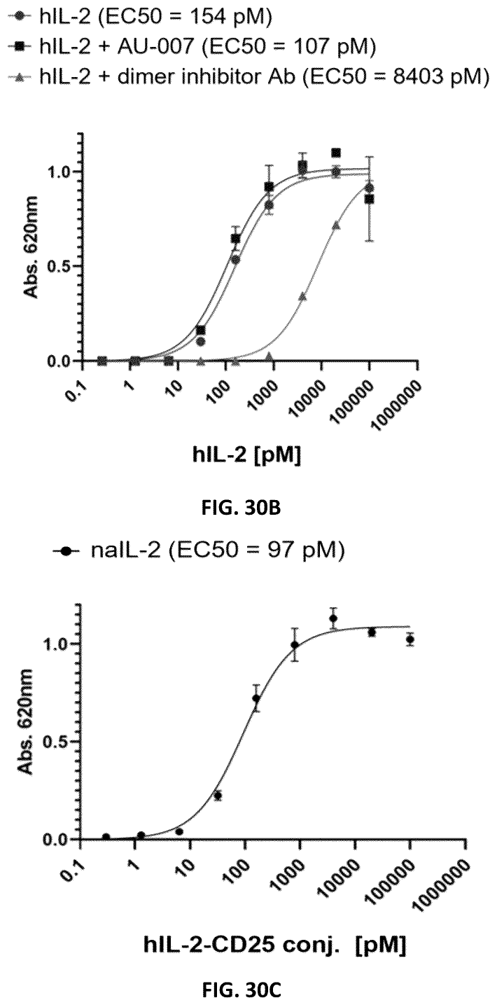

Generation of IL-2-based compounds that inhibits IL-2 binding to CD25 and allow IL-2 signaling only through the dimer receptor. To demonstrate the effect of the IL-2 negative feedback loop, two compounds were developed: i) a monoclonal antibody (AU-007) that captures endogenously secreted IL-2 and blocks its ability to bind and activate IL-2 trimeric receptor expressed over regulatory T-cells while sparing IL-2 interactions with the dimeric receptor expressed over effector cells, and ii) a non-alpha-IL-2 (naIL-2) cytokine that can only interact with the dimeric receptor yet has no effect over endogenous IL-2 that is natively secreted from activated T-cells. AU-007 was computationally designed to bind IL-2 at the CD25 binding site. The epitope differentiates the binding of the dimer on effector cells from the epitope that mediates the binding on the trimer expressed on Tregs was inferred from the crystal structure (PDB code 2B51). The surface of IL-2 that is buried under CD25 was the focus of the design and avoided surface residues that contact the dimer subunits. Using surface plasmon resonance, it was verified that AU-007 binds hIL-2 with a desired pM affinity required for capturing low levels of IL-2 that allows full competition with the high-affinity trimeric receptor (FIG. 27A). FIG. 27B presents comparative illustrative models of IL-2 binding as part of a human IL-2-Trimeric Receptor Complex compared with AU-007 (yellow) blocking the access of IL-2 to CD25. Next, it was shown that both the naIL-2 and AU-007 inhibited binding to CD25 but not to CD122 (FIGS. 27C-27H). Further, it was shown that both AU-007 and the naIL-2 cytokine preserve IL-2 functional signaling through the IL-2-dimeric receptor. To demonstrate this, a reporter cell line that stably expresses the human IL-2 dimer receptor (CD122/CD132) was used and it was demonstrated that neither AU-007 nor naIL-2 interfered with IL-2/IL-2-dimer receptor signaling (FIGS. 30A-30C).

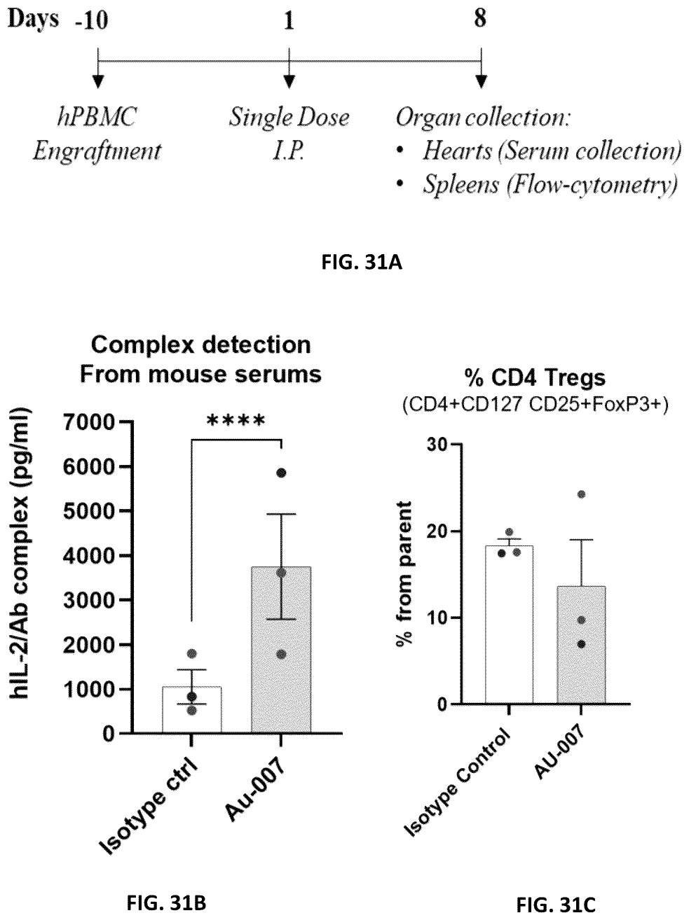

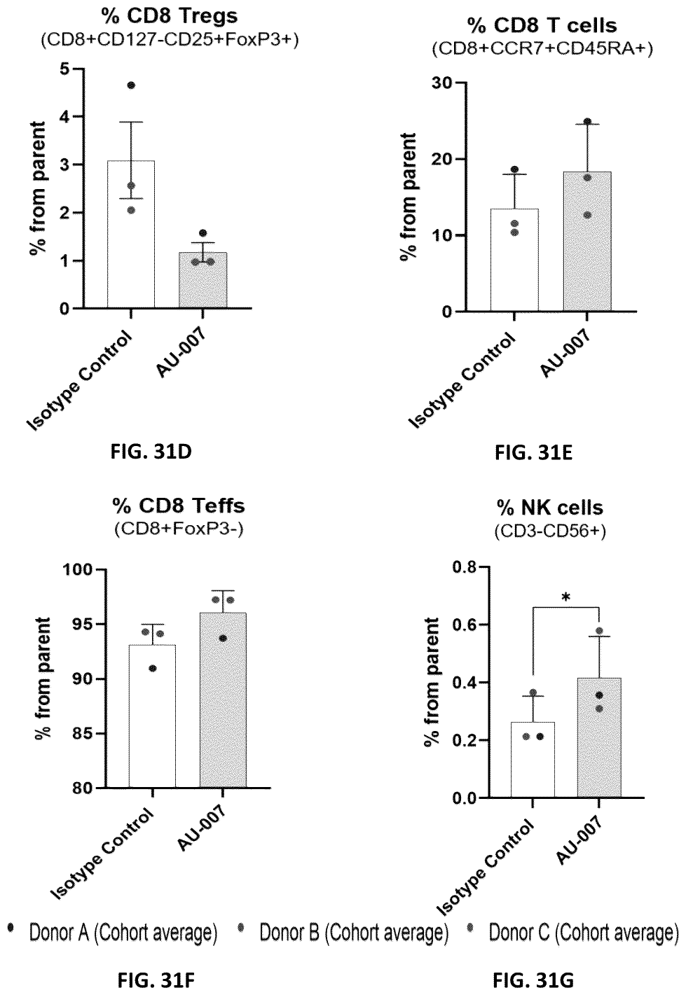

AU-007 binds to endogenous IL-2 and breaks the negative feedback loop in human PBMCs. In order to break the autoinhibitory feedback loop of IL-2 in inflamed systems, it is not sufficient to bind IL-2 with tight affinity and block its interaction with CD25. An additional and challenging requirement is that the antibody must acquire the ability to capture and control endogenous IL-2 that is being constitutively secreted by activated T cells and consumed by CD25+ Tregs or activated CD25+ Teffs. To validate that AU-007 captures endogenously secreted hIL-2 in vivo, immunodeficient NOG-EXL mice were engrafted with hPBMCs. Ten days post engraftment, mice were treated once with AU-007 or with an isotype control antibody. Eight days post-treatment hearts were terminally bled for serum collection and spleens were harvested for immune cell analysis using flow cytometry (FIG. 31A). AU-007 single-dose administration led to the accumulation of endogenously secreted hIL-2 and inhibited Tregs. Significantly high levels of AU-007 bound to endogenous IL-2 were detected from mouse serums (FIG. 31B). CD8+ T-cells and NKs were elevated compared to the isotype control-treated mice even eight days post-treatment (FIGS. 31C-31G). To further test whether AU-007 efficiently captures endogenous IL-2 in vitro, we treated hPBMCs with AU-007. Total hPBMCs were treated once with AU-007 or with an isotype control antibody, without adding exogenous IL-2. Cells were monitored daily for 7 days by flow cytometry.

While the control antibody didn't affect Tregs, AU-007 completely inhibited the expansion of Tregs (FIG. 29A) and significantly increased Teff:Treg ratio (FIG. 29B). Next, hPBMCs were activated with anti-CD3/anti-CD28 and treated the activated culture with AU-007. Under these T-cell activating conditions, AU-007 selectively inhibited Tregs without affecting Teffs or NKs. Furthermore, AU-007 downregulated the suppressive markers of CD4+ Treg (FIGS. 29D-29E, and 29J-29K). Since no exogenous hIL-2 was added, this indicates that AU-007 captures endogenous IL-2 and prevents the negative feedback loop initiated by the IL-2 secreted from activated T-cells that leads to the expansion of Tregs.

AU-007 can capture and redirect endogenous IL-2 to break the auto-inhibitory loop in hPBMCs while HD IL-2 or naIL-2 cannot. Finally, the negative feedback loop caused by IL-2 or naIL-2 was compared to the positive feedback loop caused by AU-007. Total hPBMCs were incubated in vitro for 7 days after a single treatment with either 1 nM of naIL-2 alone or 1 nM of IL-2 combined with AU-007 in two different ratios 1:1000 (1 uM of AU-007) or 1:10,000 (10 uM of AU-007). Immune cell subpopulations were analyzed daily by flow cytometry. FIGS. 28A-28H show that both AU-007 and naIL-2 expanded NKs and Teffs. However, the addition of naIL-2 led to the expansion of Tregs even several days after its administration, whereas the addition of AU-007 did not. These data demonstrate that AU-007 redirects both exogenous IL-2 and endogenous paracrine secreted IL-2 in a way that inhibits Tregs and the expression of Treg suppressive markers while increasing Teffs:Tregs ratio and expanding NKs (FIGS. 28A-28E). To maintain homeostasis upon activation of T-cells, paracrine IL-2 induces Tregs expansion. However, both autocrine and paracrine IL-2 induce RICD on activated CD25+ T-cells. To test if AU-007 protects cells from RICD, the overall viability of activated hPBMCs was examined in the presence or absence of AU-007. hPBMCs were stimulated once with anti-CD3/anti-CD28 antibodies with or without 10 uM of AU-007. Three days post-stimulation, samples were treated with IL-2 (1 nM) to induce IL-2-dependent RICD. Lymphocyte viability was monitored daily for 14 days using flow cytometry. Stimulated lymphocytes showed a 50% reduction in viability after a single dose of IL-2. The addition of AU-007 caused a ˜10% increase in viability, further supporting the ability of AU-007 to prevent the stimulus-induced negative feedback caused by endogenous IL-2 release (FIGS. 28F-28H). Taken together, these results show that activation-induced release of IL-2 produces an immunosuppressive effect by expanding Tregs and triggering RICD. This activation-induced release is triggered by IL-2 itself, producing a self-delimiting negative feedback loop, which interferes with the stimulatory effect of exogenously administered IL-2 or naIL-2. AU-007, a computationally designed antibody that prevents IL-2 binding to CD25, redirects autocrine/paracrine secreted IL-2, thereby converting the negative feedback loop into a positive feedback loop that prolongs immune stimulation.

The IL-2 autoinhibitory feedback loop described here may explain the limited efficacy of modified IL-2 therapies observed in clinical development. Moreover, the findings presented here indicate that therapeutic approaches targeting the IL-2 autoinhibitory feedback loop may hold promise for cancer immunotherapy. AU-007 is currently being evaluated in a Phase 1/2 clinical trial in several types of tumors (See, Examples herein)

These results demonstrate that treatments utilizing exogenous modified IL-2 indeed induce Treg expansion. To improve treatments results and inhibit Treg expansion, this Example used the monoclonal humanized antibody 17.069 (AU-007) that binds human IL-2 with pM affinity in a predefined epitope and completely blocks IL-2 binding to CD25 that is highly expressed over Tregs, without hindering IL-2 binding to CD122/CD132 dimer receptor expressed over effector cells. As demonstrated herein, this epitope-specific, high-affinity antibody controls endogenous IL-2 and prevents it from expanding Tregs while allowing it to expand Teffs. It was shown that controlling endogenous IL-2 using 17.069 (AU-007) abrogated the negative feedback loop and replaced it with a positive feedback loop that enhanced the expansion of NK cells and Teffs, an effect considered favorable for cancer immunotherapy.

Conclusion and Summary: IL-2 was the first successful cancer immunotherapy. As early as 1984 it had been observed that, while extremely toxic, high doses of IL-2 can cure advanced metastatic cancer in some patients. Ever since harnessing IL-2 to treat cancer has been a promising and challenging goal. Recombinant hIL-2 (Proleukin® (aldesleukin), aldesleukin) was approved as a cancer immunotherapeutic in the 1990s. It showed efficacy in some individuals that tolerated the high toxicity of the high doses required to induce antitumor effects. The basis behind the immunomodulatory biology of IL-2 is dependent on differential concentrations of the cytokine during homeostasis and inflammation. At low concentrations, the cytokine binds a trimeric IL-2 receptor on regulatory T cells, whereas at high concentrations, IL-2 will also engage the lower-affinity dimeric receptor found on effector T cells and natural killer cells.

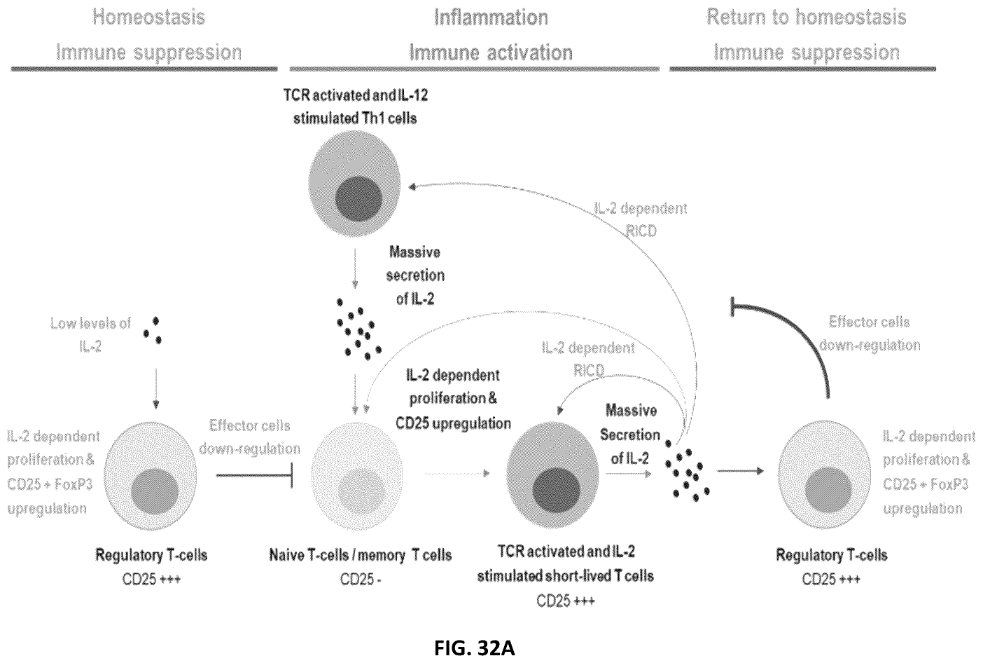

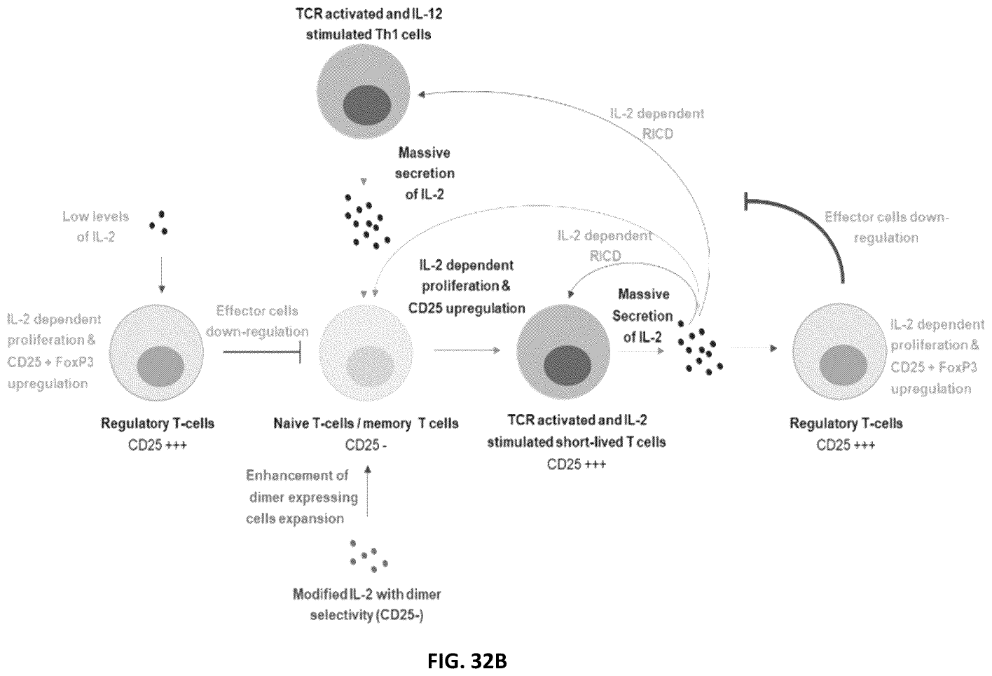

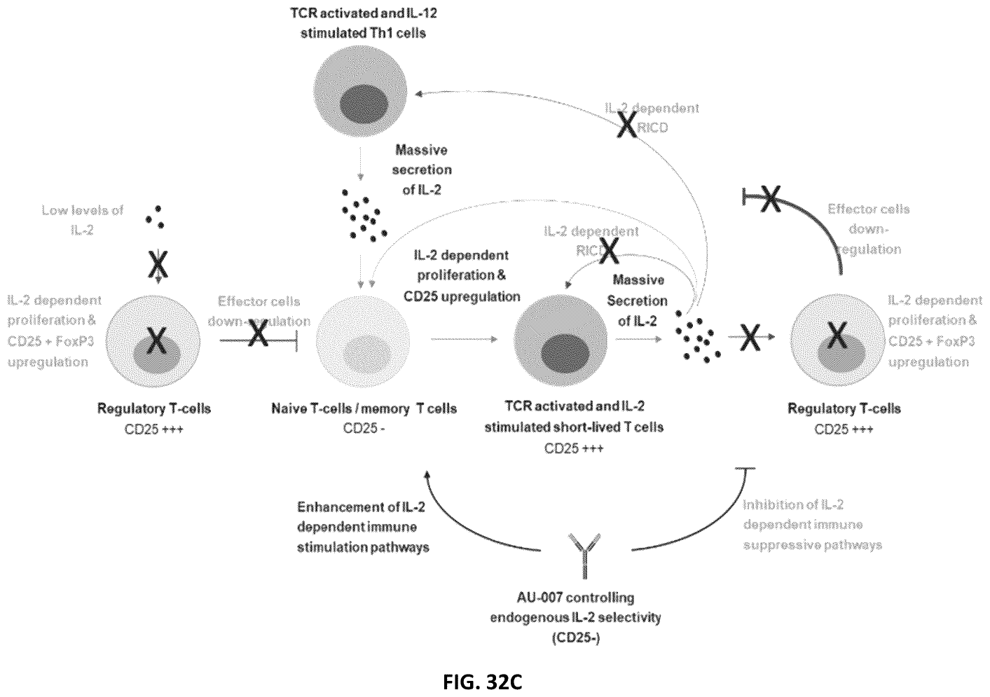

The main source of endogenous IL-2 is activated T cells. These cells secrete IL-2 while also transiently upregulating the CD25 subunit to express the high-affinity trimeric receptor. The secreted IL-2 is consumed by Tregs as well as by the activated effector cells. The effect of the consumed IL-2 is radically different between these T-cell populations. IL-2 consumption by Tregs and naive Teffs leads to their expansion, while its consumption by activated CD25+ Teffs is believed to induce apoptosis. In both cases, IL-2 helps restore homeostasis by downregulating the immune attack (FIG. 32A). Multiple attempts have been made to modify IL-2 in a way that will bias its selectivity toward the expansion of Teffs and away from Tregs. While these therapeutic approaches had shown great promise at pre-clinical stages, their clinical results have been disappointing.

Here is shown that treatments involving an external source of IL-2 or a naIL-2 with biased selectivity to effector cells, fail to handle the negative feedback loop of endogenous IL-2 that inherently drives the immune system toward immune downregulation and homeostasis. Although naIL-2 cannot interact with CD25 (FIG. 27D), binds CD122 (FIG. 27E), stimulates the dimeric receptor properly (FIGS. 30A-30C), and expands NK cells (FIG. 28C), it fails to prevent Tregs expansion (FIG. 28A). The fact that CD25+ Tregs, that rely solely on IL-2 to survive, double their percentage and remain viable even 7 days post-treatment, it clearly shows that the naIL-2 activity fails to control the natively expressed IL-2 that is free to bind CD25+ Tregs (FIG. 32B).

This Example demonstrates that AU-007, a monoclonal antibody that captures and controls the activity of endogenous secreted IL-2 produced by activated T cells, can break this autoinhibitory feedback loop of IL-2 and drive its activity towards immune stimulation. AU-007 captures and redirects endogenous IL-2, allowing it to expand Teffs cells and NK cells while completely preventing it from binding CD25+ cells. As demonstrated in FIGS. 29A-29K and FIGS. 32A-32C, activated cells that were treated once with AU-007 inhibit Tregs and maintain Teffs and NK cells even without the addition of IL-2 from an external source. These results demonstrate that AU-007 captured and controlled endogenously secreted IL-2, preventing it from stimulating Tregs, while allowing it to effectively stimulate Teffs and NK cells. Moreover, this long-term effect was also observed when cells were treated once with AU-007 followed by a single high dose of IL-2 (FIGS. 28A-28E). Additionally, FIGS. 28F-28H shows that AU-007 rescues the activated and IL-2-stimulated lymphocytes from cell death. The inhibition of Tregs, the expansion of effector cells, and the prevention of activated effector cell death all together break the autoinhibitory activity of IL-2. This allows for the expansion of the immune stimulatory stage that is considered favorable for cancer immune therapy, (FIG. 32C).

Unique in the IL-2 field, AU-007 can redirect endogenous IL-2 generated from AU-007 bound IL-2 (A/IL-2) driven T effector cell expansion in vivo, converting a Treg-mediated autoinhibitory loop into an immune stimulating loop. A/IL-2 is expected to prolong the 85-minute T1/2 of IL-2, allowing endogenous IL-2 (as A/IL-2) or low dose aldesleukin to initiate an anti-tumor response.

Dozens of engineered IL-2 therapies are still being investigated in clinical trials. Based on the results presented here it is suspected that therapeutic approaches that are based on external administrations of IL-2 will face the same limitation driven by the uncontrolled endogenously secreted IL-2. AU-007, which is also in clinical development, may remedy this by redirecting endogenous IL-2 selectively to Teffs. As shown here, AU-007, eliminates the negative feedback loop that undermines the effect of modified exogenous IL-2, and may even replace it with a positive feedback loop that reinforces the expansion of Teffs by IL-2. This control of the activity of endogenously secreted IL-2 has the potential to modulate the immune response in a more precise and targeted manner to improve treatment outcomes. Since AU-007 promotes the expansion of Teffs and NK cells, it is expected to show efficacy as a monotherapy and, it is also hypothesized that combining it with either immune checkpoints inhibitors (ICI) like anti-PD-1 or with ADCC agents (antibody-dependent cell-mediated cytotoxicity) like anti-Her2 or anti-PD-L1 will result in increased efficacy. Additionally, AU-007 may potentially also be used in combination with T-cells and NK cells therapies, such as; adoptive transfer, CAR-Ts, and engineered NK cells to improve the expansion and survival of these cells.

In conclusion, the finding of this study proposes a possible explanation for the recent setbacks of modified IL-2 compounds in immune-oncology. It is suggested that improving IL-2-based therapy must include a way to control the activity of endogenous IL-2. Epitope specific IL-2 antibodies like AU-007 are great examples of such a mechanism.

In conclusion, IL-2-based therapy can lead to significant responses in some cancer patients, but it is associated with significant toxicity. Modified IL-2 immune cytokines showed a cooperative yet limited anti-tumor effect, and recently suffered from setbacks in clinical trials. It is proposed here that exogenous administration of IL-2 cytokines is inherently undermined by endogenously secreted IL-2. Immune activation by IL-2 is under tight negative feedback regulation through the IL-2-induced IL-2 release mechanism. While exogenous IL-2-based therapies cannot prevent Tregs expansion that is driven by the endogenous IL-2, controlling endogenous IL-2 by AU-007 enhances immune stimulation and cancer clearance.

### Example 8

### In Vitro and In Vivo Data for Anit-IL2 Antibodies Including BDG17.069 (AU-007)

Objective: To further investigate the functionality of BDG17.069 (AU-007) in vitro and in vivo.

### Methods:

HEK-293 cells expressing the IL-2 dimer receptor are incubated with IL-2 (red) or IL2+ AU-007 (17.069) (blue) or a control antibody with known dimer inhibition properties (green). The read out is production of secreted embryonic alkaline phosphatase (SEAP) after stimulation.AU-007 (17.069) fully preserves human IL-2 binding and functional activity to the dimeric receptor composed of CD122 and CD132 subunits. As such, AU-007 does not affect functional EC50 using CD122/CD132 dimer expressing HEK-293 cells (surrogate for effector cell population)

pSTAT5 IC50 Assay Over Naive hPBMCs: Phosphorylated STAT5 Levels of Human Immune Cell Subsets Responding to Titrated Ab

Total naïve hPBMC culture were incubated with hIL-2 and with increasing doses of AU-007 or isotype control Ab for 15 min. Immune cells subpopulations were analyzed by flow cytometry (assay was done in 3 biological repeats using 3 different blood donor) (Blue is hIL-2+ AU-007; Black is hIL-2⁺ isotype control antibody; gating was defined using fluorescence minus one (FMO) controls.)

### In Vivo Cell Analysis

C57BL/6 healthy mice were administered a single intraperitoneal (IP) injection per day of increasing concentrations of AU-007/hIL-2 complex, on four consecutive days. On day five, splenocytes were then isolated and immune cell populations were analyzed using flow cytometry. AU-007 demonstrated in vivo potent immune stimulating effects in a dose depended manner, with no observed effect on Tregs.

To examine AU-007 pharmacodynamic effect in mice spleens, C57BL/6 healthy mice were administered once with 25 ug AU-007 complexed with 1.25 ug hIL-2. 0-72 h post treatment, splenocytes were isolated and immune cells populations were analyzed using flow cytometry.

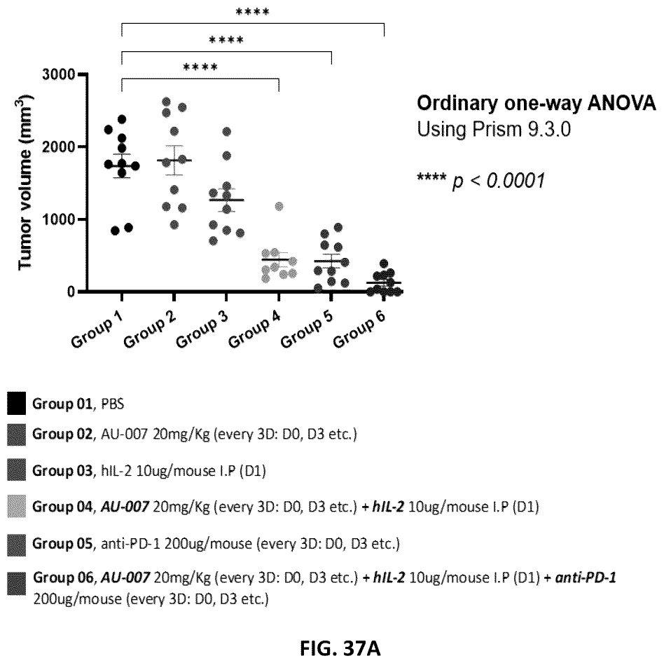

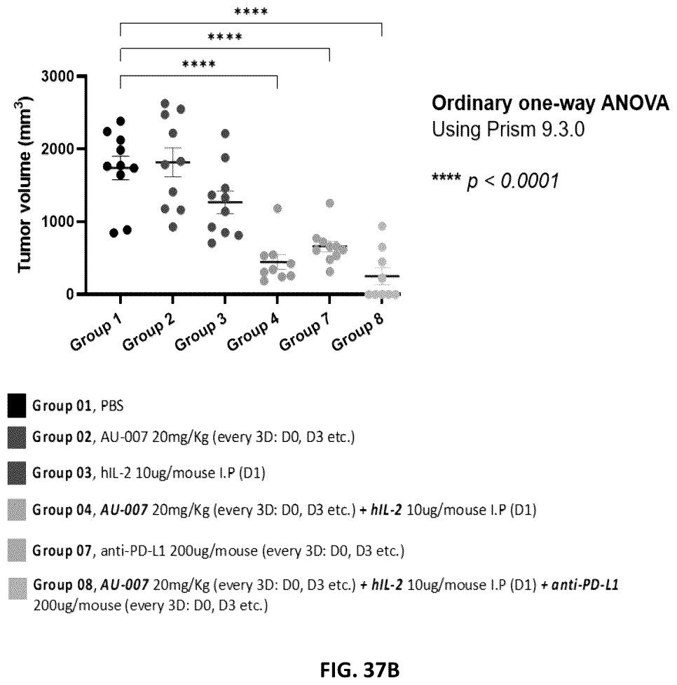

MC38 colon cancer model (syngeneic colon cancer model in C57BL/6 mice) AU-007 show inhibition of tumor growth with synergistic effect when combined with immune check point inhibitors (PD-1 or PD-L1) in colorectal cancer model (MC38). W.T. C57BL/6 mice, were inoculated with MC38 colorectal tumor cells. When tumors average volume reached ˜79 mm3 volume (day 0), mice were randomized to indicated experimental groups (n=10 per group). FIGS. 37A and 37B present tumor growth progression of experimental groups treated with: PBS (Group 1, black); AU-007 alone given every three days from day 0 (Group 2, red); single immune kick start with low dose IL-2 at day 1 (Group 3, green); 20 mg/kg of AU-007 alone given every three days from day 0 or combined with a single boost of 10 ug/mouse of human IL-2 at day 1 (Group 4, orange); or with 200 ug/mouse of anti-mouse-PD-1 antibody given every three days (Group 5, purple); AU-007 and anti-mouse PD-1 given every 3 days from day 0 with a single boost of low dose IL-2 at day 1 (Group 6, blue); or with 200 ug/mouse of anti-mouse-PD-L1 antibody given every three days (Group 7, gray); AU-007 and anti-mouse PD-L1 given every 3 days from day 0 with a single boost of low dose IL-2 at day 1 (Group 8, light green); AU-007 precomplexed with hIL-2 given with a dose of 400 ug Ab/1 ug hIL-2 at days 0-3 followed with a dose of 400 ug Ab/1.25 ug hIL-2 given every 3 days from day 6 (Group9); and hIL-2 only control group given with a dose of 1 ug hIL-2 at days 0-3 followed with a dose of 1.25 ug hIL-2 given every 3 days from day 6 (Groupi0) (Data from Group 9 and 10 is not shown). Current data is being evaluated to determine if PD-L1 provides mechanistic synergy with AU-007.

Strong anti-cancer activity was observed, including complete tumor eradications in this murine model when AU-007 was dosed in combination with a single loading dose of human interleukin-2 (hIL-2) and an anti-PD-L1 surrogate of avelumab.LL2 lung cancer model (syngeneic non-small cell lung cancer model in C57BL/6 mice)

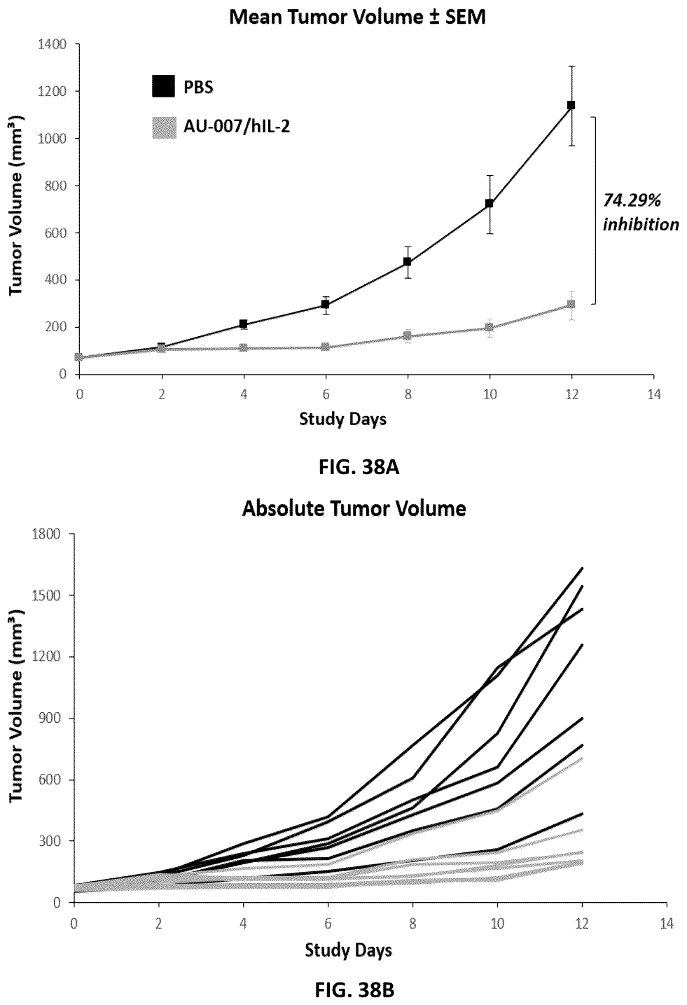

AU-007 (17.069) demonstrates tumor growth inhibition in the LL/2 Luis lung carcinoma model (FIGS. 38A and 38B). C57BL/6 healthy mice were inoculated with LL/2 lung tumor cells (day—14), at day 0 mice were randomized to experimental groups (n=10 per group) and administered I.P. with 80 ug AU-007 complexed with 1 ug of hIL-2 for four days (day 0-day 3), followed with 100 ug AU-007 complexed with 1.25 ug of hIL-2 every three days (starting from day 6). Mice were monitored daily for tumor volume.

AU-007 does not cross-react with mouse IL-2, therefore human IL-2 is co-administered. hIL-2 does bind to mouse IL-2 receptors on immune cells. Dosing regimen: (80 ug AU-007/1 ug hIL2), 200 ul/mouse, i.p., DO, D1, D2, D3, then (100 ug AU-007/1.25 ug hIL2), 200 ul/mouse, i.p., D6, D9, D12, D15, D18, D21, given as a complex.

### Results:

AU-007 (BDG17.069) does not Affect Functional EC50 Using CD122/CD132 Dimer Expressing HEK-293 Cells (Surrogate for Effector Cell Population)

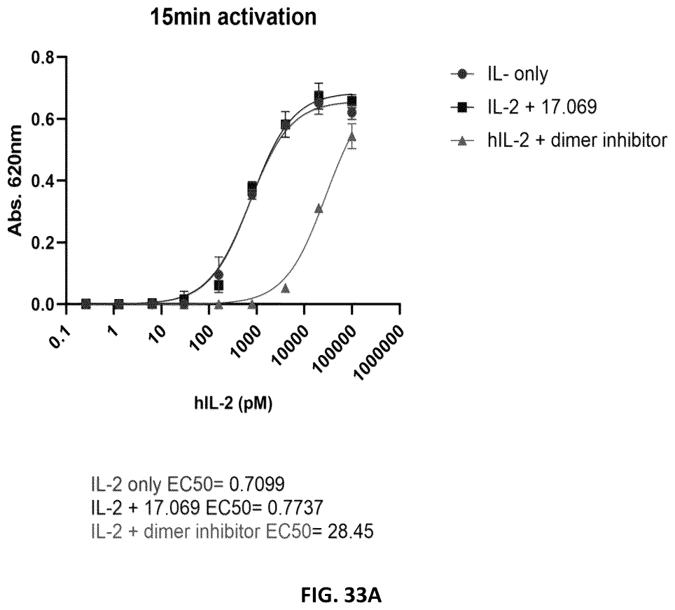

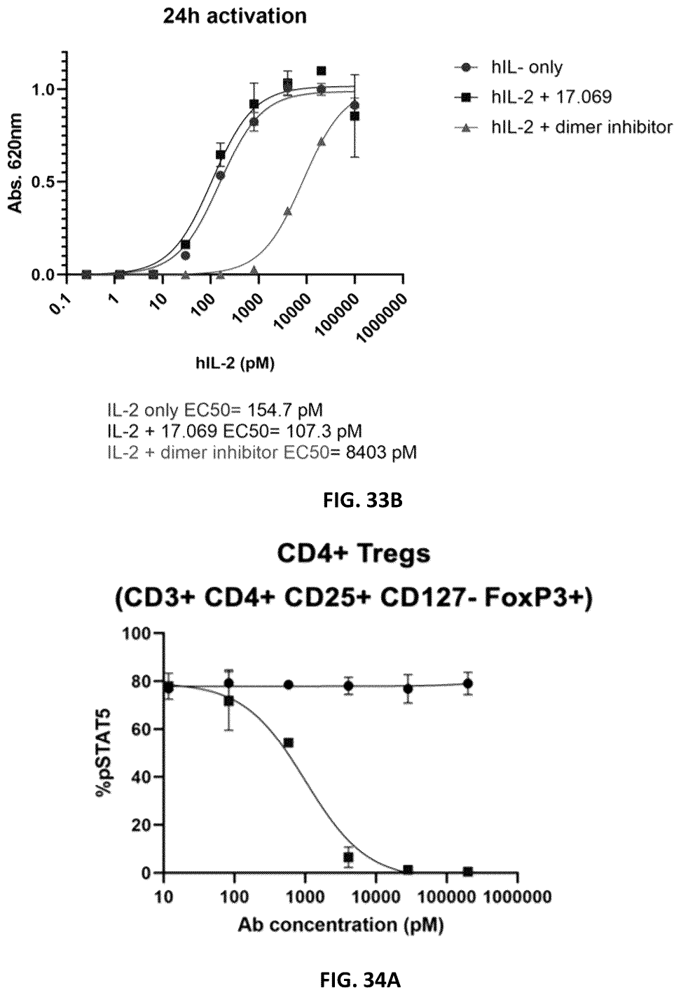

FIGS. 33A and 33B demonstrate that AU-007 and the naIL-2 do not hinder CD122/CD132-STAT5 signaling activity. A HEK239-dimer-STAT5-SEAP reporter cell line that stably expresses the human IL-2 dimer receptor (CD122/CD132) with no expression of CD25 and drives the expression of secreted embryonic alkaline phosphatase (SEAP) under a STAT5 promoter, was used to detect IL-2/IL-2-dimeric receptor signaling. FIG. 33A: CD25, CD122, and CD132 expression levels were detected using flow cytometry verifying the exclusive expression of the dimeric receptor. Dose response curves of IL-2 alone (red circles) or in the presence of 200 nM AU-007 (blue squares) or of 200 nM of an anti-IL-2 antibody that inhibits interactions with the dimeric receptor (green triangles). Dose response curve of naIL-2 (black circles). HEK239-dimmer-STAT5-SEAP reporter cells were treated with increasing concentrations of hIL-2 alone or with indicated anti-hIL-2 antibodies (FIG. 33A) or with increasing concentrations of naIL-2 (antibodies (FIG. 33B), 24 h post-treatment accumulated levels of SEAP were measured from cells media and functional EC-50 was calculated using GraphPad.

AU-007 Inhibits IL-2 Functional Affinity to Tregs while Preserving IL-2 Affinity to CD8, NK & NKTs

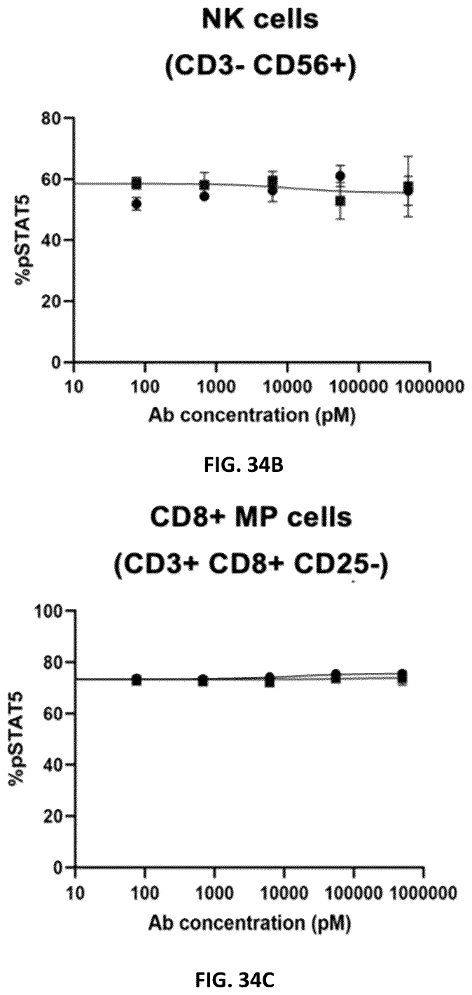

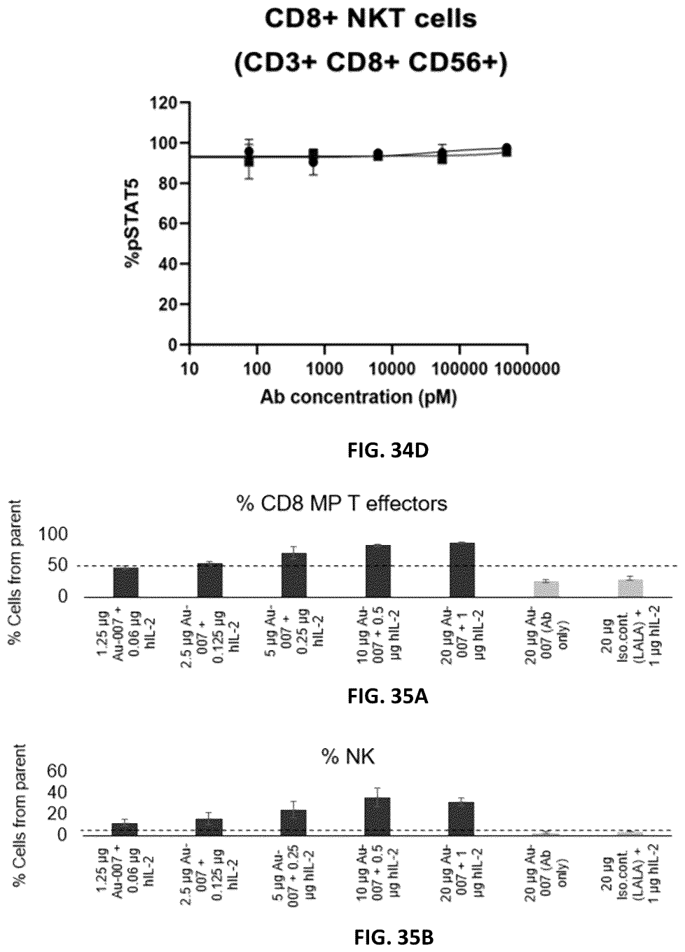

FIGS. 34A-34D show AU-007 inhibits IL-2 functional affinity to Tregs while preserving IL-2 affinity to CD8, NK & NKTs.

FIGS. 29F, 29G and 29H presented in Example 7, demonstrate that that AU-007 works with human endogenous IL-2, and the effect of AU-007 on human immune cell subsets expressing the dimeric or trimeric IL-2 receptors, wherein total hPBMCs were stimulated for 24 h with anti-CD3/anti-CD28 (stimulation only, green) or stimulated with anti-CD3/anti-CD28 in the presence of 200 nM of AU-007 mAb (red) or with 200 nM of isotype control mAb (blue). Immune cells subpopulations were analyzed by flow cytometry.

FIGS. 29J, and 29K presented in Example 7, demonstrate that AU-007 captures very low levels of endogenous IL-2 secreted from activated cells and downregulates Treg expansion. Specifically, AU-007 downregulates the suppressive markers (CD25 and FoxP3) of CD4+ regulatory Tregs.

In Vivo Administration of AU-007 Promotes Dose-Dependent Expansion and Activation of T Effector, NK and NKT Cells while Treg Population Remains Unchanged.

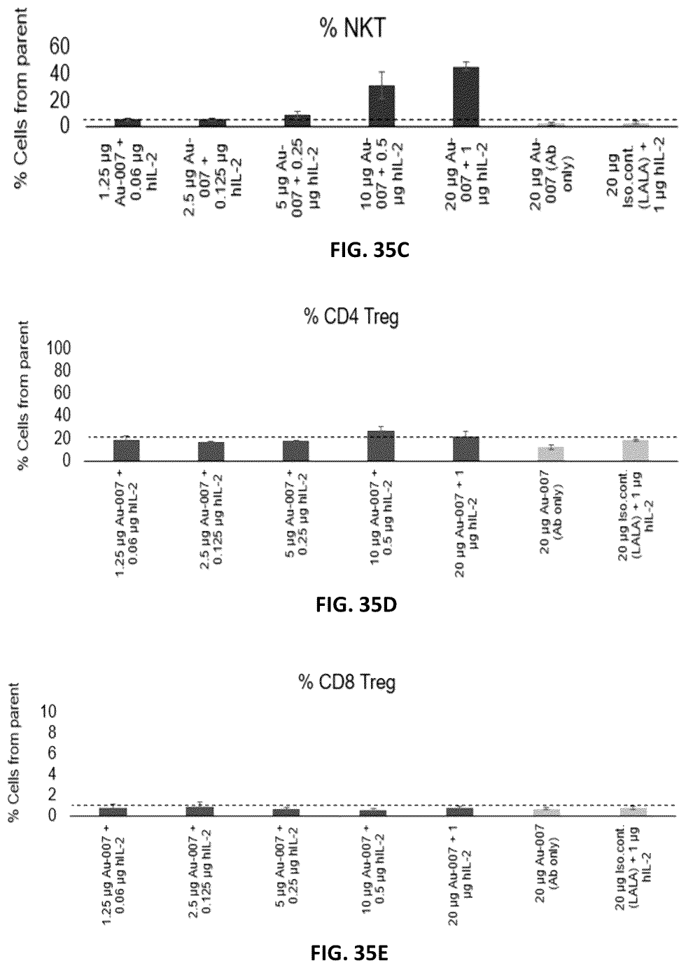

FIGS. 35A-35C show that in healthy mice, BDG17.069+ IL-2 administration promoted dose-dependent expansion and activation of Teff, NK, and NKT cells. In stark contrast, the percent Treg populations was unchanged following BDG17.069+ IL-2 administration (FIGS. 35D and 35E). To examine AU-007 pharmacodynamic effect in mice spleens, C57BL/6 healthy mice were administered once with 25 ug Uu-007 complexed with 1.25 ug hIL-2. 0-72 h post treatment splenocytes were isolated and immune cells populations were analyzed using flow cytometry.

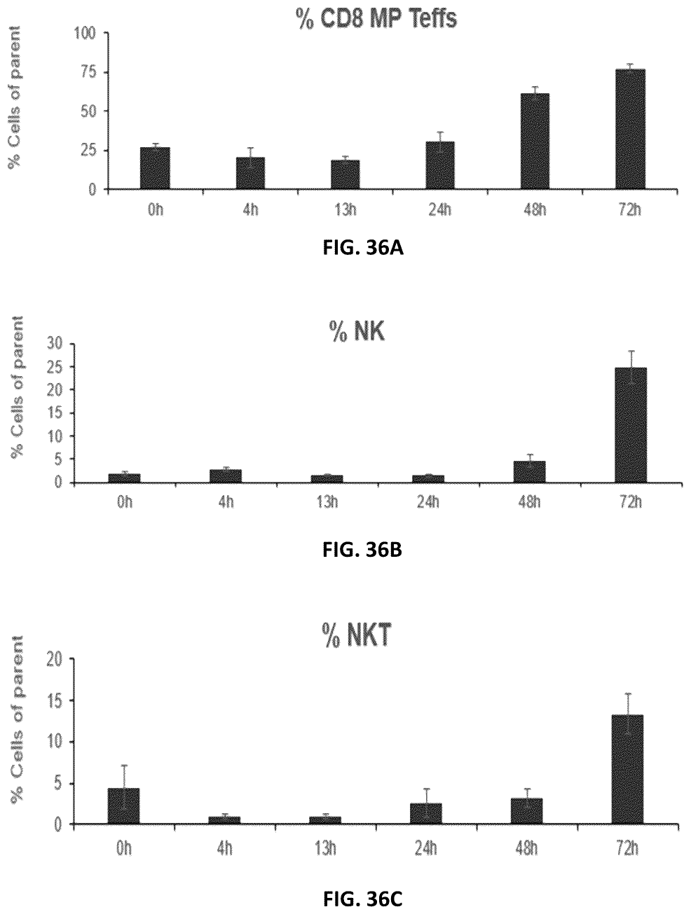

(FIGS. 36A-36C). Groups of C57Bl/6 mice were injected with antibody+IL-2 and spleens harvested at time 0, 4 h, 13 h, 24 h, 48 h, and 72 h after injection. It was observed that increases in CD8+ MP cells (FIG. 36A) could be detected as early as 24 hours after the single injection and continued to expand for the next 48 hours. Changes in NK (FIG. 36B) and NKT cells (FIG. 36C) were observed at 72 hours post injection.

### Anti-IL2 Antibody Administration Leads to Tumor Regression

Example 3 above presents data showing IL-2 mAbs disclosed herein, inhibit tumor growth in a tumor model resistant to checkpoint inhibitors (B16F10 mice melanoma model). FIGS. 37A and 37B present data showing that BDG17.069 (AU-007) induces regressions and some tumor eradications in MC38 colon cancer model in wild-type mice when combined with anti-PD-1 or with anti-PD-L1 checkpoint inhibitors. FIGS. 38A and 38B present data showing that BDG17.069 (AU-007) significantly inhibits tumor growth in LL/2 (Lung) cancer model.

### Summary and Conclusions

The in vitro data used hPBMCs to show BDG17.069 selective inhibition of Treg expansion, that BDG17.069 promotes cytotoxic cell expansion by IL-2, and that BDG17.069 captures endogenous levels of IL-2. The in vivo data used healthy mice to analyze BDG17.069 dose dependent expansion of CD8⁺, NK and NKT cells and to show that there is no effect on Tregs at all concentrations of BDG17.069. Moreover, in tumor bearing mice, BDG17.069 demonstrated significant tumor inhibition in disease models that are resistant to checkpoint inhibitors. In tumor bearing transgenic mice expressing human IL-2 BDG17.069 also demonstrated significant tumor inhibition suggesting positive feedback loop with endogenous IL-2.

### Example 9

Objective: Continued evaluation of anti-IL2 antibody clone BDG17.069 (AU-007) in subject with cancer.

### Study Design—Updates

This example provides updated data of studies described in Example 6 above and further describes studies (Clinical Phase 1/2 Trial) of the anti-IL-2 antibody in combination with IL-2 (single loading dose or co-administration). During the trial, pharmacodynamic markers in the periphery and tumor biopsies were/are being collected to investigate the activity of AU-007 or AU-007+ IL-2. Provided here is data from the ongoing studies performed evaluating anti-IL-2 antibody clone BDG17.069 (AU-007) as (1A) a monotherapy, (1B) in combination with a single loading dose of aldesleukin (Aldesleukin is the generic name for the trade drug names Proleukin® (aldesleukin)®, and (IC) with both BDG17.069 and aldesleukin given every 2 weeks (Q2w). AU-007 monotherapy evaluates doses sufficiently high to ensure enough AU-007 is available to bind all IL-2 molecules: both exogenously administered (aldesleukin) and endogenous IL-2. Aldesleukin is administered subcutaneously, at much lower doses and much less frequently than the approved IV regimen. Immunophenotyping of peripheral blood and the inflammatory cytokine interferon-gamma (IFN-γ) as well as peripheral eosinophils were examined, and preliminary results are updated here.

A further expansion of this trial will evaluate AU-007 plus low dose IL-2 (administered subcutaneously) and an anti-PD-L1 checkpoint inhibitor, e.g., avelumab.

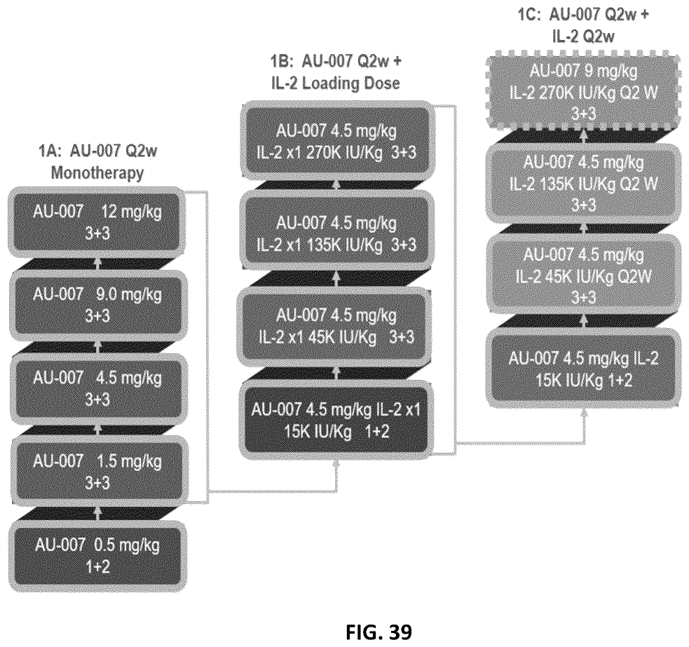

FIG. 39 presents an updated version of the Phase I Dose Escalation clinical trial scheme. (Green solid outline indicates studies in progress or completed. Green dashed outline indicates studies just begun.) Recombinant human IL-2 (aldesleukin) was, is, and will be administered subcutaneously, at much lower doses and much less frequently than the approved regimen (600,000 IU/kg every 8 hours for 14 administrations) of intravenously administered aldesleukin.

Patients enrolled in this Phase 1/2 study include but are not limited to those with unresectable locally advanced or metastatic cancer. Patient inclusion criteria specifies adults ≥18 years old with any of 20 solid tumor histologies in dose escalation. In some cases the cancer has progressed. In some cases, the subjects are not eligible for treatment with standard/approved therapies. In some cases, use of known therapies with these patients has been unsuccessful in halting the cancer.

The 20 solid tumor histologies are:
1. Urothelial cancer arising in the bladder, renal pelvis, ureter, or urethra that has progressed during or following an anti-PDx therapy and, if eligible, a platinum-containing regimen.
2. Adrenocortical carcinoma that is unresectable, locally advanced, or metastatic.
3. Clear cell renal cell carcinoma (ccRCC) progressing during or following at least 2 approved therapeutic regimens (e.g., small molecule inhibitors, anti-PDx therapy).
4. Melanoma that is either locally unresectable or metastatic:
   a. BRAF wt: progressed after receiving anti-PD-1 containing therapy with or without an anti-CTLA-4; or
   b. BRAF mut: progressed after a BRAF+MEK inhibitor.
5. Triple-negative breast cancer that is unresectable locally advanced or metastatic and that is refractory to standard 1^{st} line therapy, which may include cytotoxic chemotherapy alone and/or poly ADP ribose polymerase (PARP) inhibitors for breast cancer gene (BRCA), 1 or 2 mutations, and/or anti-PDx therapy in MSI-H/dMMR positive tumors.
6. Head and neck squamous cell carcinoma (HNSCC) that has progressed during or following treatment with an anti-PDx (unless ineligible, e.g., patients failing chemotherapy and PD-L1 combined positive score (CPS)<1) and platinum-based chemotherapy (unless ineligible for platinum chemotherapy) for metastatic or recurrent disease.
7. Gastric or gastro-esophageal cancer progressing during or after cytotoxic chemotherapy (e.g., paclitaxel, fluoropyrimidine, platinum agents) with or without trastuzumab (for HER2 overexpressing adenocarcinoma) and with or without anti PD-1 inhibitor therapy. Patients with a CPS ≥1 should have received an anti PD-1 containing regimen (unless intolerant or therapy unavailable).
8. Esophageal squamous cell carcinoma progressing during or after cytotoxic chemotherapy (e.g., paclitaxel, fluoropyrimidine, platinum agents) with an anti PD-1 therapy. Patients with a CPS ≥10 should have received an anti PD-1 containing regimen (unless intolerant or therapy unavailable).
9. Cutaneous squamous cell carcinoma (cSCC): recurrent or metastatic cSCC that is not curable by surgery or radiation.
10. Pancreatic adenocarcinoma that is unresectable locally advanced or metastatic and received at least one line of chemotherapy (e.g., FOLFIRINOX) (unless ineligible or not feasible).
11. Cholangiocarcinoma that is unresectable locally advanced or metastatic in patients who have had ≥1 line of systemic chemotherapy, unless the patient is ineligible for chemotherapy.
12. Hepato-cellular carcinoma (HCC) progressing during or following an approved therapeutic regimen (unless ineligible).
13. Colorectal cancer (CRC):
   a. K-Ras wild type: Patients who have progressed during or after, or are ineligible for, both irinotecan-based and oxaliplatin-based chemotherapy and who are relapsed or refractory to at least 1 prior systemic therapy that included an anti-epidermal growth factor receptor (EGFR) antibody, such as cetuximab or panitumumab; or
   b. K-Ras mutant: Patients who have progressed during or after, or are ineligible for, both irinotecan and oxaliplatin based chemotherapy (±bevacizumab).
14. Epithelial ovarian cancer progressing during or following at least one prior cytotoxic chemotherapeutic regimen (unless ineligible), and subsequent poly ADP ribose polymerase (PARP) inhibitor therapy in BRCA mutation positive patients (unless ineligible).
15. Cervical cancer progressing during or following first-line cytotoxic chemotherapy and second-line cytotoxic chemotherapy or anti-PDx therapy in PD-L1 positive (CPS ≥1) or MSI-H/dMMR positive tumors (unless ineligible).
16. Endometrial cancer in patients progressing on or following either cytotoxic chemotherapy (±trastuzumab) or hormone therapy, and an anti-PDx therapy in MSI-H/dMMR positive tumors.
17. Thyroid cancer (follicular or papillary histology) that is iodine refractory.
18. Non-small cell lung cancer (NSCLC) that has progressed during or following treatment with platinum-based chemotherapy and an anti-PDx therapy for unresectable locally advanced or metastatic disease. NSCLC harboring an activating EGFR mutation (excluding Exon 20 insertion mutations) or anaplastic lymphoma kinase (ALK) rearrangement must have progressed following available EGFR or ALK-targeted therapy in addition to treatment with platinum-based chemotherapy (unless ineligible for platinum therapy).
19. Merkel Cell Carcinoma: metastatic Merkel cell carcinoma that is not curable by surgery or radiation.
20. Nasopharyngeal cancer.

In addition to the 20 solid tumor histologies listed above, patient's entered into the trial may be suffering any of the solid tumors including a melanoma, a metastatic melanoma, a primary melanoma and metastatic melanoma, a renal cell carcinoma (RCC), a non-small cell lung cancer (NSCLC), a bladder cancer, a head and neck cancer, a head and neck squamous cell carcinoma (HNSCC), a nasopharyngeal carcinoma, a urothelial cancer, an adrenal cortical carcinoma, a clear cell renal cell carcinoma (ccRCC), a triple-negative breast cancer, a gastric or gastro-esophageal cancer, an esophageal squamous cell carcinoma, a cutaneous squamous cell carcinoma (cSCC), a pancreatic cancer, a pancreatic adenocarcinoma, a cholangiocarcinoma (bile duct cancer), a hepato-cellular carcinoma (HCC), a colorectal cancer (CRC), an epithelial ovarian cancer, a cervical cancer, an endometrial cancer, a thyroid cancer (follicular or papillary histology), a lung cancer, a uterine cancer, a gallbladder cancer, or a Merkel cell carcinoma, or any tumors that are microsatellite instabilities (MSI)-high tumors. In some cases, the cancer may be unresectable locally advanced or metastatic cancer.

Adverse events were graded by the Common Terminology Criteria for Adverse Events (CTCAE). Efficacy evaluation was based on PD (Pharmacodynamic) markers of immune stimulation, total IL-2 (bound to AU-007+ free IL-2) and objective responses.

Efficacy based on PD markers of immune stimulation, total IL-2 (bound to AU-007+ free IL-2), and objective response.

Tumor assessments occur at the end of each 8-week cycle.

42 patients enrolled as of Oct. 13, 2023: Arm TA: 15; Arm 1B: 12; Arm 1C: 15.

| Patient Demographics | | Tumor Histologies Evaluated in the Trial | |
|---|---|---|---|
| Patient Characteristics n = 42 | | Cancer Diagnosis (n, %) | |
| Mean age, years (range) | 63 (44- [symbol] ) | Melanoma (includes 1 Uveal/1 Acral) | 10 (23.8) |
| Gender, n (%) | | Pancreatic cancer | 7 (16.7) |
| Male | 23 (55) | Head and neck [symbol] quamous cell carcinoma | 5 (11.9) |
| Female | 19 (45) | Renal cell carcinoma | 5 (11.9) |
| Race, n (%) | | Colorectal cancer | 4 (9.5) |
| White | 34 (8 [symbol] .9) | Non-small cell lung cancer | 3 (7.1) |
| Asian | 3 (7.1) | Cutaneous [symbol] quamous carcinoma | 3 (7.1) |
| Black | 2 (4.8) | Bladder cancer | 2 (4.0) |
| American Indian/Alaska native | 1 (2.4) | Other | 3 (7.1) |
| Other | 1 (2.4) | | |
| Unknown | 1 (2.4) | | |
| ECOG performance status, n (%) | | | |
| 0 | 22 (52) | | |
| 1 | 20 (4 [symbol] ) | | |
| Mean number of prior therapies, n (range) | 3 [symbol] -8) | | |

[symbol]

indicates data missing or illegible when filed

### Methods:

Briefly, peripheral blood samples were taken prior to dosing on Day 1 and following dosing at 4 hours, and on Days 2, 3, 15, 29, 43 of cycle 1 and pre-dose/Day 29 on all other cycles. Dosing with AU-007 was done via intravenous dosing by weight-based dosing (mg/kg). Proleukin® (aldesleukin) was dosed subcutaneously by weight-based dose (U/kg). Whole blood was stained for CD4+ T cells, CD8+ T cells, CD4+ Tregs, NK cells, B cells, and monocytes. Samples were analyzed by flow cytometry using TruCount™ for absolute cell counts. Changes from baseline values were determined off absolute counts. In addition, differential hematology counts were taken on Days 1, 15, and 29 of all cycles (or as needed per treating physician's decision) for safety evaluation and to examine eosinophil levels (eosinophils express the IL-2 trimeric receptor). Serum was taken prior to dosing on Day 1 and at 2 hours, 6 hours, and Days 2, 3, 15 pre/post, and 6 hours, 29 pre/post, 43 pre/post, and subsequent Day 1 of each cycle at pre/post. Samples were analyzed for INF-γ (LOQ 31 fg/ml), IL-2 (LOQ 31 fg/ml), and sCD25 (10 fg/ml) using the ECL Mesoscale Dynamics platform.

FIG. 39 graphically presents the administration and dosage schemes of BDG17.069 (AU-007)Q2w Monotherapy (left-TA dosages of 0.5 mg/kg, 1.5 mg/kg, 4.5 mg/kg, 9.0 mg/kg, and 12 mg/kg), BDG17.069 (AU-007)+a single IL-2 loading dose (center 1B dosages of 4.5 mg/kg AU-007+15K IU/Kg IL-2, 4.5 mg/kg AU-007+45K IU/Kg IL-2, 4.5 mg/kg AU-007+135K IU/Kg IL-2, and 4.5 mg/kg AU-007+270K IU/Kg IL-2), and combination therapy BDG17.069 (AU-007)+IL-2 (right 1C dosages of 4.5 mg/kg AU-007+15K IU/Kg IL-2, 4.5 mg/kg AU-007+45K IU/Kg IL-2, 4.5 mg/kg AU-007+135K IU/Kg IL-2, and 9.0 mg/kg AU-007+270K IU/Kg IL-2).

Recombinant human IL-2 (aldesleukin) was administered subcutaneously. Note that the doses and frequency are much lower and less frequent than the approved regimen of intravenously administered aldesleukin.

As described in Example 6, in FIG. 39, the terms “3+3” and “1+2” refer to the size of the cohort at a given dose level. It means in the case of 3+3 that the first 3 enrolled patients were or will be administered BDG 17.069 at that dose level. If no dose-limiting toxicities (DLTs) are seen, then administration may escalate to the next highest dose level. However, if any of the first 3 patients do have a dose-limiting toxicity that is drug-related, then an additional three more patients need to receive treatment at that same dose level to see if they have DLTs before escalating. 1+2 follows the same principle—start with one patient, if DLTs are observed, add two more, or if no DLTs are observed in the first single patient, then dosages can be escalated. The trial continues to progress and the fourth dose escalation Cohort of Arm 1C has opened wherein AU-007 is being evaluated in the range of 4.5-12 mg/kg and with IL-2 at 270×10³ IU/kg. Future dose escalations of IL-2 up to about 500×10³ IU/kg may be evaluated.

The DLT evaluation period is the first 28 days of the 1^{st} cycle. Tumor assessment by computed tomography scan occurs with each 8-week cycle. The AU-007 and aldesleukin dose and schedule for Phase 2 expansion, will be based on safety, efficacy, pharmacokinetics (PK), and pharmacodynamics (PD).

Peripheral blood samples were taken at pre dose followed at 4 hours, and on days 2, 3, 15, 29, 43, of cycle 1 and pre-dose/day 29 on all other cycles. Whole blood was stained for CD4+ T cells, CD8+ T cells, CD4+ Tregs, NK cells, B cells, and monocytes . . . . In addition, differential hematology counts were taken on days 1, 15 and 29 of all cycles (or ad hoc as per treating physician's needs) to examine eosinophil levels. Serum was taken at pre dose and at 2 hrs, 6 hrs and days 2, 3, 15 pre/post and 6 hours, 29 pre/post, 43 pre/post, and subsequent day one of each cycle at pre/post. Samples were analyzed for INF-gamma (LOQ 31 fg/ml), IL-2 (LOQ 31 fg/ml), and sCD25 (10 fg/ml) using the ECL Mesoscale Dynamics platform.

### Results:

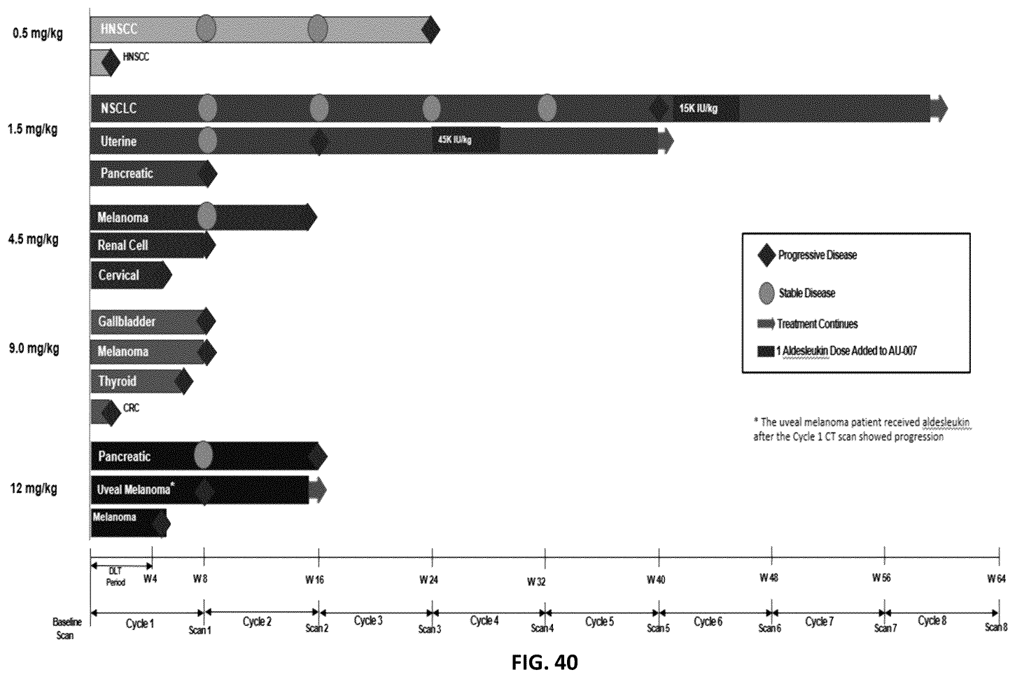

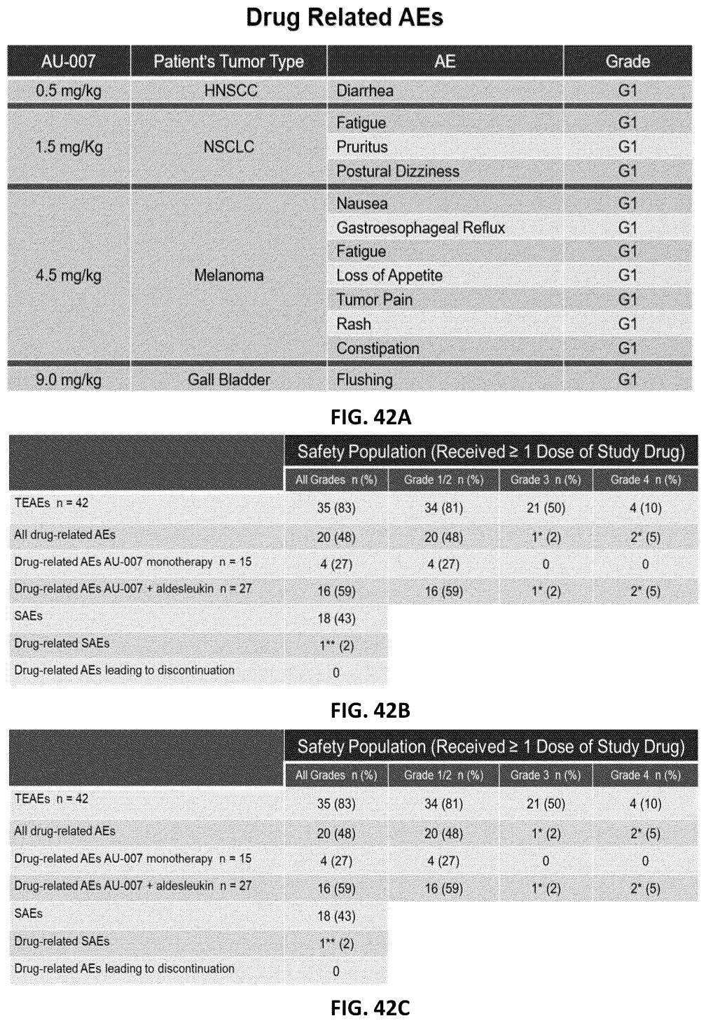

FIG. 40 presents updated data from 15 patients (as of Oct. 13, 2023) enrolled in 5 cohorts of Arm 1A (0.5 mg/kg, 1.5 mg/kg, 4.5 mg/kg, 9.0 mg/kg, and 12 mg/kg). For patients in cohorts 1-4, AU-007 was well tolerated with no DLTs and all treatment-related adverse effects were Grade 1 (FIG. 42A). For Arm 1A, a best response of stable disease was noted in several patients (FIG. 42A) and three patients continued treatment. Three (3) patients in Arm 1A received a single aldesleukin dose following progression: NSCLC, uterine leiomyosarcoma, and uveal melanoma. The NSCLC and leiomyosarcoma patients had tumor scans following aldesleukin administration and are evaluated with the AU-007+ aldesleukin cohorts for efficacy. Other patients were discontinued with either objective progression or clinical progression.

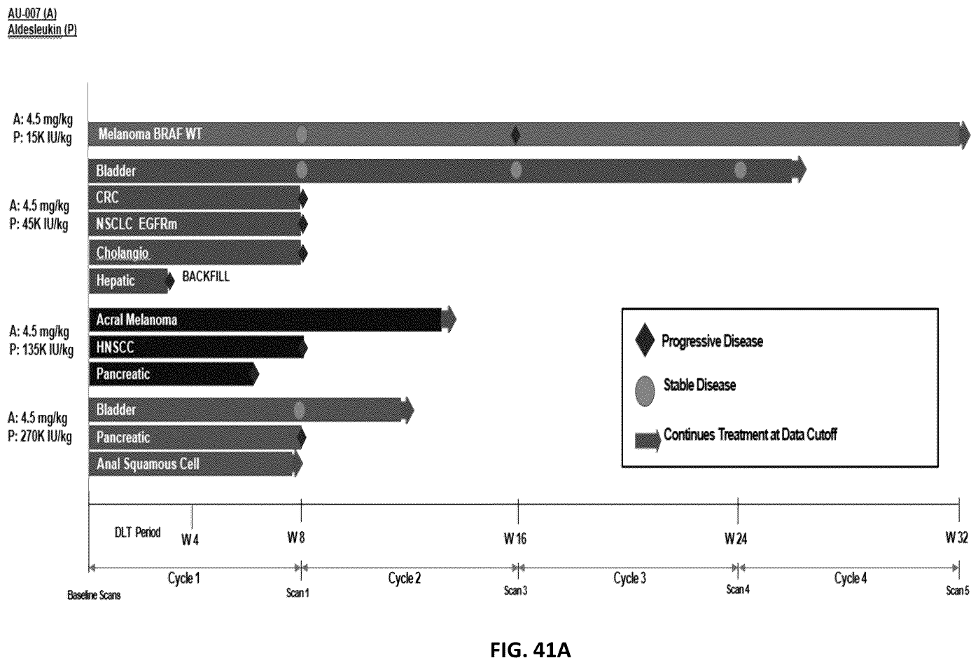

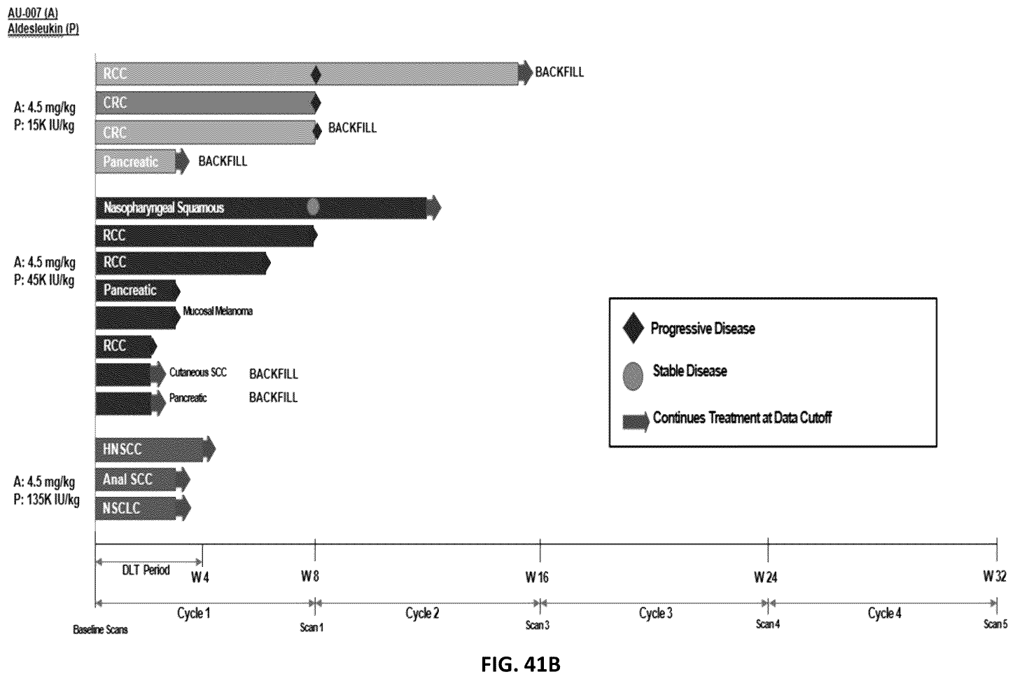

FIG. 41A presents the current data from 12 patients enrolled in the 4 cohorts of Arm 1B (4.5 mg/kg AU-007+15K IU/kg IL-2 or 45K IU/kg IL-2 or 135 IU/kg IL-2 or 270 IU/kg IL-2). FIG. 41B presents the current data from 15 patients enrolled in the first 3 cohorts of Arm 1C (4.5 mg/kg AU-007+15K IU/kg IL-2 or 45K IU/kg IL-2 or 135 IU/kg IL-2). 27 total patients enrolled into Arms 1B and 1C: Q2W AU-007+1 loading dose of aldesleukin and Q2W AU-007⁺ Q2W aldesleukin, respectively.

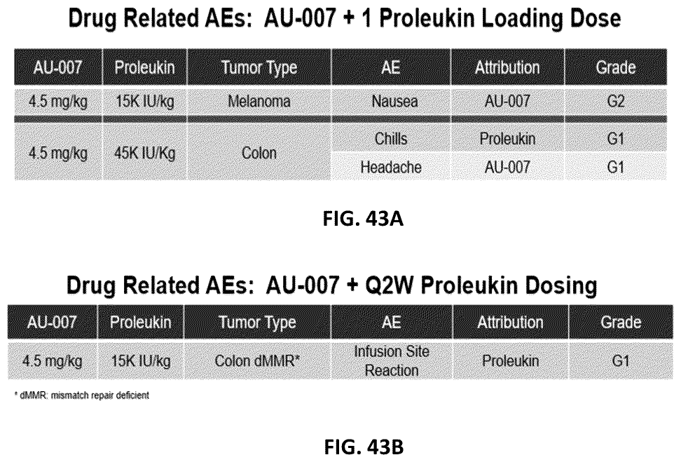

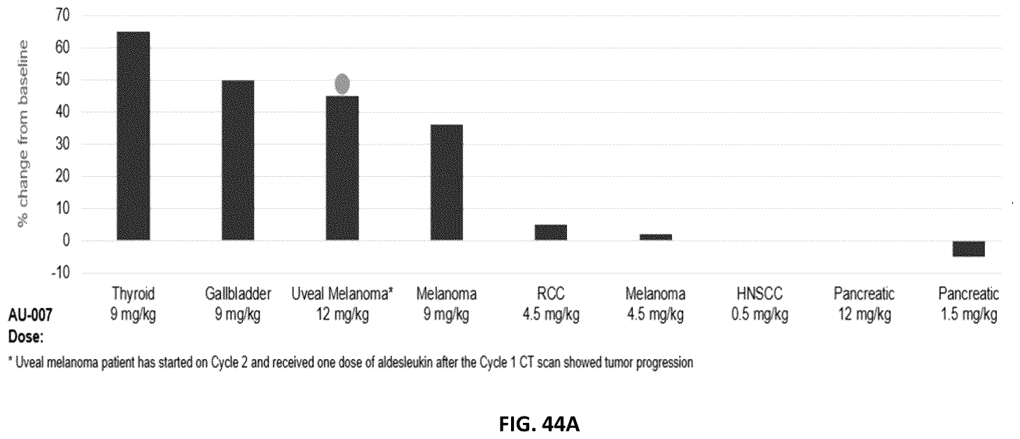

For the patients in the first 2 cohorts of Arm 1B and 2 cohorts of Arm 1C, AU-007 was well tolerated with no DLTs and all but one treatment-related adverse effects were Grade 1 (FIGS. 443A and 43B). All five patients continued treatment in Arm 1B, the two patients continued treatment in Arm 1C, and additional patients joined Arm 1B and Arm 1C; additional patients are targeted to join Arm 1C and are in screening.

A summary of all adverse effects for Arms 1A, 1B, and 1C is presented in FIG. 42B and FIG. 42C. All drug-related Adverse Events (AEs) were Grade 1 or 2 except for 3 patients receiving AU-007+ aldesleukin with transient (3-7 days) Grade 3 or 4 lymphopenia that were not associated with adverse outcomes. Transient lymphopenia is a known effect of IL-2 treatment. No patients discontinued for a drug-related AE; no dose-limiting toxicities (DLTs) were observed.

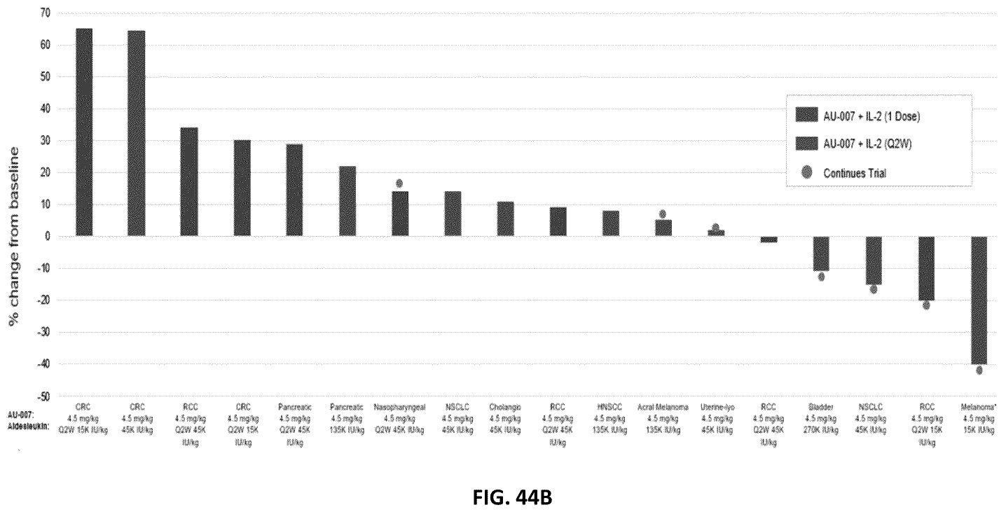

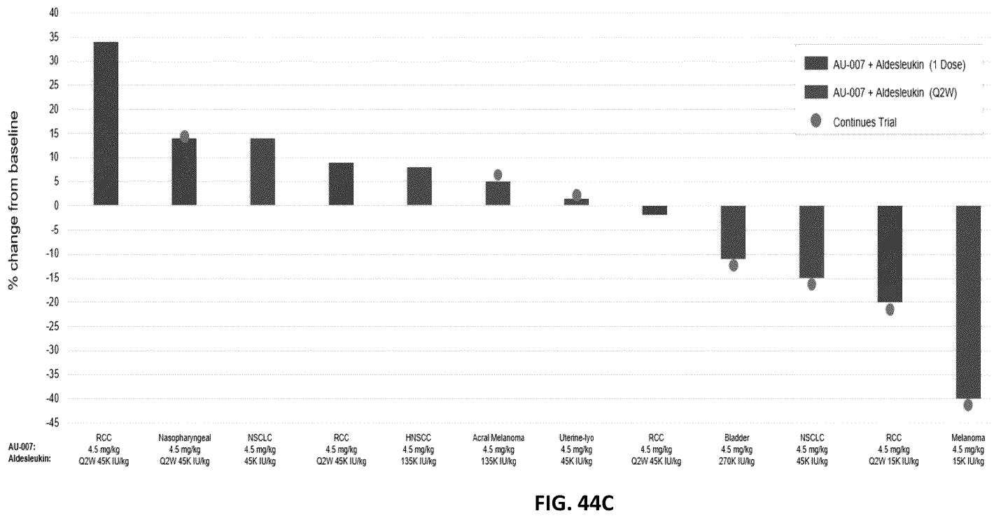

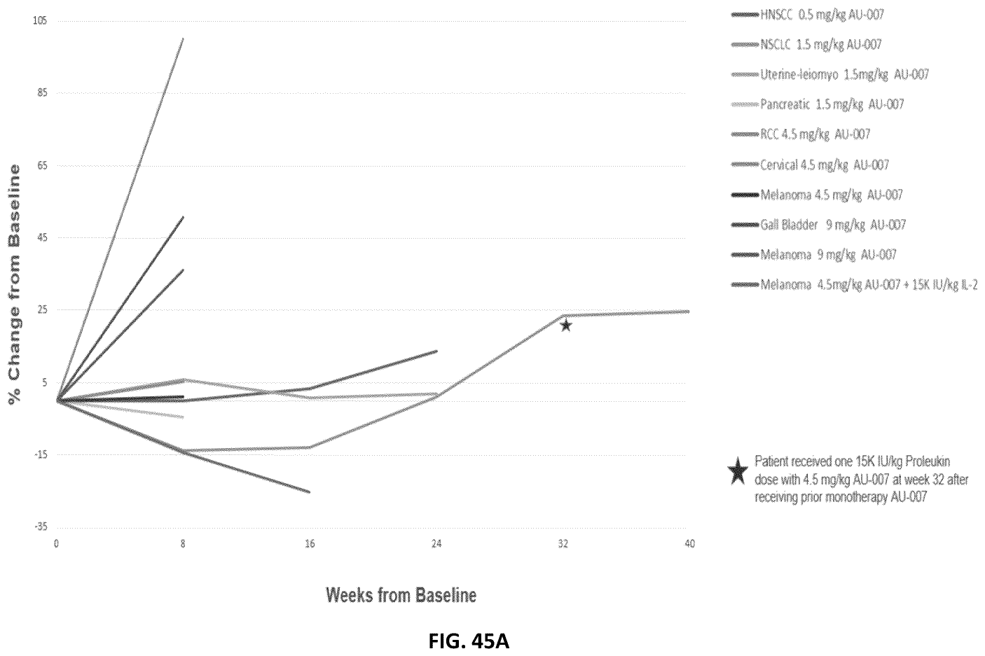

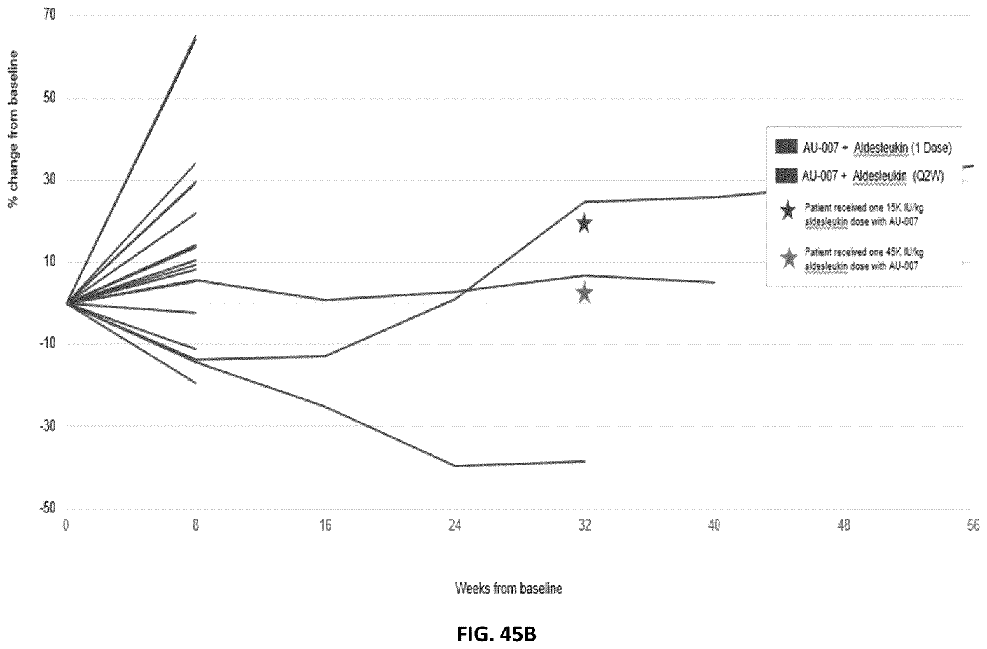

FIGS. 44A-44C and FIGS. 45A and 45B present blots of percent change of tumor burden vs. baseline in view of treatment regime and tumor type (FIGS. 44A-44C) or over time (FIGS. 45A-445B). The clinical details indicate that two patients (Patient AU01-0009—NSCLC with No Response to Chemo+anti-PD-L1 (dosage AU-007 1.5 mg/kg; See below) and Patient AU06-0014—Melanoma with No Response to PD-1⁺ CTLA-4 (dosage AU-007 4.5 mg/kg+15K IU/kg)) who were unresponsive to checkpoint inhibitor therapy, are not only continuing in the present trial but their tumors have shown a negative percent change from baseline (reduced size) Two patients (NSCLC and uterine leiomyosarcoma) began therapy on AU-007 monotherapy (Arm 1A) and received one aldesleukin dose, 15K and 45K IU/kg respectively, after having progressed (stars on the graph; FIG. 45B). The velocity of tumor growth decreased in both patients after receiving aldesleukin with AU-007.

At this point in early development (Oct. 13, 2023), the greatest anti-tumor activity is observed with AU-007 in combination with aldesleukin, in patients with tumors known to be sensitive to immune-modulating drugs. This subset of patients is shown in the waterfall plot where the G.I. cancers are excluded (FIG. 44C). A best response of stable disease was observed in 9 of 33 (27%) evaluable for response. Specifically, 1 patient with bladder cancer had Non-target lesions (NTL) disease only and has stable disease (SD) through 3 cycles, with tumor reduction observed after Cycle 1. In a melanoma patient, there was a 40% decrease in target tumors that had progressed/not responded when treated with anti-CTLA-4⁺ anti-PD-1 therapy; brain metastasis noted after Cycle 2 and treated with radiation, and the patient continues on treatment.

Patient AU01-0009: NSCLC with No Response to Chemo+Anti-PD-L1

The patient Cancer History: 83 year old man diagnosed with stage 3 squamous NSCLC in 2020. In January 2021 he received Chemo+anti-PD-L1. In June 2021 progression of the disease was observed in the opposite lung, wherein a watch and wait strategy was taken. By July 2022 further progression in lungs was observed. This subject received was enrolled in Arm 1A and received his first dose of AU-007 (1.5 mg/kg) Aug. 15, 2022. Table 15 presents clinical details and tumor size measurements.

TABLE 15

| Target | Pre- | Base- | 8 Week | 16 Week | 24 Week | 32 Week | 40 Week |
|---|---|---|---|---|---|---|---|
| Lesions | treatment | line | Scan | Scan | Scan | Scan* | Scan |
| (mm) | Jul. 14, 2022 | Aug. 9, 2022 | Oct. 7, 2022 | Dec. 5, 2022 | Jan. 30, 2023 | Apr. 5, 2023 | Jun. 5, 2023 |
| LUL Lung | 47 | 58 | 53 | 55 | 74 | 93 | 95 |
| L Adrenal | Not | 20 | 17 | 17 | 13 | 16 | 16 |
| | mentioned | | | | | | |
| R Adrenal | Not | 23 | 17 | 16 | 15 | 17 | 16 |
| | mentioned | | | | | | |
| Sum | 47 | 101 | 87 | 88 | 102 | 126 | 127 |

Progressive disease was noted due to the earlier positive trend and in consultation with medical staff, this patient received: I aldesleukin dose (15K IU/kg) at Week 32 and AU-007 dose was increased to 4.5 mg/kg.

Patient AU06-0014: Melanoma with No Response to Anti-PD-1/CTLA4; Significant Shrinkage in Two Lesions

The patient Cancer History: 2020, diagnosed with stage 3C melanoma: BRAF/NRAS WT

On July 2020, L arm lesion excised with lymph node biopsy. Between August 2020-April 2021 9 cycles adjuvant Nivolumab (anti-PD-1) were administered. On April 2021 recurrence L axillary node was noted and on May 2021 the axillary LN was excised. Between October-November 2021 the subject received adjuvant radiation and between September-November 2022 the patient received 4 Cycles Ipilimumab (anti-CTLA4)+Nivolumab (anti-PD-1). December 2022 progressive disease (PD) in the liver.

This patient was enrolled in Arm 1B and received a first dose of AU-007 (4.5 mg/kg)+Proleukin® (aldesleukin) (15K IU/kg) on Feb. 6, 2023. Table 16 presents clinical details and tumor size measurements.

TABLE 16

| | Baseline | 8 Week Scan | 16 Week Scan | 24 Week Scan |
|---|---|---|---|---|
| Target Lesions | Jan. 17, 2023 | Apr. 3, 2023 | May 29, 2023 | Jul. 24, 2023 |
| Liver Seg VII | 34 | 19 | 13 | 8 |
| Liver Seg IVA-B | 32 | 18 | 16 | 11 |
| LN - Portacaval | 17 | 34 | 33 | 31 |
| Sum | 83 | 71 | 62 | 50 |
| | | −14% | −25% | −40% |

The increase in lymph node (LN) may be driven by T cell infiltration, while a 14% total decrease in target lesions was observed.

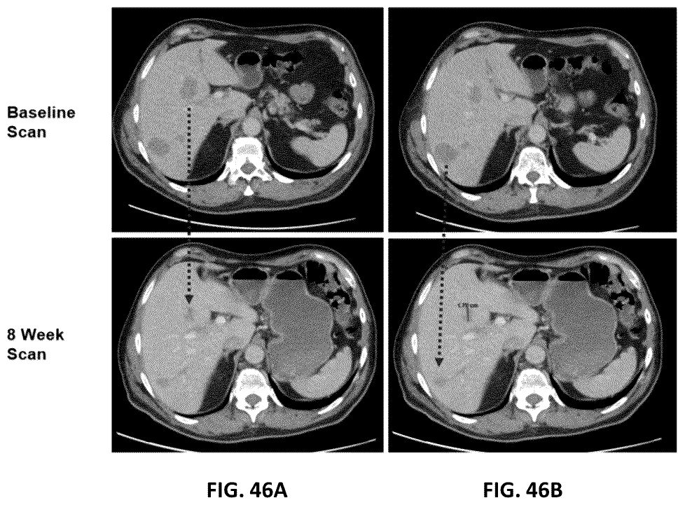

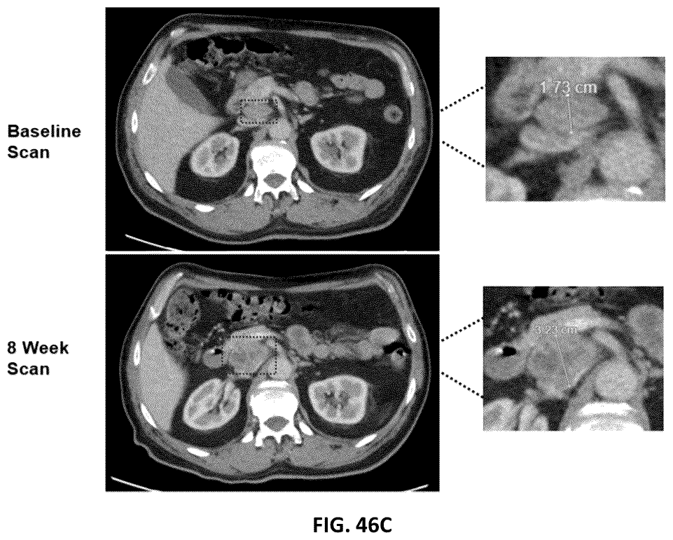

FIGS. 46A, 46B, and 46C present the Baseline and 8-week scans. Portacaval lymph node: initial growth (with development of necrotic center) followed by stabilization may represent lymphocytic tumor infiltration (pseudo progression). Considering the lack of response to the former therapies, the scans showing 40% shrinkage in the target lesions of this melanoma patient demonstrate a significant improvement and therapeutic outcome. A new brain lesion was noted at the end of the 2nd cycle (16 weeks) and treated with radiation therapy. The patient continues on treatment.

Renal Cell Carcinoma (RCC) Patient, 68-Year-Old Man, Who Progressed on Prior Anti-PD-1 Treatment with Objective Progression in Mediastinal and Hilar LNs, June 2022.

Treatment of this RCC patient was initiated July 2023 at AU-007 (4.5 mg/kg)+15K IU/kg Q2W aldesleukin doses. Rapid shrinkage in the lymph node (LN) lesions was observed on initial scan (8 weeks). Comparison of baseline and 8-week scans showed 20% shrinkage in the Target Lesions (data not shown). The primary renal cancer remains in situ and was stable with no change in dimensions on the 8-week scan (not shown). A new cervical bone metastasis was noted at the end of the 1st cycle and will be surgically excised. The patient will continue on treatment following surgery.

### AU007 Pharmacokinetic (PK) Data Demonstrates IgG1 Therapeutic Characteristics

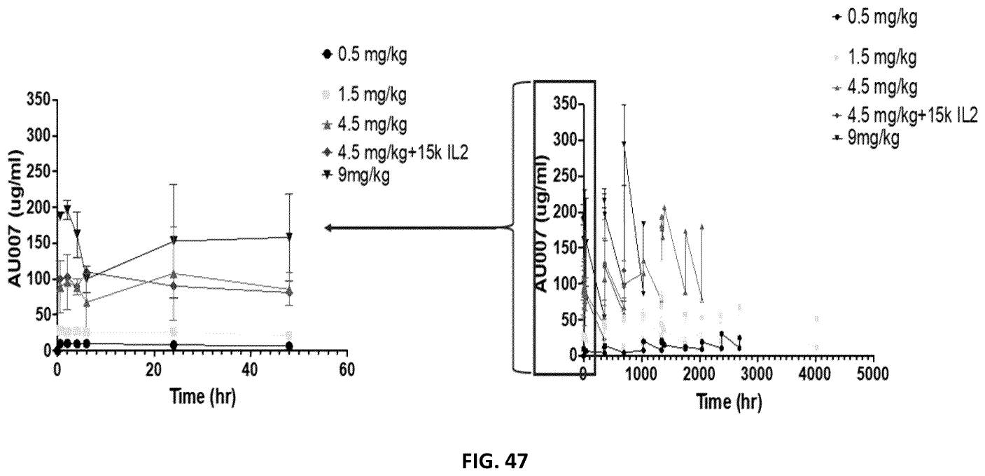

FIG. 47 presents PK data showing the pharmacokinetic data of patients based on dosage and regime. This data continues to demonstrate AU-007 concentration is dose proportionality and has PK characteristics similar to a standard IgG1 therapeutic human monoclonal antibody. Thus, AU-007 demonstrates low potential for immunogenicity.

Table 17 presents Cmax and step close to that predicted.

TABLE 17

| | est Cmax | | Actual | Calculated |
|---|---|---|---|---|
| Dose | (70 kg) | Step | Cmax | Step |
| 0.5 | 14 ug/ml | | 9.9+/−3 | |
| 1.5 | 42 ug/ml | 3 | 28.7+/−4 | 2.89 |
| 4.5 | 126 ug/ml | 3 | 91.9+/−29 | 3.2 |
| 9 | 252 ug/ml | 2 | 187.8+/−6 | 2 |

Table 18 presents data showing AU-007 PK and IL-2 Coverage (For Binding and Redirecting to Dimeric Receptors on Effector Cells)

TABLE 18

| | | | | Coverage of Highest |
|---|---|---|---|---|
| | | | | IL-2 Dose |
| AU-007 | | Serum | Serum IL-2 | (Proleukin ® |
| Dose | | AU-007 | Coverage | (aldesleukin) |
| mg/kg | Time Point | ug/ml | pM | 270 IU/kg) |
| 0.5 | Initial Peak | 12 | 82192 | 205 X |
| | Initial Trough | 4 | 27397 | 68 X |
| | Steady State | 20 | 136986 | 342 X |
| 1.5 | Initial Peak | 29 | 198630 | 496 X |
| | Initial Trough | 12 | 82192 | 205 X |
| | Steady State | 55 | 376712 | 941 X |
| 4.5 | Initial Peak | 110 | 753425 | 1883 X |
| | 50 hours | 90 | 616438 | 1541 X |
| | Steady State | | Data not yet available | Data not yet available |
| 9 | Initial Peak | 200 | 1369863 | 3424 X |
| | 50 hours | 150 | 1027397 | 2568 X |
| | Steady State | | Data not yet available | Data not yet available |
| 12 | Initial Peak | | Data not yet available | Data not yet available |
| | 50 hours | | Data not yet available | Data not yet available |
| | Steady State | | Data not yet available | Data not yet available |

### AU-007 PD Continues to Demonstrate Decrease in Tregs

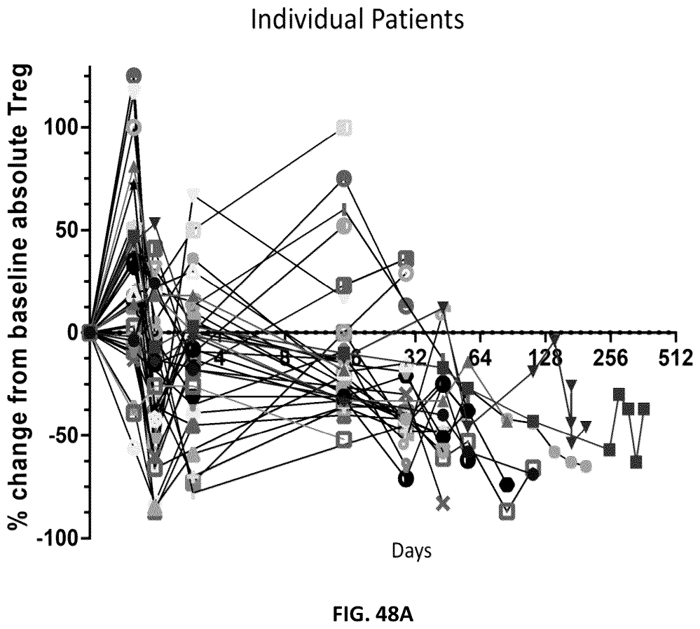

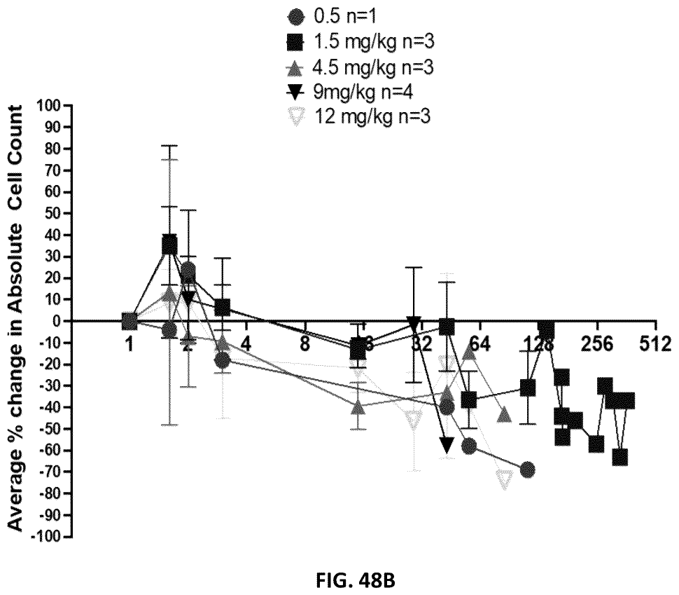

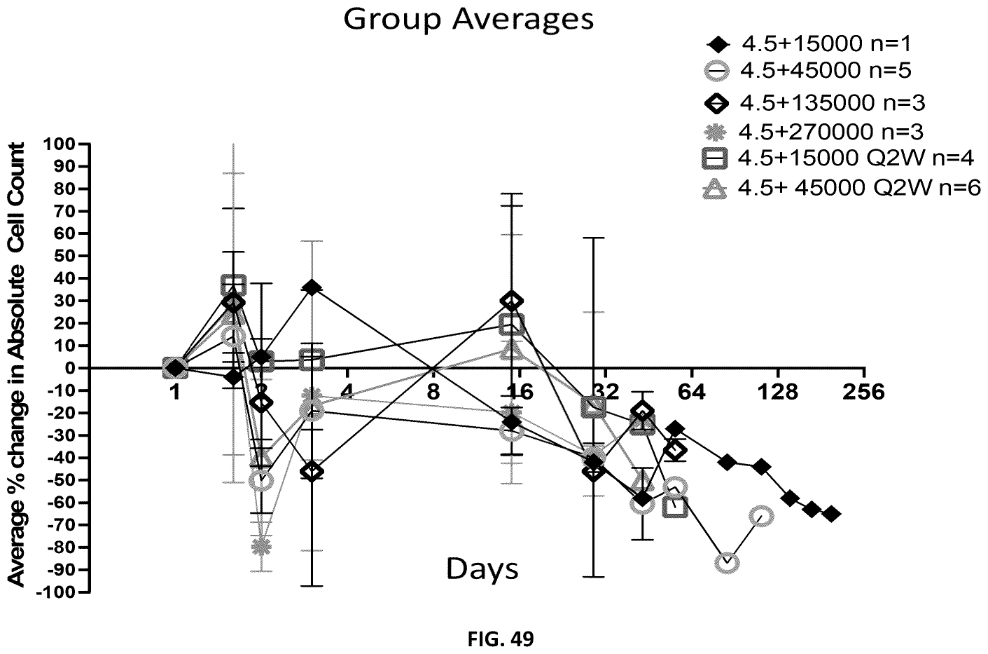

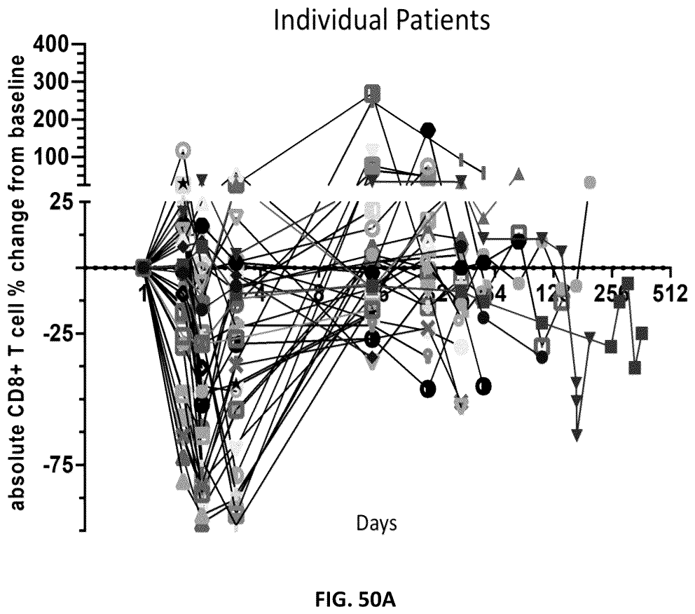

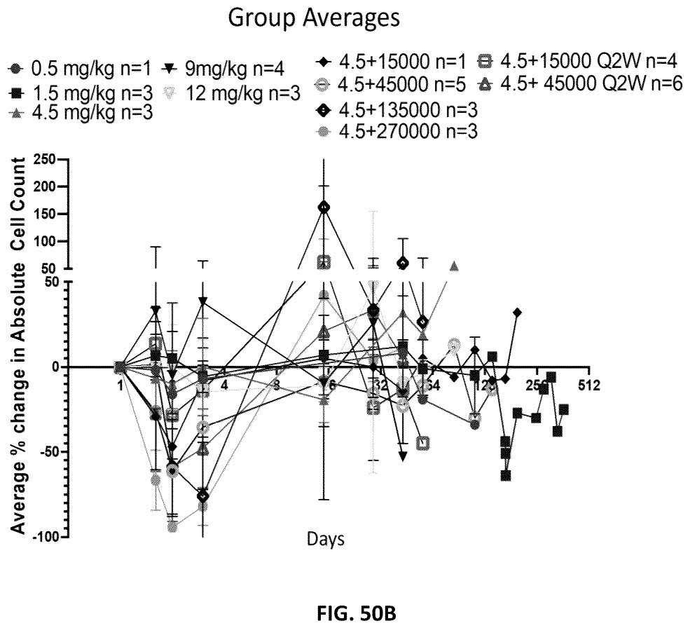

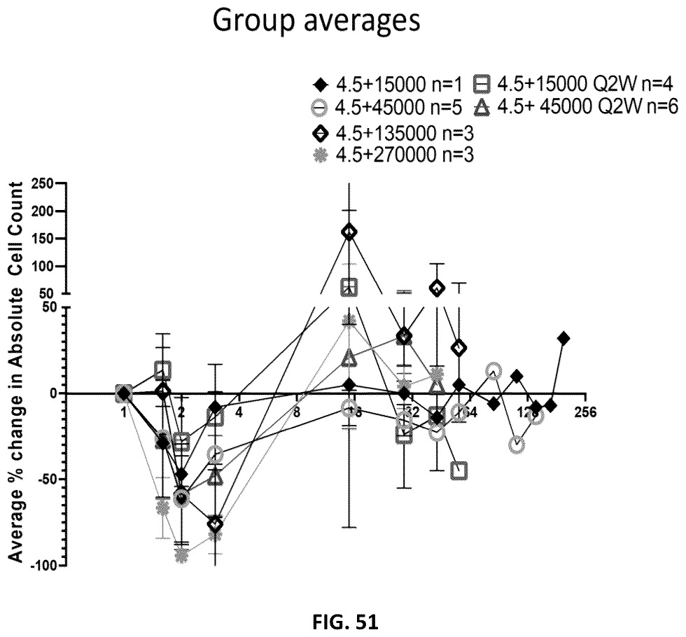

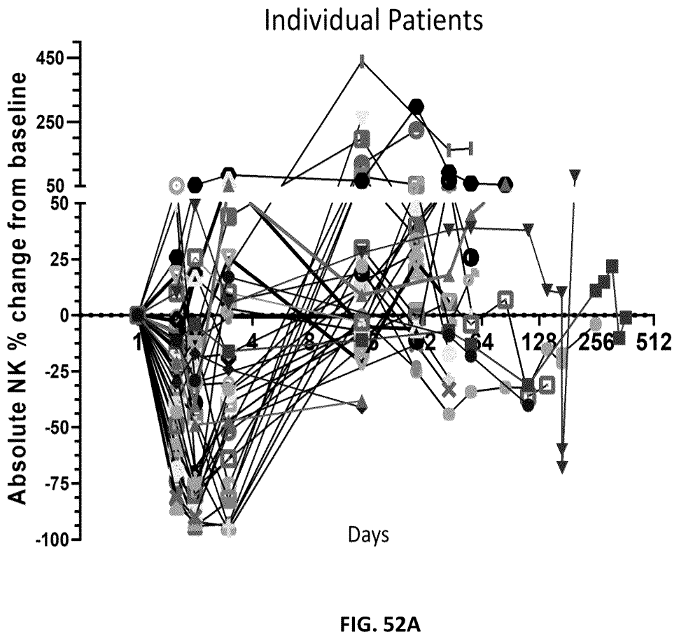

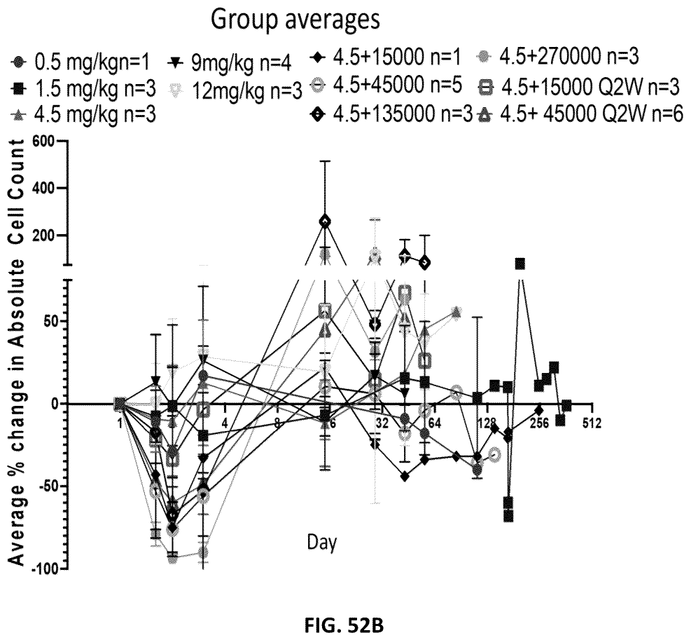

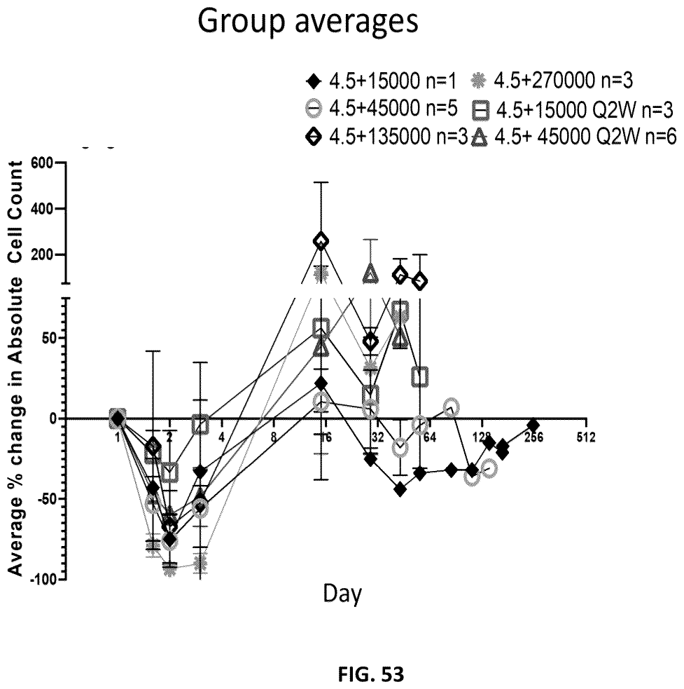

FIGS. 48A-481B, 50A-501B, 51, 52A-52B, 53, 54A-54B, and 55A-55B graphically present PD data showing the effect of AU-007 and the mechanism of action, including percent change in the absolute number of circulating regulatory T cells. Regulatory T cells were defined as CD3+ CD4+ CD25+ CD 1271o CD45+ cells. Consistent with the mechanism of action of inhibiting IL-2 from interacting with the trimeric receptor, regulatory T cells decreased in the peripheral circulation. This was observed in both the monotherapy arm and the arms which also included aldesleukin (Proleukin) and was consistent among patients. FIG. 481B: Cohorts receiving only AU-007; FIG. 49: Cohorts with both AU-007 and at least 1 dose of aldesleukin. Percent change in the absolute number of circulating (FIGS. 50B and 51) CD8+ T cells and (FIGS. 52B and 53) NK cells. Increases in circulating effector cells were observed over time with monotherapy and lowest doses of IL-2 showing lower levels of peripheral effector cell expansion. Expansions were observed the longer the patient stayed on study. This is consistent with the mechanism of action of AU-007 stabilizing low levels of IL-2 and the requirement to build to an activation threshold of IL-2. In the presence of higher levels of IL-2, both CD8 and NK cells increased higher and earlier. Higher doses of IL-2 are being further explored and results are expected to be consistent with the present findings.

FIGS. 48A-48B show flow cytometry characterization of circulating Tregs (CD3+ CD4+ CD25+ CD127 dim, Foxp3+) demonstrating an overall trend toward decreasing percentage of circulating Tregs in patients as measured either individually (FIG. 48A) or by group averages (FIG. 48B) for all dosed groups. FIG. 49 shows changes in peripheral blood Tregs for cohorts receiving AU-007+ Proleukin® (aldesleukin). FIGS. 50A and 50B show the changes in absolute peripheral blood CD8 cells per individual patient (FIG. 50A) or based on group averages based on dose and regime (FIG. 50B). FIG. 51 presents the change in peripheral blood CD8 for cohorts receiving AU-007+ Proleukin® (aldesleukin). When comparing this data to knowledge in the art is known that IL-2 causes initial dose dependent decreases in lymphocytes (likely migration into tissue) (Todd et al. (2016) Regulatory T Cell Responses in Participants with Type 1 Diabetes after a Single Dose of Interleukin2: A Non-Randomised, Open Label, Adaptive Dose Finding Trial. PLoS Med 13 (10): e1002139.) FIGS. 52A and 52B show changes in Absolute Peripheral Blood NK cells per individual patient (FIG. 52A) or as a group average based on dose and regime (FIG. 52B), while FIG. 53 shows change in peripheral blood NK cells for cohorts receiving AU-007+ Proleukin® (aldesleukin). As demonstrated in FIGS. 50A, 50B, 51, 52A, 52B, and 53, increases in circulating effector cells were observed over time with monotherapy and lowest doses of IL-2 showing lower levels of peripheral effector cell expansion. Expansions were observed the longer the patient stayed on study. This is consistent with the mechanism of action of AU-007 stabilizing low levels of IL-2 and the requirement to build to an activation threshold of IL-2. In the presence of higher levels of IL-2, both CD8+ T cells and NK cells increased higher and earlier. Higher doses of IL-2 are being further explored and results will be reported at a later date.

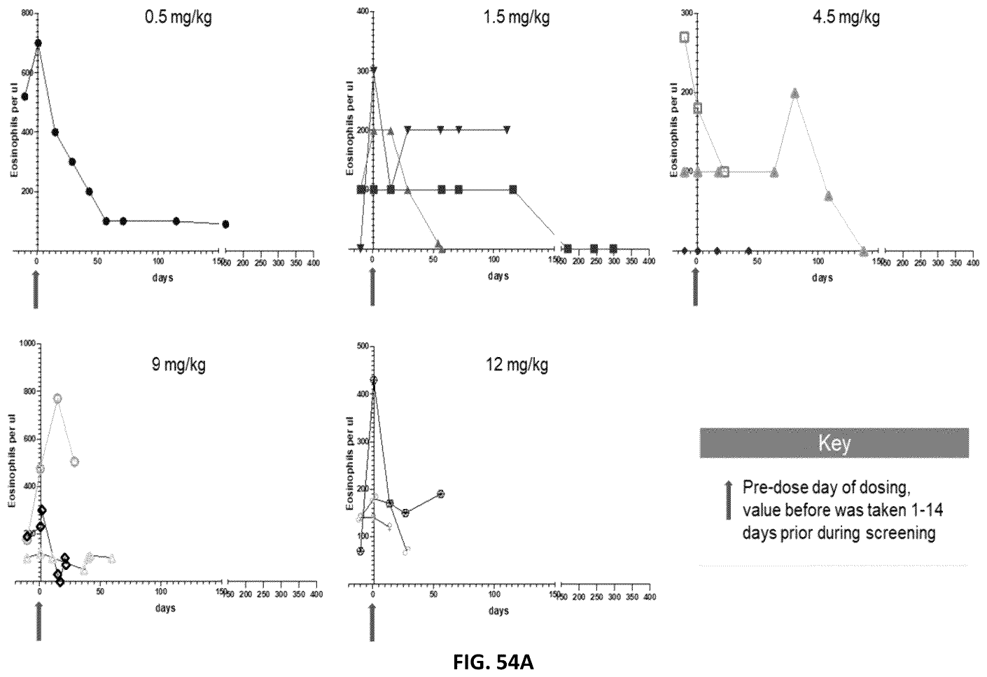

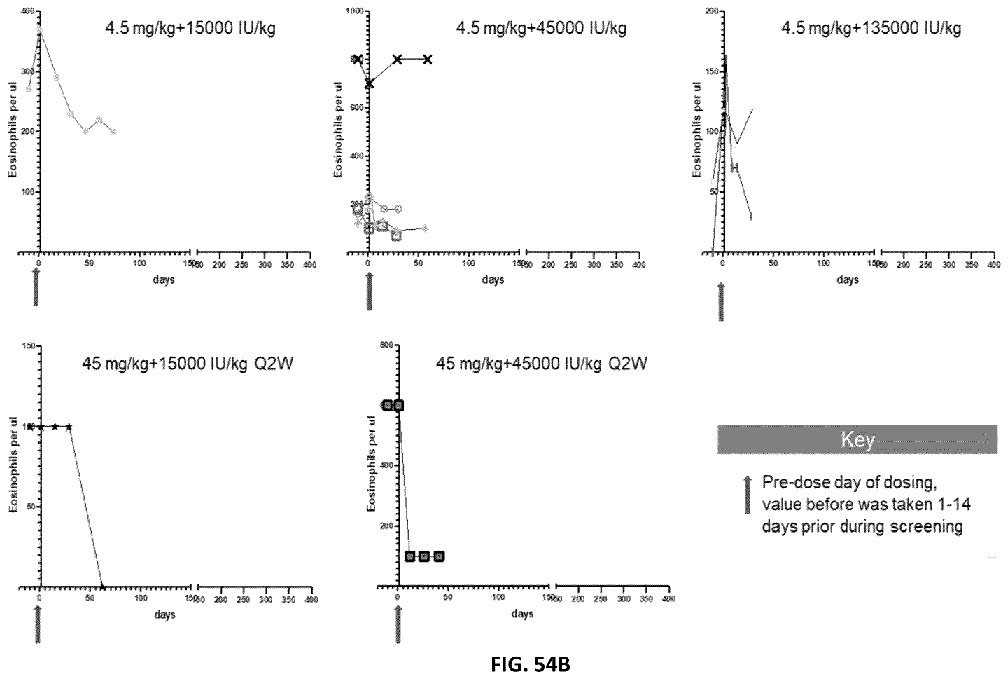

FIGS. 54A and 54B show that even though eosinophils express the same trimeric, CD25⁺ IL-2 receptor as Tregs and vascular endothelium, no increase in the absolute eosinophil counts were observed (FIG. 54A—all available data; FIG. 54B AU-007 and Proleukin® (aldesleukin) dosed patients), with patients having decreasing trends.

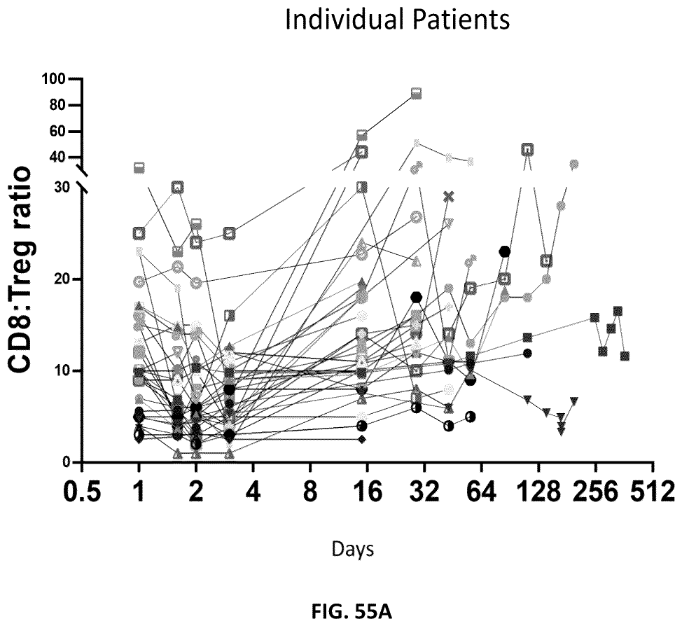

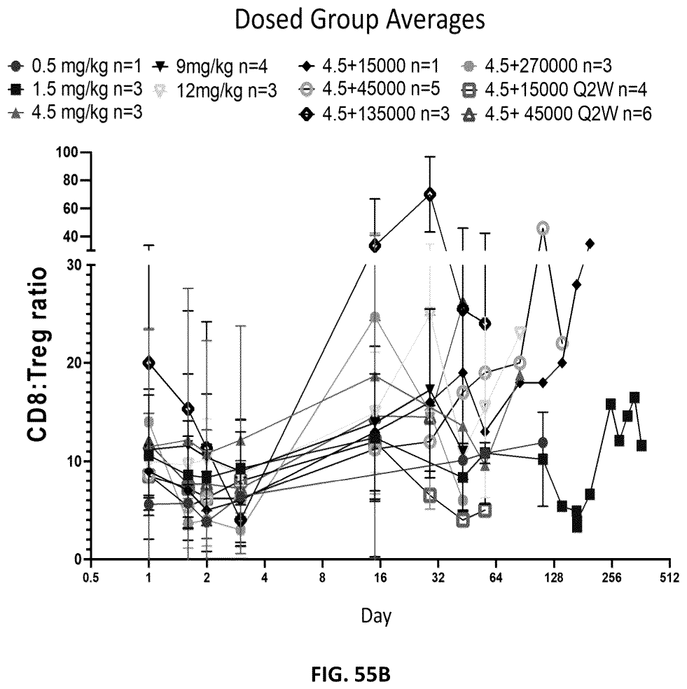

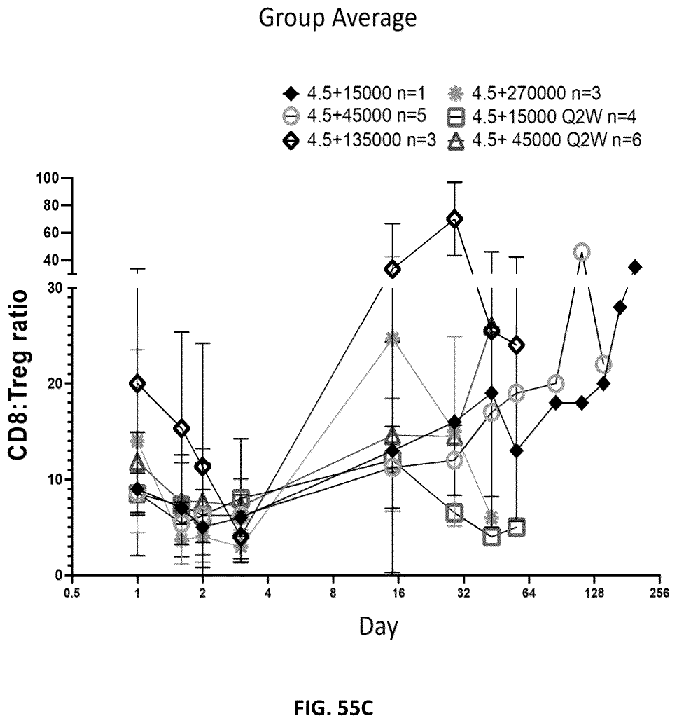

FIGS. 55A-55C show change in the ratio of CD8+ T cells to Tregs observed in the periphery, wherein the decrease in Tregs and changes in CD8+ T cells resulted in an increase in the CD8/Treg ratio when measured per patient (FIG. 55A), by dose group averages (FIG. 55B), or in patients receiving Proleukin® (aldesleukin) (FIG. 55C). Consistent with the observations observed with the changes in Tregs and CD8s, there is an observed trend to an increase in the CD8+/Treg ratio with monotherapy. In the presence of aldesleukin, an increase in the CD8+/Treg ratio was observed, particularly at higher doses of aldesleukin. Consistent with the mechanism of action, higher doses (of low dose IL2) and longer exposure trend to higher CD8/Treg ratios with no observed drug related toxicity. It is anticipated that increasing doses of aldesleukin will further enhance the peripheral response.

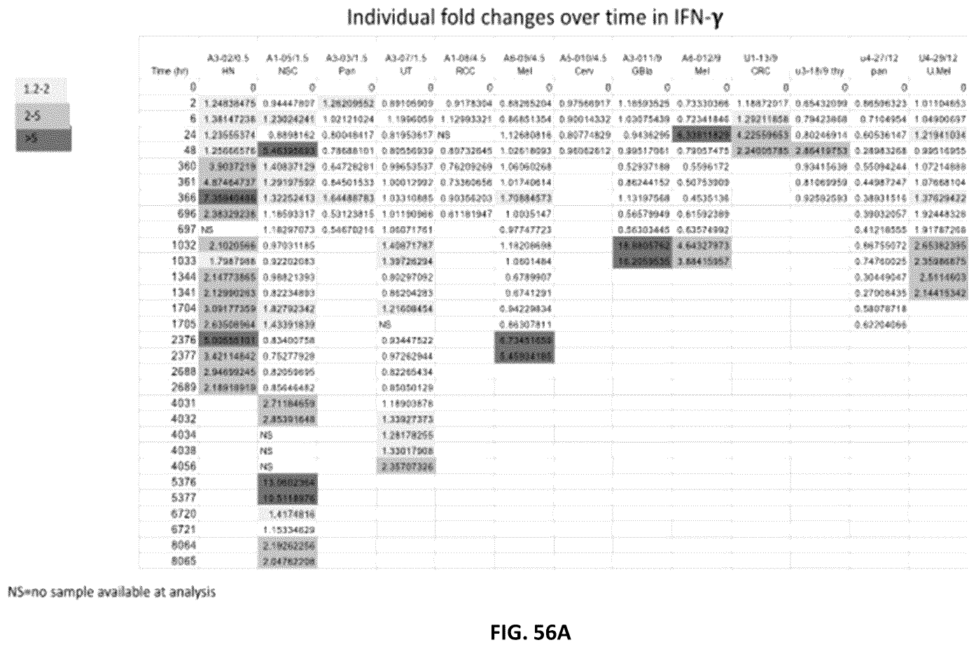

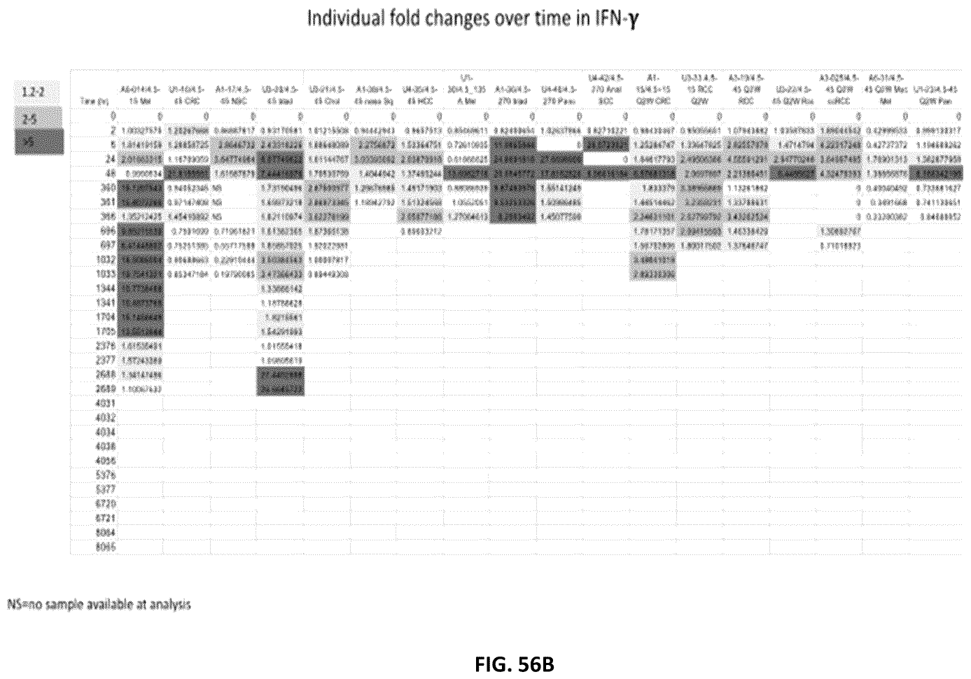

FIGS. 56A and 56B are heat maps showing the changes in circulating interferon gamma (IFN-γ). Changes greater than 5 fold are represented by dark green. FIG. 56A are patients receiving AU-007 and FIG. 56B are patients receiving AU-007 and Proleukin® (aldesleukin). Overall, the longer patients stay on trial or higher doses of AU-007, or addition of Proleukin® (aldesleukin) appears to be associated with more circulating IFN-γ.

### Summary and Conclusion

Ongoing PK data demonstrates dose proportional AU-007 serum concentrations with typical characteristics of an IgG1 human mAb. Current available PD data demonstrates overall decreasing Tregs (% change and absolute) and eosinophils, which both express the trimeric IL-2 receptor. AU-007 monotherapy at AU-007 doses up to 9.0 mg/kg and combination therapy (with IL-2 low dose up to 45K IU/kg) is safe and well tolerated, with initial signs of immune modulation consistent with AU-007's mechanism of action. The clinical trial is continuing with higher doses of AU-007 up to 12 mg/kg, and IL-2 up to 270K IU/kg, with similar positive results expected.

This preliminary pharmacodynamic data from the AU-007 Phase 1 trial in multiple solid tumors show treatment with AU-007 as monotherapy or in combination with Proleukin induces changes consistent with the mechanism of action (i.e., redirecting of IL-2 away from regulatory T cells and toward effectors) and consistent with an increase in the overall inflammatory profile of patients. The overall effect increases with time on therapy or with the addition of higher levels of Proleukin. These changes were not associated with any drug-related events (data not shown). AU-007 treatment, with or without Proleukin, led to decreases in eosinophils. Proleukin administered as a single agent is known to raise eosinophil counts, and such increases have been associated with an adverse event profile. No changes were observed in circulating sCD25 levels (data not shown). IL-2 levels are still being investigated.

The preliminary data presented here support the overall mechanism of action for AU-007. The observed decreases in circulating regulatory T cells and increases in IFN-γ in the presence of the low doses of IL-2 assessed in this trial administered with AU-007 counters what is typically observed if low doses of IL-2 are given in the absence of AU-007. Low dose IL-2 given as a single agent increases Tregs and decreases IFN-γ in circulation and hence is being investigated as a treatment for autoimmune diseases. Proleukin given as a single agent also has a well-characterized adverse event of increasing circulating levels of eosinophils, and lung toxicity can ensue from the eosinophilia. IL-2 interacting with the trimeric receptor on the eosinophils is thought to be the major contributor of this increase. Here, again consistent with the mechanism of action of AU-007 redirecting IL-2 away from the trimeric receptor, AU-007 or AU-007+ Proleukin produce decreases in circulating levels of eosinophils, supporting the proposed activity of the antibody. While the overall number of patients per group is small and each cohort has multiple cancer types, the trends observed here with AU-007 and increasing Proleukin doses demonstrate favorable pharmacodynamic effects, including reductions in regulatory T cells, increases in CD8+ T cells and NK cells, increases in CD8+/Treg ratios, and increases in IFN-γ, in a broad range of cancer types. Further investigations with higher doses of Proleukin administered with AU-007 are currently being investigated. Efficacy and safety data for the study are to be reported at a later date.

The ongoing investigation shows that AU-007 Q2W has a tolerable and manageable safety profile as a monotherapy evaluated up to 12 mg/kg, in combination with one aldesleukin loading dose evaluated up to 4.5 mg/kg AU-007+270K IU/kg aldesleukin, and in combination with aldesleukin Q2W evaluated up to 4.5K mg/kg AU-007+135K IU/kg aldesleukin.

Preliminary evidence of anti-tumor activity has now been observed in multiple heavily pre-treated patients, including progression through checkpoint inhibitors in patients with melanoma (anti-PD-1/CTLA-4), RCC (anti-PD-1), and NSCLC (anti-PD-1).

Trends toward decreasing Treg cells with concordant increases in CD8:Treg ratio, initial interferon-gamma (IFN-γ) increases, and absolute eosinophil decreases are consistent with the novel mechanism of action of AU-007.

Enrollment continues in Arm 1C (4.5 mg/kg AU-007+270K IU/kg aldesleukin Q2W). Further development of AU-007 will continue in combination with aldesleukin, and Phase 2 expansion cohorts are planned at least in melanoma, RCC, and NSCLC patients.

### Example 10

### BDG17.069 (AU-007) Inhibits the “Negative FeedbackLoop”, In Vitro.

Objective: Evaluation of the mechanism of anti-IL2 antibody clone BDG17.069 (AU-007). The goal of this study was to perform in vitro functional analysis to support the negative feedback loop that occurs when treating with IL-2 or a non-alpha IL-2/CD25 fusion protein and compare these results with administration of AU-007 results.

### Methods:

Methods used analyzed and compared the functional activity of AU-007 vs. hIL-2/CD25 conjugated cytokine (similar to nemvaleukin).
Step1: hIL-2/CD25 conjugated cytokine purification (endotoxin free)
Step2: Verify trimer inhibition using hPBMCs-pSTAT5 (short term assay, AU-007/hIL-2 vs. hIL-2/CD25 conj.)
Step3: naïve hPBMCs expansion assay (AU-007/hIL-2 vs. hIL-2/CD25 conj.)
Step4: Long term effect on activated hPBMCs—Assay development
Step5: Examine long term effect on activated hPBMCs AU-007 alone vs. hIL-2/CD25 conj.
   hPBMCs Expansion Assay Protocol

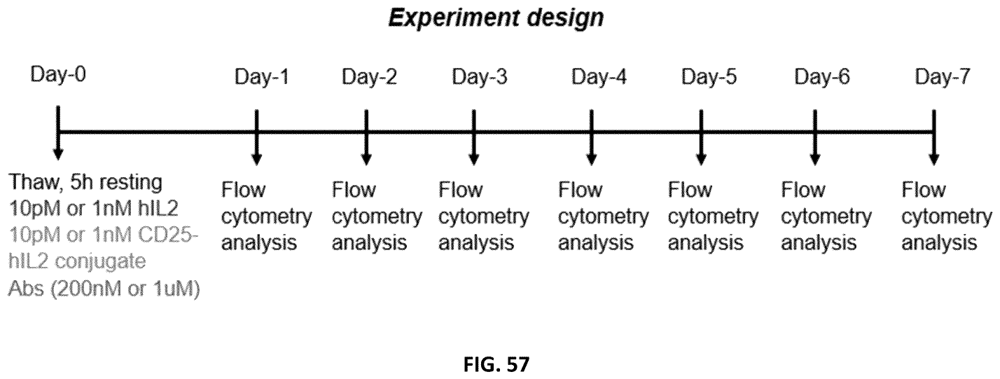

Human PBMC were added to 96 well plates and treated with the indicated conditions (red, green, and blue) (FIG. 57). hPBMCs were stimulated once a day-0, with low dose (10 pM) or high dose (1 nM) of modified IL-2 (IL-2-CD25 conj) and nonmodified hIL-2+ Ab (AU-007 or isotype control). Percentage of regulatory T-cells were analyzed daily for 7 days by flow cytometry; gating was defined based on FMOs (fluorescence minus one control).

To examine NK cells and CD4+ Regulatory T cells (CD3+ CD4+ CD127-CD25+ FoxP3+), hPBMCs were stimulated once a day-0, with high dose (1 nM) of modified IL-2 (IL-2-CD25 conjugate) and nonmodified hIL-2+ Ab (AU-007 or isotype control). The percentage of immune cells subsets were analyzed daily for 7 days by flow cytometry, gating was defined based on FMOs (fluorescence minus one control).

For analysis of CD25, FOXP3, and CD56 markers, hPBMCs were stimulated once a day-0, with high dose (1 nM) of modified IL-2 (IL-2-CD25 conjugate) and nonmodified hIL-2+ Ab (AU-007 or isotype control). Percentage of immune cells subsets were analyzed daily for 7 days by flow cytometry; gating was defined based on FMOs (fluorescence minus one control).

For analysis of ΔCD8+ Teffs: ΔTregs, hPBMCs were stimulated once a day-0, with low dose (10 pM) or with high dose (1 nM) of modified IL-2 (IL-2-CD25 conjugate) and nonmodified hIL-2+ Ab (AU-007 or isotype control). Percentage of immune cells subsets were analyzed daily for 7 days by flow cytometry; gating was defined based on FMOs (fluorescence minus one control).

The experimental design assaying restimulation-induced cell death (RICD) is shown in FIG. 63. hPBMCs were activated with anti-CD3/anti-CD28 and stimulated with high dose hIL-2 with 100 nM Abs (AU-007 or isotype control). Percentage of immune cells subsets were analyzed daily for 7 days by flow cytometry; gating was defined based on FMOs (fluorescence minus one control).

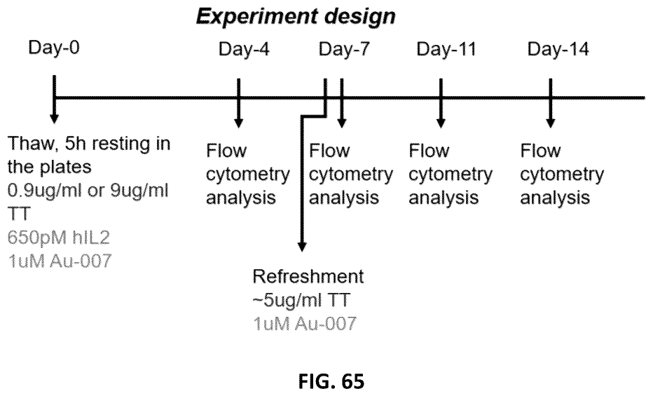

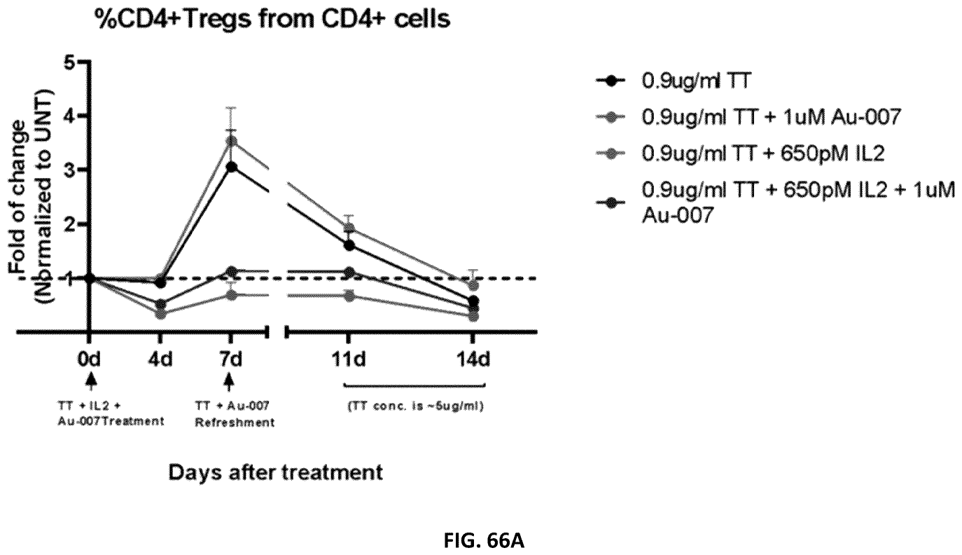

A tetanus toxoid assay was used to examine the effect of AU-007 when antigen specific response is evaluated. The experimental design of this assay is presented in FIG. 65.

### Results:

AU-007 Prevents the Negative Feedback Loop of Autocrine Secreted IL-2 while Modified IL-2 (IL-2-CD25 Conjugate, i.e., Non-Alpha) does not.

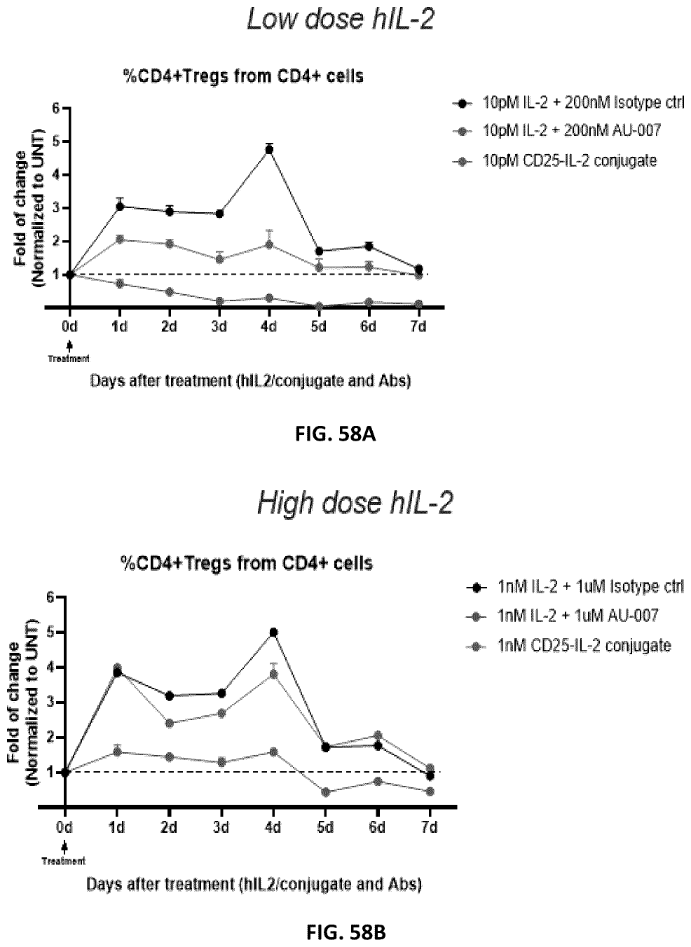

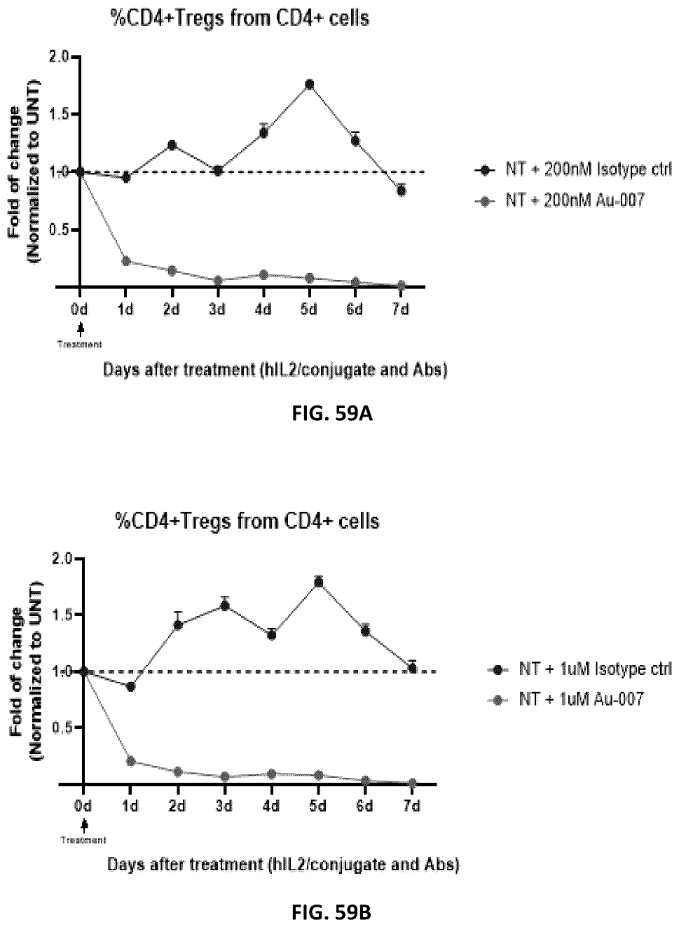

The data presented in FIGS. 58A and 58B demonstrate that AU-007 inhibits the downstream effect of autocrine secreted IL-2 on Tregs expansion while modified IL-2 (CD25-IL-2 conjugate) does not. AU-007 advantageously binds to endogenous IL-2 and breaks the negative feedback loop to Tregs in human PBMCs.

Interestingly, when observing Tregs in the absences of IL-2, the data showed that AU-007 inhibits spontaneously expanding Tregs in PBMC cultures (FIGS. 58A-58B).

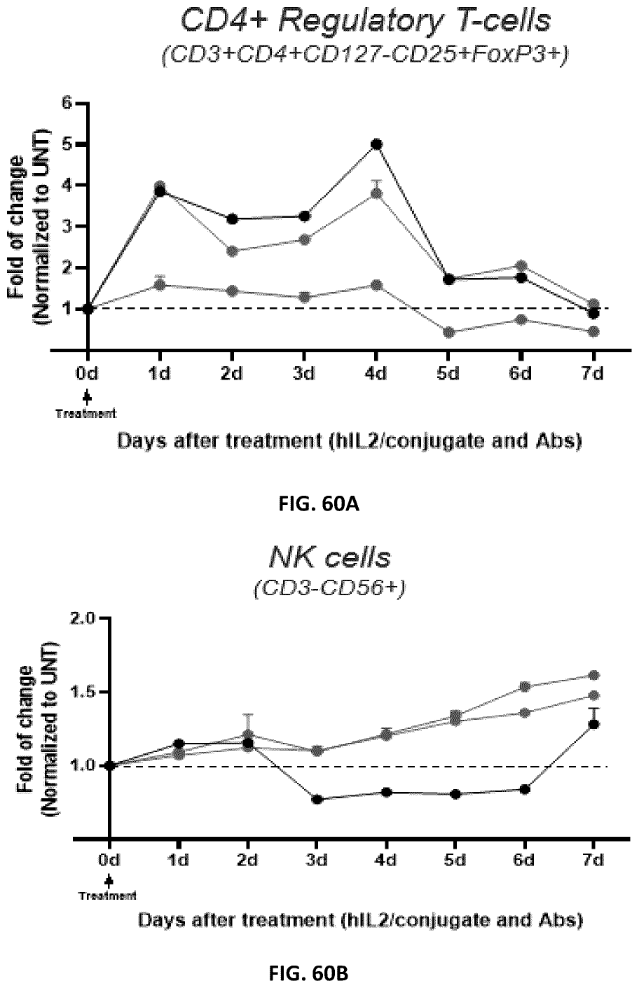

FIGS. 60A and 60B show that both AU-007 and a modified IL-2 cytokine (IL-2-CD25 conjugate) enhance the expansion of NK cells in a similar manner (FIG. 60B), while only AU-007 inhibits the downstream effect of autocrine secreted IL-2 on Tregs expansion observed 4 days post-treatment (FIG. 60A).

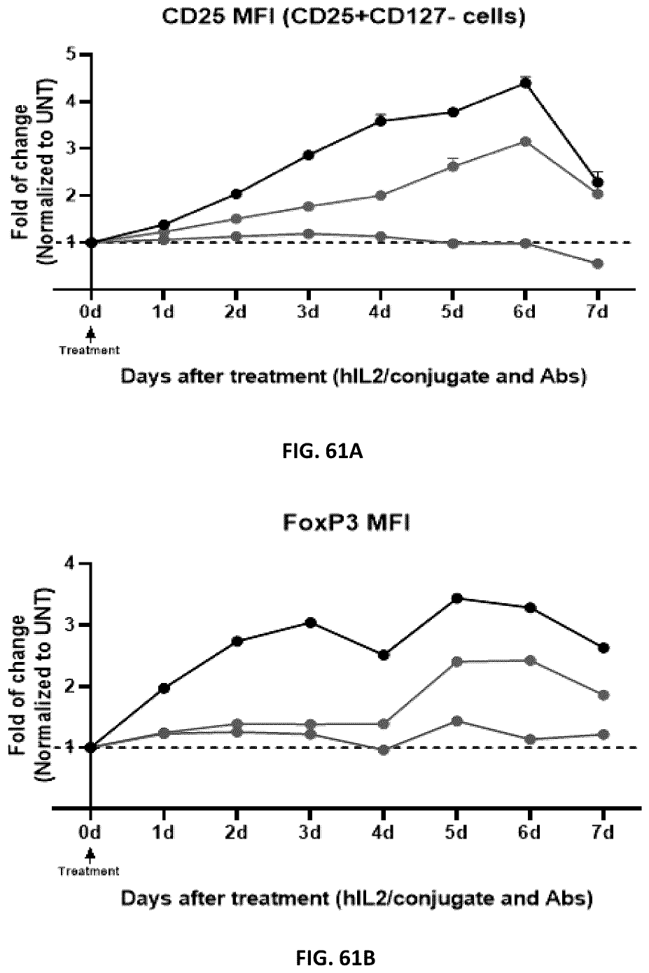

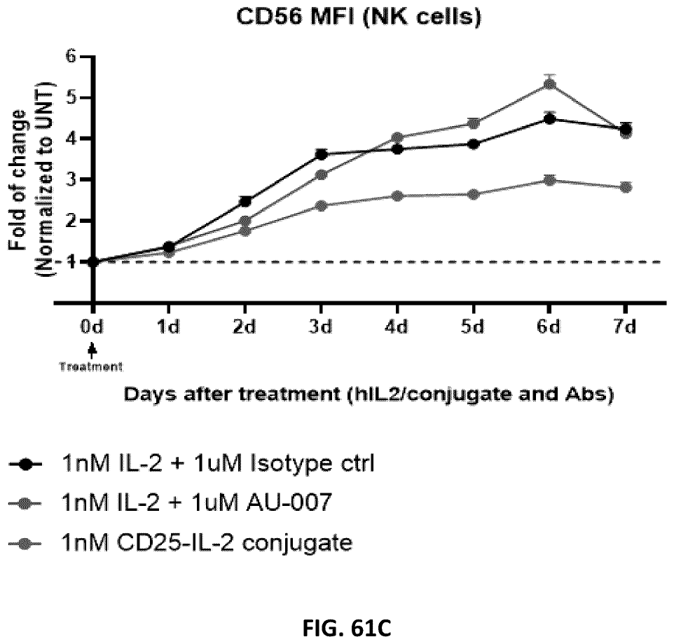

Further analysis showed that only AU-007 downregulates Tregs' suppressive markers (CD25 & FoxP3; FIGS. 61A and 61B) while preserving the upregulation of NK cells activation marker (CD56; FIG. 61C) observed 3-6 days post-treatment.

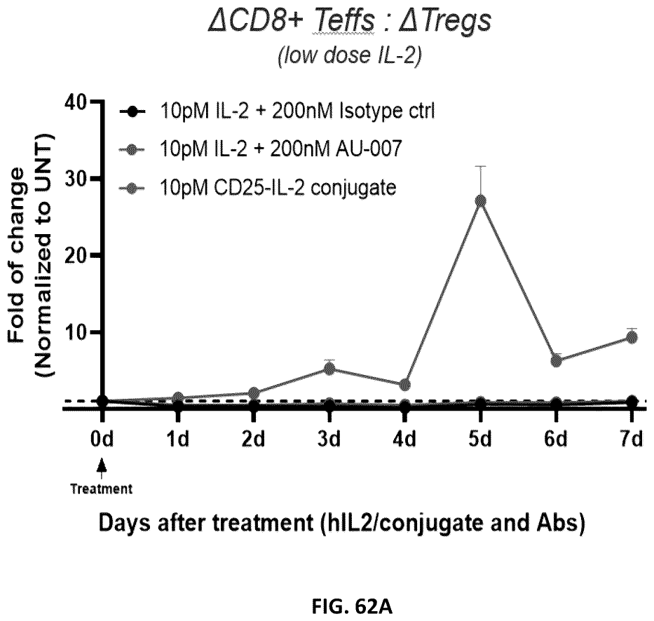

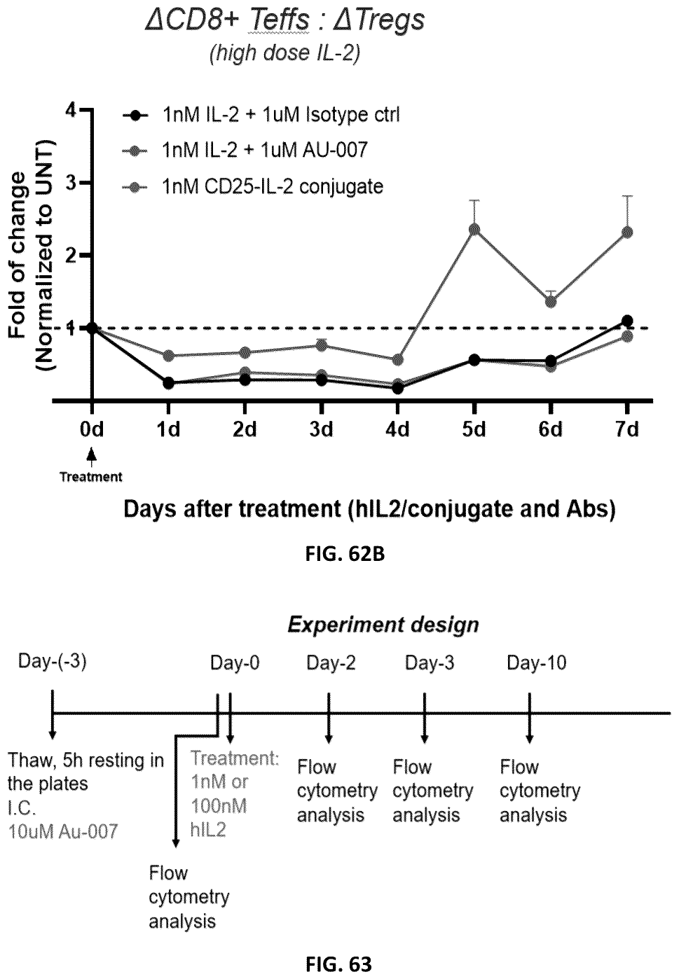

AU-007 was unique compared with the IL-2-CD25 conjugate in that only AU-007 increased significantly the ratio of CD8+ T-effs:Tregs observed 3-6 days post-treatment with low dose IL-2 (FIG. 62A) or high dose IL-2 (FIG. 62B) (similar results were obtained for CD4+ Teffs: Tregs ratio; data not shown).

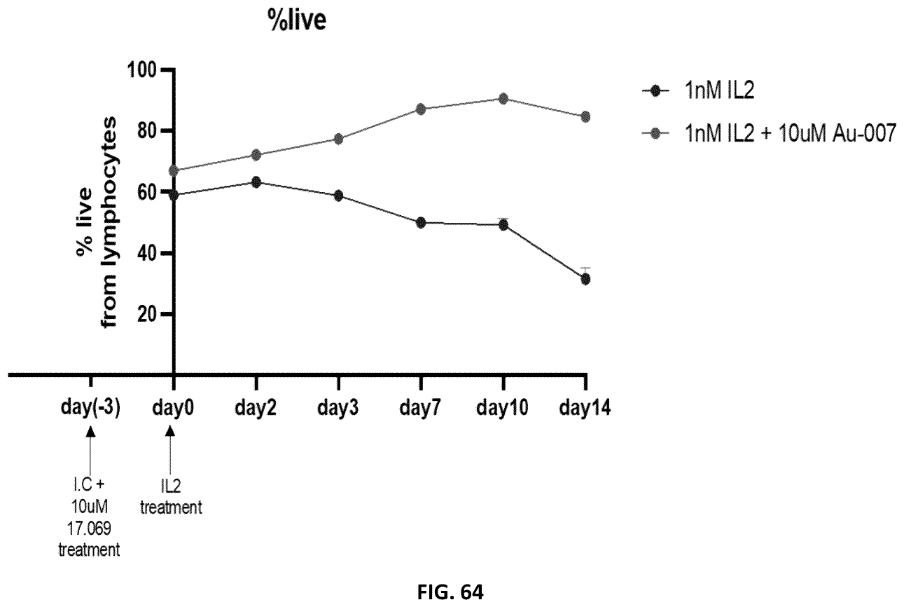

Restimulation-induced cell death (RICD) is an apoptotic program triggered in activated T cells when an abundance of antigen and IL-2 are present, imposing a negative feedback mechanism that constrains the growing T cell population. Preliminary data suggesting AU-007 rescues activated lymphocyte from IL-2 induced cell death fate (FIG. 64).

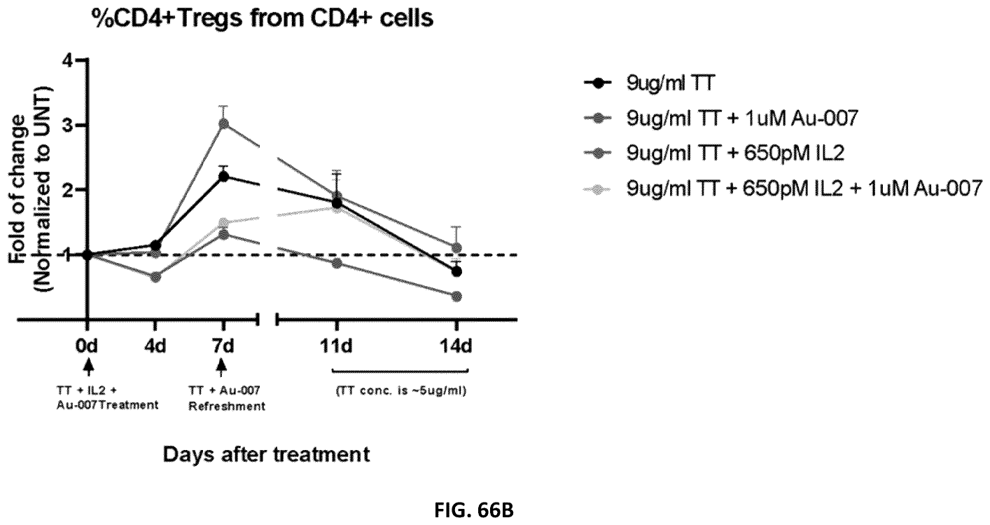

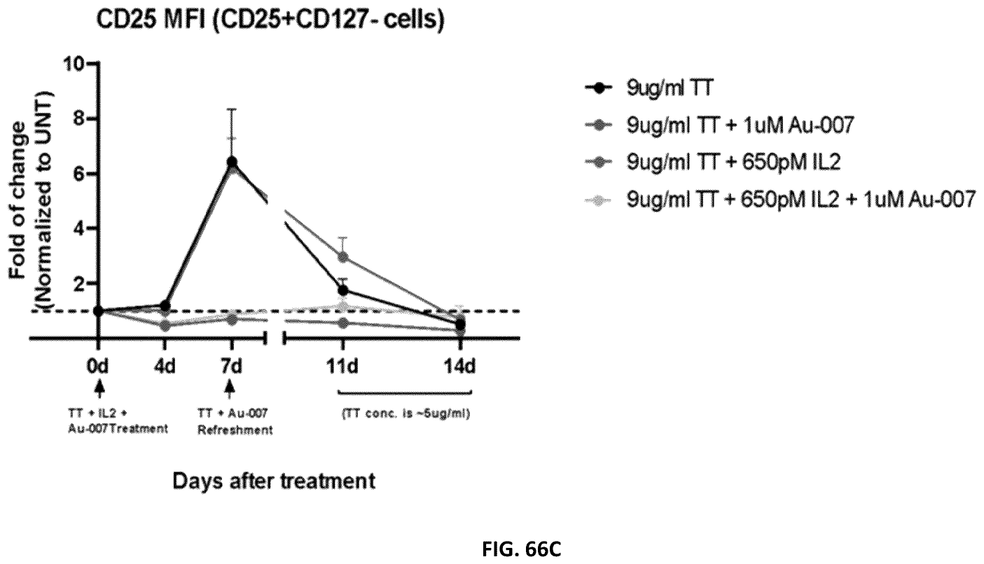

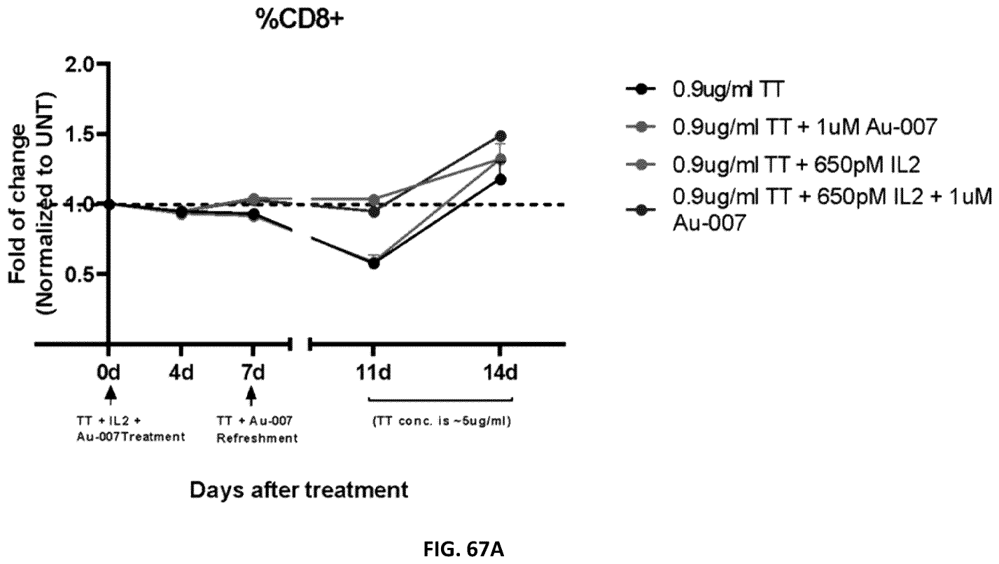

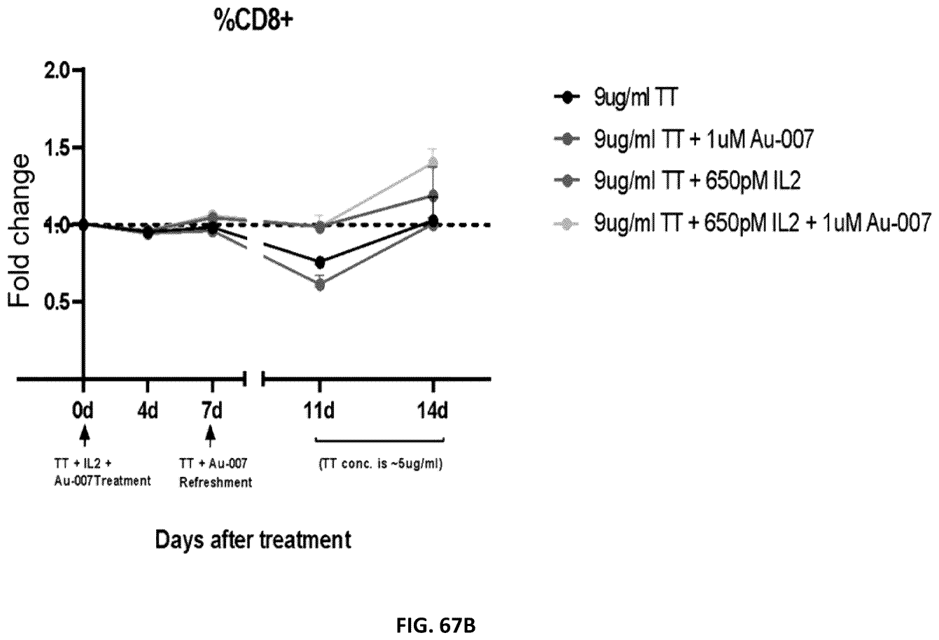

The effect of AU-007 when antigen specific response is evaluated (human PBMCs are reactive to tetanus toxoid (TT) due to widespread use of the tetanus vaccine) was evaluated as shown in FIG. 65. The results showed that AU-007 inhibits expansion of Tregs in cultures stimulated with a specific protein antigen at low dose of the antigen (0.9 μg/ml TT; FIG. 66A) and high dose of the antigen (9.0 pg/ml TT; FIG. 66B). FIG. 66C shows that Tregs are not as active in wells with AU-007. Moreover, FIGS. 67A and 67B show that AU-007 prevented the RICD cell death fate of CD8+ cells in cultures stimulated with a specific protein antigen, at low dose (0.9 μg/ml TT; FIG. 67A) or high dose (9.0 μg/ml; FIG. 67B) antigen.

### SUMMARY

The data demonstrate that AU-007 sequesters endogenously generated IL-2 away from regulatory T cells but allows the expansion of effector cells. Beneficially, the addition of AU-007 prevents cells from dying as the result of adding extra IL-2 (i.e., restimulation induced cell death).

### Example 11

### BDG17.069 (AU-007) Dose Evaluation/Selection and Toxicology Analysis

Objective: To evaluate anti-IL-2 antibody AU-007 for toxic effects and determine dose range.

Methods: An acute toxicity study was completed in cyno monkeys based on the following regime: single dose AU-007 at 5 mg/kg, 25 mg/kg, 100 mg/kg, or 100 mg/kg plus (+) low dose Proleukin® (aldesleukin) (15000 IU/kg).

A repeat dose toxicity study was completed in cyno monkeys based on the following regime: 8 weeks of intravenous (IV) dosing, 2× per week followed by a 4-week recovery (doses were 5 mg/kg, 25 mg/kg, or 100 mg/kg). A Good Laboratory Practices (GLP) immunohistochemistry assessment of human tissue cross reactivity completed.

Comparison studies between AU-007 and placebo (PBS) were performed in cyno monkeys at a range of dosages, 5 mg/kg, 25 mg/kg, and 100 mg/kg, wherein percentage of relevant cell types were measured overtime, e.g., CD4⁺ cells and NK cells.

Concentration of IL2 was also measured in these studies. Based on the assay description from the vendor, AU-007 is hypothesized to interfere with the detection of IL-2 and therefore in the presence of AU-007, total IL-2 levels (free+bound) may be higher than measured in the assay.

Concentration of AU-007 was also measured overtime during Q2W dosing regimens.

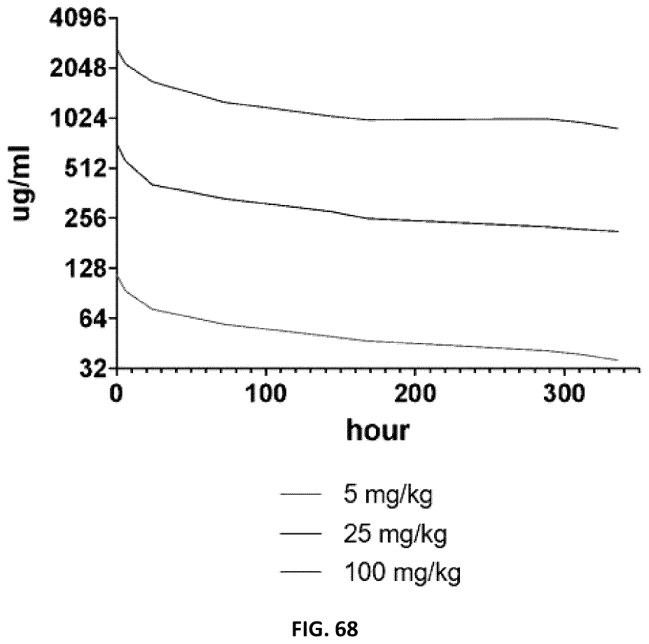

Results: In the acute single dose toxicity study, no observed toxicity was seen in any animals. FIG. 68 demonstrates dose proportional pharmacokinetics were observed in this acute toxicity study. Additionally, no changes in peripheral immune cells were observed following the single dose, wherein measurements were taken after 24 hours, and no cytokine changes or cytokine storms were observed. (Data not shown)

In the repeat dose toxicity study, there were no significant clinical observations and nothing unusual on necropsy. No cytokine storms were observed. (Data not shown) In a tissue cross reactivity study, no observed cross reactivity on any human tissue was observed. (Data not shown)

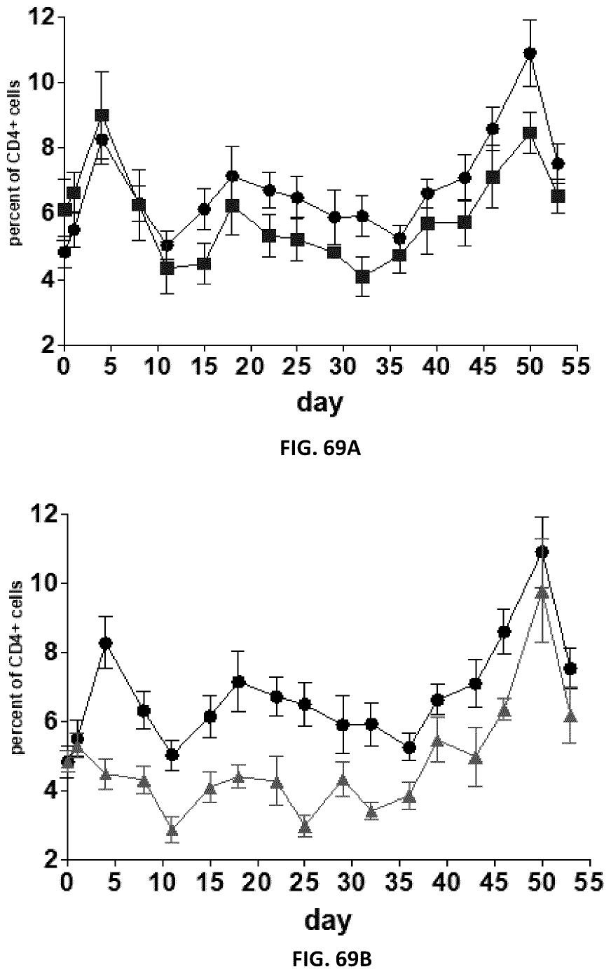

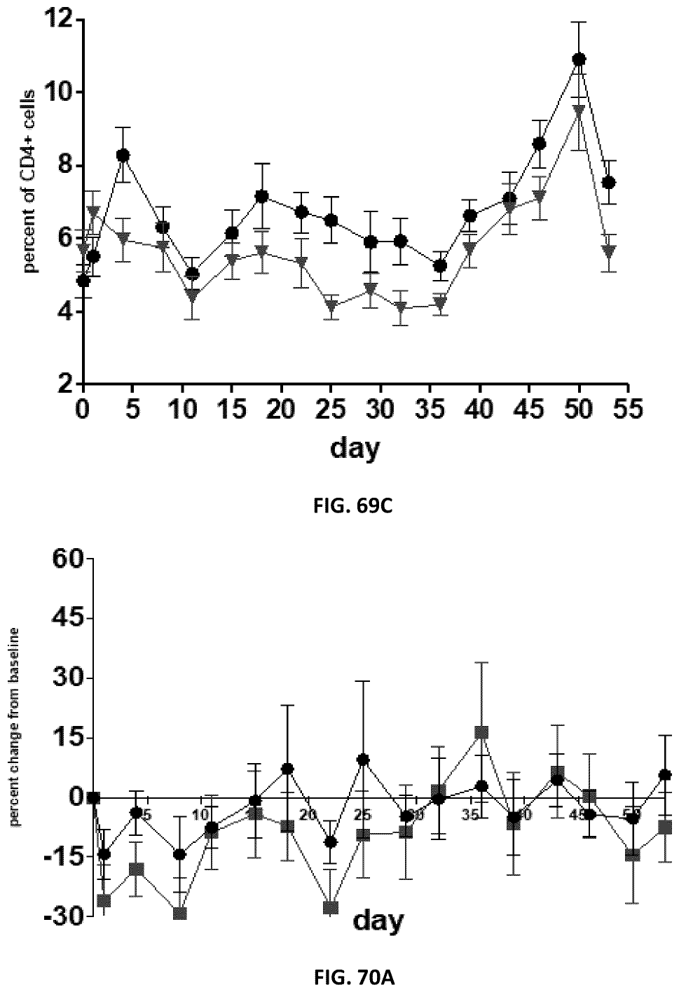

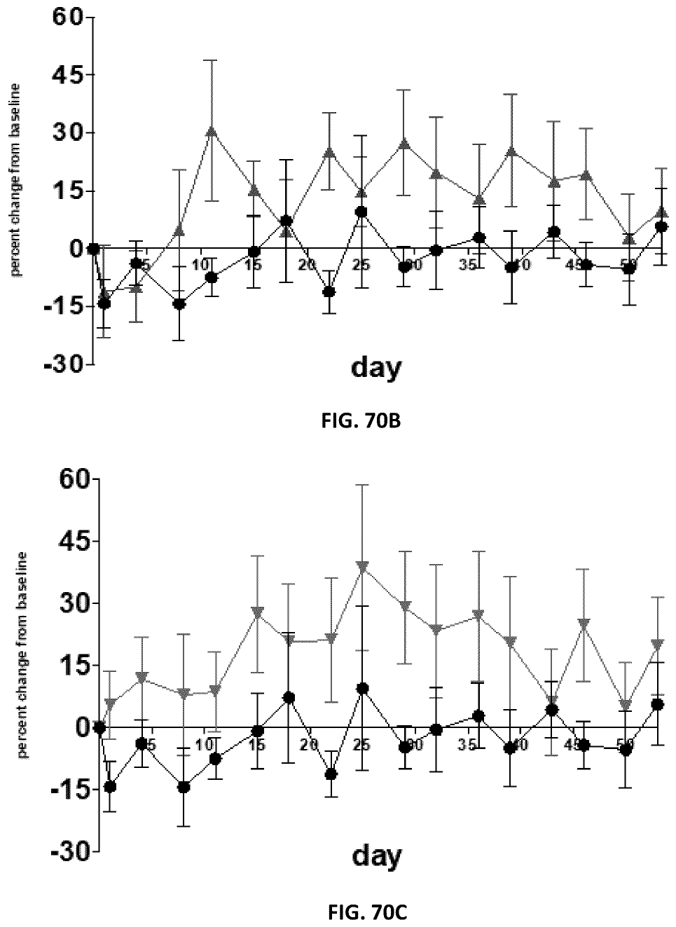

FIGS. 69A-69C show that relative to placebo, AU-007 decreased CD4+ CD25+ cells relative to placebo across all doses. Concurrently, FIGS. 70A-70C show that in cyno monkeys, AU-007 increased peripheral NK cells relative to baseline. While variable, there is a dose dependent increase in NK cells in the periphery with relatively no consistent change in the PBS groups.

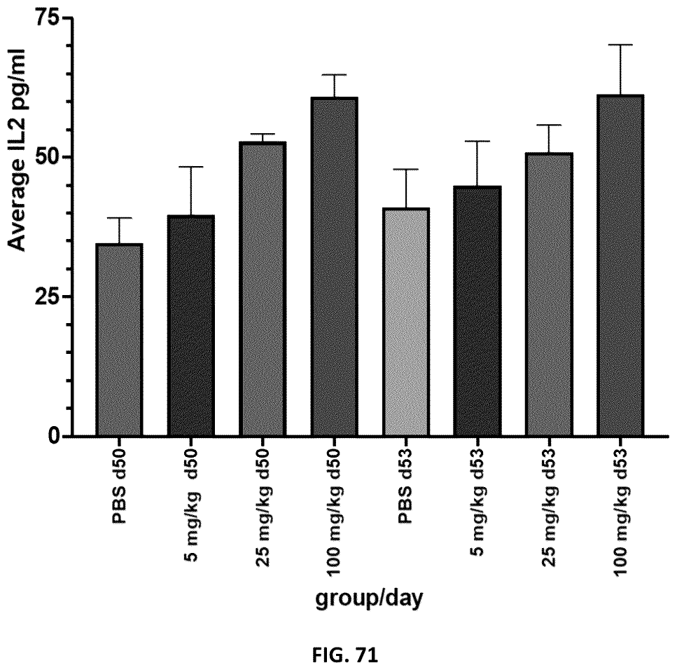

In cyno monkeys administered AU-007, IL-2 increased in a dose dependent manner over saline (PBS) Control (FIG. 71). Free IL-2 was increased in the peripheral blood of cyno monkeys in a dose dependent manner. The effect was seen best at late stages when IL-2 begins to accumulate.

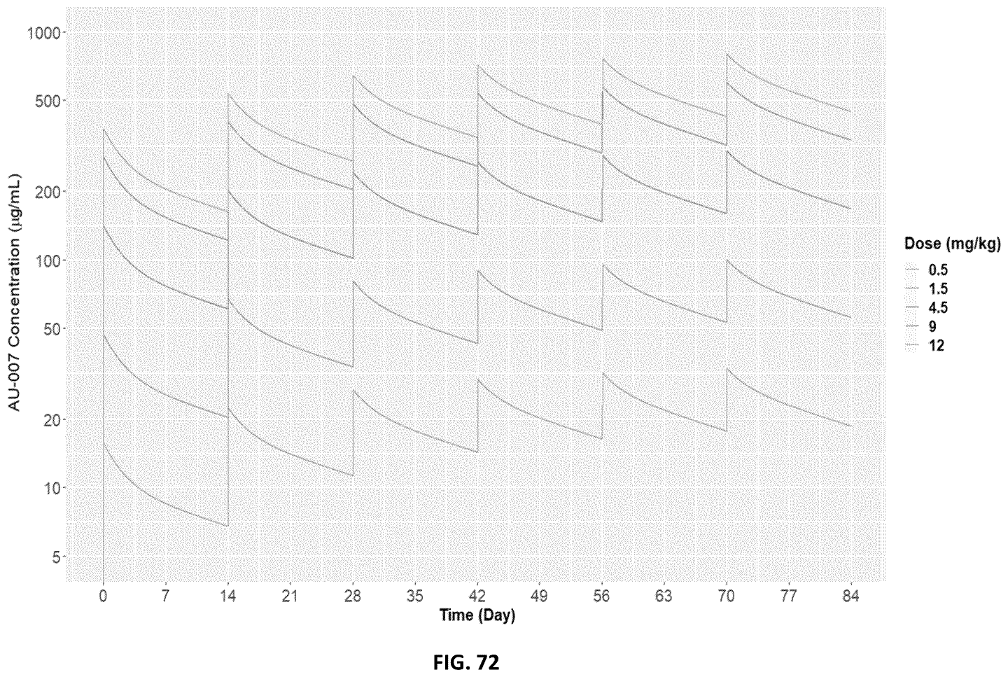

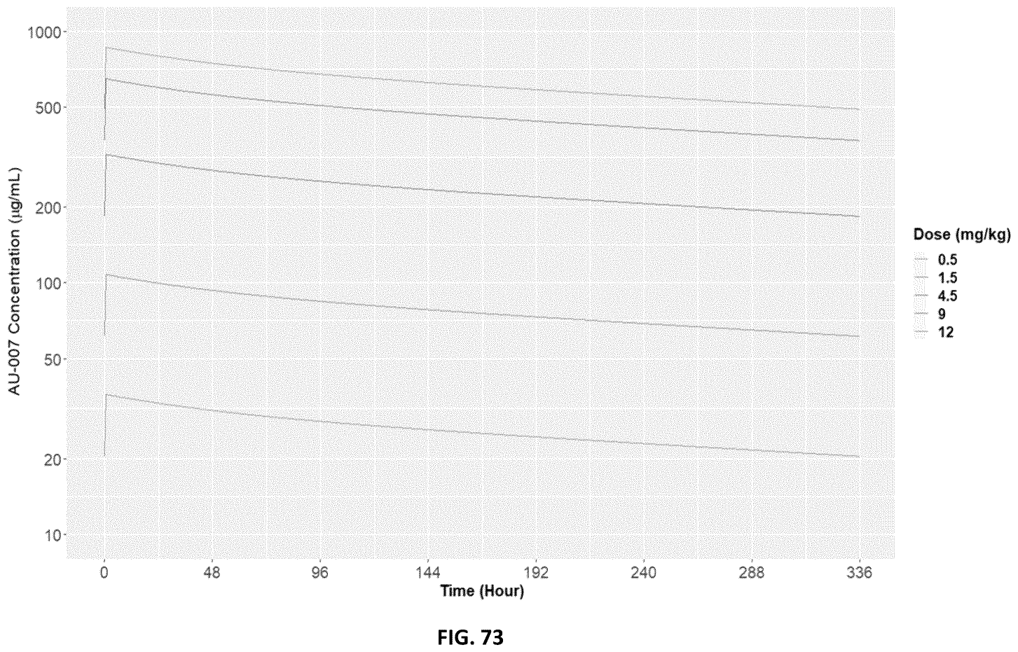

FIG. 72 shows simulated human PK profiles and FIG. 73 shows simulated steady-state human PK profiles, following Q2W dosing regimens of 0.5 mg/kg, 1.5 mg/kg, 4.5 mg/kg, 9 mg/kg, and 12 mg/kg.

### SUMMARY

The selected AU-007 dose range was evaluated in dose escalation studies for toxicity and effective dosage. The AU-007 dose range (0.5, 1.5, 4.5, 9 and 12 mg/kg) evaluation in dose escalation was based on:
Pre-clinical cynomolgus monkey safety data that:
   Demonstrated a very clean toxicity profile, wherein the maximum tolerable dose (MTD) was not reached; and
   Allowed for a safe starting dose that also provided serum levels high enough to bind all endogenous and endogenously administered IL-2 (as Proleukin® (aldesleukin)) by large overage;
Preclinical cynomolgus monkey TK predicted that 1.5 mg/kg AU-007 (second dose level evaluated in escalation) would bind >500-fold the projected IL-2 serum concentrations achieved with the highest Proleukin® (aldesleukin) dose (270K IU/kg).
Initial PK from patients aligns with the pre-clinical TK data.

AU-007 doses 1.5 mg/kg or 4.5 mg/kg are currently projected to provide blood concentrations adequate to cover all endogenous and exogenously administered IL-2 (Proleukin® (aldesleukin)) with overages of >500 and >1000 fold, respectively.

TABLE 19
Amount of IL-2 That AU-007 Can Bind at Steady State

| | Amount of IL-2 AU-007 Can Bind | | |
|---|---|---|---|
| AU-007 Dose | at Steady State IU/ml (nM) | | |
| (mg/kg) | | Day 1 | Day 14 |

| | | | | |
|---|---|---|---|---|
| 0.5 | 57.4K | (234) | 32.7K | (133) |
| 1.5 | 180K | (733) | 106K | (431) |
| 4.5 | 574K | (2.3K) | 295K | (1.2K) |
| 9 | 1.1M | (4.5K) | 656K | (2.7K) |
| 12 | 1.5M | (6.1K) | 820K | (3.3K) |

### Example 12

### IL-2 (Proleukin® (Aldesleukin) Dose Evaluation

Objective: To evaluate IL-2 to determine dose range.

Methods: Review and evaluate IL-2 dosing in known NIH clinical trials.

Results: A review of NIH clinical trials showed for treatment of Renal Cell Cancer (RCC) at least three dose regimens were assessed (high dose 720,00 IU/kg, intravenous (IV) administration; low dose 72,00 IU/kg, intravenous (IV) administration; and low dose 250,00-125,00 IU/kg, subcutaneous (Sub-Q) administration). Results demonstrated that Sub-Q low dose IL-2 administration was very safely administered as outpatient therapy and resulted in a low rate of severe toxicity, which may be an advantage and improves convenience, reduces resources and costs (Yang (2003) J Clin Oncol. 21(16): 3127-3132).

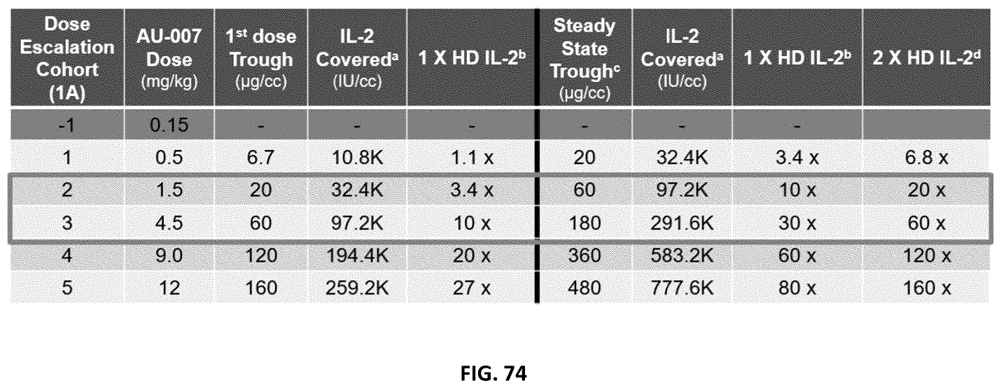

Based on the results presented in this Examples section, a human PK modeling chart of Q2W AU-007 dosing and IL-2 coverage was developed (FIG. 74). (a) is the amount of IL-2 (in IU/cc) that could bind to AU-007 on a straight one to one molecule stoichiometric basis; (b) is the amount of high-dose (HD) IL-2 (600,000 IU/kg) based on 80 kg patient (5 L blood volume) that could bind to AU-007 on a straight one to one molecule stoichiometric basis; (c) indicates the trough between 6th and 7th dose at between 12-14 weeks; and (d) presents the amount of HD IL-2 (600,000 IU/kg) based on 80 kg patient (5 L blood volume) that could bind to AU-007 considering 2 IL-2 molecules to 1 AU-007 molecule. Subcutaneous routes of administration for IL-2 are advantageous as IL-2 is preferentially absorbed into the central lymph compartment, shows a safer toxicity profile, and there is an ease of administration with less time spent in the clinic.

IL-2 doses of 15K, 45K, 135, and 270K IU/kg will be evaluated in dose escalation studies, with the knowledge that AU-007 bound IL-2 is redirected away from trimeric IL-2 receptors to dimeric IL-2 receptors on Teff and NK cells. A Treg “sink” for IL-2 is not available in the presence of AU-007 bound IL-2, which was the reason for high-dose IL-2. Thus, use of AU-007 reduces the need for high-dose IL-2. AU-007 bound IL-2 is anticipated to prolong the short Proleukin® (aldesleukin) T1/2 of 85 minutes closer to a T1/2 of an IgG monoclonal molecule.

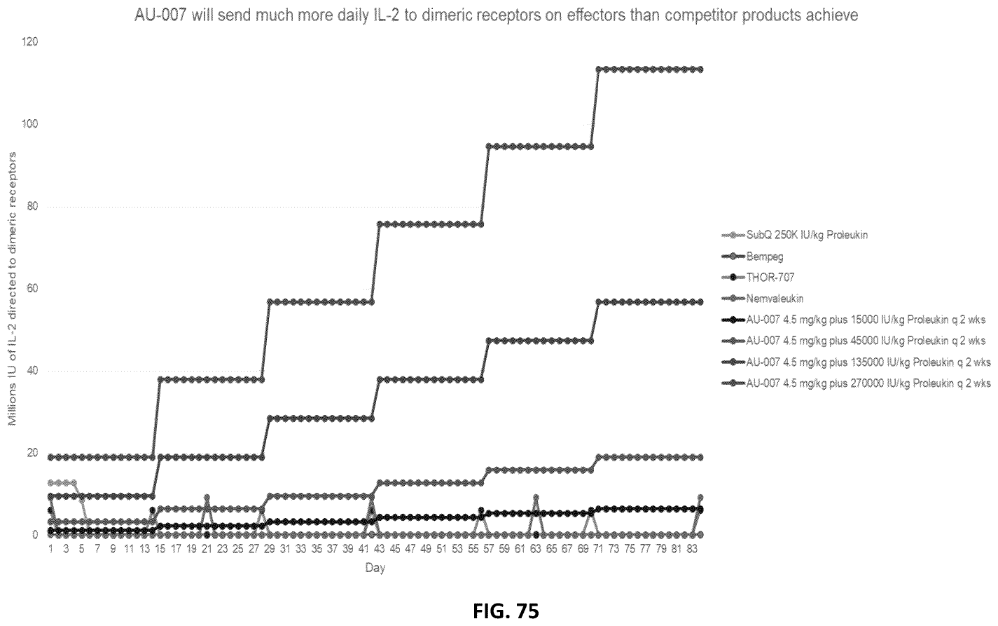

Future studies to assess pharmacodynamics (PD) of AU-007+/− added IL-2 include analysis of circulating lymphocytes and circulating CD8+ T cells, concentration of soluble CD25, and concentration of interferon-γ. AU-007 and subcutaneous administration of IL-2 in Q2W is projected to send much more daily IL-2 to dimeric receptors on T effectors and NK cells than competing products can achieve due to accumulation and redirection of IL-2 in complex with antibody (FIG. 75). It is expected that AU-007 will send much more daily IL-2 to dimeric receptors on effectors than previous antibody and small molecule “anti-IL-2” products have achieved. The graph presented in FIG. 75 does not account for endogenous IL-2 accumulation.

### Example 13

### Phase 1 Dose Escalation Portion of Ongoing Phase 1/2 Clinical Trial of AU-007

Objective: Continued evaluation of anti-IL2 antibody clone BDG17.069 (AU-007) in subjects with cancer.

### Study Design—Updates

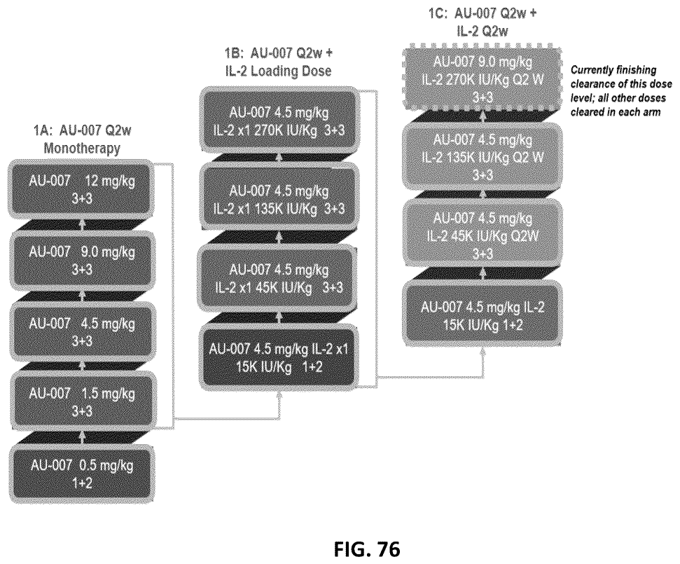

This example provides updated data of the single dose (1B) and dose escalation (1C) studies of the Clinical Phase 1/2 Trial of the anti-IL-2 antibody in combination with IL-2, described in Example 9. During the trial, pharmacodynamic markers in the periphery and tumor biopsies were/are being collected to investigate the activity of AU-007 or AU-007⁺ IL-2. Provided here is data from the ongoing studies performed, evaluating anti-IL-2 antibody clone BDG17.069 (AU-007) with both BDG17.069 and aldesleukin (FIG. 76; Arms 1B and 1C).

Recombinant human IL-2 (aldesleukin) was/and is being administered subcutaneously, at much lower doses and much less frequently than the approved regimen (approved regimen: 600,000 IU/kg every 8 hours for up to 14 administrations) of intravenously administered aldesleukin. In these studies, human IL-2 (aldesleukin) was/and is being administered subcutaneously either as a single dose or every two weeks, at the doses shown in FIG. 76, i.e., at 15K IU/kg, 45K IU/kg, 135K IU/kg, or 270K IU/kg,

### Results:

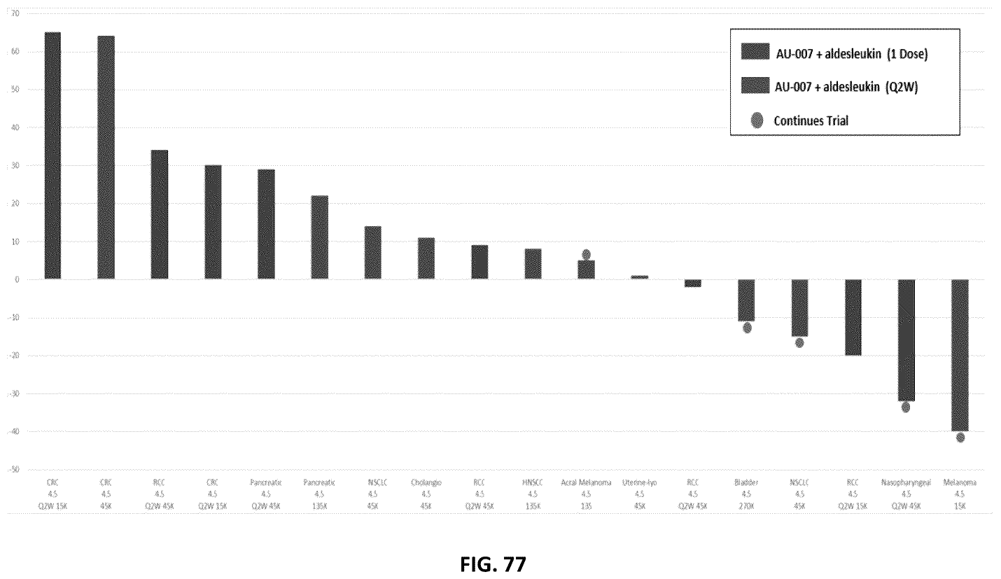

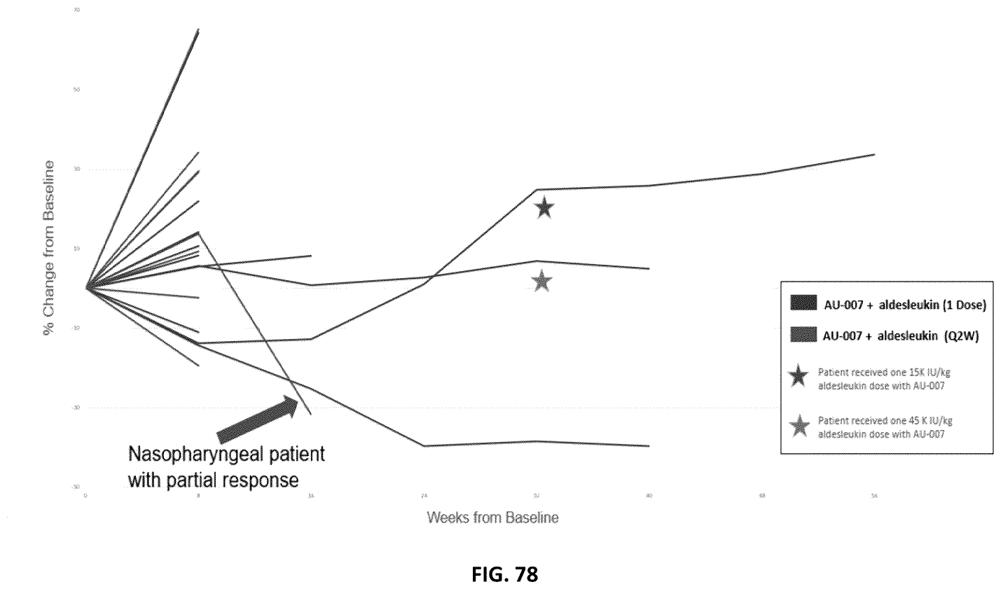

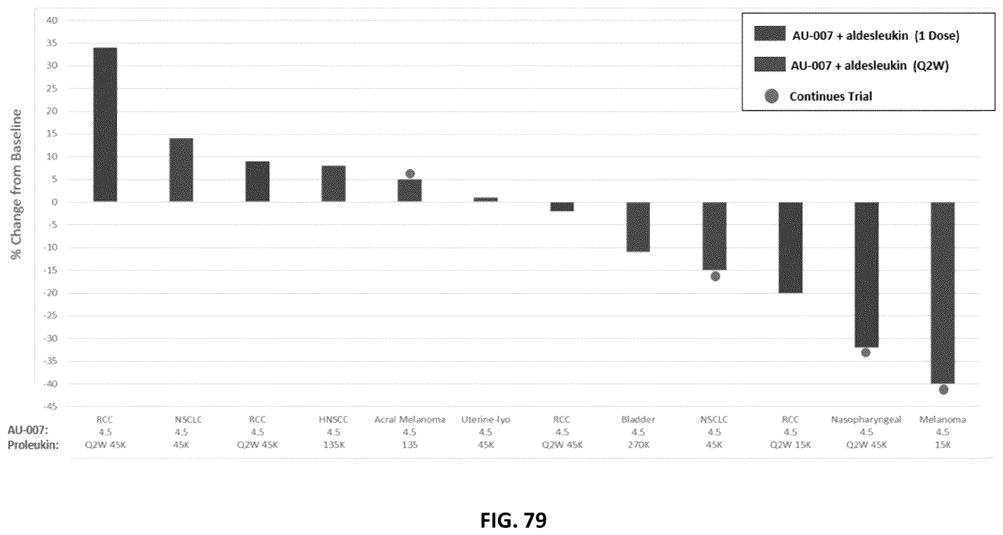

FIG. 77 presents an updated AU-007⁺ Aldesleukin Waterfall Plot: Best % Change vs. Baseline. FIG. 78 presents an updated AU-007⁺ Aldesleukin Spider Plot: % Change vs. Baseline Over Time. FIG. 79 presents an updated AU-007⁺ Aldesleukin Waterfall Plot: Best % Change in non-G.I. Immune Sensitive Tumors (RCC, NSCLC, HNSCC, Acral Melanoma, Uterine-lyo (Leiomyosarcoma in the uterus), Bladder, Nasopharyngeal, and melanoma).

As can be observed in FIGS. 77-79, significant anti-tumor activity is observed with AU-007 in combination with aldesleukin, in patients suffering from bladder cancer (AU-007+ aldesleukin (1 Dose), NSCLC (AU-007+ aldesleukin (1 Dose), melanoma (AU-007+ aldesleukin (1 Dose), RCC (AU-007+ aldesleukin (Q2W)), and nasopharyngeal cancer (AU-007+ aldesleukin (Q2W)). The subject suffering from RCC patient had to terminate the study secondary to complications due to the underlying cancer, necessitating rapid surgical intervention due to a lesion that was threatening to impinge on his spinal cord. At the time of his having to leave the study, there was ˜20% tumor shrinkage. In particular, FIG. 78 shows a greater than 30% reduction of cancer (reduction of tumor size) in a nasopharyngeal patient, while FIGS. 77 and 79 show that the patient suffering from melanoma has around a 40% tumor shrinkage.

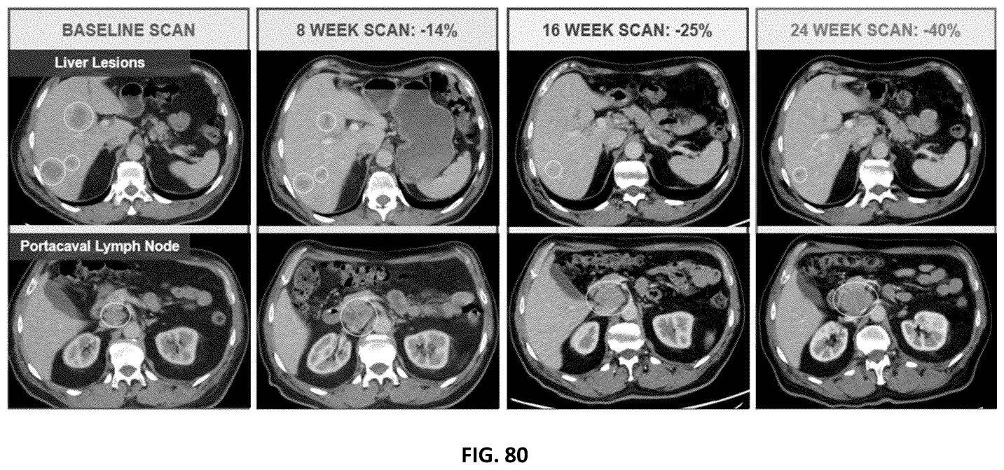

Computed tomography scans of tumor lesions at baseline 24-weeks show a 40% shrinkage in the target lesions of a melanoma patient whose tumors progressed through prior anti-PD-1+ CTLA4 therapy (FIG. 80; See, Example 9 and FIGS. 46A-46C, update for Patient AU06-0014). The patient initially presented as a 62-year-old man with progression in the liver, December 2022. By February 2023, he entered the study wherein an initial Q2W AU-007 (4.5 mg/kg) dose+one (and only) 15K IU/kg aldesleukin dose were administered. Initial portacaval lymph node (LN) growth with necrotic center was followed by stabilization, which may represent pseudoprogression.

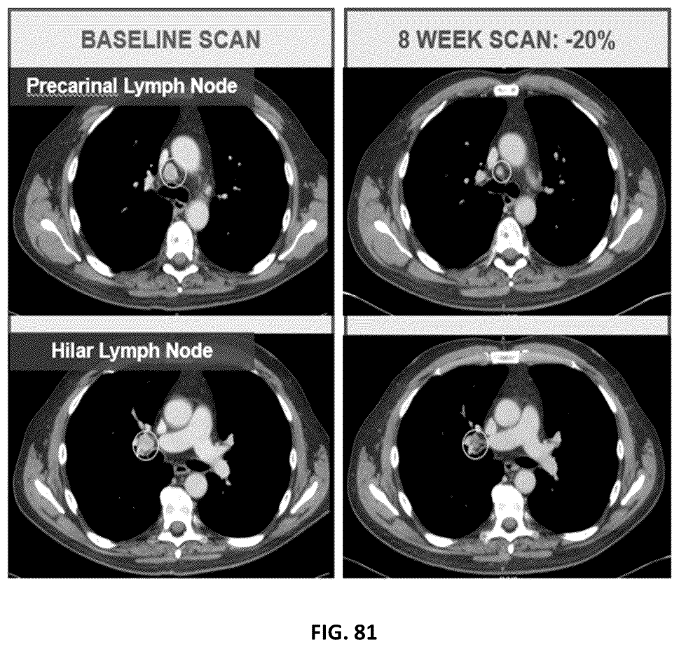

Scans of tumor lesions at based line and 20-weeks in an RCC patient show 20% shrinkage in the first 8 weeks in the target lesions. The RCC Patient's tumors has previously progressed through prior Anti-PD-1 therapy (FIG. 81; See, Example 9, update for RCC Patient). The patient initially presented as a 68-year-old man who had progressed on anti-PD-1 treatment June 2022. July 2023, he entered the study with initial AU-007 (4.5 mg/kg)+15K IU/kg Q2W aldesleukin doses. The primary renal cancer remains in situ and was stable.

Summary With the continued positive trends, these studies are continuing. Anti-tumor activity has been observed in heavily pre-treated patients with several types of cancer. Further development of AU-007 therapy in combination with aldesleukin (IL-2), and Phase 2 expansion cohorts are planned at least in patients suffering from melanoma, RCC, and NSCLC.

What is claimed is:

1. A method of treating a solid cancer in a subject comprising a step of administering to the subject a composition comprising an anti-IL-2 antibody, said IL-2 antibody comprising a heavy chain variable region (VH) and a light chain variable region (VL), wherein said VH comprises heavy chain complementarity determining regions (HCDRs) HCDR1, HCDR2 and HCDR3, said VL comprises light chain complementarity determining regions (LCDRs) LCDR1, LCDR2 and LCDR3, wherein said CDRs have the amino acid sequences of
HCDR1 comprises the amino acid sequence of SEQ ID NO:62, HCDR2 comprises the amino acid sequence of SEQ ID NO:63, HCDR3 comprises the amino acid sequence of SEQ ID NO:64, LCDR1 comprises the amino acid sequence of SEQ ID NO:65, LCDR2 comprises the amino acid sequence of DAS, and LCDR3 comprises the amino acid sequence of SEQ ID NO:67,
wherein the dose of said anti-IL-2 antibody is between about 0.5 mg/kg-12 mg/kg of said subject, and wherein said antibody promotes differential growth of subsets of immune cells and decreases undesirable effects caused by IL-2, thereby treating said cancer in said subject.

2. The method of claim 1, wherein multiple doses of said composition comprising anti-IL2 antibody are administered.

3. The method of claim 1, wherein said method further comprises administering at least a single low dose of IL-2, wherein said low dose of IL-2 comprises between about 15×10³ IU/Kg-500×10³ IU/Kg of said subject.

4. The method of claim 3, wherein said administration of IL-2 comprises subcutaneous administration.

5. The method of claim 3, wherein said IL-2 is administered prior to, concurrent with, or following the administration of said anti-IL-2 antibody.

6. The method according to claim 3, wherein said IL-2 is administered as multiple doses.

7. The method of claim 6, wherein said multiple doses of IL-2 are administered prior to, concurrent with, or following the administration of said anti-IL-2 antibody, or any combination thereof.

8. The method of claim 3, wherein said method further comprises administering a checkpoint inhibitor.

9. The method of claim 8, wherein said checkpoint comprises PD-L1, PD-1, CTLA-4, TIGIT, TIM-3, B7-H3, CD73, LAG3, CD27, CD70, 4-1BB, GITR, OX40, SIRP-alpha (CD47), CD39, ILDR2, VISTA, BTLA, or VTCN-1.

10. The method according to claim 1, wherein said solid cancer comprises an unresectable locally advanced or metastatic cancer.

11. The method according to claim 10, wherein said unresectable locally advanced or metastatic cancer comprises a melanoma, a renal cell carcinoma (RCC), a non-small cell lung cancer (NSCLC), a head and neck squamous cell carcinoma (HNSCC), gastric or gastro-esophageal cancer, esophageal squamous cell carcinoma, cutaneous squamous cell carcinoma (cSCC), pancreatic adenocarcinoma, cholangiocarcinoma (bile duct cancer), hepato-cellular carcinoma (HCC), colorectal cancer (CRC), epithelial ovarian cancer, cervical cancer, endometrial cancer, thyroid cancer having follicular or papillary histology, urothelial cancer, bladder cancer, uterine cancer, gallbladder cancer, or Merkel cell carcinoma.

12. The method according to claim 1, wherein said solid cancer comprises a melanoma, a metastatic melanoma, a primary melanoma and metastatic melanoma, a renal cell carcinoma (RCC), a non-small cell lung cancer (NSCLC), a bladder cancer, a head and neck cancer, a head and neck squamous cell carcinoma (HNSCC), anasopharyngeal carcinoma, a urothelial cancer, an adrenal cortical carcinoma, a clear cell renal cell carcinoma (ccRCC), a triple-negative breast cancer, a gastric or gastro-esophageal cancer, an esophageal squamous cell carcinoma, a cutaneous squamous cell carcinoma (cSCC), a pancreatic cancer, a pancreatic adenocarcinoma, a cholangiocarcinoma (bile duct cancer), a hepato-cellular carcinoma (HCC), a colorectal cancer (CRC), an epithelial ovarian cancer, a cervical cancer, an endometrial cancer, a thyroid cancer (follicular or papillary histology), a lung cancer, a uterine cancer, a gallbladder cancer, or a Merkel cell carcinoma, or any tumors that are microsatellite instabilities (MSI)-high tumors.

13. The method according to claim 1, wherein said method comprises a first line treatment, a second line treatment, or a third line treatment, or a combination thereof.

14. The method according to claim 1, wherein treating said subject reduces the size of the tumor, inhibits or reduces growth of the tumor, or inhibits or reduces metastases of said tumor, or any combination thereof.

15. The method according to claim 1, wherein the VH and VL have the amino acid sequences of
(a) the VH comprises the amino acid sequence of SEQ ID NO:26, the VL comprises the amino acid sequence of SEQ ID NO:27;
(b) the VH comprises the amino acid sequence of SEQ ID NO:20, the VL comprises the amino acid sequence of SEQ ID NO:21;
(c) the VH comprises the amino acid sequence of SEQ ID NO:22, the VL comprises the amino acid sequence of SEQ ID NO:23;
(d) the VH comprises the amino acid sequence of SEQ ID NO:24, the VL comprises the amino acid sequence of SEQ ID NO:25; or
(e) the VH comprises the amino acid sequence of SEQ ID NO:36, the VL comprises the amino acid sequence of SEQ ID NO:37.

16. The method according to claim 1, wherein the antibody comprises an IgG, IgA, IgM, IgE, IgD, a Fv, a scFv, a Fab, a F(ab′)₂, a minibody, a diabody, or a triabody.

17. The method according to claim 1, wherein said antibody comprises a heavy chain comprising a mutation that reduces binding to an Fcγ receptor.

18. The method according to claim 17, wherein said mutation comprises L234A, L235A mutations.

19. The method according to claim 1, wherein the amino acid sequence of the full length heavy chain is set forth in SEQ ID NO: 72 and the amnio acid sequence of the full length light chain is set forth in SEQ ID NO: 73.

20. A method of treating cancer in a subject comprising a step of administering to the subject an anti-IL-2 antibody and a IL-2, said IL-2 antibody comprising a heavy chain variable region (VH) and a light chain variable region (VL), wherein said VH comprises heavy chain complementarity determining regions (HCDRs) HCDR1, HCDR2 and HCDR3, said VL comprises light chain complementarity determining regions (LCDRs) LCDR1, LCDR2 and LCDR3, wherein said CDRs have the amino acid sequences of
HCDR1 comprises the amino acid sequence of SEQ ID NO:62, HCDR2 comprises the amino acid sequence of SEQ ID NO:63, HCDR3 comprises the amino acid sequence of SEQ ID NO:64, LCDR1 comprises the amino acid sequence of SEQ ID NO:65, LCDR2 comprises the amino acid sequence of DAS, and LCDR3 comprises the amino acid sequence of SEQ ID NO:67,
wherein said IL-2 is administered by subcutaneous injection, wherein the dose of said anti-IL-2 antibody is between about 0.5 mg/kg-12 mg/kg of said subject, and wherein said antibody promotes differential growth of subsets of immune cells and decreases undesirable effects caused by IL-2, thereby treating said cancer in said subject.

21. A method of treating solid cancer in a subject comprising a step of administering to the subject an anti-IL-2 antibody, an IL-2, and a checkpoint inhibitor, said IL-2 antibody comprising a heavy chain variable region (VH) and a light chain variable region (VL), wherein said VH comprises heavy chain complementarity determining regions (HCDRs) HCDR1, HCDR2 and HCDR3, said VL comprises light chain complementarity determining regions (LCDRs) LCDR1, LCDR2 and LCDR3, wherein said CDRs have the amino acid sequences of
HCDR1 comprises the amino acid sequence of SEQ ID NO:62, HCDR2 comprises the amino acid sequence of SEQ ID NO:63, HCDR3 comprises the amino acid sequence of SEQ ID NO:64, LCDR1 comprises the amino acid sequence of SEQ ID NO:65, LCDR2 comprises the amino acid sequence of DAS, LCDR3 comprises the amino acid sequence of SEQ ID NO:67,
wherein said IL-2 is administered by subcutaneous injection, wherein the dose of said anti-IL-2 antibody is between about 0.5 mg/kg-12 mg/kg of said subject, wherein said antibody promotes differential growth of subsets of immune cells and decreases undesirable effects caused by IL-2, and wherein said checkpoint inhibitor comprises PD-L1, PD-1, CTLA-4, TIGIT, TIM-3, B7-H3, CD73, LAG3, CD27, CD70, 4-1BB, GITR, OX40, SIRP-alpha (CD47), CD39, ILDR2, VISTA, BTLA, or VTCN-1, thereby treating said cancer in said subject.

22. The method according to claim 21, wherein said checkpoint inhibitor comprises a PD-L1 checkpoint inhibitor.


SEQUENCE LISTING (73 sequences)

SEQ ID NO: 1 - DNA, 747 bp, synthetic construct
  misc_feature 1..747: note = scFv clone 17.021
  source 1..747: mol_type = other DNA
gagattcagctgcagcagagcggtccagaactgcgtcgtccaggctcctccgttaaactgagctgcaaagcctctggttataacatcaccgataacctgattcattgggttcgtcaccgtccagaacacggtctggaatggatcggctggattgatccagaggacggtgaaacccgttacgcccaaaaattccagtctaaagcaaccctgactgccgacactagctccaacgccgcttacatgcagctgagcagcctgacccctgaggataccgccacctatttctgcgcacgttctctggattccacctatatttacccattcgcatactggggccaaggcacccttgtcaccgtttcctccggcggtggtggtagcggaggcggaggatcaggtggaggcggcagtgacattgttatgactcagtccccattctccctggccgtttctgagggtgaaatggtaaccattaactgcaaatcatcccagtccctgctgcggtctggcaaccagaaaaactatctggcctggtaccagcagaaaccaggtcagtccccaaaactgctgatctactacgcatctactggccagtctggtgtcccagaccgtttcattggctctggctctggtaccgatttcaccctgaccatcagcgatgttcaagctgaagacctggccgattactattgtctgcagcactaccagagcccaccaactttcggtgcaggcaccaaactggaactgaaa

SEQ ID NO: 2 - DNA, 747 bp, synthetic construct
  misc_feature 1..747: note = scFv clone 17.022
  source 1..747: mol_type = other DNA
gagattcagctgcagcagagcggtccagaactgcgtcgtccaggctcctccgttaaactgagctgcaaagcctctggttataacatcaccgattacctgattcattgggttcgtcaccgtccagaacacggtctggaatggatcggctggattgatccagaggacggtgaaacccgttacgcccaaaaattccagtctaaagcaaccctgactgccgacactagctccaacgccgcttacatgcagctgagcagcctgacccctgaggataccgccacctatttctgcgcacgttctctggattccacctggatttacccattcgcatactggggccaaggcacccttgtcaccgtttcctccggcggtggtggtagcggaggcggaggatcaggtggaggcggcagtgacattgttatgactcagtccccattctccctggccgtttctgagggtgaaatggtaaccattaactgcaaatcatcccagtccctgctgcggtctggcaaccagaaaaactatctggcctggtaccagcagaaaccaggtcagtccccaaaactgctgatctactacgcatctactggccagtctggtgtcccagaccgtttcattggctctggctctggtaccgatttcaccctgaccatcagcgatgttcaagctgaagacctggccgattactattgtctgcagcactacattaccccaccaactttcggtgcaggcaccaaactggaactgaaa

SEQ ID NO: 3 - DNA, 747 bp, synthetic construct
  misc_feature 1..747: note = scFv clone 17.023
  source 1..747: mol_type = other DNA
gagattcagctgcagcagagcggtccagaactgcgtcgtccaggctcctccgttaaactgagctgcaaagcctctggttataacatcaccgattacctgattcattgggttcgtcaccgtccagaacacggtctggaatggatcggctggattgatccagaggacggtgaaacccgttacgcccaaaaattccagtctaaagcaaccctgactgccgacactagctccaacgccgcttacatgcagctgagcagcctgacccctgaggataccgccacctatttctgcgcacgtcagctggattccacctatatttacccattcgcatactggggccaaggcacccttgtcaccgtttcctccggcggtggtggtagcggaggcggaggatcaggtggaggcggcagtgacattgttatgactcagtccccattctccctggccgtttctgagggtgaaatggtaaccattaactgcaaatcatcccagtccctgctgcggtctggcaaccagaaaaactatctggcctggtaccagcagaaaccaggtcagtccccaaaactgctgatctactacgcatctactggccagtctggtgtcccagaccgtttcattggctctggctctggtaccgatttcaccctgaccatcagcgatgttcaagctgaagacctggccgattactattgtctgcagcactacattaccccaccaactttcggtgcaggcaccaaactggaactgaaa

SEQ ID NO: 4 - DNA, 747 bp, synthetic construct
  misc_feature 1..747: note = scFv clone 17.030
  source 1..747: mol_type = other DNA
gagattcagctgcagcagagcggtccagaactgcgtcgtccaggctcctccgttaaactgagctgcaaagcctctggttataacatcaccgattacctgattcattgggttcgtcaccgtccagaacacggtctggaatggatcggctggattgatccagaggacggtgaaacccgttacgcccaaaaattccagtctaaagcaaccctgactgccgacactagctccaacgccgcttacatgcagctgagcagcctgacccctgaggataccgccacctatttctgcgcacgttctctggattccacctatgactacccattcgcatactggggccaaggcacccttgtcaccgtttcctccggcggtggtggtagcggaggcggaggatcaggtggaggcggcagtgacattgttatgactcagtccccattctccctggccgtttctgagggtgaaatggtaaccattaactgcaaatcatcccagtccctgctgcggtctggcaaccagaaaaactatctggcctggtaccagcagaaaccaggtcagtccccaaaactgctgatctactacgcatctactggccagtctggtgtcccagaccgtttcattggctctggctctggtaccgatttcaccctgaccatcagcgatgttcaagctgaagacctggccgattactattgtctgcagcactacatttcgccaccaactttcggtgcaggcaccaaactggaactgaaa

SEQ ID NO: 5 - DNA, 747 bp, synthetic construct
  misc_feature 1..747: note = scFv clone 17.035
  source 1..747: mol_type = other DNA
gagattcagctgcagcagagcggtccagaactgcgtcgtccaggctcctccgttaaactgagctgcaaagcctctggttataacatcaccgattacctgattcattgggttcgtcaccgtccagaacacggtctggaatggatcggctggattgatccagaggacggtgaaacccgttacgcccaaaaattccagtctaaagcaaccctgactgccgacactagctccaacgccgcttacatgcagctgagcagcctgacccctgaggataccgccacctatttctgcgcacgttctctggattccacctataactacccattcgcatactggggccaaggcacccttgtcaccgtttcctccggcggtggtggtagcggaggcggaggatcaggtggaggcggcagtgacattgttatgactcagtccccattctccctggccgtttctgagggtgaaatggtaaccattaactgcaaatcatcccagtccctgctgcggtctggcaaccagaaaaactatctggcctggtaccagcagaaaccaggtcagtccccaaaactgctgatctactacgcatctactggccagtctggtgtcccagaccgtttcattggctctggctctggtaccgatttcaccctgaccatcagcgatgttcaagctgaagacctggccgattactattgtctgcagcactggattagcccaccaactttcggtgcaggcaccaaactggaactgaaa

SEQ ID NO: 6 - protein, 121 aa, synthetic construct
  REGION 1..121: note = Heavy chain variable region of JES6.1
EIQLQQSGPELRRPGSSVKLSCKASGYNITDYLIHWVRHRPEHGLEWIGWIDPEDGETRYAQKFQSKATLTADTSSNAAYMQLSSLTPEDTATYFCARSLDSTYIYPFAYWGQGTLVTVSS

SEQ ID NO: 7 - protein, 113 aa, synthetic construct
  REGION 1..113: note = Light chain variable region of JES6.1
DIVMTQSPFSLAVSEGEMVTINCKSSQSLLSSGNQKNYLAWYQQKPGQSPKLLIYYASTGQSGVPDRFIGSGSGTDFTLTISDVQAEDLADYYCLQHYISPPTFGAGTKLELK

SEQ ID NO: 8 - protein, 121 aa, synthetic construct; identical to SEQ ID NO: 6
  REGION 1..121: note = Heavy chain variable region of JES6.1RMC

SEQ ID NO: 9 - protein, 113 aa, synthetic construct; identical to SEQ ID NO: 7
  REGION 1..113: note = Light chain variable region of JES6.1RMC

SEQ ID NO: 10 - protein, 121 aa, synthetic construct
  REGION 1..121: note = Heavy chain variable region of clone 17.021
EIQLQQSGPELRRPGSSVKLSCKASGYNITDNLIHWVRHRPEHGLEWIGWIDPEDGETRYAQKFQSKATLTADTSSNAAYMQLSSLTPEDTATYFCARSLDSTYIYPFAYWGQGTLVTVSS

SEQ ID NO: 11 - protein, 113 aa, synthetic construct
  REGION 1..113: note = Light chain variable region of clone 17.021
DIVMTQSPFSLAVSEGEMVTINCKSSQSLLRSGNQKNYLAWYQQKPGQSPKLLIYYASTGQSGVPDRFIGSGSGTDFTLTISDVQAEDLADYYCLQHYQSPPTFGAGTKLELK

SEQ ID NO: 12 - protein, 121 aa, synthetic construct
  REGION 1..121: note = Heavy chain variable region of clone 17.022
EIQLQQSGPELRRPGSSVKLSCKASGYNITDYLIHWVRHRPEHGLEWIGWIDPEDGETRYAQKFQSKATLTADTSSNAAYMQLSSLTPEDTATYFCARSLDSTWIYPFAYWGQGTLVTVSS

SEQ ID NO: 13 - protein, 113 aa, synthetic construct
  REGION 1..113: note = Light chain variable region of clone 17.022
DIVMTQSPFSLAVSEGEMVTINCKSSQSLLRSGNQKNYLAWYQQKPGQSPKLLIYYASTGQSGVPDRFIGSGSGTDFTLTISDVQAEDLADYYCLQHYITPPTFGAGTKLELK

SEQ ID NO: 14 - protein, 121 aa, synthetic construct
  REGION 1..121: note = Heavy chain variable region of clone 17.023
EIQLQQSGPELRRPGSSVKLSCKASGYNITDYLIHWVRHRPEHGLEWIGWIDPEDGETRYAQKFQSKATLTADTSSNAAYMQLSSLTPEDTATYFCARQLDSTYIYPFAYWGQGTLVTVSS

SEQ ID NO: 15 - protein, 113 aa, synthetic construct; identical to SEQ ID NO: 13
  REGION 1..113: note = Light chain variable region of clone 17.023

SEQ ID NO: 16 - protein, 121 aa, synthetic construct
  REGION 1..121: note = Heavy chain variable region of clone 17.030
EIQLQQSGPELRRPGSSVKLSCKASGYNITDYLIHWVRHRPEHGLEWIGWIDPEDGETRYAQKFQSKATLTADTSSNAAYMQLSSLTPEDTATYFCARSLDSTYDYPFAYWGQGTLVTVSS

SEQ ID NO: 17 - protein, 113 aa, synthetic construct
  REGION 1..113: note = Light chain variable region of clone 17.030
DIVMTQSPFSLAVSEGEMVTINCKSSQSLLRSGNQKNYLAWYQQKPGQSPKLLIYYASTGQSGVPDRFIGSGSGTDFTLTISDVQAEDLADYYCLQHYISPPTFGAGTKLELK

SEQ ID NO: 18 - protein, 121 aa, synthetic construct
  REGION 1..121: note = Heavy chain variable region of clone 17.035
EIQLQQSGPELRRPGSSVKLSCKASGYNITDYLIHWVRHRPEHGLEWIGWIDPEDGETRYAQKFQSKATLTADTSSNAAYMQLSSLTPEDTATYFCARSLDSTYNYPFAYWGQGTLVTVSS

SEQ ID NO: 19 - protein, 113 aa, synthetic construct
  REGION 1..113: note = Light chain variable region of clone 17.035
DIVMTQSPFSLAVSEGEMVTINCKSSQSLLRSGNQKNYLAWYQQKPGQSPKLLIYYASTGQSGVPDRFIGSGSGTDFTLTISDVQAEDLADYYCLQHWISPPTFGAGTKLELK

SEQ ID NO: 20 - protein, 121 aa, synthetic construct
  REGION 1..121: note = Heavy chain variable region of clone 17.038
QVQLVQSGAEVKKPGSSVKVSCKASGYSITDSLIHWVRQAPGQGLEWMGWIDPEDGEINYAQKFQGRVTLTADTSTSTAYMELSSLRSEDTAVYYCARSLDSYPIDPFAYWGQGTLVTVSS

SEQ ID NO: 21 - protein, 113 aa, synthetic construct
  REGION 1..113: note = Light chain variable region of clone 17.038
DIVMTQSPDSLAVSLGERATINCKSSQSLLRNGNQQNYLAWYQQKPGQPPKLLIYYASTGQSGVPDRFSGSGSGTDFTLTISSLQAEDVAVYYCQQHYVTPPTFGAGTKVEIK

SEQ ID NO: 22 - protein, 121 aa, synthetic construct
  REGION 1..121: note = Heavy chain variable region of clone 17.043
QVQLVQSGAEVKKPGSSVKVSCKASGYSITDYLIHWVRQAPGQGLEWMGWIDPEDGVTAYAQKFQGRVTLTADTSTSTAYMELSSLRSEDTAVYYCARSLDYDPIYPFAYWGQGTLVTVSS

SEQ ID NO: 23 - protein, 113 aa, synthetic construct
  REGION 1..113: note = Light chain variable region of clone 17.043
DIVMTQSPDSLAVSLGERATINCKSSQSLLRNGNQKNYLAWYQQKPGQPPKLLIYYASTGQSGVPDRFSGSGSGTDFTLTISSLQAEDVAVYYCQQHYVTPPTFGAGTKVEIK

SEQ ID NO: 24 - protein, 121 aa, synthetic construct
  REGION 1..121: note = Heavy chain variable region of clone 17.053
QVQLVQSGAEVKKPGSSVKVSCKASGYSITDYLIHWVRQAPGQGLEWMGWIDPEDGETNYAQKFQGRVTLTADTSTSTAYMELSSLRSEDTAVYYCARSLDYDPIYPFAYWGQGTLVTVSS

SEQ ID NO: 25 - protein, 113 aa, synthetic construct
  REGION 1..113: note = Light chain variable region of clone 17.053
DIVMTQSPDSLAVSLGERATINCKSSQSLLRNGNQKNYLAWYQQKPGQPPKLLIYYASTGQSGVPDRFSGSGSGTDFTLTISSLQAEDVAVYYCQQHYITPPTFGAGTKVEIK

SEQ ID NO: 26 - protein, 121 aa, synthetic construct
  REGION 1..121: note = Heavy chain variable region of clone 17.054
QVQLVQSGAEVKKPGSSVKVSCKASGYSITDDLIHWVRQAPGQGLEWMGWIDPEDGETNYAQKFQGRVTLTADTSTSTAYMELSSLRSEDTAVYYCARSLDSTWIYPFAYWGQGTLVTVSS

SEQ ID NO: 27 - protein, 113 aa, synthetic construct
  REGION 1..113: note = Light chain variable region of clone 17.054
DIVMTQSPDSLAVSLGERATINCKSSQSLLRRGNQKNHLAWYQQKPGQPPKLLIYDASTGQSGVPDRFSGSGSGTDFTLTISSLQAEDVAVYYCLQSYITPPTFGAGTKVEIK

SEQ ID NO: 28 - DNA, 747 bp, synthetic construct
  misc_feature 1..747: note = scFv clone 17.002
  source 1..747: mol_type = other DNA
gagattcagctgcagcagagcggtccagaactgcgtcgtccaggctcctccgttaaactgagctgcaaagcctctggttataacatcaccgattacctgattcattgggttcgtcaccgtccagaacacggtctggaatggatcggctggattgatccagaggacggtgaaacccgttacgcccaaaaattccagtctaaagcaaccctgactgccgacactagctccaacgccgcttacatgcagctgagcagcctgacccctgaggataccgccacctatttctgcgcacgttctctggattccaccccgatttacccattcgcatactggggccaaggcacccttgtcaccgtttcctccggcggtggtggtagcggaggcggaggatcaggtggaggcggcagtgacattgttatgactcagtccccattctccctggccgtttctgagggtgaaatggtaaccattaactgcaaatcatcccagtccctgctgcggtctggcaaccagaaaaactatctggcctggtaccagcagaaaccaggtcagtccccaaaactgctgatctactacgcatctactggccagtctggtgtcccagaccgtttcattggctctggctctggtaccgatttcaccctgaccatcagcgatgttcaagctgaagacctggccgattactattgtctgcagcactacattacgccaccaactttcggtgcaggcaccaaactggaactgaaa

SEQ ID NO: 29 - protein, 121 aa, synthetic construct
  REGION 1..121: note = Heavy chain variable region of clone 17.002
EIQLQQSGPELRRPGSSVKLSCKASGYNITDYLIHWVRHRPEHGLEWIGWIDPEDGETRYAQKFQSKATLTADTSSNAAYMQLSSLTPEDTATYFCARSLDSTPIYPFAYWGQGTLVTVSS

SEQ ID NO: 30 - protein, 113 aa, synthetic construct; identical to SEQ ID NO: 13
  REGION 1..113: note = Light chain variable region of clone 17.002

SEQ ID NO: 31 - DNA, 747 bp, synthetic construct
  misc_feature 1..747: note = scFv clone 17.014
  source 1..747: mol_type = other DNA
caggtccaactggtgcagtccggtgccgaagttaaaaaacctgggtcttccgttaaagtttcttgcaaagcctctggctacagcatcaccgattatctgattcactgggtccgtcaggctccaggtcaaggtctggaatggatgggttggatcgatccagaagacggtgaaaccaactatgcccagaaattccagggtcgtgtaaccctgaccgccgacacctccacctctaccgcctacatggagttaagtagcctgcgttcagaggataccgcagtgtactactgcgctcgttcactggactccaccccaatctacccattcgcatactggggtcagggcaccctggtaaccgttagtagcggcggtggtggtagcggaggcggaggatcaggtggaggcggcagtgacatcgtgatgacccagtctcctgactccttggccgtctctctgggcgaacgtgcaactatcaactgcaaatccagccagagcttactgcgttctggtaatcagaaaaactaccttgcatggtatcagcagaaaccaggtcagccaccaaaattactgatctattatgcatctaccggtcaaagcggtgtcccagatcgtttcagcggttccggctccggtactgacttcaccctgaccatctcttcccttcaggccgaagatgtggccgtgtattactgcctgcagcactacatcaccccacctactttcggtgctggtactaaagttgaaatcaaa

SEQ ID NO: 32 - DNA, 747 bp, synthetic construct
  misc_feature 1..747: note = scFv clone 17.038
  source 1..747: mol_type = other DNA
caggtccaactggtgcagtccggtgccgaagttaaaaaacctgggtcttccgttaaagtttcttgcaaagcctctggctacagcatcaccgattctctgattcactgggtccgtcaggctccaggtcaaggtctggaatggatgggttggatcgatccagaagacggtgaaattaactatgcccagaaattccagggtcgtgtaaccctgaccgccgacacctccacctctaccgcctacatggagttaagtagcctgcgttcagaggataccgcagtgtactactgcgctcgttcactggactcctacccaatcgatccattcgcatactggggtcagggcaccctggtaaccgttagtagcggcggtggtggtagcggaggcggaggatcaggtggaggcggcagtgacatcgtgatgacccagtctcctgactccttggccgtctctctgggcgaacgtgcaactatcaactgcaaatccagccagagcttactgcgtaatggtaatcagcaaaactaccttgcatggtatcagcagaaaccaggtcagccaccaaaattactgatctattatgcatctaccggtcaaagcggtgtcccagatcgtttcagcggttccggctccggtactgacttcaccctgaccatctcttcccttcaggccgaagatgtggccgtgtattactgccagcagcactacgtcaccccacctactttcggtgctggtactaaagttgaaatcaaa

SEQ ID NO: 33 - DNA, 747 bp, synthetic construct
  misc_feature 1..747: note = scFv clone 17.043
  source 1..747: mol_type = other DNA
caggtccaactggtgcagtccggtgccgaagttaaaaaacctgggtcttccgttaaagtttcttgcaaagcctctggctacagcatcaccgattatctgattcactgggtccgtcaggctccaggtcaaggtctggaatggatgggttggatcgatccagaagacggtgtgaccgcctatgcccagaaattccagggtcgtgtaaccctgaccgccgacacctccacctctaccgcctacatggagttaagtagcctgcgttcagaggataccgcagtgtactactgcgctcgttcactggactacgacccaatctacccattcgcatactggggtcagggcaccctggtaaccgttagtagcggcggtggtggtagcggaggcggaggatcaggtggaggcggcagtgacatcgtgatgacccagtctcctgactccttggccgtctctctgggcgaacgtgcaactatcaactgcaaatccagccagagcttactgcgtaacggtaatcagaaaaactatcttgcatggtatcagcagaaaccaggtcagccaccaaaattactgatctattatgcatctaccggtcaaagcggtgtcccagatcgtttcagcggttccggctccggtactgacttcaccctgaccatctcttcccttcaggccgaagatgtggccgtgtattactgccagcagcactacgtcaccccacctactttcggtgctggtactaaagttgaaatcaaa

SEQ ID NO: 34 - DNA, 747 bp, synthetic construct
  misc_feature 1..747: note = scFv clone 17.053
  misc_feature 726: note = n is a, c, g, or t
  source 1..747: mol_type = other DNA
caggtccaactggtgcagtccggtgccgaagttaaaaaacctgggtcttccgttaaagtttcttgcaaagcctctggctacagcatcaccgattatctgattcactgggtccgtcaggctccaggtcaaggtctggaatggatgggttggatcgatccagaagacggtgaaactaactatgcccagaaattccagggtcgtgtaaccctgaccgccgacacctccacctctaccgcctacatggagttaagtagcctgcgttcagaggataccgcagtgtactactgcgctcgttcactggactacgacccaatctacccattcgcatactggggtcagggcaccctggtaaccgttagtagcggcggtggtggtagcggaggcggaggatcaggtggaggcggcagtgacatcgtgatgacccagtctcctgactccttggccgtctctctgggcgaacgtgcaactatcaactgcaaatccagccagagcttactgcgtaacggtaatcagaaaaactaccttgcatggtatcagcagaaaccaggtcagccaccaaaattactgatctattatgcatctaccggtcaaagcggtgtcccagatcgtttcagcggttccggctccggtactgacttcaccctgaccatctcttcccttcaggccgaagatgtggccgtgtattactgccagcagcactacattaccccacctactttcggtgcnggtactaaagttgaaatcaaa

SEQ ID NO: 35 - DNA, 747 bp, synthetic construct
  misc_feature 1..747: note = scFv clone 17.054
  source 1..747: mol_type = other DNA
caggtccaactggtgcagtccggtgccgaagttaaaaaacctgggtcttccgttaaagtttcttgcaaagcctctggctacagcatcaccgatgacctgattcactgggtccgtcaggctccaggtcaaggtctggaatggatgggttggatcgatccagaagacggtgaaaccaactatgcccagaaattccagggtcgtgtaaccctgaccgccgacacctccacctctaccgcctacatggagttaagtagcctgcgttcagaggataccgcagtgtactactgcgctcgttcactggactccacctggatctacccattcgcatactggggtcagggcaccctggtaaccgttagtagcggcggtggtggtagcggaggcggaggatcaggtggaggcggcagtgacatcgtgatgacccagtctcctgactccttggccgtctctctgggcgaacgtgcaactatcaactgcaaatccagccagagcttactgcgtcgcggtaatcagaaaaaccaccttgcatggtatcagcagaaaccaggtcagccaccaaaattactgatctatgacgcatctaccggtcaaagcggtgtcccagatcgtttcagcggttccggctccggtactgacttcaccctgaccatctcttcccttcaggccgaagatgtggccgtgtattactgcctgcagagctacatcaccccacctactttcggtgctggtactaaagttgaaatcaaa

SEQ ID NO: 36 - protein, 121 aa, synthetic construct
  REGION 1..121: note = Heavy chain variable region of clone 17.014
QVQLVQSGAEVKKPGSSVKVSCKASGYSITDYLIHWVRQAPGQGLEWMGWIDPEDGETNYAQKFQGRVTLTADTSTSTAYMELSSLRSEDTAVYYCARSLDSTPIYPFAYWGQGTLVTVSS

SEQ ID NO: 37 - protein, 113 aa, synthetic construct
  REGION 1..113: note = Light chain variable region of clone 17.014
DIVMTQSPDSLAVSLGERATINCKSSQSLLRSGNQKNYLAWYQQKPGQPPKLLIYYASTGQSGVPDRFSGSGSGTDFTLTISSLQAEDVAVYYCLQHYITPPTFGAGTKVEIK

SEQ ID NO: 38 - protein, 8 aa, synthetic construct
  REGION 1..8: note = 17.014 H- CDR1
GYSITDYL

SEQ ID NO: 39 - protein, 8 aa, synthetic construct
  REGION 1..8: note = 17.014 H- CDR2
IDPEDGET

SEQ ID NO: 40 - protein, 14 aa, synthetic construct
  REGION 1..14: note = 17.014 H- CDR3
ARSLDSTPIYPFAY

SEQ ID NO: 41 - protein, 12 aa, synthetic construct
  REGION 1..12: note = 17.014 L- CDR1
QSLLRSGNQKNY

SEQ ID NO: 42 - skipped

SEQ ID NO: 43 - protein, 9 aa, synthetic construct
  REGION 1..9: note = 17.014 L- CDR3
LQHYITPPT

SEQ ID NO: 44 - protein, 8 aa, synthetic construct
  REGION 1..8: note = 17.038 H- CDR1
GYSITDSL

SEQ ID NO: 45 - protein, 8 aa, synthetic construct
  REGION 1..8: note = 17.038 H- CDR2
IDPEDGEI

SEQ ID NO: 46 - protein, 14 aa, synthetic construct
  REGION 1..14: note = 17.038 H- CDR3
ARSLDSYPIDPFAY

SEQ ID NO: 47 - protein, 12 aa, synthetic construct
  REGION 1..12: note = 17.038 L- CDR1
QSLLRNGNQQNY

SEQ ID NO: 48 - skipped

SEQ ID NO: 49 - protein, 9 aa, synthetic construct
  REGION 1..9: note = 17.038 L- CDR3
QQHYVTPPT

SEQ ID NO: 50 - protein, 8 aa, synthetic construct
  REGION 1..8: note = 17.043 H- CDR1
GYSITDYL

SEQ ID NO: 51 - protein, 8 aa, synthetic construct
  REGION 1..8: note = 17.043 H- CDR2
IDPEDGVT

SEQ ID NO: 52 - protein, 14 aa, synthetic construct
  REGION 1..14: note = 17.043 H- CDR3
ARSLDYDPIYPFAY

SEQ ID NO: 53 - protein, 12 aa, synthetic construct
  REGION 1..12: note = 17.043 L- CDR1
QSLLRNGNQKNY

SEQ ID NO: 54 - skipped

SEQ ID NO: 55 - protein, 9 aa, synthetic construct
  REGION 1..9: note = 17.043 L- CDR3
QQHYVTPPT

SEQ ID NO: 56 - protein, 8 aa, synthetic construct
  REGION 1..8: note = 17.053 H- CDR1
GYSITDYL

SEQ ID NO: 57 - protein, 8 aa, synthetic construct
  REGION 1..8: note = 17.053 H- CDR2
IDPEDGET

SEQ ID NO: 58 - protein, 14 aa, synthetic construct
  REGION 1..14: note = 17.053 H- CDR3
ARSLDYDPIYPFAY

SEQ ID NO: 59 - protein, 12 aa, synthetic construct
  REGION 1..12: note = 17.053 L- CDR1
QSLLRNGNQKNY

SEQ ID NO: 60 - skipped

SEQ ID NO: 61 - protein, 9 aa, synthetic construct
  REGION 1..9: note = 17.053 L- CDR3
QQHYITPPT

SEQ ID NO: 62 - protein, 8 aa, synthetic construct
  REGION 1..8: note = 17.054 H- CDR1
GYSITDDL

SEQ ID NO: 63 - protein, 8 aa, synthetic construct
  REGION 1..8: note = 17.054 H- CDR2
IDPEDGET

SEQ ID NO: 64 - protein, 14 aa, synthetic construct
  REGION 1..14: note = 17.054 H- CDR3
ARSLDSTWIYPFAY

SEQ ID NO: 65 - protein, 12 aa, synthetic construct
  REGION 1..12: note = 17.054 L- CDR1
QSLLRRGNQKNH

SEQ ID NO: 66 - skipped

SEQ ID NO: 67 - protein, 9 aa, synthetic construct
  REGION 1..9: note = 17.054 L- CDR3
LQSYITPPT

SEQ ID NO: 68 - protein, 451 aa, synthetic construct
  REGION 1..451: note = BDG17.066 heavy Chain LALA
QVQLVQSGAEVKKPGSSVKVSCKASGYSITDSLIHWVRQAPGQGLEWMGWIDPEDGEINYAQKFQGRVTLTADTSTSTAYMELSSLRSEDTAVYYCARSLDSYPIDPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 220 aa, synthetic construct
  REGION 1..220: note = BDG17.066 Light Chain
DIVMTQSPDSLAVSLGERATINCKSSQSLLRNGNQQNYLAWYQQKPGQPPKLLIYYASTGQSGVPDRFSGSGSGTDFTLTISSLQAEDVAVYYCQQHYVTPPTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 451 aa, synthetic construct
  REGION 1..451: note = BDG17.067 heavy Chain LALA
QVQLVQSGAEVKKPGSSVKVSCKASGYSITDYLIHWVRQAPGQGLEWMGWIDPEDGVTAYAQKFQGRVTLTADTSTSTAYMELSSLRSEDTAVYYCARSLDYDPIYPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 220 aa, synthetic construct
  REGION 1..220: note = BDG17.067 Light Chain
DIVMTQSPDSLAVSLGERATINCKSSQSLLRNGNQKNYLAWYQQKPGQPPKLLIYYASTGQSGVPDRFSGSGSGTDFTLTISSLQAEDVAVYYCQQHYVTPPTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 451 aa, synthetic construct
  REGION 1..451: note = BDG17.069 heavy Chain LALA
QVQLVQSGAEVKKPGSSVKVSCKASGYSITDDLIHWVRQAPGQGLEWMGWIDPEDGETNYAQKFQGRVTLTADTSTSTAYMELSSLRSEDTAVYYCARSLDSTWIYPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 220 aa, synthetic construct
  REGION 1..220: note = BDG17.069 Light Chain
DIVMTQSPDSLAVSLGERATINCKSSQSLLRRGNQKNHLAWYQQKPGQPPKLLIYDASTGQSGVPDRFSGSGSGTDFTLTISSLQAEDVAVYYCLQSYITPPTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC